(19) United States
(12) Patent Application Publication
(10) Pub. No.: US 2026/0176379 A1
(43) Pub. Date: Jun. 25, 2026

(54) T-Cell Modulatory Polypeptides and Methods of Use Thereof

(71) Applicant: Cue Biopharma, Inc., Boston, MA (US)

(72) Inventors: Chee Meng Low, Boston, MA (US); Ahmet Selim Vakkasoglu, Boston, MA (US)

(21) Appl. No.: 19/422,106

(22) Filed: Dec. 16, 2025

Related U.S. Application Data
(63) Continuation of application No. PCT/US2024/035141, filed on Jun. 21, 2024.
(60) Provisional application No. 63/522,866, filed on Jun. 23, 2023.

(51) Int. Cl. C07K 19/00; A61K 40/11; C07K 14/005; C12N 7/00

(52) U.S. Cl. CPC C07K 19/00; A61K 40/11; C07K 14/005; C12N 7/00; C07K 2317/92

(57) ABSTRACT

The present disclosure provides a peptide-major histocompatibility complex (pMHC) polypeptide comprising a peptide epitope and class I MHC polypeptides. The present disclosure provides fusion molecules comprising a pMHC polypeptide and a heterologous fusion partner. The present disclosure provides single-chain T-cell modulatory polypeptides that comprise a pMHC polypeptide, one or more immunomodulatory polypeptide, and an immunoglobulin (Ig) Fc or a non-Ig scaffold. A TMP is useful for modulating the activity of a T cell, and for modulating an immune response in an individual.

### CROSS-REFERENCE

This application is a continuation of PCT Application No. PCT/US2024/035141, filed Jun. 21, 2024, which claims the benefit of U.S. Provisional Patent Application No. 63/522,866, filed Jun. 23, 2023, which applications are incorporated herein by reference in their entirety.

### INCORPORATION-BY-REFERENCE OF MATERIAL ELECTRONICALLY SUBMITTED

A Sequence Listing is provided herewith as a Sequence Listing XML, “CUEB-152WO_SEQLIST” created on Jun. 21, 2024 and having a size of 392,350 bytes. The contents of the Sequence Listing XML are incorporated by reference herein in their entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell modulation (activation or inhibition) and effector function. Following epitope-specific cell targeting, the targeted T cells are activated through engagement of costimulatory proteins found on the APC with counterpart costimulatory proteins the T cells. Both signals—epitope/TCR binding and engagement of APC costimulatory proteins with T cell costimulatory proteins—are required to drive T cell specificity and activation or inhibition. The costimulatory proteins on the APC also are referred to as “immunomodulatory” proteins because they modulate the activity of the T cell when they bind the costimulatory protein on the T cell, with the specific modulation being a function of which immunomodulatory protein on the APC binds to which costimulatory protein on the T cell. The TCR is specific for a given epitope; however, the T cell's costimulatory proteins are not epitope-specific and instead is generally expressed on all T cells or on large T cell subsets.

### SUMMARY

The present disclosure provides a peptide-major histocompatibility complex (pMHC) polypeptide comprising a peptide epitope and class I MHC polypeptides. The present disclosure also provides pMHC complexes such as pMHC fusion molecules that comprise a pMHC polypeptide and a heterologous fusion partner. The present disclosure also provides a single-chain T-cell modulatory polypeptide (TMP) that comprises a pMHC polypeptide, one or more immunomodulatory polypeptide, and an immunoglobulin (Ig) Fc or a non-Ig scaffold. A TMP is useful for modulating the activity of a T cell, and for modulating an immune response in an individual.

### BRIEF DESCRIPTION OF THE DRAWINGS

FIG. 1A-1J provide schematic depictions of disulfide-linked pMHC polypeptides.

FIGS. 2A-2B provide an amino acid sequence of a wild-type human β2M polypeptide (FIG. 2A; SEQ ID NO:42) and an amino acid sequence of a β2M polypeptide with an R12C substitution (FIG. 2B; SEQ ID NO:43).

FIGS. 3A-3E provide amino acid sequences of wild-type HLA-A*0201 (FIG. 3A; SEQ ID NO:44) and variants (FIG. 3B-3E; SEQ ID NOs:45-48, respectively).

FIGS. 4A-4E provide amino acid sequences of wild-type HLA-A*1101 (FIG. 4A; SEQ ID NO:49) and variants (FIG. 4B-4E; SEQ ID NOs:50-53, respectively).

FIGS. 5A-5E provide amino acid sequences of wild-type HLA-A*2402 (FIG. 5A; SEQ ID NO:54) and variants (FIG. 5B-5E; SEQ ID NOs:55-58, respectively).

FIGS. 6A-6E provide amino acid sequences of wild-type HLA-A*3303 (FIG. 6A; SEQ ID NO:59) and variants (FIG. 6B-6E; SEQ ID NOs:60-63, respectively).

FIGS. 7A-7E provide amino acid sequences of wild-type HLA-A*0301 (FIG. 7A; SEQ ID NO:64) and variants (FIG. 7B-7E; SEQ ID NOs:65-68, respectively).

FIGS. 8A-8D provide an alignment of amino acid sequences of wild-type (FIGS. 8A and continued in 8B; SEQ ID NOs:69-77, respectively) and variant (FIGS. 8C and continued in 8D; SEQ ID NO:78-86, respectively) HLA-A polypeptides.

FIGS. 9A-9D provide an alignment of amino acid sequences of wild-type (FIGS. 9A and continued in 9B; SEQ ID NOs:87-93, respectively) and variant (FIGS. 9C and continued in 9D; SEQ ID NOs:94-100, respectively) HLA-B polypeptides.

FIGS. 10A-10D provide an alignment of amino acid sequences of wild-type (FIGS. 10A and continued in 10B; SEQ ID NOs:101-109, respectively) and variant (FIGS. 10C and continued in 10D; SEQ ID NOs:110-118, respectively) HLA-C polypeptides.

FIGS. 11A-11E provide amino acid sequences of wild-type (FIG. 11A; SEQ ID NO: 119) HLA-E heavy chains and variants (FIGS. 11B-11E; SEQ ID NOs:120-123, respectively).

FIGS. 12A-12E provide amino acid sequences of wild-type (FIG. 12A; SEQ ID NO: 124) HLA-E heavy chains and variants (FIGS. 12B-12E; SEQ ID NOs:125-128, respectively).

FIGS. 13A-13D provide amino acid sequences of wild-type (FIG. 13A; SEQ ID NO:129 and FIG. 13C; SEQ ID NO:131) and variant (FIG. 13B; SEQ ID NO:130 and FIG. 13D; SEQ ID NO: 132) HLA-G heavy chains.

FIG. 14 provides schematic depictions of examples of positions of immunomodulatory polypeptides in TMPs.

FIGS. 15A-15J provide schematic depictions of disulfide-linked TMPs.

FIGS. 16A-16L provide amino acid sequences of immunoglobulin Fc polypeptides (SEQ ID NOs:133-144, respectively).

FIG. 17 provides an amino acid sequence of an alpha-feto protein (SEQ ID NO: 145).

FIGS. 18A-18E provide amino acid sequences of WT-1 polypeptides (SEQ ID NOs:146-150, respectively).

FIGS. 19A-19B provide amino acid sequences of an HPV E6 polypeptide (FIG. 19A; SEQ ID NO:151) and an HPV E7 polypeptide (FIG. 19B; SEQ ID NO: 152).

FIGS. 20A-20M provide amino acid sequences of MUC-1 polypeptides (SEQ ID NOs:153-165, respectively).

FIGS. 21A-21C provide amino acid sequences of survivin polypeptides (SEQ ID NOs:166-168, respectively).

FIGS. 22A-22B provides amino acid sequences of PRAME polypeptides (SEQ ID NOs:169-170, respectively).

FIGS. 23A-23E provide amino acid sequences of some examples of single-chain TMPs of the present disclosure (SEQ ID NOs:171-175, respectively) (“G2C”, “(G4S)3”, “AAAGG”, “(AP)4”, “(G4S)4”, “GCGGS(GGGS)2”, “(GGGGS)3”, and “(GGGGS)4” linkers are SEQ ID NOs:176-183, respectively) (“KRAS epitope” is SEQ ID NO:35; “KRAS(71-6; G12D)” is SEQ ID NO: 184).

FIGS. 24A-24D provide amino acid sequences of IL-2Rα (FIG. 24A; SEQ IS NO: 185), IL-2Rβ (FIG. 24B; SEQ IS NO: 186), and IL-2Rγ (FIG. 24C; SEQ IS NO: 187), and an amino acid sequence of wild-type IL-2 (FIG. 24D; SEQ IS NO: 188).

FIGS. 25A-25F provide amino acid sequences of various constructs (SEQ ID NOs:189-194, respectively) used in the Examples (“(G4S)3”, “AAAGG”, “(AP)4”, “(G4S)4”, and “GCGGS(GGGS)2” linkers are SEQ ID NOs:177-181, respectively) (“KRAS (G12D)” and “KRAS (G12V)” are SEQ ID NOs: 184 and 195, respectively).

FIG. 26 depicts temperature stability of various constructs described in the Examples.

FIG. 27 depicts expansion of peptide-specific T cells induced by various constructs described in the Examples.

FIGS. 28A-28B depict expansion of peptide-specific T cells induced by various constructs described in Example 3.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide are understood to refer to the amino acid at that position in the wild-type polypeptide. To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if an MHC class I heavy chain polypeptide includes a substitution of A139, the substitution of A139 is understood to refer to a substitution of an amino acid for the amino acid at position 139 in a wild-type MHC class I heavy chain polypeptide. As another example, if an MHC class I polypeptide is altered by the addition of one amino acid at the N-terminus, then a reference to position 84 or a specific residue at position 84, will be understood to indicate the amino acids that are at position 85 on the altered polypeptide. Likewise, a reference herein to substitution of a specific amino acid at a specific position, e.g., Y84, is understood to refer to a substitution of an amino acid for the amino acid at position 84 in the wild-type polypeptide. A Y84A substitution is thus understood to be a substitution of an Ala residue for the Tyr residue that is present in the wild-type sequence. If, e.g., the wild-type polypeptide is altered to change the amino acid at position 84 from its wild-type amino acid to an alternate amino acid, then the substitution for the amino acid at position 84 will be understood to refer to the substitution for the alternate amino acid. If in such case the polypeptide is also altered by the addition or deletion of one or more amino acids, then the reference to the substitution will be understood to refer to the substitution for the alternate amino acid at the altered position number. A reference to a “non-naturally occurring Cys residue” in a polypeptide, e.g., an MHC class I polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Biol. 215:403-10. Unless otherwise stated, “sequence identity” as referred to herein is determined by BLAST (Basic Local Alignment Search Tool) using default parameters.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to herein as a “MOD”), as used herein, means a polypeptide that specifically binds a cognate costimulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. As discussed herein, a MOD can include, but is not limited to, wild-type or variants of wild-type polypeptides such as a cytokine (e.g., IL-2), CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor, and a ligand that specifically binds with B7-H3. A MOD of a TMP can bind a cognate costimulatory polypeptide (i.e., a “co-MOD”) that is present on a target T cell.

As used herein the term “in vivo” refers to any process or procedure occurring inside of the body.

As used herein, “in vitro” refers to any process or procedure occurring outside of the body.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding,” as used herein (e.g., with reference to binding of a pMHC polypeptide to a TCR or a MOD to a co-MOD), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Affinity” refers to the strength of non-covalent binding, increased binding affinity being correlated with a lower K_{D}. “Specific binding” generally refers to binding of a ligand to a moiety that is than its designated binding site or receptor. “Non-specific binding” generally refers to binding of a ligand to a moiety other than its designated binding site or receptor. “Covalent binding” or “covalent bond,” as used herein, refers to the formation of one or more covalent chemical binds between two different molecules.

The terms “antibody” and “antibodies” include antibodies or immunoglobulins of any isotype, fragments of antibodies that retain specific binding to one or more targe antigens, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (1996) Nature Structural Biol. 3:803; and Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may or may not be predisposed to acquiring the disease or symptom but has not yet been diagnosed as having it; (b) inhibiting the disease or one or more symptoms associated with the disease, e.g., arresting its development; and/or (c) relieving the disease, i.e., causing regression of the disease. The therapeutic agent may be administered before, during and/or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc. Unless otherwise indicated, the terms “individual,” “subject,” “host,” and “patient,” refer to a human.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely does not induce cell lysis.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

As used herein, the term “MHC heavy chain polypeptide” means collectively the domains of an MHC heavy chain polypeptide that are present in a pMHC polypeptide. For example, an MHC heavy chain polypeptide can comprise α1, α2 and α3 domains.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of this disclosure will be limited only by the appended claims.

Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of this disclosure, the preferred methods and materials are now described. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. Thus, for example, reference to a “pMHC polypeptide” includes a plurality of such polypeptides and reference to “the immunomodulatory polypeptide” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The terms “comprising,” “having,” “including,” and “containing” are to be construed as open-ended terms (i.e., meaning “including, but not limited to,”) unless otherwise noted. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of this disclosure described herein include “comprising,” “consisting,” and “consisting essentially of” aspects and embodiments.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication. Further, the dates of publication provided may be different from the actual publication dates which may need to be independently confirmed.

### DETAILED DESCRIPTION

The present disclosure provides a peptide-major histocompatibility complex (pMHC) polypeptide comprising a peptide epitope and class I MHC polypeptides. The present disclosure also provides pMHC complexes (including pMHC fusion molecules) comprising a pMHC polypeptide and one or more additional components, such as one or more heterologous polypeptides, a nucleic acid component, or other component. The present disclosure also provides single-chain T-cell modulatory polypeptides (TMPs) that comprise a pMHC polypeptide, one or more immunomodulatory polypeptide, and an immunoglobulin (Ig) Fc or a non-Ig scaffold. A TMP is useful for modulating the activity of a T cell, and for modulating an immune response in an individual.

### Peptide-Major Histocompatibility Complex (PMHC) Polypeptides

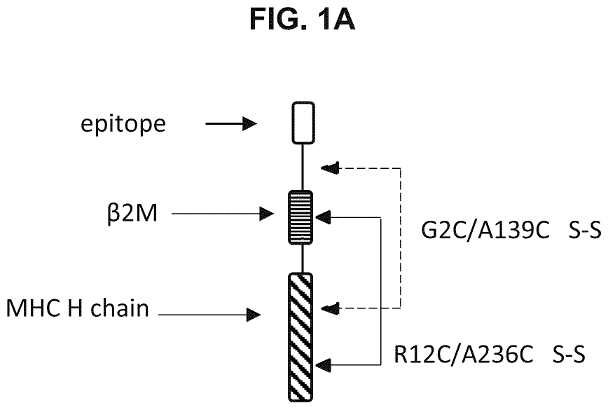

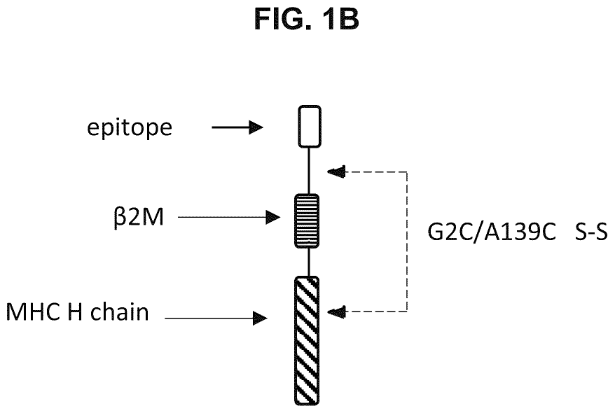

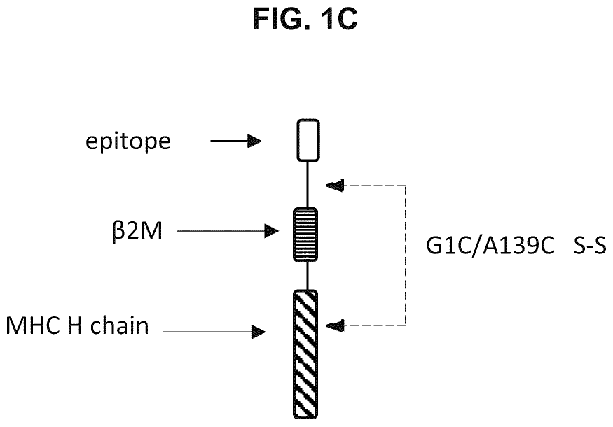

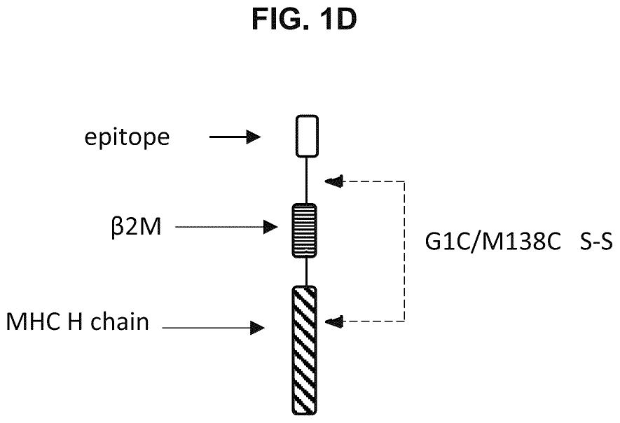

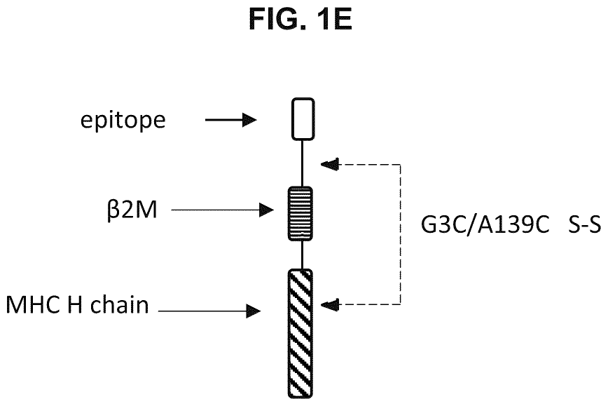

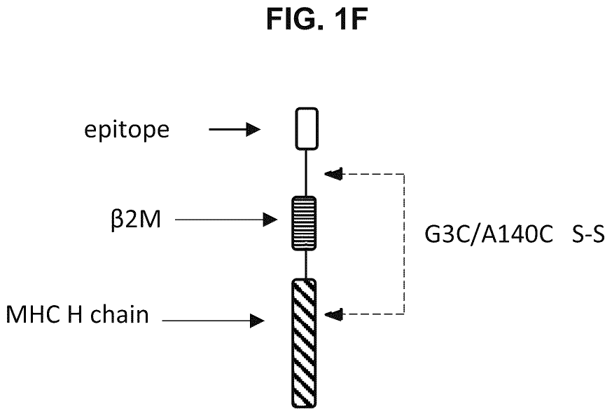

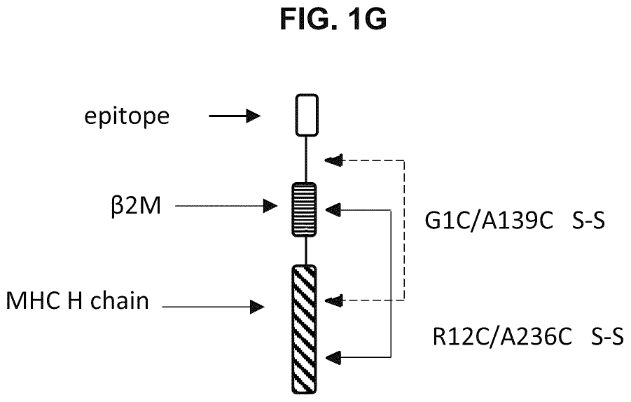

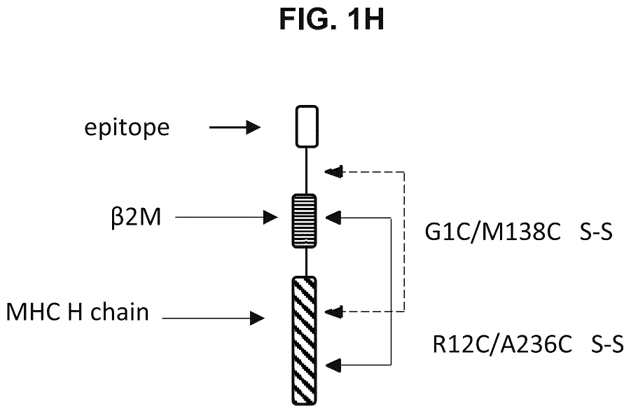

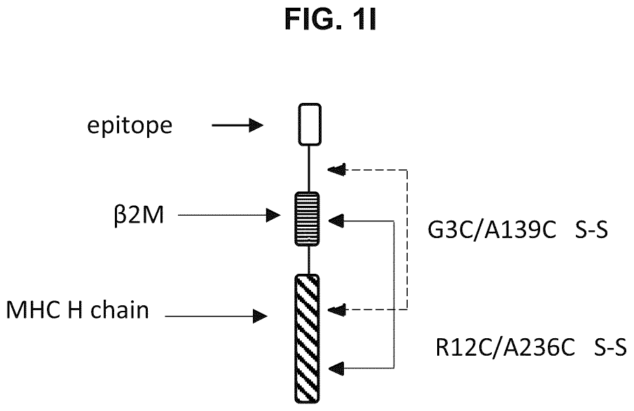

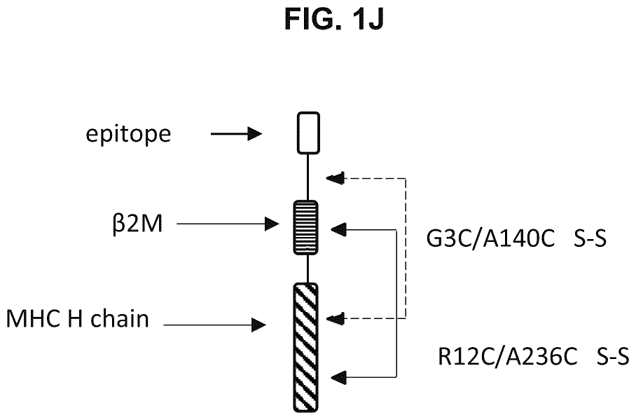

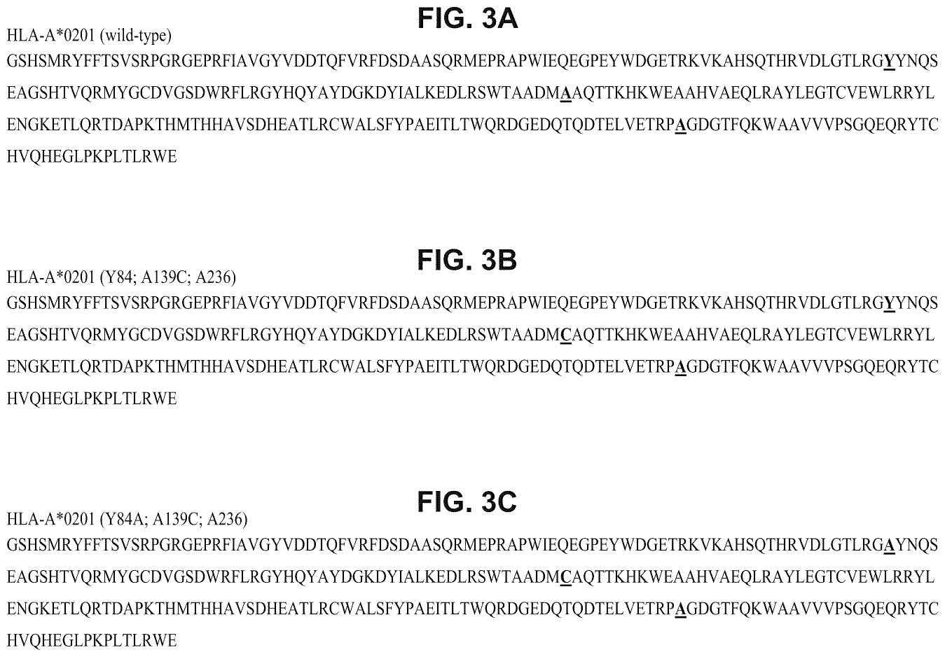

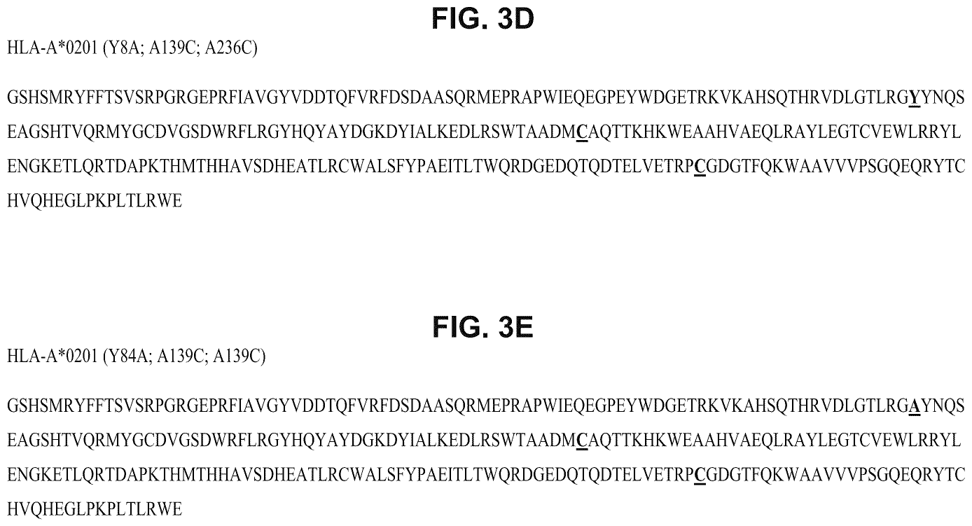

The present disclosure provides pMHC polypeptides. A pMHC polypeptide of this disclosure comprises: a) a peptide epitope having a length of from 4 amino acids to 25 amino acids (e.g., from 8-12 amino acids); b) a first peptide linker, where the first peptide linker comprises a cysteine (Cys) residue; c) a beta-2 microglobulin (β2M) polypeptide; d) a second peptide linker; and e) a major histocompatibility complex (MHC) class I heavy chain polypeptide. The MHC class I heavy chain polypeptide comprises: i) a Cys at any one of amino acids 135-143, based on the numbering of the MHC class I heavy chain polypeptide depicted in FIG. 3A; and ii) an amino acid other than Cys at position 84, based on the numbering of the MHC class I heavy chain polypeptide depicted in FIG. 3A. The pMHC polypeptide comprises an intrachain disulfide bond formed between the Cys present in the first peptide linker and the Cys at any one of amino acids 135-143 of the MHC class I heavy chain polypeptide. The pMHC polypeptide presents the epitope for binding to a T cell receptor (TCR). FIG. 1A-1J provide schematic depictions of non-limiting examples of configurations of pMHC polypeptides. In some cases, a pMHC polypeptide comprises, in order from N-terminus to C-terminus: a) a peptide epitope having a length of from 4 amino acids to 25 amino acids (e.g., from 8-12 amino acids); b) a first peptide linker, where the first peptide linker comprise a Cys residue; c) a β2M polypeptide; d) a second peptide linker; and e) an MHC class I heavy chain polypeptide (where the MHC class I heavy chain polypeptide comprises: i) a Cys at any one of amino acids 135-143, based on the numbering of the MHC class I heavy chain polypeptide depicted in FIG. 3A; and ii) an amino acid other than Cys at position 84, based on the numbering of the MHC class I heavy chain polypeptide depicted in FIG. 3A).

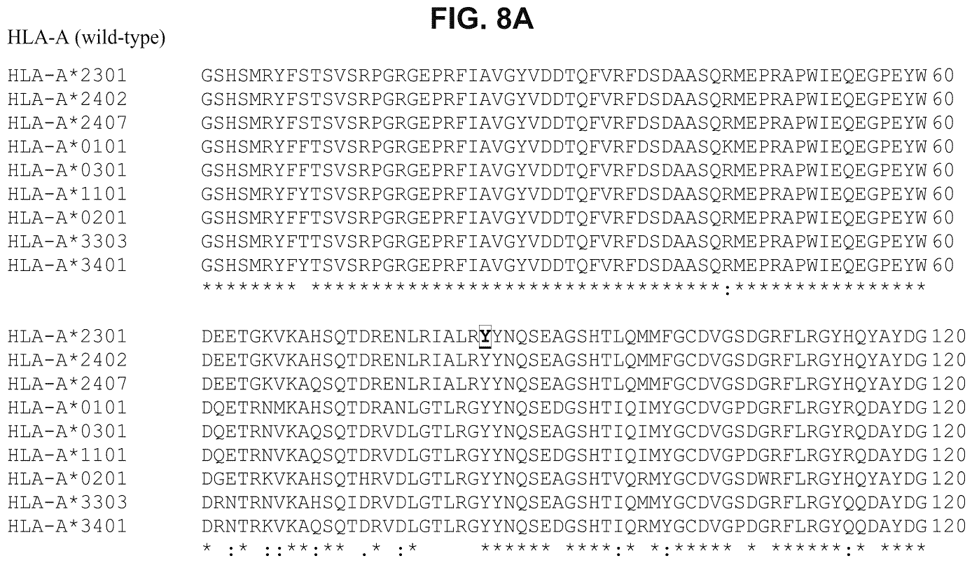

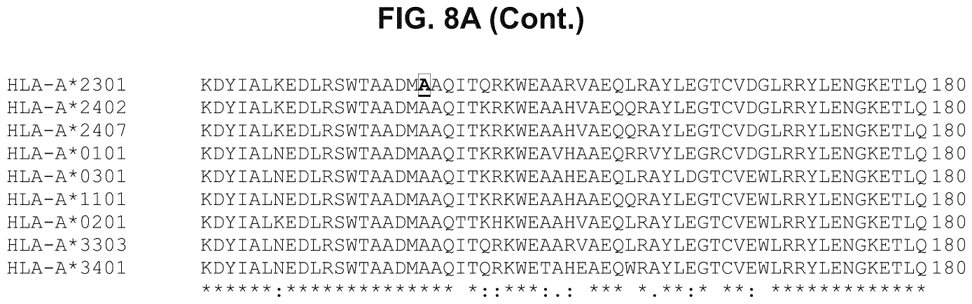

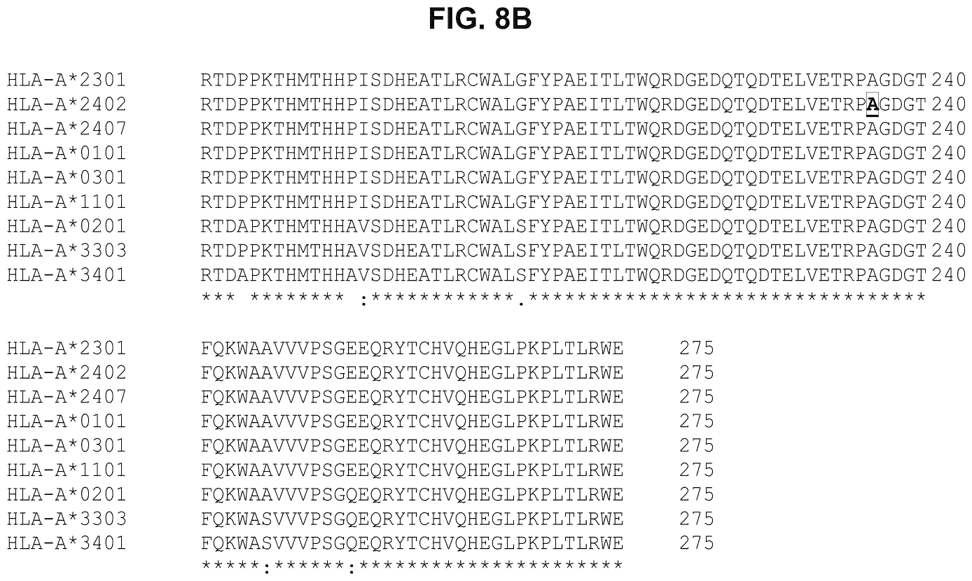

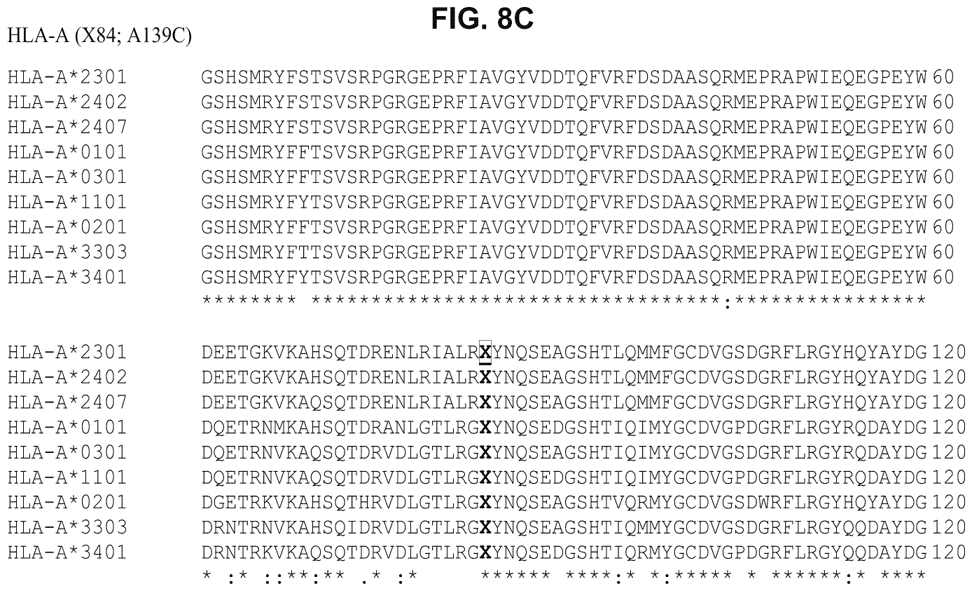

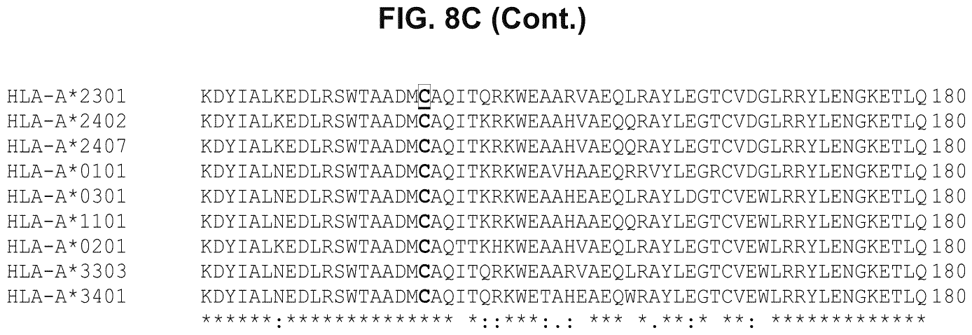

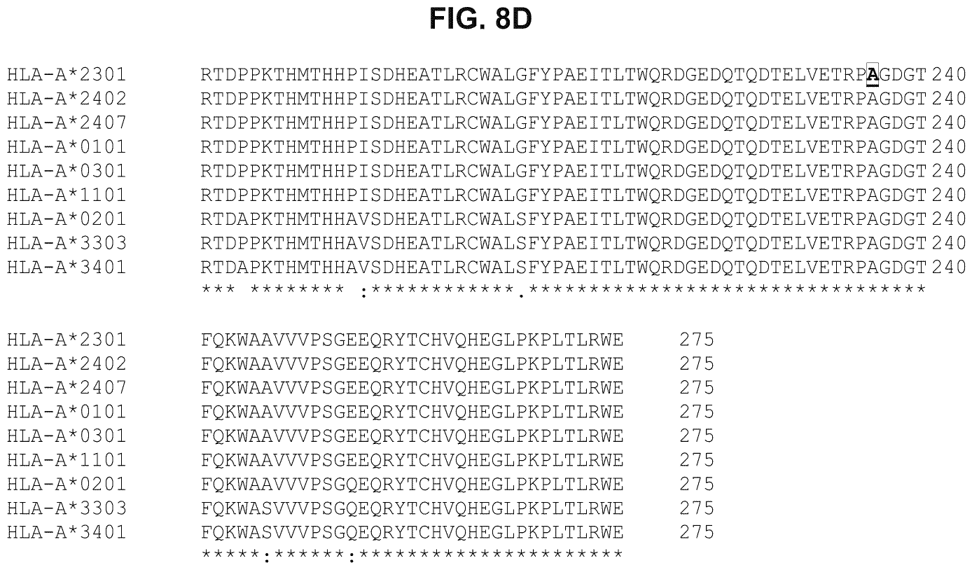

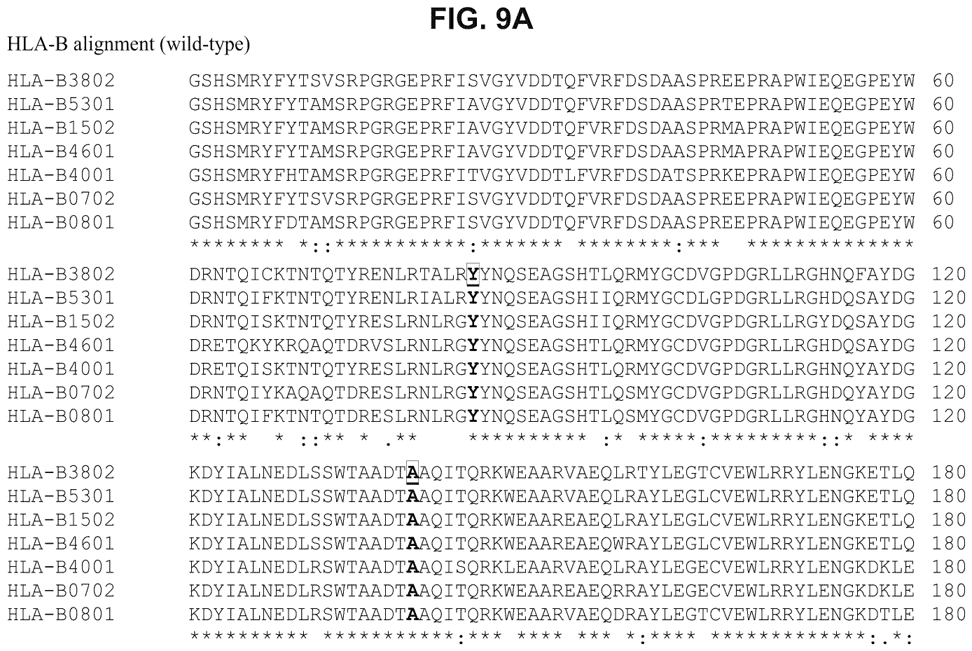

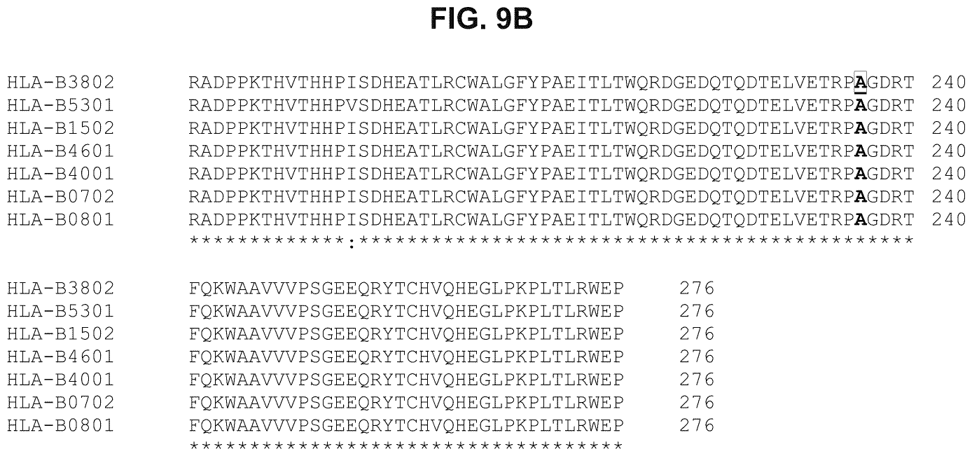

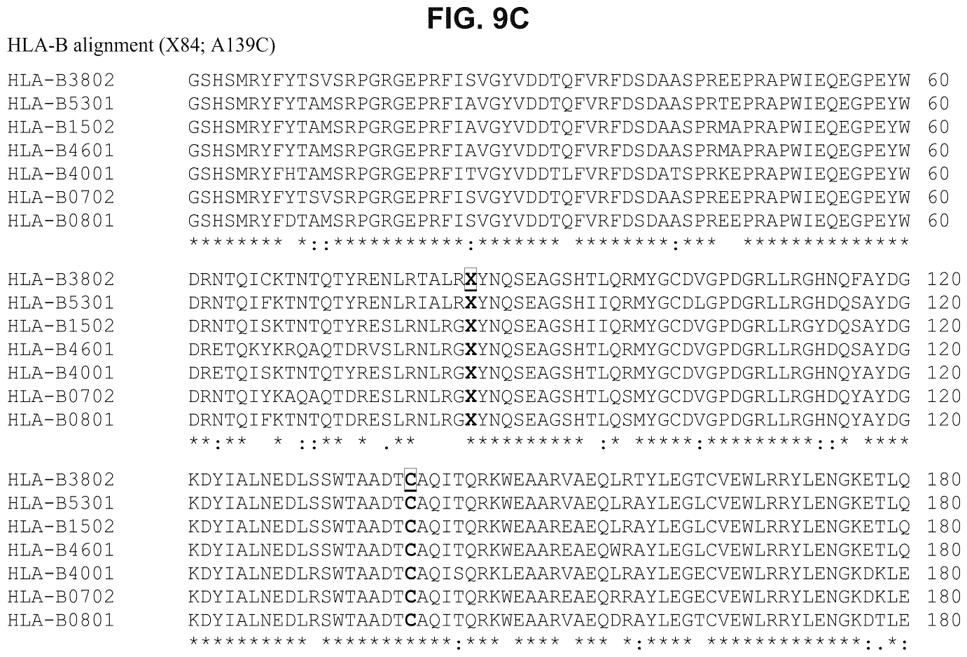

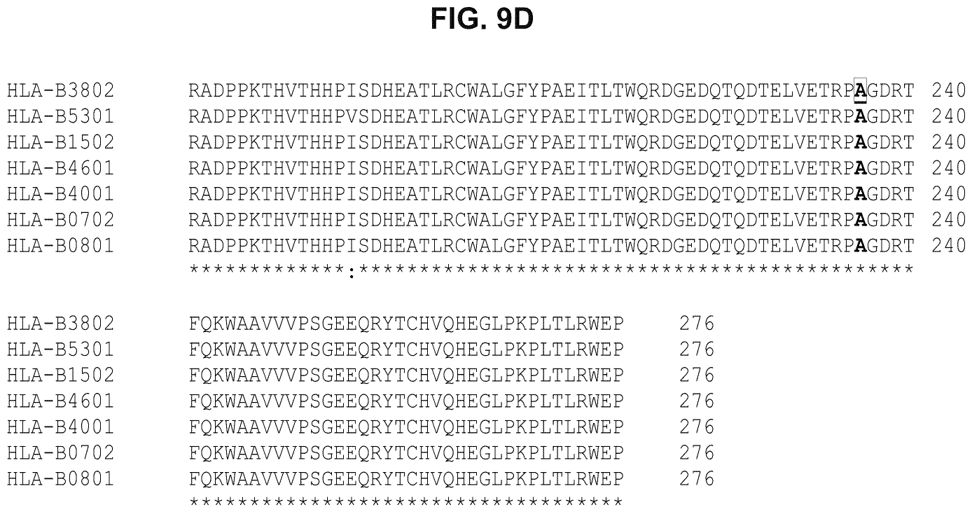

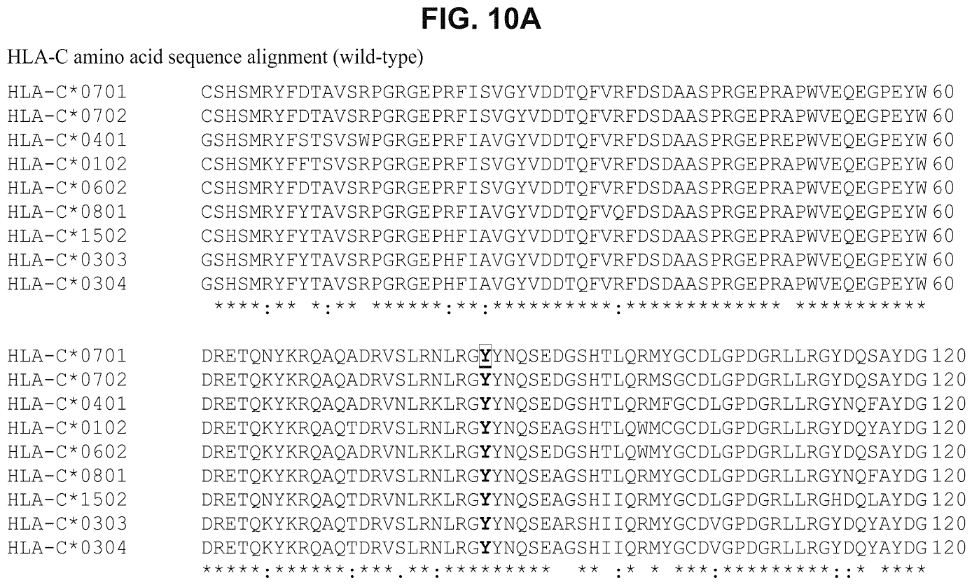

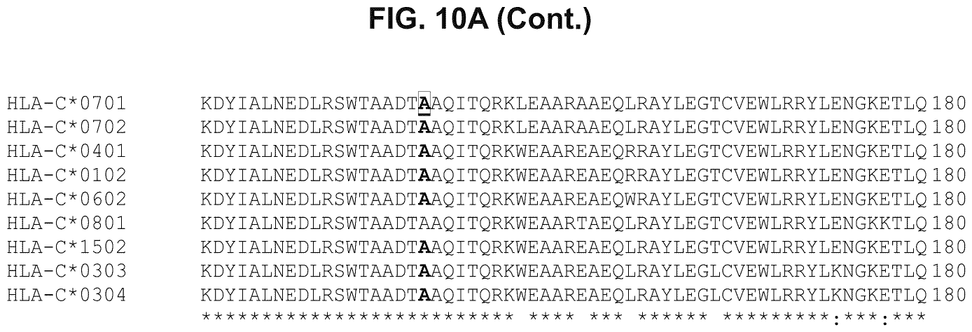

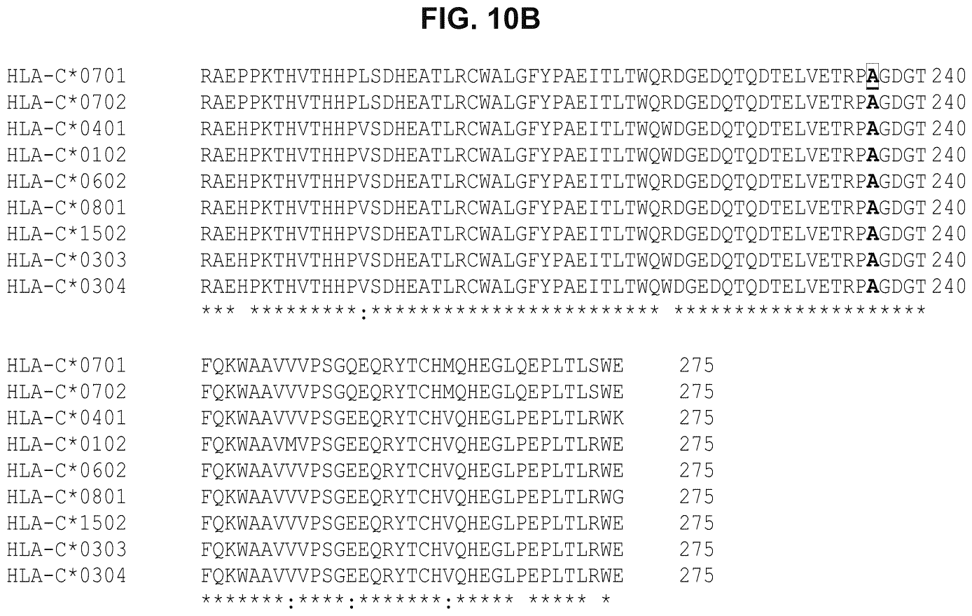

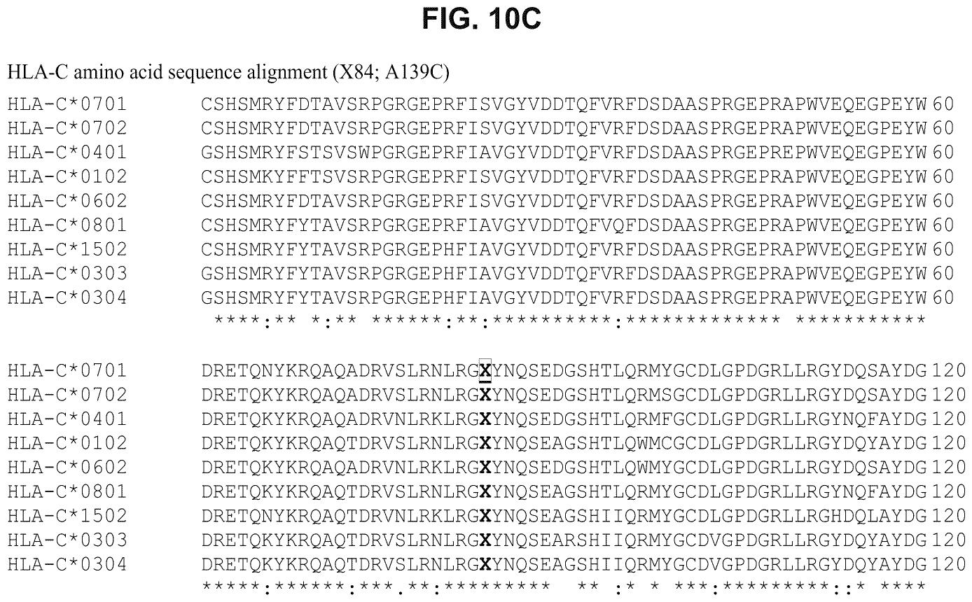

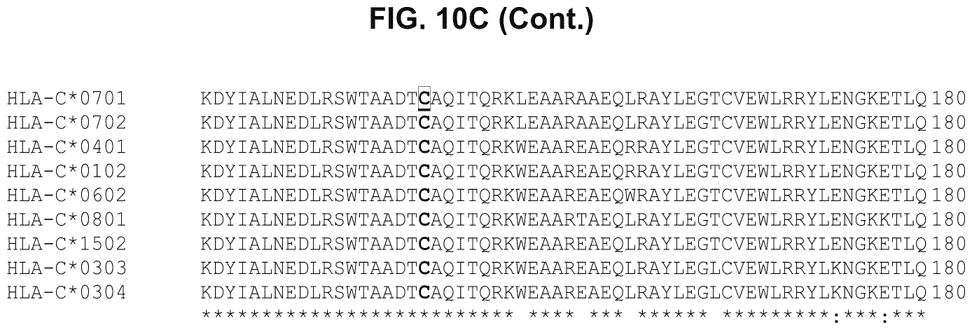

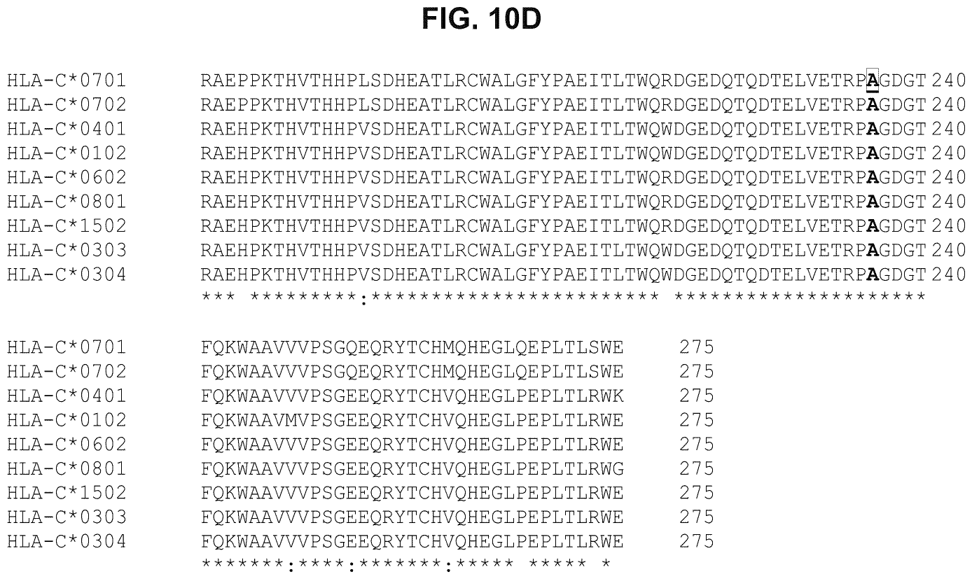

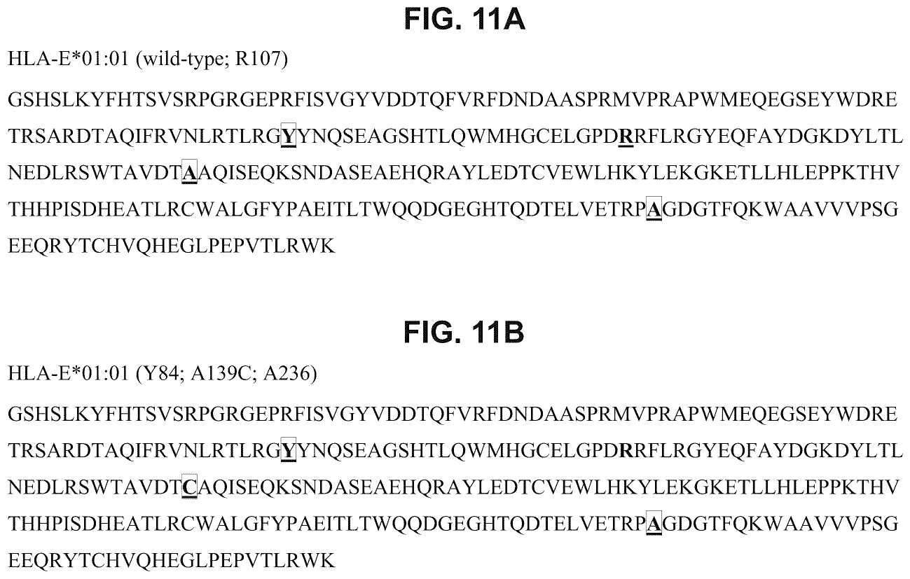

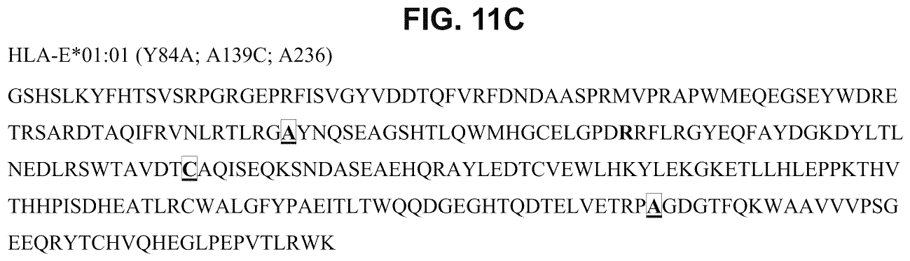

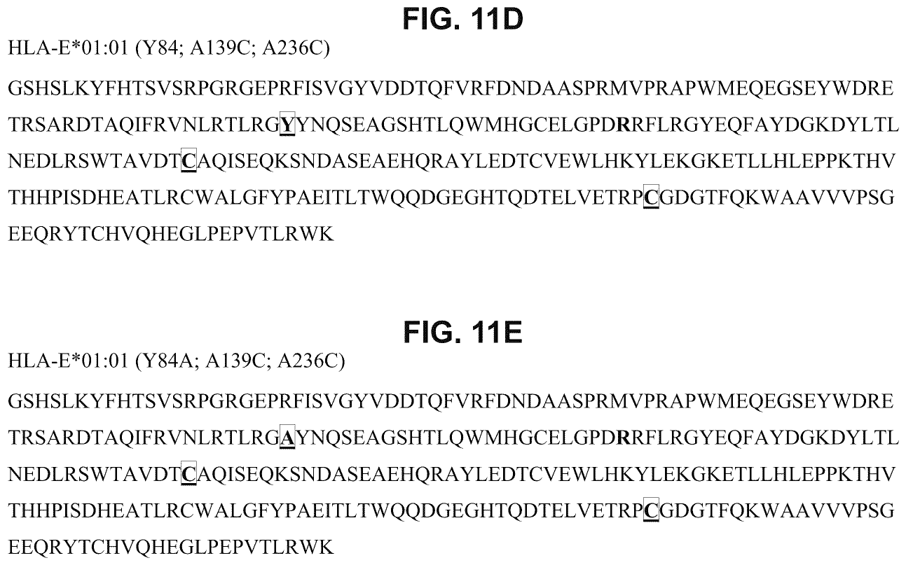

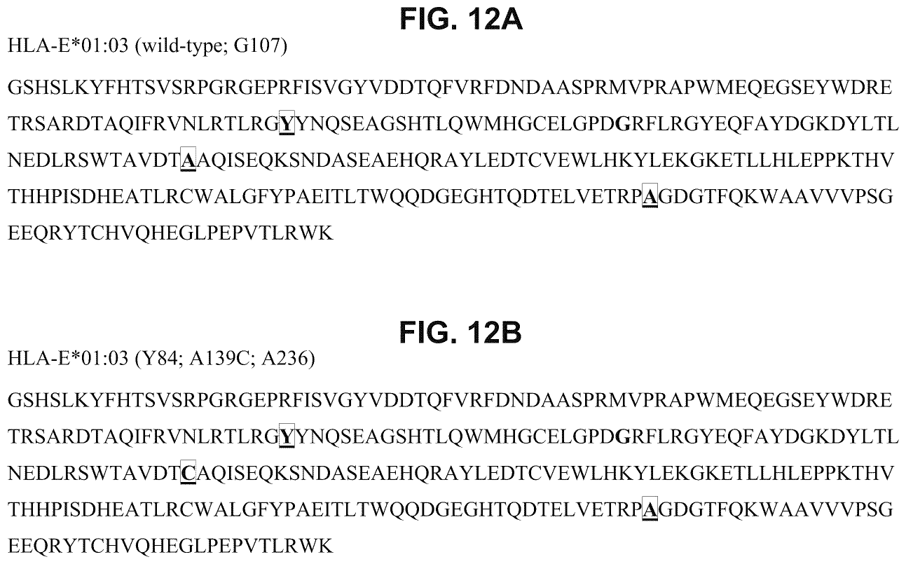

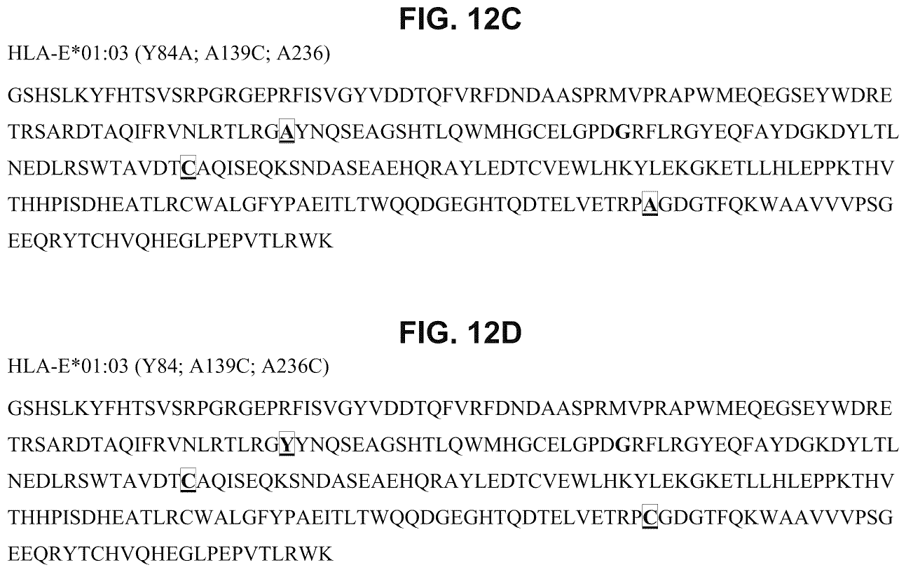

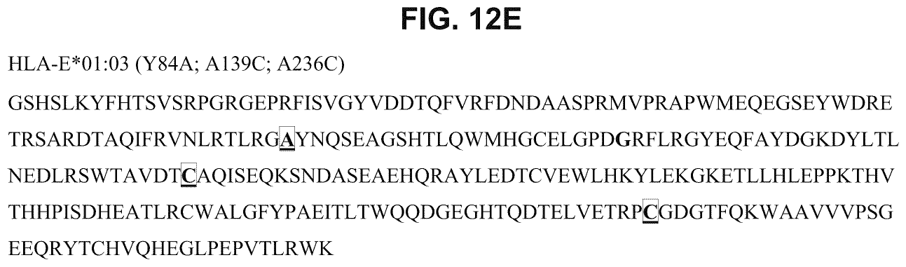

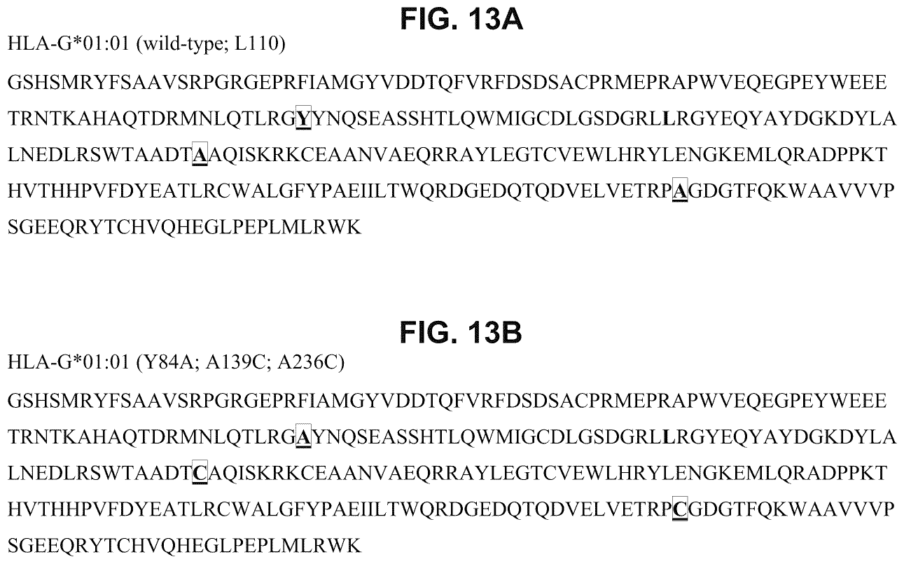

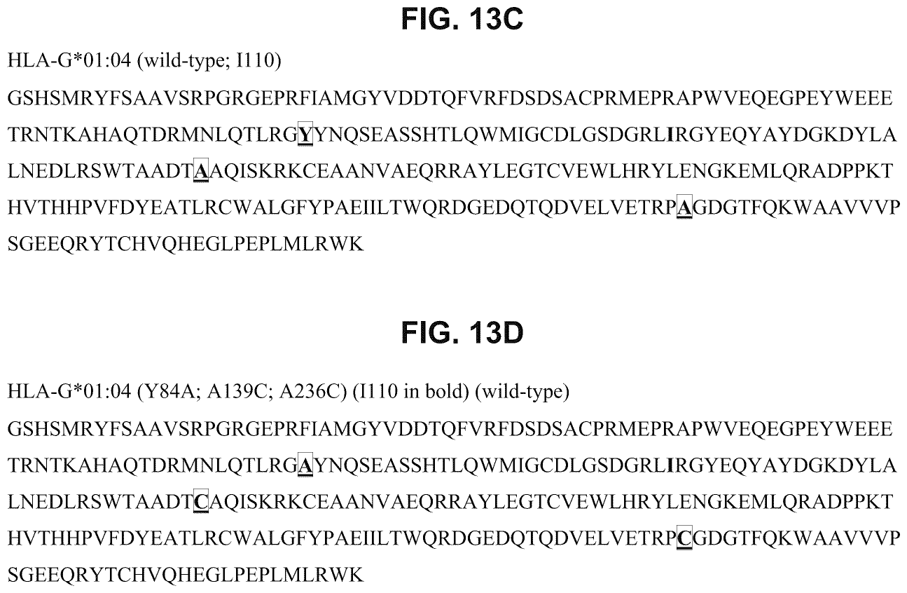

Unless otherwise specified below, the amino acid numbering of an MHC class I heavy chain polypeptide present in a pMHC polypeptide is based on the amino acid numbering of the MHC class I heavy chain polypeptide depicted in FIG. 3A. It should be noted that the amino acid numbering of the MHC class I heavy chain polypeptide depicted in FIG. 3A applies equally to the amino acid numbering of the MHC class I heavy chain polypeptides depicted in FIGS. 8A-8B (alignment of amino acid sequences of wild-type HLA-A heavy chain polypeptides), FIGS. 9A-9B (alignment of amino acid sequences of wild-type HLA-B heavy chain polypeptides), FIGS. 10A-10B (alignment of amino acid sequences of wild-type HLA-C heavy chain polypeptides), FIG. 11A and FIG. 12A (amino acid sequences of wild-type HLA-E heavy chain polypeptides), and FIGS. 13A and 13C (amino acid sequences of wild-type HLA-G heavy chain polypeptides).

In some cases, a pMHC polypeptide does not comprise a polypeptide other than (i) the peptide epitope, (ii) the first and second peptide linkers, (iii) the β2M polypeptide, and (iv) the MHC class I heavy chain polypeptide. In some cases, a pMHC polypeptide has a length of from about 400 amino acids to about 450 amino acids; for example, in some cases, pMHC polypeptide has a length of from 400 amino acids to 410 amino acids, from 410 amino acids to 420 amino acids, from 420 amino acids to 430 amino acids, from 430 amino acids to 450 amino acids, from 410 amino acids to 415 amino acids, from 415 amino acids to 420 amino acids, or from 420 amino acids to 425 amino acids. In some cases, a pMHC polypeptide has a length of from 410 amino acids to 415 amino acids.

### MHC Class I Heavy Chain Polypeptides

A pMHC polypeptide comprises an MHC class I heavy chain polypeptide. Suitable MHC class I heavy chain polypeptides include a human MHC class I heavy chain polypeptide, where human MHC polypeptides are also referred to as “human leukocyte antigen” (“HLA”) polypeptides. Class I HLA heavy chain polypeptides include HLA-A heavy chain polypeptides, HLA-B heavy chain polypeptides, HLA-C heavy chain polypeptides, HLA-E heavy chain polypeptides, HLA-F heavy chain polypeptides, and HLA-G heavy chain polypeptides.

Unless expressly stated otherwise, a pMHC polypeptide does not include membrane anchoring domains (transmembrane regions) of an MHC class I heavy chain, or a part of MHC class I heavy chain sufficient to anchor the resulting pMHC to a cell (e.g., eukaryotic cell such as a mammalian cell) in which it is expressed. In some cases, the MHC class I heavy chain present in a pMHC does not include a signal peptide, a transmembrane domain, or an intracellular domain (cytoplasmic tail) associated with a native MHC class I heavy chain. Thus, e.g., in some cases, the MHC class I heavy chain present in a pMHC polypeptide includes only the α1, α2, and α3 domains of an MHC class I heavy chain. In some cases, the MHC class I heavy chain present in a pMHC has a length of from about 270 amino acids (aa) to about 290 aa. In some cases, the MHC class I heavy chain present in a pMHC has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, an MHC class I heavy chain polypeptide present in a pMHC polypeptide comprises an amino acid sequence having at least 85%, at least 90%, at least 95%, at least 97%, at least 98%, at least 99%, or 100%, amino acid sequence identity to all or part (e.g., 50, 75, 100, 150, 200, or 250 contiguous amino acids) of the amino acid sequence of any of the human HLA heavy chain polypeptides depicted in FIGS. 3-13, where the MHC class I heavy chain polypeptide comprises: i) a Cys at any one of amino acids 135-143; and ii) an amino acid other than Cys at position 84. In some cases, the MHC class I heavy chain has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, a pMHC polypeptide comprises an HLA-A heavy chain polypeptide with the above-noted amino acid substitution(s). The HLA-A heavy chain polypeptides, or portions thereof, that may be that may be incorporated into a pMHC polypeptide include, but are not limited to, the alleles: A*0101, A*0201, A*0301, A*1101, A*2301, A*2402, A*2407, A*3303, and A*3401. In some cases, a pMHC polypeptide comprises an HLA-B heavy chain polypeptide with the above-noted amino acid substitution(s). In some cases, a pMHC polypeptide comprises an HLA-C heavy chain polypeptide with the above-noted amino acid substitution(s). In some cases, a pMHC polypeptide comprises an HLA-E heavy chain polypeptide with the above-noted amino acid substitution(s).

### Intrachain Disulfide Bonds

A pMHC polypeptide of this disclosure is a single-chain polypeptide that comprises one or more intrachain disulfide bonds. A pMHC polypeptide comprises one intrachain disulfide bond formed between: i) a Cys at any one of amino acids 135-143, based on the numbering of the MHC class I heavy chain polypeptide depicted in FIG. 3A; and a Cys present in the first peptide linker, where the first peptide linker is interposed between the peptide epitope and the β2M polypeptide. A Cys is substituted for one of amino acids 135-143, in the peptide binding groove of the MHC class I heavy chain polypeptide. In some cases, the MHC class I heavy chain polypeptide in a pMHC polypeptide comprises a Cys at position 135. In some cases, the MHC class I heavy chain polypeptide in a pMHC polypeptide comprises a Cys at position 136. In some cases, the MHC class I heavy chain polypeptide in a pMHC polypeptide comprises a Cys at position 137. In some cases, the MHC class I heavy chain polypeptide in a pMHC polypeptide comprises a Cys at position 138. In some cases, the MHC class I heavy chain polypeptide in a pMHC polypeptide comprises a Cys at position 139. In some cases, the MHC class I heavy chain polypeptide in a pMHC polypeptide comprises a Cys at position 140. In some cases, the MHC class I heavy chain polypeptide in a pMHC polypeptide comprises a Cys at position 141. In some cases, the MHC class I heavy chain polypeptide in a pMHC polypeptide comprises a Cys at position 142. In some cases, the MHC class I heavy chain polypeptide in a pMHC polypeptide comprises a Cys at position 143.

Thus, for example, in some cases, amino acids 135-143 of the MHC class I heavy chain of a pMHC polypeptide comprises the amino acid sequence CADMAAQTT (SEQ ID NO: 196). As another example, in some cases, amino acids 135-143 of the MHC class I heavy chain of a pMHC polypeptide comprises the amino acid sequence ACDMAAQTT (SEQ ID NO: 197). As another example, in some cases, amino acids 135-143 of the MHC class I heavy chain of a pMHC polypeptide comprises the amino acid sequence AACMAAQTT (SEQ ID NO: 198). As another example, in some cases, amino acids 135-143 of the MHC class I heavy chain of a pMHC polypeptide comprises the amino acid sequence AADCAAQTT (SEQ ID NO: 199). As another example, in some cases, amino acids 135-143 of the MHC class I heavy chain of a pMHC polypeptide comprises the amino acid sequence AADMCAQTT (SEQ ID NO:200). As another example, in some cases, amino acids 135-143 of the MHC class I heavy chain of a pMHC polypeptide comprises the amino acid sequence AADMACQTT (SEQ ID NO:201). As another example, in some cases, amino acids 135-143 of the MHC class I heavy chain of a pMHC polypeptide comprises the amino acid sequence AADMAACTT (SEQ ID NO:202). As another example, in some cases, amino acids 135-143 of the MHC class I heavy chain of a pMHC polypeptide comprises the amino acid sequence AADMAAQCT (SEQ ID NO:203). As another example, in some cases, amino acids 135-143 of the MHC class I heavy chain of a pMHC polypeptide comprises the amino acid sequence AADMAAQTC (SEQ ID NO:204).

As another example, in some cases, amino acids 135-143 of the MHC class I heavy chain of a pMHC polypeptide comprises the amino acid sequence CADMAAQIT (SEQ ID NO:205). As another example, in some cases, amino acids 135-143 of the MHC class I heavy chain of a pMHC polypeptide comprises the amino acid sequence ACDMAAQIT (SEQ ID NO:206). As another example, in some cases, amino acids 135-143 of the MHC class I heavy chain of a pMHC polypeptide comprises the amino acid sequence AACMAAQIT (SEQ ID NO:207). As another example, in some cases, amino acids 135-143 of the MHC class I heavy chain of a pMHC polypeptide comprises the amino acid sequence AADCAAQIT (SEQ ID NO:208). As another example, in some cases, amino acids 135-143 of the MHC class I heavy chain of a pMHC polypeptide comprises the amino acid sequence AADMCAQIT (SEQ ID NO:209). As another example, in some cases, amino acids 135-143 of the MHC class I heavy chain of a pMHC polypeptide comprises the amino acid sequence AADMACQIT (SEQ ID NO:210). As another example, in some cases, amino acids 135-143 of the MHC class I heavy chain of a pMHC polypeptide comprises the amino acid sequence AADMAACIT (SEQ ID NO:211). As another example, in some cases, amino acids 135-143 of the MHC class I heavy chain of a pMHC polypeptide comprises the amino acid sequence AADMAAQCT (SEQ ID NO:212). As another example, in some cases, amino acids 135-143 of the MHC class I heavy chain of a pMHC polypeptide comprises the amino acid sequence AADMAAQIC (SEQ ID NO:213).

As another example, in some cases, amino acids 135-143 of the MHC class I heavy chain of a pMHC polypeptide comprises the amino acid sequence CADTAAQIT (SEQ ID NO:214). As another example, in some cases, amino acids 135-143 of the MHC class I heavy chain of a pMHC polypeptide comprises the amino acid sequence ACDTAAQIT (SEQ ID NO:215). As another example, in some cases, amino acids 135-143 of the MHC class I heavy chain of a pMHC polypeptide comprises the amino acid sequence AACTAAQIT (SEQ ID NO:216). As another example, in some cases, amino acids 135-143 of the MHC class I heavy chain of a pMHC polypeptide comprises the amino acid sequence AADCAAQIT (SEQ ID NO:217). As another example, in some cases, amino acids 135-143 of the MHC class I heavy chain of a pMHC polypeptide comprises the amino acid sequence AADTCAQIT (SEQ ID NO:218). As another example, in some cases, amino acids 135-143 of the MHC class I heavy chain of a pMHC polypeptide comprises the amino acid sequence AADTACQIT (SEQ ID NO:219). As another example, in some cases, amino acids 135-143 of the MHC class I heavy chain of a pMHC polypeptide comprises the amino acid sequence AADTAACIT (SEQ ID NO:220). As another example, in some cases, amino acids 135-143 of the MHC class I heavy chain of a pMHC polypeptide comprises the amino acid sequence AADTAAQCT (SEQ ID NO:221). As another example, in some cases, amino acids 135-143 of the MHC class I heavy chain of a pMHC polypeptide comprises the amino acid sequence AADTAAQIC (SEQ ID NO:222).

As another example, in some cases, amino acids 135-143 of the MHC class I heavy chain of a pMHC polypeptide comprises the amino acid sequence CVDTAAQIS (SEQ ID NO:223). As another example, in some cases, amino acids 135-143 of the MHC class I heavy chain of a pMHC polypeptide comprises the amino acid sequence ACDTAAQIS (SEQ ID NO:224). As another example, in some cases, amino acids 135-143 of the MHC class I heavy chain of a pMHC polypeptide comprises the amino acid sequence AVCTAAQIS (SEQ ID NO:225). As another example, in some cases, amino acids 135-143 of the MHC class I heavy chain of a pMHC polypeptide comprises the amino acid sequence AVDCAAQIS (SEQ ID NO:226). As another example, in some cases, amino acids 135-143 of the MHC class I heavy chain of a pMHC polypeptide comprises the amino acid sequence AVDTCAQIS (SEQ ID NO:227). As another example, in some cases, amino acids 135-143 of the MHC class I heavy chain of a pMHC polypeptide comprises the amino acid sequence AVDTACQIS (SEQ ID NO:228). As another example, in some cases, amino acids 135-143 of the MHC class I heavy chain of a pMHC polypeptide comprises the amino acid sequence AVDTAACIS (SEQ ID NO:229). As another example, in some cases, amino acids 135-143 of the MHC class I heavy chain of a pMHC polypeptide comprises the amino acid sequence AVDTAAQCS (SEQ ID NO:230). As another example, in some cases, amino acids 135-143 of the MHC class I heavy chain of a pMHC polypeptide comprises the amino acid sequence AVDTAAQIC (SEQ ID NO:231).

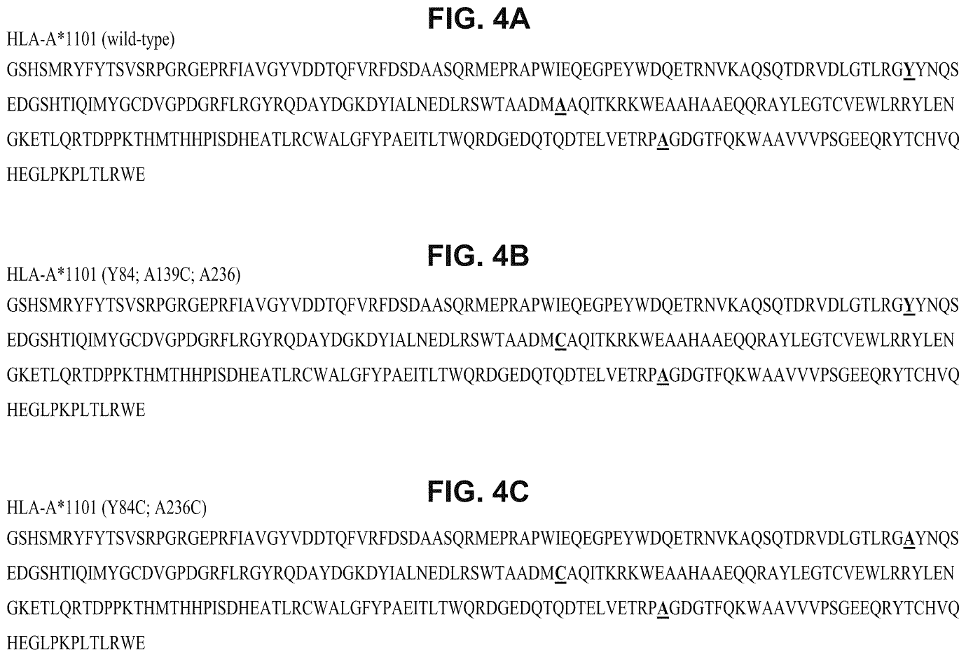

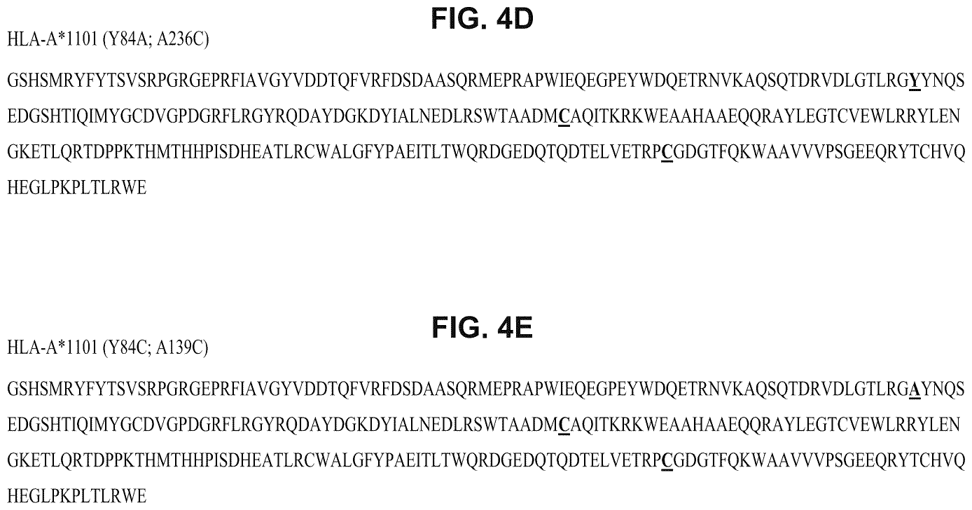

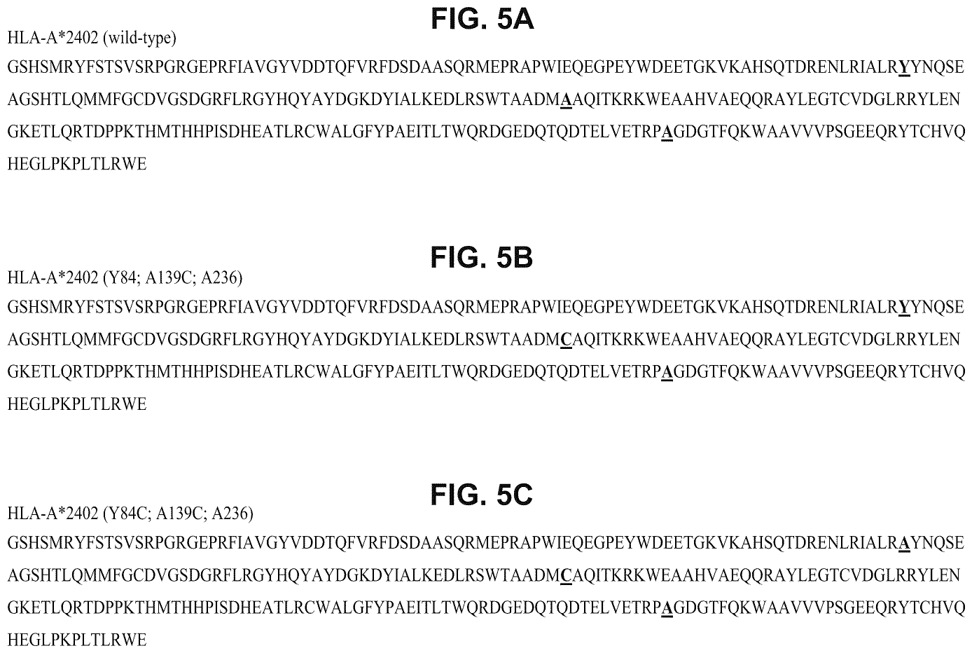

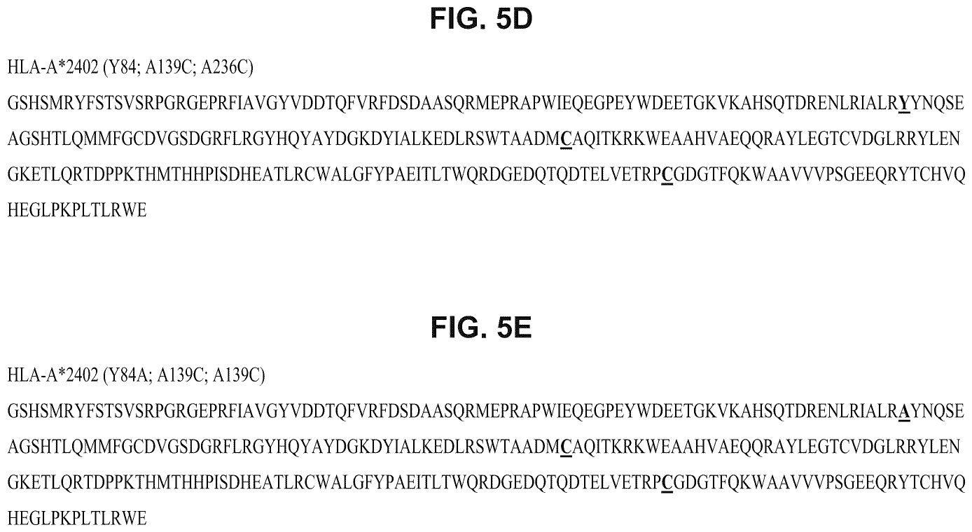

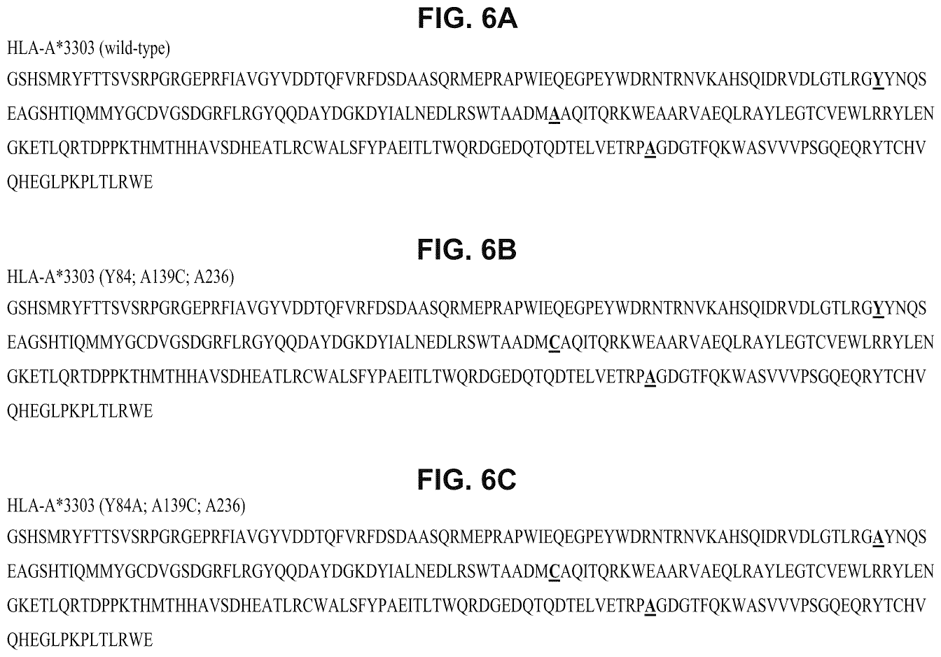

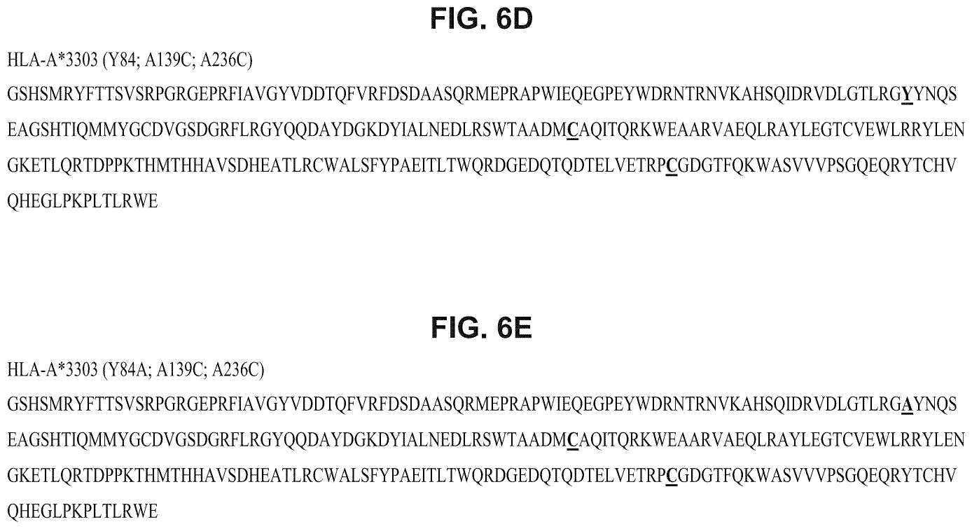

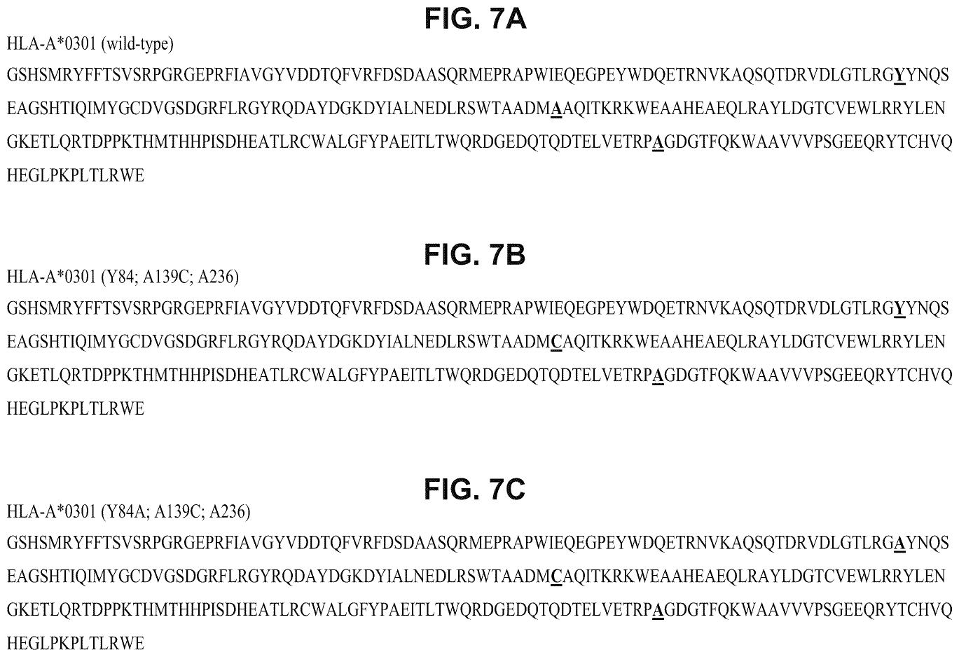

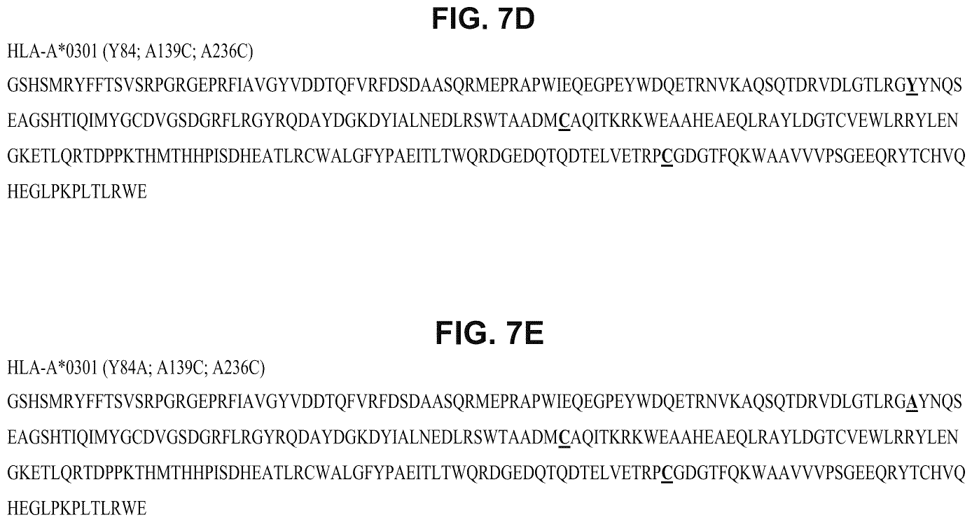

In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIGS. 8C-8D, where amino acid 139 is Cys, and where amino acid 84 is other than a Cys (e.g., where amino acid 84 is Ala, Gly, or Val). In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, and where amino acid 84 is other than a Cys (e.g., where amino acid 84 is Tyr) such that amino acid 84 is unable to form a disulfide bond with the Cys in the linker that is interposed between the peptide epitope and the β2M polypeptide. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, where amino acid 84 is Tyr, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, where amino acid 84 is Tyr, and where amino acid 236 is Cys. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, where amino acid 84 is Ala, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, where amino acid 84 is Ala, and where amino acid 236 is Cys. In some cases, the MHC class I heavy chain polypeptide in a pMHC polypeptide comprises an amino acid having at least 85%, at least 90%, at least 95%, at least 97%, at least 98%, at least 99%, or 100%, amino acid sequence identity to any one of the HLA-A amino acid sequence depicted in FIG. 3B-3E, FIG. 4B-4E, FIG. 5B-5E, FIG. 6B-6E, and FIG. 7B-7E.

In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIGS. 9C-9D, where amino acid 139 is Cys, and where amino acid 84 is other than a Cys (e.g., where amino acid 84 is Ala, Gly, or Val). In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIGS. 9C-9D, where amino acid 139 is Cys, and where amino acid 84 is other than a Cys (e.g., where amino acid 84 is Tyr. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIGS. 9C-9D, where amino acid 139 is Cys, where amino acid 84 is Tyr, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIGS. 9C-9D, where amino acid 139 is Cys, where amino acid 84 is Tyr, and where amino acid 236 is Cys. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIGS. 9C-9D, where amino acid 139 is Cys, where amino acid 84 is Ala, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIGS. 9C-9D, where amino acid 139 is Cys, where amino acid 84 is Ala, and where amino acid 236 is Cys.

In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIGS. 10C-10D, where amino acid 139 is Cys, and where amino acid 84 is other than a Cys (e.g., where amino acid 84 is Ala, Gly, or Val). In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIGS. 10C-10D, where amino acid 139 is Cys, and where amino acid 84 is other than a Cys (e.g., where amino acid 84 is Tyr. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIGS. 10C-10D, where amino acid 139 is Cys, where amino acid 84 is Tyr, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIGS. 10C-10D, where amino acid 139 is Cys, where amino acid 84 is Tyr, and where amino acid 236 is Cys. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIGS. 10C-10D, where amino acid 139 is Cys, where amino acid 84 is Ala, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIGS. 10C-10D, where amino acid 139 is Cys, where amino acid 84 is Ala, and where amino acid 236 is Cys.

In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, and where amino acid 84 is other than a Cys (e.g., where amino acid 84 is Ala, Gly, or Val). In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, and where amino acid 84 is other than a Cys (e.g., where amino acid 84 is Tyr. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, where amino acid 84 is Tyr, and where amino acid 236 is Ala. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, where amino acid 84 is Tyr, and where amino acid 236 is Cys. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, where amino acid 84 is Ala, and where amino acid 236 is Ala. In some cases, the MHC class I heavy chain polypeptide in a pMHC polypeptide comprises an amino acid having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, where amino acid 84 is Ala, and where amino acid 236 is Cys.

In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to the HLA-G amino acid sequence depicted in FIG. 13B or FIG. 13D, where amino acid 139 is Cys, and where amino acid 84 is other than a Cys (e.g., where amino acid 84 is Ala, Gly, or Val). In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to amino acid sequence identity to the HLA-G amino acid sequence depicted in FIG. 13B or FIG. 13D, where amino acid 139 is Cys, and where amino acid 84 is other than a Cys (e.g., where amino acid 84 is Tyr. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to the HLA-G amino acid sequence depicted in FIG. 13B or FIG. 13D, where amino acid 139 is Cys, where amino acid 84 is Tyr, and where amino acid 236 is Ala. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to the HLA-G amino acid sequence depicted in FIG. 13B or FIG. 13D, where amino acid 139 is Cys, where amino acid 84 is Tyr, and where amino acid 236 is Cys. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to the HLA-G amino acid sequence depicted in FIG. 13B or FIG. 13D where amino acid 139 is Cys, where amino acid 84 is Ala, and where amino acid 236 is Ala. In some cases, the MHC class I heavy chain polypeptide in a pMHC polypeptide comprises an amino acid having at least 85%, at least 90%, at least 95%, at least 97%, at least 98%, at least 99%, or 100%, amino acid sequence identity to the HLA-G amino acid sequence depicted in FIG. 13B or FIG. 13D, where amino acid 139 is Cys, where amino acid 84 is Ala, and where amino acid 236 is Cys.

The first peptide linker in a pMHC polypeptide of this disclosure comprises a single Cys, which forms an intrachain disulfide bond with a Cys at one of amino acids 135-143 of the MHC class I heavy chain polypeptide. The first peptide linker present in a pMHC polypeptide can have a length of from about 5 amino acids to about 50 amino acids, e.g., from about 5 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids.

In some cases, the first peptide linker comprises the amino acid sequence CGGGS(GGGGS)n (SEQ ID NO:232), GCGGS(GGGGS)n (SEQ ID NO:233), or GGCGS(GGGGS)n (SEQ ID NO:234), where n is an integer from 1-10 (i.e., where n is 1, 2, 3, 4, 5, 6, 7, 8, 9, or 10). In some cases, the first peptide linker comprises the amino acid sequence CGGGS(GGGGS)n (SEQ ID NO:232), where n is an integer from 1-10 (i.e., where n is 1, 2, 3, 4, 5, 6, 7, 8, 9, or 10); such a peptide linker is referred to as a “GIC” linker. In some cases, the first peptide linker comprises the amino acid sequence CGGGS(GGGGS)n (SEQ ID NO:235), where n is 1. In some cases, the first peptide linker comprises the amino acid sequence CGGGS(GGGGS)n (SEQ ID NO:236), where n is 2. In some cases, the first peptide linker comprises the amino acid sequence CGGGS(GGGGS)n (SEQ ID NO:237), where n is 3. In some cases, the first peptide linker comprises the amino acid sequence GCGGS(GGGGS)n (SEQ ID NO:233), where n is an integer from 1-10 (i.e., where n is 1, 2, 3, 4, 5, 6, 7, 8, 9, or 10); such a peptide linker is referred to as a “G2C” linker. In some cases, the first peptide linker comprises the amino acid sequence GCGGS(GGGGS)n (SEQ ID NO:238), where n is 1. In some cases, the first peptide linker comprises the amino acid sequence GCGGS(GGGGS)n (SEQ ID NO:239), where n is 2. In some cases, the first peptide linker comprises the amino acid sequence GCGGS(GGGGS)n (SEQ ID NO:240), where n is 3. In some cases, the first peptide linker comprises the amino acid sequence GGCGS(GGGGS)n (SEQ ID NO:234), where n is an integer from 1-10 (i.e., where n is 1, 2, 3, 4, 5, 6, 7, 8, 9, or 10); such a linker is referred to as a “G3C” linker. In some cases, the first peptide linker comprises the amino acid sequence GGCGS(GGGGS)n (SEQ ID NO:241), where n is 1. In some cases, the first peptide linker comprises the amino acid sequence GGCGS(GGGGS)n (SEQ ID NO:242), where n is 2. In some cases, the first peptide linker comprises the amino acid sequence GGCGS(GGGGS)n (SEQ ID NO:243), where n is 3.

### (G2C; A139C)

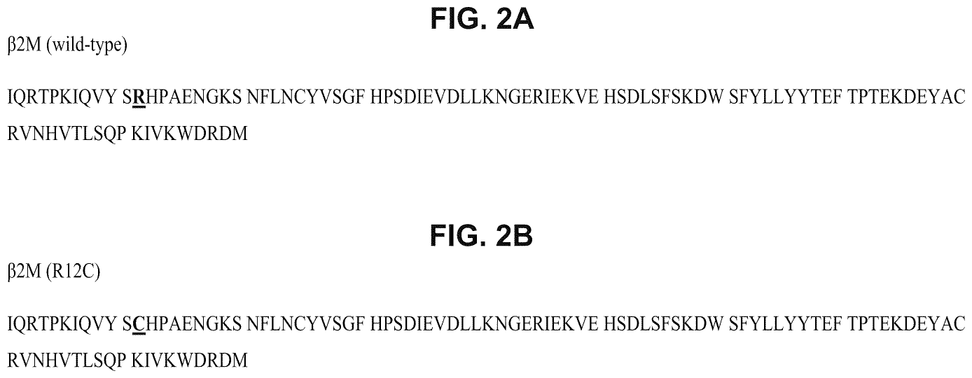

In some cases, a pMHC polypeptide comprises an intrachain disulfide bond between: i) a Cys at amino acid 139 of the MHC class I heavy chain polypeptide; and ii) a Cys in the first peptide linker, where the peptide linker is a “G2C” linker and comprises the amino acid sequence GCGGS(GGGGS)n (SEQ ID NO:233), where n is an integer from 1 to 10. In some cases, for example, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GCGGS(GGGGS)n (SEQ ID NO:238), where n is 1. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GCGGS(GGGGS)n (SEQ ID NO:239), where n is 2. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GCGGS(GGGGS)n (SEQ ID NO:240), where n is 3. And so on where n is 4, 5, 6, 7, 8, 9 or 10. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. FIG. 1A provides a schematic depiction of such a pMHC. In some cases, the pMHC polypeptide comprises only a single intrachain disulfide bond, where the single intrachain disulfide bond is between: i) a Cys at amino acid 139 of the MHC class I heavy chain polypeptide; and ii) a Cys in the first peptide linker, where the peptide linker comprises the amino acid sequence GCGGS(GGGGS)n (SEQ ID NO:233), where n is an integer from 1 to 10. FIG. 1B provides a schematic depiction of such a pMHC. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G2C; M138C or T138C)

In some cases, a pMHC polypeptide comprises an intrachain disulfide bond between: i) a Cys at amino acid 138 of the MHC class I heavy chain polypeptide; and ii) a Cys in the first peptide linker, where the peptide linker is a “G2C” linker and comprises the amino acid sequence GCGGS(GGGGS)n (SEQ ID NO:233), where n is an integer from 1 to 10. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence GCGGS(GGGGS)n (SEQ ID NO:238), where n is 1. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence GCGGS(GGGGS)n (SEQ ID NO:239), where n is 2. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence GCGGS(GGGGS)n (SEQ ID NO:240), where n is 3. In some cases, n is 4, 5, 6, 7, 8, 9 or 10. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. In some cases, the pMHC polypeptide comprises only a single intrachain disulfide bond, where the single intrachain disulfide bond is between: i) a Cys at amino acid 138 of the MHC class I heavy chain polypeptide; and ii) a Cys in the first peptide linker, where the peptide linker comprises the amino acid sequence GCGGS(GGGGS)n (SEQ ID NO:233), where n is an integer from 1 to 10. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G2C; A140C)

In some cases, a pMHC polypeptide comprises an intrachain disulfide bond between: i) a Cys at amino acid 140 of the MHC class I heavy chain polypeptide; and ii) a Cys in the first peptide linker, where the peptide linker is a “G2C” linker and comprises the amino acid sequence GCGGS(GGGGS)n, (SEQ ID NO:233) where n is an integer from 1 to 10. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GCGGS(GGGGS)n (SEQ ID NO:238), where n is 1. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GCGGS(GGGGS)n (SEQ ID NO:239), where n is 2. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GCGGS(GGGGS)n (SEQ ID NO:240), where n is 3. In some cases, n is 4, 5, 6, 7, 8, 9 or 10. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. In some cases, the pMHC polypeptide comprises only a single intrachain disulfide bond, where the single intrachain disulfide bond is between: i) a Cys at amino acid 140 of the MHC class I heavy chain polypeptide; and ii) a Cys in the first peptide linker, where the peptide linker comprises the amino acid sequence GCGGS(GGGGS)n (SEQ ID NO:233), where n is an integer from 1 to 10. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G1C; A139C)

In some cases, a pMHC polypeptide comprises an intrachain disulfide bond between: i) a Cys at amino acid 139 of the MHC class I heavy chain polypeptide; and ii) a Cys in the first peptide linker, where the peptide linker is a “G1C” linker and comprises the amino acid sequence CGGGS(GGGGS)n (SEQ ID NO:232), where n is an integer from 1 to 10. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence CGGGS(GGGGS)n (SEQ ID NO:235), where n is 1. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence CGGGS(GGGGS)n (SEQ ID NO:236), where n is 2. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence CGGGS(GGGGS)n (SEQ ID NO:237), where n is 3. In some cases, n is 4, 5, 6, 7, 8, 9 or 10. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. FIG. 1G provides a schematic depiction of such a pMHC. In some cases, the pMHC polypeptide comprises only a single intrachain disulfide bond, where the single intrachain disulfide bond is between: i) a Cys at amino acid 139 of the MHC class I heavy chain polypeptide; and ii) a Cys in the first peptide linker, where the peptide linker comprises the amino acid sequence CGGGS(GGGGS)n (SEQ ID NO:232), where n is an integer from 1 to 10. FIG. 1C provides a schematic depiction of such a pMHC. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G1C; M138C or T138C)

In some cases, a pMHC polypeptide comprises an intrachain disulfide bond between: i) a Cys at amino acid 138 of the MHC class I heavy chain polypeptide; and ii) a Cys in the first peptide linker, where the peptide linker is a “G1C” linker and comprises the amino acid sequence CGGGS(GGGGS)n (SEQ ID NO:232), where n is an integer from 1 to 10. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence CGGGS(GGGGS)n (SEQ ID NO:235), where n is 1. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence CGGGS(GGGGS)n (SEQ ID NO:236), where n is 2. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence CGGGS(GGGGS)n (SEQ ID NO:237), where n is 3. In some cases, n is 4, 5, 6, 7, 8, 9 or 10. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. FIG. 1H provides a schematic depiction of such a pMHC. In some cases, the pMHC polypeptide comprises only a single intrachain disulfide bond, where the single intrachain disulfide bond is between: i) a Cys at amino acid 138 of the MHC class I heavy chain polypeptide; and ii) a Cys in the first peptide linker, where the peptide linker comprises the amino acid sequence CGGGS(GGGGS)n (SEQ ID NO:232), where n is an integer from 1 to 10. FIG. 1D provides a schematic depiction of such a pMHC. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G3C; A139C)

In some cases, a pMHC polypeptide comprises an intrachain disulfide bond between: i) a Cys at amino acid 139 of the MHC class I heavy chain polypeptide; and ii) a Cys in the first peptide linker, where the peptide linker is a “G3C” linker and comprises the amino acid sequence GGCGS(GGGGS)n (SEQ ID NO:234), where n is an integer from 1 to 10. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GGCGS(GGGGS)n (SEQ ID NO:241), where n is 1. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GGCGS(GGGGS)n (SEQ ID NO:242), where n is 2. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GGCGS(GGGGS)n (SEQ ID NO:243), where n is 3. In some cases, n is 4, 5, 6, 7, 8, 9, or 10. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. FIG. 1I provides a schematic depiction of such a pMHC. In some cases, the pMHC polypeptide comprises only a single intrachain disulfide bond, where the single intrachain disulfide bond is between: i) a Cys at amino acid 139 of the MHC class I heavy chain polypeptide; and ii) a Cys in the first peptide linker, where the peptide linker comprises the amino acid sequence GGCGS(GGGGS)n (SEQ ID NO:234), where n is an integer from 1 to 10. FIG. 1E provides a schematic depiction of such a pMHC. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G3C; A140C)

In some cases, a pMHC polypeptide comprises an intrachain disulfide bond between: i) a Cys at amino acid 140 of the MHC class I heavy chain polypeptide; and ii) a Cys in the first peptide linker, where the peptide linker is a “G3C” linker and comprises the amino acid sequence GGCGS(GGGGS)n (SEQ ID NO:234), where n is an integer from 1 to 10. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GGCGS(GGGGS)n (SEQ ID NO:241), where n is 1. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GGCGS(GGGGS)n (SEQ ID NO:242), where n is 2. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GGCGS(GGGGS)n (SEQ ID NO:243), where n is 3. In some cases, n is 4, 5, 6, 7, 8, 9 or 10. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. FIG. 1J provides a schematic depiction of such a pMHC. In some cases, the pMHC polypeptide comprises only a single intrachain disulfide bond, where the single intrachain disulfide bond is between: i) a Cys at amino acid 140 of the MHC class I heavy chain polypeptide; and ii) a Cys in the first peptide linker, where the peptide linker comprises the amino acid sequence GGCGS(GGGGS)n (SEQ ID NO:234), where n is an integer from 1 to 10. FIG. 1F provides a schematic depiction of such a pMHC. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

The MHC class I heavy chain polypeptide present in a pMHC polypeptide comprises an amino acid other than Cys at position 84, based on the numbering of the MHC class I heavy chain polypeptide depicted in FIG. 3A. A wild-type MHC class I heavy chain polypeptide comprises a Tyr at position 84. In some cases, the MHC class I heavy chain polypeptide present in a pMHC polypeptide comprises a Tyr at position 84. Substitution of the Tyr at position 84 with an amino acid comprising a small, nonpolar side chain enhances access of the peptide epitope to the peptide binding site of the β2M/MHC class I heavy chain polypeptide complex. In some cases, the MHC class I heavy chain polypeptide present in a pMHC polypeptide comprises an amino acid selected from Ala, Gly, and Val at position 84, based on the numbering of the MHC class I heavy chain polypeptide depicted in FIG. 3A. In some cases, the MHC class I heavy chain polypeptide present in a pMHC polypeptide comprises an Ala at position 84, based on the numbering of the MHC class I heavy chain polypeptide depicted in FIG. 3A. In some cases, the MHC class I heavy chain polypeptide present in a pMHC polypeptide comprises a Gly at position 84, based on the numbering of the MHC class I heavy chain polypeptide depicted in FIG. 3A. In some cases, the MHC class I heavy chain polypeptide present in a pMHC polypeptide comprises a Val at position 84, based on the numbering of the MHC class I heavy chain polypeptide depicted in FIG. 3A.

### β2M Polypeptides

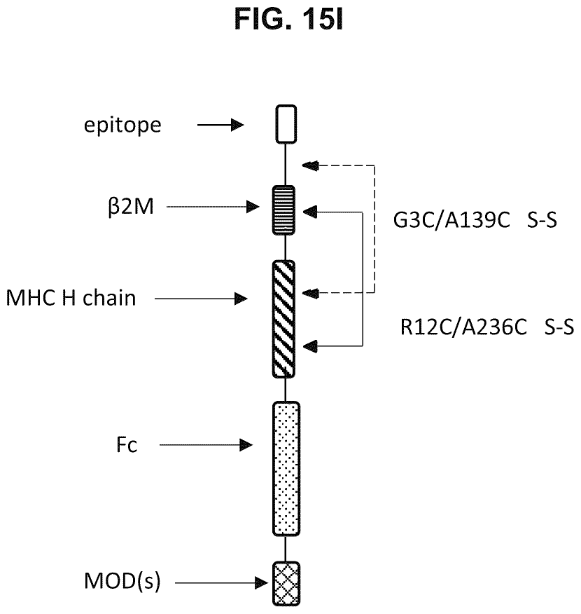

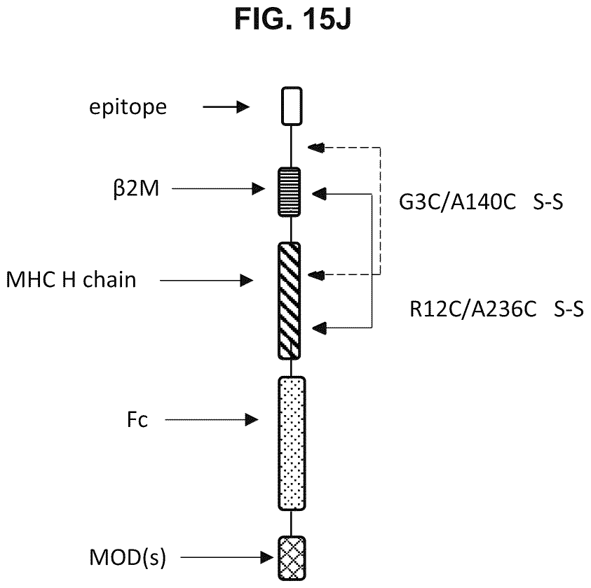

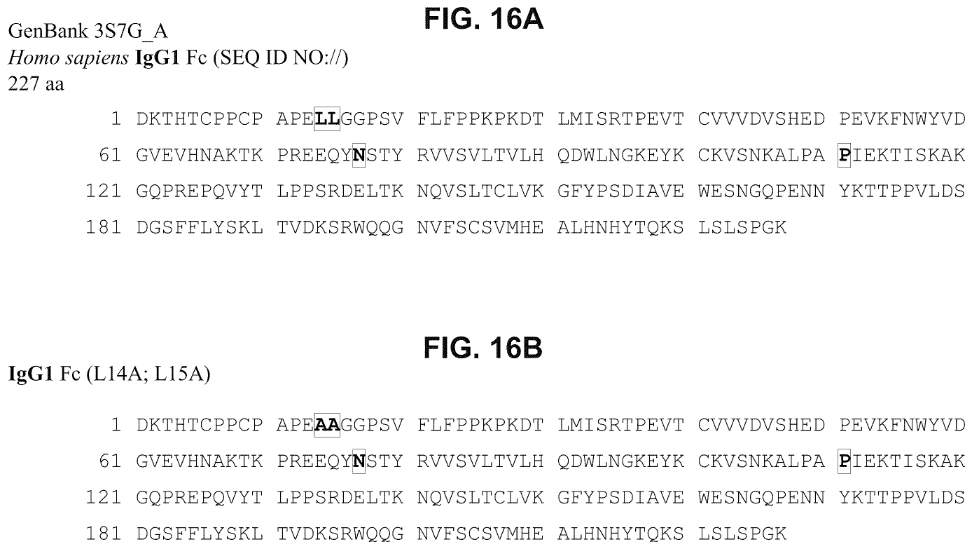

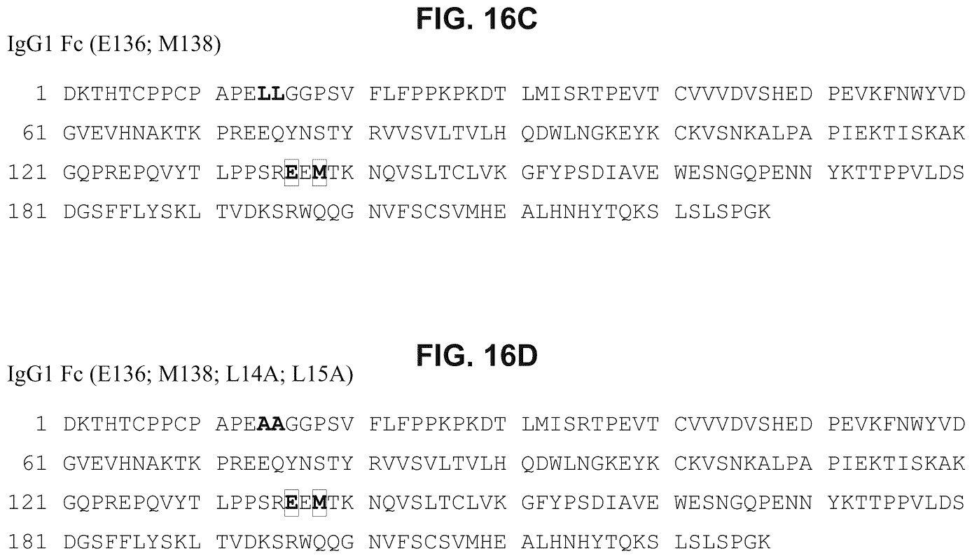

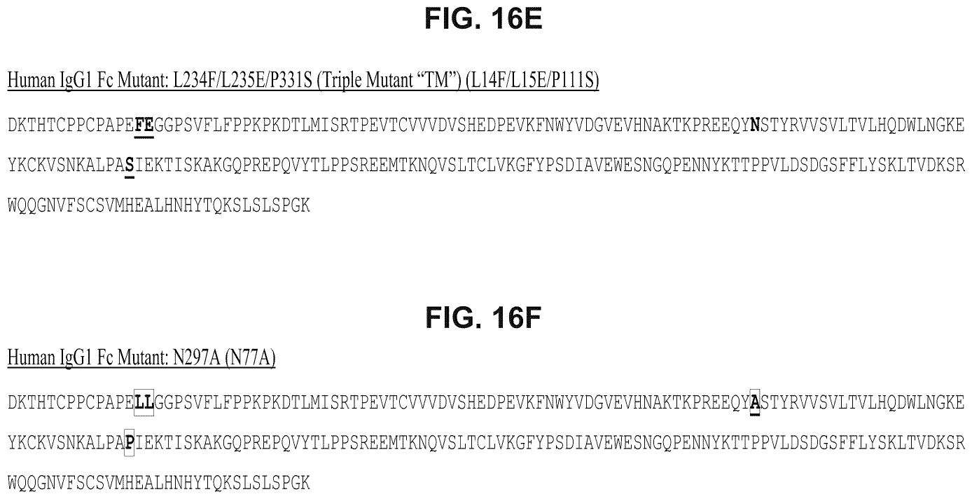

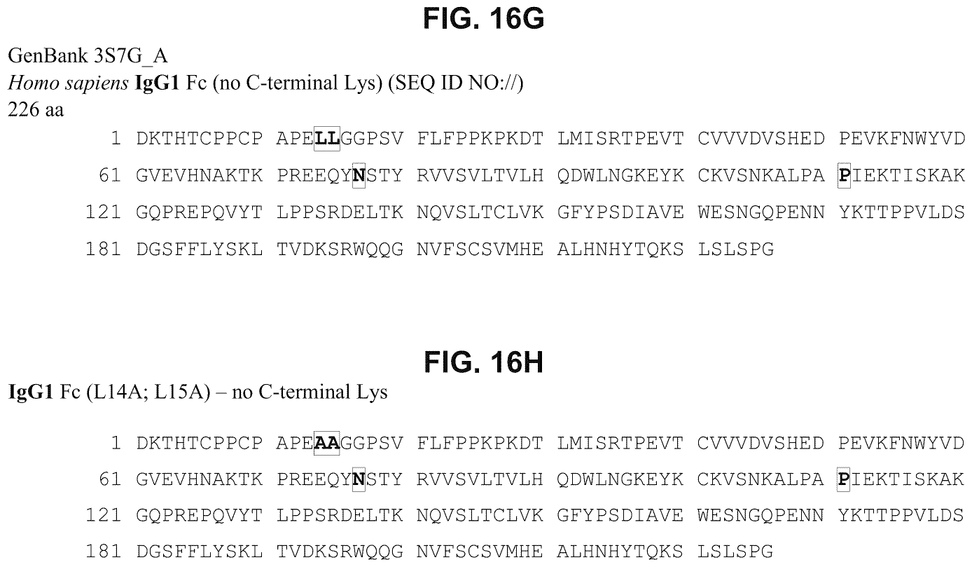

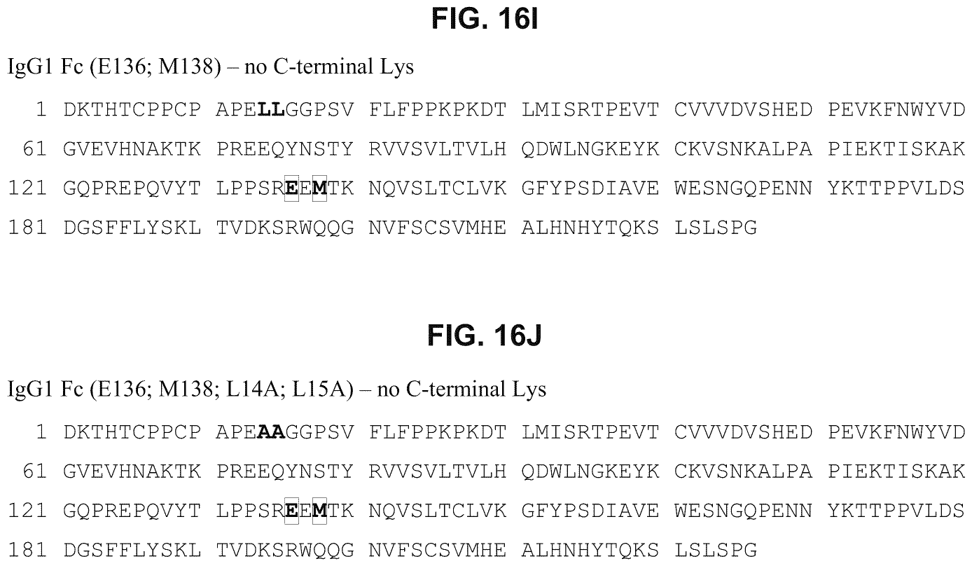

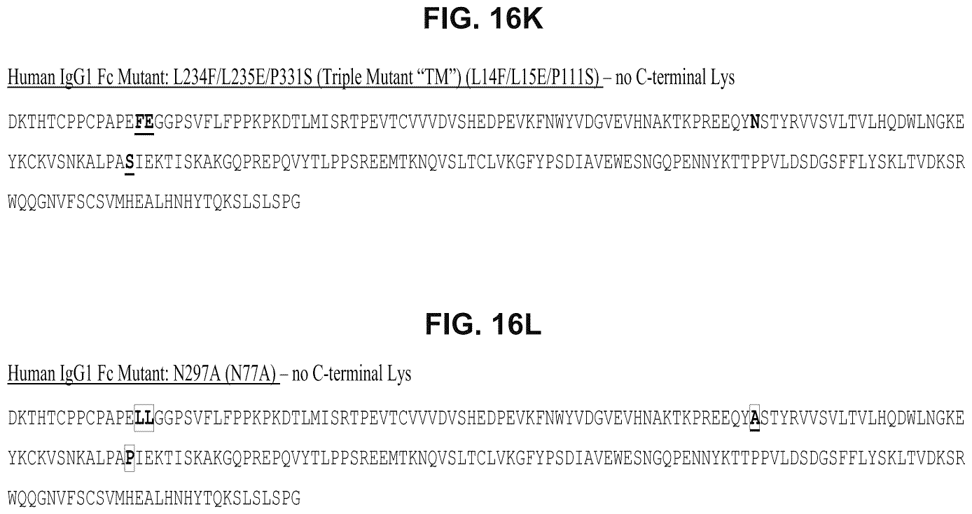

In some cases, a β2M polypeptide present in a pMHC polypeptide can have an amino acid sequence having at least 85%, at least 90%, at least 95%, at least 97%, at least 98%, at least 99%, or 100%, amino acid sequence identity to the β2M amino acid sequence depicted in FIG. 16A. In some cases, a β2M polypeptide present in a pMHC polypeptide can have an amino acid sequence having at least 85%, at least 90%, at least 95%, at least 97%, at least 98%, at least 99%, or 100%, amino acid sequence identity to the β2M amino acid sequence depicted in FIG. 16B, where the β2M polypeptide comprises a Cys at position 12.

### Peptide Epitopes

A pMHC polypeptide of this disclosure comprises a peptide epitope. As used herein, the term “peptide epitope” means a peptide that can be bound to MHC polypeptides (e.g., MHC class I polypeptides) in the pMHC, such that the pMHC presents an epitope that can be specifically bound to a TCR of a T cell. An epitope presented by a pMHC polypeptide thus can be specifically bound by a T-cell that has a TCR that is specific for the epitope presented by the pMHC polypeptide.

A peptide epitope present in a pMHC polypeptide can have a length of at least 4 amino acids, e.g., from 4-20 aa (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa or 20 aa), including a range of from 6-15 aa, 8-12 aa, 8-16 aa, 8-10 aa, 9-11 aa, 5-10 aa, 10-15 aa, and 15-20 aa in length. In some cases, the peptide epitope is 8, 9, 10, or 11 amino acids in length.

In some cases, the pMHC polypeptide presents an epitope specific to an HLA-A, -B, -C, -E, -F, or -G allele. In an embodiment, the pMHC presents an epitope restricted to HLA-A*0101, A*0201, A*0301, A*1101, A*2301, A*2402, A*2407, A*3303, and/or A*3401. In an embodiment, the pMHC polypeptide presents an epitope restricted to HLA-B*0702, B*0801, B*1502, B*3802, B*4001, B*4601, and/or B*5301. In an embodiment, the pMHC polypeptide presents an epitope restricted to C*0102, C*0303, C*0304, C*0401, C*0602, C*0701, C*702, C*0801, and/or C*1502.

A pMHC polypeptide of this disclosure comprises a peptide epitope other than a KRAS peptide associated with a cancer. In other words, the peptide present in a pMHC polypeptide is not a KRAS peptide associated with a cancer. For example, a peptide epitope present in a pMHC polypeptide is not a peptide of from about 4 amino acids to about 20 amino acids of a KRAS polypeptide, where a KRAS polypeptide can have the following amino acid sequence: MTEYKLVVVG AGGVGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:1). For example, a peptide epitope present in a pMHC polypeptide is not any of the following peptides:

(SEQ ID NO: 2)
VVVGAGDVGK;
(SEQ ID NO: 3)
VVGAGDVGK;
(SEQ ID NO: 4)
VVVGARGVGK;
(SEQ ID NO: 5)
VVGARGVGK;
(SEQ ID NO: 6)
LVVVGADGV,
(SEQ ID NO: 7)
LVVVGAVGV,
(SEQ ID NO: 8)
LVVVGACGV,
(SEQ ID NO: 9)
KLVVVGADGV,
(SEQ ID NO: 10)
KLVVVGAVGV,
(SEQ ID NO: 11)
KLVVVGACGV,
(SEQ ID NO: 12)
LLVVGADGV,
(SEQ ID NO: 13)
LLVVGAVGV,
(SEQ ID NO: 14)
LLVVGACGV,
(SEQ ID NO: 15)
FLVVVGADGV,
(SEQ ID NO: 16)
FLVVVGAVGV,
(SEQ ID NO: 17)
FLVVVGACGV;
(SEQ ID NO: 18)
KLVVVGAGDV;
(SEQ ID NO: 19)
KLVVVGARGV;
(SEQ ID NO: 20)
GAGDVGKSAL;
(SEQ ID NO: 21)
AGDVGKSAL;
(SEQ ID NO: 22)
DVGKSALTI;
(SEQ ID NO: 23)
GAVGVGKSAL;
(SEQ ID NO: 24)
AVGVGKSAL;
(SEQ ID NO: 25)
YKLVVVGAV;
(SEQ ID NO: 26)
ARGVGKSAL;
(SEQ ID NO: 27)
GARGVGKSAL;
(SEQ ID NO: 28)
EYKLVVVGAR;
(SEQ ID NO: 29)
RGVGKSALTI;
(SEQ ID NO: 30)
LVVVGARGV;
(SEQ ID NO: 31)
GADGVGKSAL;
(SEQ ID NO: 32)
ACGVGKSAL;
(SEQ ID NO: 33)
GACGVGKSAL;
(SEQ ID NO: 34)
VVGAVGVGK,
(SEQ ID NO: 35)
VVVGAVGVGK,
(SEQ ID NO: 36)
VGAVGVGKS,
(SEQ ID NO: 37)
VGAVGVGKSA,
(SEQ ID NO: 38)
AVGVGKSALT,
(SEQ ID NO: 39)
GAVGVGKSA,
(SEQ ID NO: 40)
LVVVGAVGVG,
and
(SEQ ID NO: 41)
KLVVVGAVG.

Suitable peptide epitopes for inclusion in a pMHC polypeptide include, but are not limited to, peptide epitopes of a cancer-associated antigen. A pMHC polypeptide comprising such a peptide epitope presents an epitope of a cancer-associated antigen to a TCR. Cancer-associated antigens are known in the art; see, e.g., Cheever et al. (2009) Clin. Cancer Res. 15:5323. Cancer-associated antigens include, but are not limited to, α-folate receptor; carbonic anhydrase IX (CAIX); CD19; CD20; CD22; CD30; CD33; CD44v7/8; carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); vascular endothelial growth factor receptor-2 (VEGF-R2). See, e.g., Vigneron et al. (2013) Cancer Immunity 13:15; and Vigneron (2015) BioMed Res. Int'l Article ID 948501; and epidermal growth factor receptor (EGFR) vIII polypeptide (see, e.g., Wong et al. (1992) Proc. Natl. Acad. Sci. USA 89:2965; and Miao et al. (2014) PLoSOne 9:e94281).

In some cases, a suitable peptide epitope is a peptide fragment of from 4-20 amino acids (aa), e.g., 6-15 aa, 8-12 aa, 8-16 aa, 8-10 aa, 9-11 aa, 5-10 aa, 10-15 aa, or 15-20 aa in length, of a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, a Wilms tumor (WT-1) polypeptide, a MAGE-A4 polypeptide, a Preferentially Expressed Antigen of Melanoma (PRAME) polypeptide, or a Fos-related antigen-1 (FOSL) polypeptide.

Amino acid sequences of cancer-associated antigens are known in the art; see, e.g., MUC1 (GenBank CAA56734); LMP2 (GenBank CAA47024); EGFRvIII (GenBank NP_001333870); HER-2/neu (GenBank AAI67147); MAGE-A3 (GenBank AAH11744); p53 (GenBank BAC16799); NY-ESO-1 (GenBank CAA05908); PSMA (GenBank AAH25672); CEA (GenBank AAA51967); melan/MART1 (GenBank NP_005502); Ras (GenBank NP_001123914); gp100 (GenBank AAC60634); bcr-abl (GenBank AAB60388); tyrosinase (GenBank AAB60319); survivin (GenBank AAC51660); PSA (GenBank CAD54617); hTERT (GenBank BAC11010); SSX (GenBank NP_001265620); Eph2A (GenBank NP_004422); PAP (GenBank AAH16344); ML-IAP (GenBank AAH14475); EpCAM (GenBank NP_002345); ERG (TMPRSS2 ETS fusion) (GenBank ACA81385); PAX3 (GenBank AAI01301); ALK (GenBank NP_004295); androgen receptor (GenBank NP_000035); cyclin B1 (GenBank CA099273); MYCN (GenBank NP_001280157); RhoC (GenBank AAH52808); TRP-2 (GenBank AAC60627); mesothelin (GenBank AAH09272); PSCA (GenBank AAH65183); MAGE A1 (GenBank NP_004979); CYP1B1 (GenBank AAM50512); PLAC1 (GenBank AAG22596); BORIS (GenBank NP_001255969); ETV6 (GenBank NP_001978); NY-BRI (GenBank NP 443723); SART3 (GenBank NP_055521); carbonic anhydrase IX (GenBank EAW58359); PAX5 (GenBank NP_057953); OY-TES1 (GenBank NP_115878); sperm protein 17 (GenBank AAK20878); LCK (GenBank NP_001036236); HMW-MAA (GenBank NP_001888); AKAP-4 (GenBank NP_003877); SSX2 (GenBank CAA60111); XAGE1 (GenBank NP_001091073; XP_001125834; XP_001125856; and XP_001125872); B7H3 (GenBank NP_001019907; XP_947368; XP_950958; XP_950960; XP_950962; XP_950963; XP_950965; and XP_950967); LGMN1 (GenBank NP_001008530); TIE-2 (GenBank NP_000450); PAGE4 (GenBank NP_001305806); VEGFR2 (GenBank NP_002244); MAD-CT-1 (GenBank NP_005893 NP_056215); FAP (GenBank NP_004451); PDGFP (GenBank NP_002600); MAD-CT-2 (GenBank NP_001138574); and FOSL (GenBank NP_005429). These polypeptides are also discussed in, e.g., Cheever et al. (2009) Clin. Cancer Res. 15:5323, and references cited therein; Wagner et al. (2003) J. Cell. Sci. 116:1653; Matsui et al. (1990) Oncogene 5:249; Zhang et al. (1996) Nature 383:168. Some exemplary peptide epitopes are provided below.

### AFP Peptide Epitopes

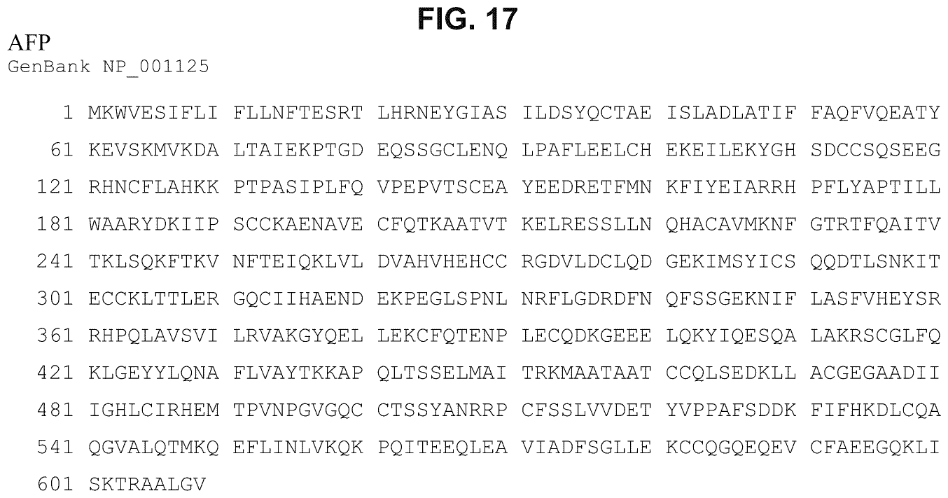

In some cases, a pMHC polypeptide comprises, as the peptide epitope, an alpha-feto protein (AFP) peptide. In some cases, an AFP peptide epitope present in a pMHC polypeptide can be a peptide of from 4-20 aa, e.g., 6-15 aa, 8-16 aa, 8-12 aa, 8-10 aa, 9-11 aa, 5-10 aa, 10-15 aa, or 15-20 aa in length of an amino acid sequence having at least 90%, at least 95%, at least 97%, at least 98%, at least 99%, or 100%, amino acid sequence identity to the AFP amino acid sequence depicted in FIG. 17.

Examples of AFP peptide epitopes suitable for inclusion in a pMHC polypeptide are described in published PCT application WO 2022/197970 (Cue Biopharma, Inc.), the disclosure of which as it pertains to AFP peptide epitopes is expressly incorporated herein by reference, including specifically paragraphs [0072]-[0078]. In some cases, for example, the pMHC polypeptide presents an HLA-A*2402-restricted epitope. Non-limiting examples of AFP peptide epitopes that can be incorporated into a pMHC polypeptide to present an HLA-A*2402-restricted epitope include: KYIQESQAL (SEQ ID NO:244); EYYLQNAFL (SEQ ID NO:245); AYTKKAPQL (SEQ ID NO:246); EYSRRHPQL (SEQ ID NO:247); RSCGLFQKL (SEQ ID NO:248) and AYEEDRETF (SEQ ID NO:249).

In some cases, for example, the pMHC polypeptide presents an HLA-A*0201-restricted epitope. Non-limiting examples of AFP peptide epitopes that can be incorporated into a pMHC polypeptide to present an HLA-A*0201-restricted epitope include: FMNKFIYEI (SEQ ID NO:250); and GLSPNLNRFL (SEQ ID NO:251).

### WT-1 Peptides

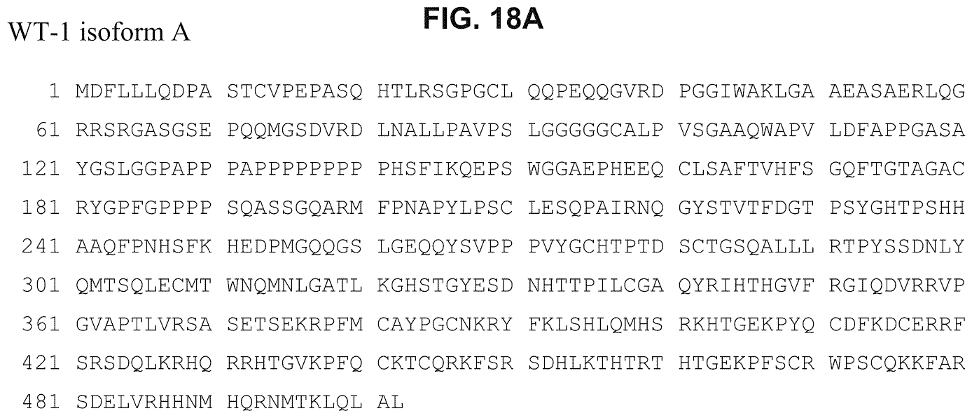

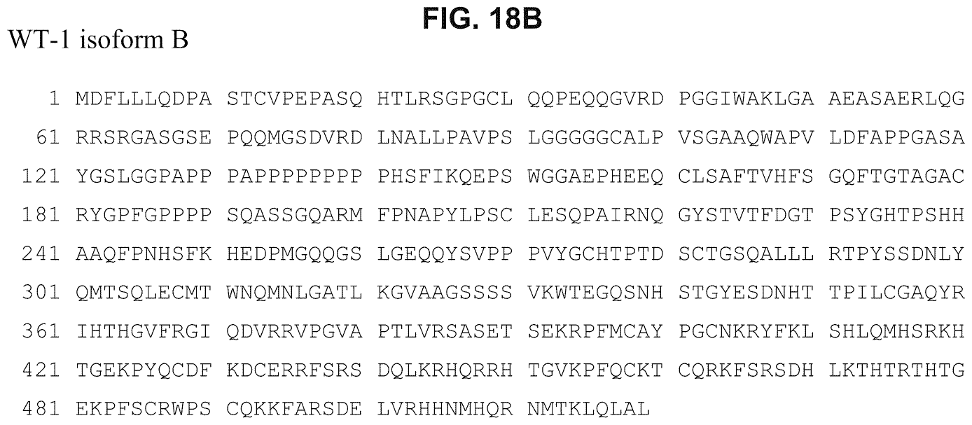

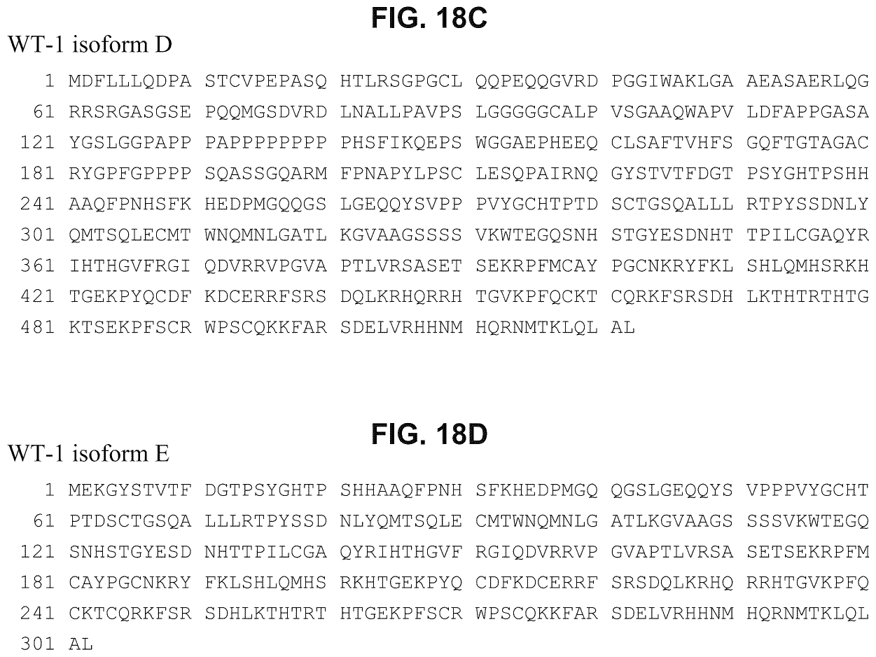

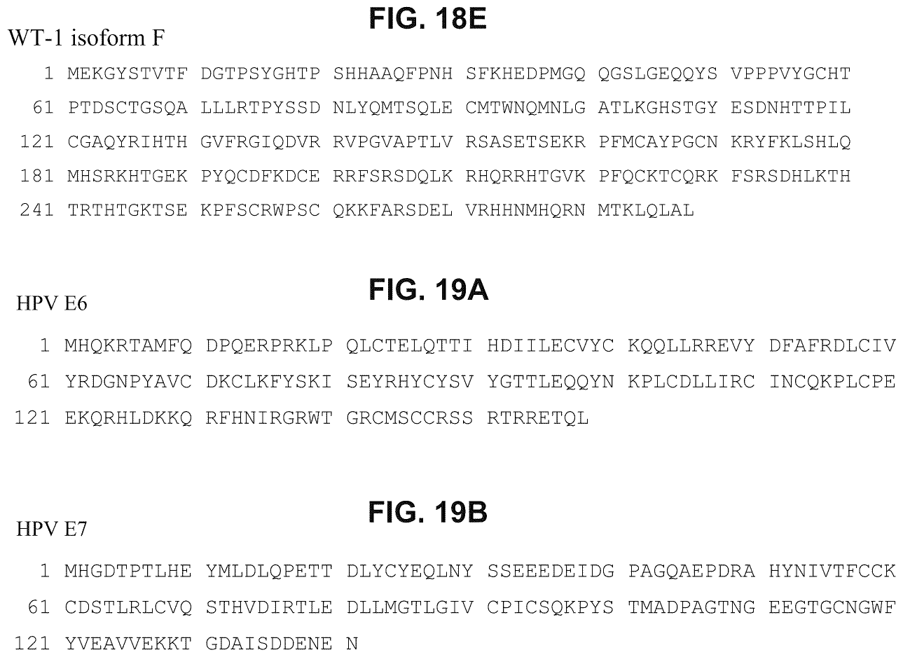

In some cases, a pMHC polypeptide comprises, as the peptide epitope, a Wilms tumor-1 (WT-1) peptide. Amino acid sequences of WT-1 isoforms are presented in FIGS. 18A-18E. In some cases, a WT-1 peptide epitope present in a pMHC polypeptide can be a peptide of from 4-20 aa, e.g., 6-15 aa, 8-16 aa, 8-12 aa, 8-10 aa, 9-11 aa, 5-10 aa, 10-15 aa, or 15-20 aa in length of an amino acid sequence having at least 90%, at least 95%, at least 97%, at least 98%, at least 99%, or 100%, amino acid sequence identity to the WT-1 amino acid sequence depicted in any one of FIGS. 18A-18E.

Examples of WT1 peptide epitopes suitable for inclusion in a pMHC polypeptide are described in published PCT applications WO 2022/197970 (Cue Biopharma, Inc.), WO/2021/231376 (Cue Biopharma, Inc.), and WO 2021/230638 (LG Chem, Ltd.), the disclosures of which as they pertain to specific WT1 peptide epitopes is expressly incorporated herein by reference.

In some cases, for example, a pMHC polypeptide comprising a WT-1 peptide epitope presents an HLA-A*2402-restricted epitope. Such WT-1 peptide epitopes include, e.g., CMTWNQMN (SEQ ID NO:252); NYMNLGATL (SEQ ID NO:253) (WT-1 239-247; Q240Y); CYTWNQMNL (SEQ ID NO:254) (WT-1 235-243); CMTWNQMNL (SEQ ID NO:255) (WT-1 235-243); NQMNLGATL (SEQ ID NO:256) (WT-1 239-247); and NLMNLGATL (SEQ ID NO:257) (WT-1 239-247; Q240L), RVPGVAPTL (SEQ ID NO:258); RYPGVAPTL (SEQ ID NO:259); RYFPNAPYL (SEQ ID NO:260); and RYPSCQKKF (SEQ ID NO:261).

### HPV Peptides

In some cases, a pMHC polypeptide comprises, as the peptide epitope, a human papilloma virus (HPV) peptide. An HPV peptide suitable for inclusion in a pMHC polypeptide can be a peptide of an HPV E6 polypeptide or an HPV E7 polypeptide. The HPV epitope can be an epitope of HPV of any of a variety of genotypes, including, e.g., HPV16, HPV18, HPV31, HPV33, HPV35, HPV39, HPV45, HPV51, HPV52, HPV56, HPV58, HPV59, HPV68, HPV73, or HPV82. In some cases, the epitope is an HPV E6 epitope. In some cases, the epitope is an HPV E7 epitope. An amino acid sequence of an HPV E6 polypeptide is presented in FIG. 19A. In some cases, an HPV peptide epitope present in a pMHC polypeptide is a peptide of from 4-20 aa, e.g., 6-15 aa, 8-12 aa, 8-10 aa, 9-11 aa, 5-10 aa, 10-15 aa, or 15-20 aa in length of an amino acid sequence having at least 90%, at least 95%, at least 97%, at least 98%, at least 99%, or 100%, amino acid sequence identity to the HPV E6 amino acid sequence depicted in FIG. 19A or the HPV E7 amino acid sequence depicted in FIG. 19B.

Examples of HPV peptide epitopes suitable for inclusion in a pMHC polypeptide are described in published PCT application WO 2022/197970 (Cue Biopharma, Inc.), the disclosure of which as it pertains to HPV peptide epitopes is expressly incorporated herein by reference, including specifically paragraphs [0083]-[0088]. In some cases, for example, the HPV peptide epitope, when part of a pMHC polypeptide, presents an HLA-A*2402-restricted epitope. Such HPV peptide epitopes include, e.g., VYDFAFRDL (SEQ ID NO:262); RAHYNIVTF (SEQ ID NO:263); CDSTLRLCV (SEQ ID NO:264); and LCVQSTHVDI (SEQ ID NO:265).

### MUC1 Peptides

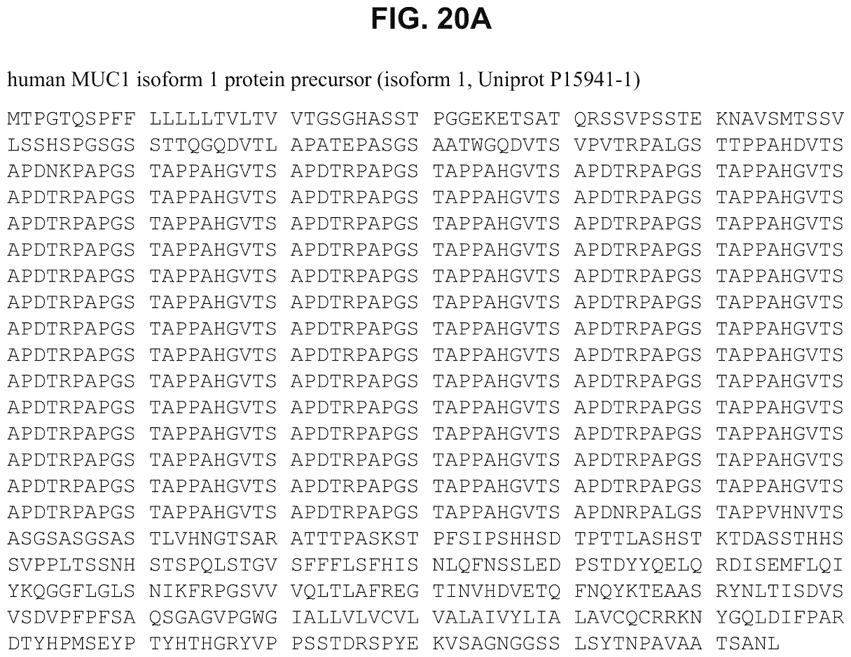

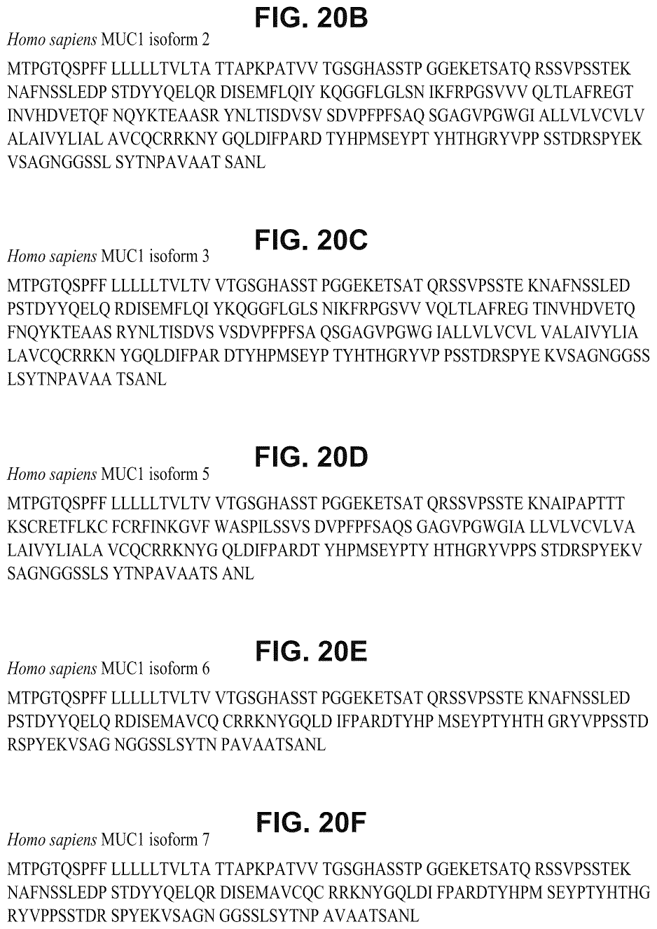

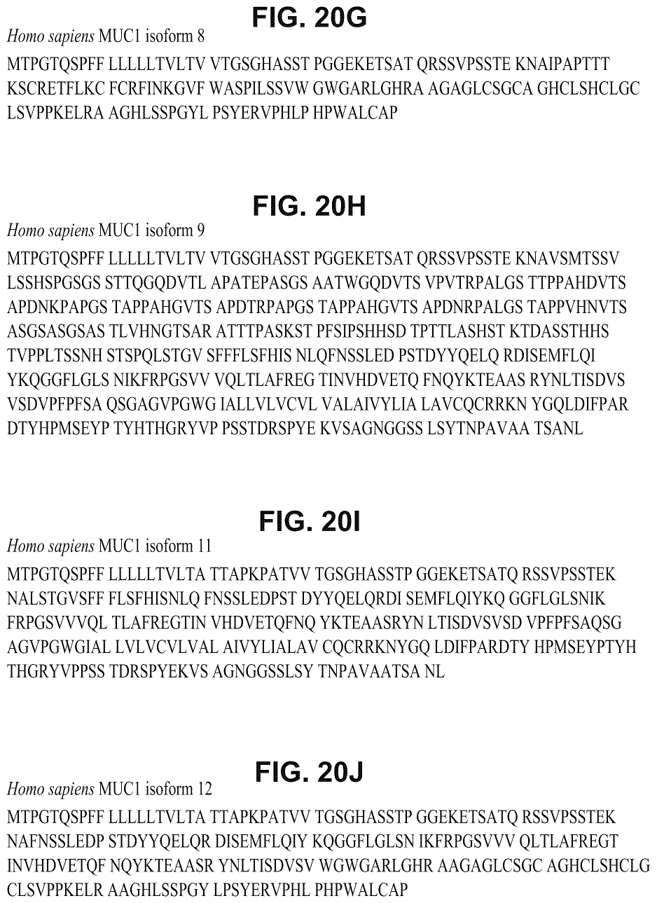

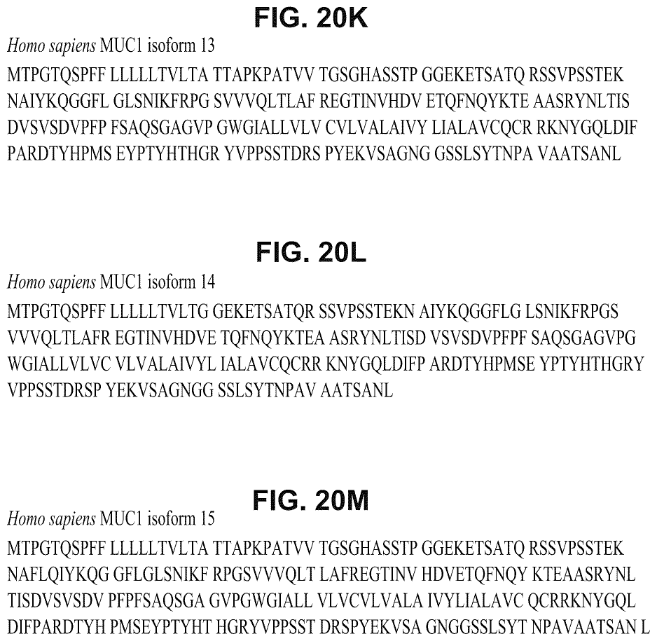

In some cases, a pMHC polypeptide comprises, as the peptide epitope, a mucin-1 (MUC-1) peptide. In some cases, a suitable MUC1 peptide is a peptide of from 4-20 aa, e.g., 6-15 aa, 8-16 aa, 8-12 aa, 8-10 aa, 9-11 aa, 5-10 aa, 10-15 aa, or 15-20 aa in length, of a MUC1 polypeptide comprising an amino acid sequence having at least 85%, at least 90%, at least 95%, at least 97%, at least 98%, at least 99%, or 100%, amino acid sequence identity to the MUC1 amino acid sequence depicted in any one of FIGS. 20A-20M.

Examples of MUC1 peptide epitopes suitable for inclusion in a pMHC polypeptide are described in published PCT application WO 2022/197970 (Cue Biopharma, Inc.), the disclosure of which as it pertains to MUC1 peptide epitopes is expressly incorporated herein by reference, including specifically paragraphs [0089]-[0092]. Non-limiting examples of suitable MUC1 peptide epitopes include: i) STAPPAHGV (SEQ ID NO:266); ii) STAPPVHNV (SEQ ID NO:267); iii) SLAPPVHNV (SEQ ID NO:268); iv) SLAPPAHGV (SEQ ID NO:269); v) SAPDTRPAP (SEQ ID NO:270); vi) VTSAPDTRPAPGSTAPPAHG (SEQ ID NO: 271); vii) PDTRPAPGSTAPPAHGVTSA (SEQ ID NO:272); and viii) LLLLTVLTV (SEQ ID NO:273).

As one example, a pMHC polypeptide comprising the MUC1 peptide epitope STAPPAHGV (SEQ ID NO:274) presents an HLA-A A*1101-restricted epitope. As another example, pMHC polypeptides comprising the MUC1 peptide epitopes STAPPAHGV (SEQ ID NO:275), STAPPVHNV (SEQ ID NO:276), SLAPPVHNV (SEQ ID NO:277), and SLAPPAHGV (SEQ ID NO:278) present HLA-A A*0201 restricted epitopes.

### MAGE-A4 Peptides

In some cases, a pMHC polypeptide comprises, as the peptide epitope, a melanoma-associated antigen 4 (MAGE-A4) peptide. In some cases, a suitable MAGE-A4 peptide is a peptide fragment of from 4-20 aa, e.g., 6-15 aa, 8-12 aa, 8-10 aa, 9-11 aa, 5-10 aa, 10-15 aa, or 15-20 aa in length of a MAGE-A4 polypeptide comprising an amino acid sequence having at least 85%, at least 90%, at least 95%, at least 97%, at least 98%, at least 99%, or 100%, amino acid sequence identity to the following Homo sapiens MAGE-A4 amino acid sequence:

(SEQ ID NO: 279)
MSSEQKSQHC KPEEGVEAQE EALGLVGAQA PTTEEQEAAV
SSSSPLVPGT LEEVPAAESA GPPQSPQGAS ALPTTISFTC
WRQPNEGSSS QEEEGPSTSP DAESLFREAL SNKVDELAHF
LLRKYRAKEL VTKAEMLERV IKNYKRCFPV IFGKASESLK
MIFGIDVKEV DPASNTYTLV TCLGLSYDGL LGNNQIFPKT
GLLIIVLGTI AMEGDSASEE EIWEELGVMG VYDGREHTVY
GEPRKLLTQD WVQENYLEYR QVPGSNPARY EFLWGPRALA
ETSYVKVLEH VVRVNARVRI AYPSLREAAL LEEEEGV.

Examples of MAGE-A4 peptide epitopes suitable for inclusion in a pMHC polypeptide are described in published PCT application WO 2022/197970 (Cue Biopharma, Inc.), the disclosure of which as it pertains to MAGE-A4 peptide epitopes is expressly incorporated herein by reference, including specifically paragraphs [0093]-[0096]. For example, suitable MAGE-A4 peptide epitopes include GVYDGREHTV (SEQ ID NO:280), NYKRCFPVI (SEQ ID NO:281), EVDPASNTY (SEQ ID NO:282), SESLKMIF (SEQ ID NO:283), and SESLICMIF (SEQ ID NO:284); and has a length of 9 amino acids. As another example, a pMHC polypeptide comprising the peptide epitope GVYDGREHTV (SEQ ID NO:285) presents an HLA-A02 restricted epitope.

### NY-ESO-1 Peptides

In some cases, a pMHC polypeptide comprises, as the peptide epitope, a Cancer/Testis Antigen-1 (CTAG1B) peptide. CTAG1B is also known as LAGE2, LAGE3, or NY-ESO-1 (New York Esophageal Squamous Cell Carcinoma 1). Thus, in some cases, a pMHC polypeptide comprises, as the peptide epitope, an NY-ESO-1 peptide. In some cases, a suitable NY-ESO-1 peptide is a peptide fragment of from 4-20 aa, e.g., 6-15 aa, 8-16 aa, 8-12 aa, 8-10 aa, 9-11 aa, 5-10 aa, 10-15 aa, or 15-20 aa in length of an NY-ESO-1 polypeptide comprising an amino acid sequence having at least 85%, at least 90%, at least 95%, at least 97%, at least 98%, at least 99%, or 100%, amino acid sequence identity to the following NY-ESO-1 amino acid sequence:

(SEQ ID NO: 286)
MQAEGRGTGG STGDADGPGG PGIPDGPGGN AGGPGEAGAT
GGRGPRGAGA ARASGPGGGA PRGPHGGAAS GLNGCCRCGA
RGPESRLLEF YLAMPFATPM EAELARRSLA QDAPPLPVPG
VLLKEFTVSG NILTIRLTAA DHRQLQLSIS SCLQQLSLLM
WITQCFLPVF LAQPPSGQRR.

Examples of NY-ESO-1 peptide epitopes suitable for inclusion in a pMHC polypeptide are described in published PCT application WO 2022/197970 (Cue Biopharma, Inc.), the disclosure of which as it pertains to specific NY-ESO-1 peptide epitopes is expressly incorporated herein by reference, including specifically paragraphs [0097]-[0098]. Such peptide epitopes include, e.g., SLLMWITQCFL (SEQ ID NO:287), SLLMWITQC (SEQ ID NO:288), QLSLLMWIT SEQ ID NO:289), SLLMWITQCFLPVF (SEQ ID NO:290), MLMAQEALAFL (SEQ ID NO:291), YLAMPFATPME (SEQ ID NO:292), ASGPGGGAPR (SEQ ID NO:293), LAAQERRVPR (SEQ ID NO:294), TVSGNILTIR (SEQ ID NO:295), APRGPHGGAASGL (SEQ ID NO:296), MPFATPMEAEL (SEQ ID NO:297), KEFTVSGNLLTI (SEQ ID NO:298), MPFATPMEA (SEQ ID NO:299), FATPMEAELAR (SEQ ID NO:300), LAMPFATPM (SEQ ID NO:301), and ARGPESRLL (SEQ ID NO:302).

### Survivin Peptides

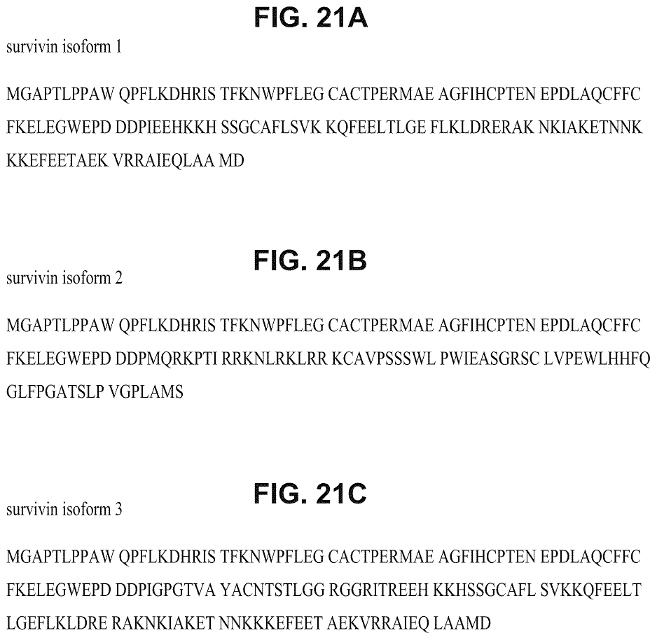

In some cases, a pMHC polypeptide comprises, as the peptide epitope, a survivin peptide. Survivin is also known in the art as Baculoviral IAP Repeat Containing 5 (BIRC5) and apoptosis inhibitor 4 (IAP4). In some cases, a suitable survivin peptide is a peptide fragment of from 4-20 aa, e.g., 6-15 aa, 8-16 aa, 8-12 aa, 8-10 aa, 9-11 aa, 5-10 aa, 10-15 aa, or 15-20 aa in length, of a survivin polypeptide comprising an amino acid sequence having at least 85%, at least 90%, at least 95%, at least 97%, at least 98%, at least 99%, or 100%, amino acid sequence identity to the amino acid sequence depicted in any one of FIGS. 21A-21C.

Examples of survivin peptide epitopes include: ELTLGEFLKL (SEQ ID NO:303); TLGEFLKLDRERAKN (SEQ ID NO:304); a peptide epitope comprising QMFFCF (SEQ ID NO:305) and having a length of from 6 to 10 amino acids; DLAQMFFCFKELEGW (SEQ ID NO:306); AQMFFCFKEL (SEQ ID NO:307); and QMFFCFKEL (SEQ ID NO:308).

### Mesothelin Peptides

In some cases, a pMHC polypeptide comprises, as the peptide epitope, a mesothelin peptide. In some cases, a suitable mesothelin peptide is a peptide fragment of from 4-20 aa, e.g., 6-15 aa, 8-16 aa, 8-12 aa, 8-10 aa, 9-11 aa, 5-10 aa, 10-15 aa, or 15-20 aa in length of a mesothelin polypeptide comprising an amino acid sequence having at least 85%, at least 90%, at least 95%, at least 97%, at least 98%, at least 99%, or 100%, amino acid sequence identity to the following mesothelin amino acid sequence:

(SEQ ID NO: 309)
MALPTARPLL GSCGTPALGS LLFLLFSLGW VQPSRTLAGE
TGQEAAPLDG VLANPPNISS LSPRQLLGFP CAEVSGLSTE
RVRELAVALA QKNVKLSTEQ LRCLAHRLSE PPEDLDALPL
DLLLFLNPDA FSGPQACTRF FSRITKANVD LLPRGAPERQ
RLLPAALACW GVRGSLLSEA DVRALGGLAC DLPGRFVAES
AEVLLPRLVS CPGPLDQDQQ EAARAALQGG GPPYGPPSTW
SVSTMDALRG LLPVLGQPII RSIPQGIVAA WRQRSSRDPS
WRQPERTILR PRFRREVEKT ACPSGKKARE IDESLIFYKK
WELEACVDAA LLATQMDRVN AIPFTYEQLD VLKHKLDELY
PQGYPESVIQ HLGYLFLKMS PEDIRKWNVT SLETLKALLE
VNKGHEMSPQ VATLIDRFVK GRGQLDKDTL DTLTAFYPGY
LCSLSPEELS SVPPSSIWAV RPQDLDTCDP RQLDVLYPKA
RLAFQNMNGS EYFVKIQSFL GGAPTEDLKA LSQQNVSMDL
ATFMKLRTDA VLPLTVAEVQ KLLGPHVEGL KAEERHRPVR
DWILRQRQDD LDTLGLGLQG GIPNGYLVLD LSMQEALSGT
PCLLGPGPVL TVLALLLAST LA

Non-limiting examples of suitable mesothelin peptide epitopes include the following: Mesothelin A2 (20-28) peptide SLLFLLFSL (SEQ ID NO:310); mesothelin A2 (530-538) peptide VLPLTVAEV (SEQ ID NO:311); mesothelin A3 (83-91) peptide ELAVALAQK (SEQ ID NO:312); mesothelin A3 (225-233) peptide ALQGGGPPY (SEQ ID NO:313); mesothelin A24 (435-443) peptide FYPGYLCSL (SEQ ID NO:314); and mesothelin A24 (475-483) peptide LYPKARLAF (SEQ ID NO:315).

### MART-1 Peptides

In some cases, a pMHC polypeptide comprises, as the peptide epitope, a Melanoma Antigen Recognized by T cells-1 (MART-1) peptide. In some cases, a suitable MART-1 peptide is a peptide fragment of from 4-20 aa, e.g., 6-15 aa, 8-12 aa, 8-10 aa, 9-11 aa, 5-10 aa, 10-15 aa, or 15-20 aa in length of a MART-1 polypeptide comprising an amino acid sequence having at least 85%, at least 90%, at least 95%, at least 97%, at least 98%, at least 99%, or 100%, amino acid sequence identity to the following MART-1 amino acid sequence: MPREDAHFIY GYPKKGHGHS YTTAEEAAGI GILTVILGVL LLIGCWYCRR RNGYRALMDK SLHVGTQCAL TRRCPQEGFD HRDSKVSLQE KNCEPVVPNA PPAYEKLSAE QSPPPYSP (SEQ ID NO:316). As one non-limiting example, a suitable MART-1 peptide epitope has the following amino acid sequence: ELAGIGILTV (SEQ ID NO:317); and has a length of 10 amino acids.

### PRAME Peptides

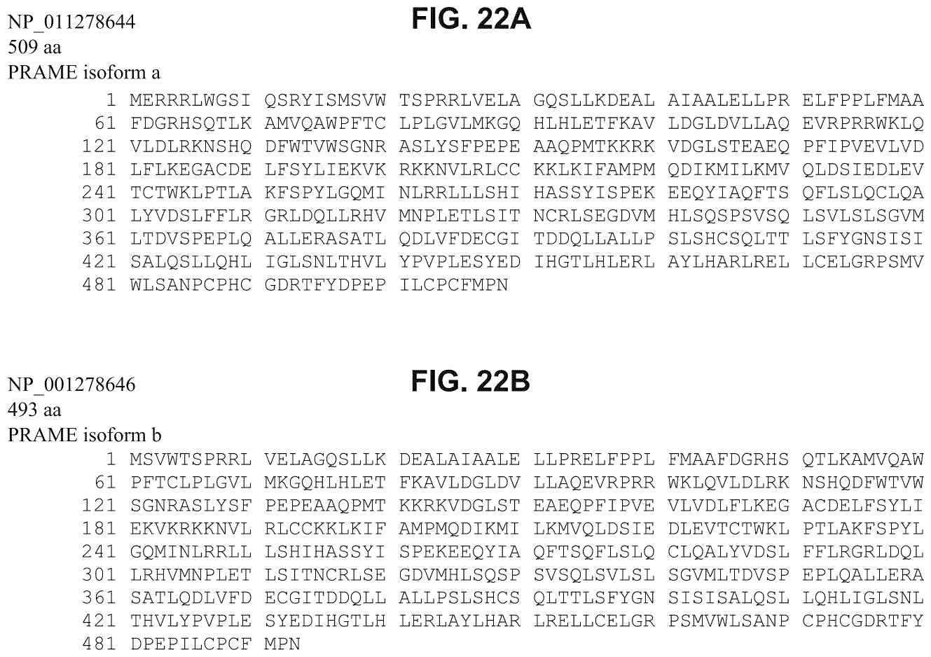

In some cases, a pMHC polypeptide comprises, as the peptide epitope, a Preferentially Expressed Antigen of Melanoma (PRAME) polypeptide. PRAME polypeptides are known in the art; see, e.g., Kaczorowski et al. (2022) Am. J. Surg. Pathol. 46:1467. PRAME epitopes are known in the art; see, e.g., Kessler et al. (2001) J. Exp. Med. 193:73. In some cases, a suitable PRAME peptide is a peptide fragment of from 4-20 aa, e.g., 6-15 aa, 8-16 aa, 8-12 aa, 8-10 aa, 9-11 aa, 5-10 aa, 10-15 aa, or 15-20 aa in length of a PRAME polypeptide comprising an amino acid sequence having at least 85%, at least 90%, at least 95%, at least 97%, at least 98%, at least 99%, or 100%, amino acid sequence identity to the PRAME amino acid sequence depicted in FIG. 22A. In some cases, a suitable PRAME peptide epitope is a peptide fragment of from 4-20 aa, e.g., 6-15 aa, 8-12 aa, 8-10 aa, 9-11 aa, 5-10 aa, 10-15 aa, or 15-20 aa in length of a PRAME polypeptide comprising an amino acid sequence having at least 85%, at least 90%, at least 95%, at least 97%, at least 98%, at least 99%, or 100%, amino acid sequence identity to the PRAME amino acid sequence depicted in FIG. 22B.

Non-limiting examples of suitable PRAME peptide epitopes include, e.g., VLDGLDVLL (SEQ ID NO:318); a PRA¹⁰⁰⁻¹⁰⁸ peptide); SLYSFPEPEA (SEQ TD NO:319); a PRA¹⁴²⁻¹⁵¹ peptide); ALYVDSLFFL (SEQ ID NO:320); a PRA³⁰⁰⁻³⁰⁹ peptide); and SLLQHLIGL (SEQ ID NO:321); a PRA⁴²⁵⁻⁴³³ peptide).

### Viral Peptide Epitopes

In some cases, a pMHC polypeptide comprises, as the peptide epitope, a viral peptide epitope. Examples of viral peptide epitopes suitable for inclusion in a pMHC polypeptide are described in published PCT application WO 2022/197970 (Cue Biopharma, Inc.), the disclosure of which as it pertains to viral peptides is expressly incorporated herein by reference, including specifically paragraphs [00107]-[00117]. Examples of viral peptide epitopes include peptide epitopes of influenza virus Matrix Protein (M1) peptide, e.g., M1 (58-66), having the amino acid sequence GILGFVFTL (SEQ ID NO:322), peptide epitopes of an EBV nuclease antigen 3B (EBNA3B), e.g., IVTDFSVIK (SEQ ID NO:323), and AVFDRKSDAK (SEQ ID NO:324), CMV peptide epitopes, e.g., from CMV pp65 or CMV gB (glycoprotein B), and SARS-CoV-2 peptide epitopes. Numerous examples of CMV and SARS-CoV-2 peptide epitopes suitable for inclusion in a pMHC polypeptide are described in published PCT application WO 2022/197970 (Cue Biopharma, Inc.).

### HLA/Peptide Binding Assays

Whether a given peptide binds a class I HLA complex (comprising an HLA heavy chain and a β2M polypeptide), and, when bound to the HLA complex, can effectively present an epitope to a TCR, can be determined using any of a number of well-known methods. Assays include binding assays and T-cell activation assays, including cell-based binding assays, biochemical binding assays, T-cell activation assays, ELISPOT assays, cytotoxicity assays and Detection of Antigen-specific T cells with peptide-HLA tetramers. Such assays are described in the published scientific literature as well as in published PCT application WO2020132138 (Cue Biopharma, Inc.), the disclosure of which as it pertains to specific binding assays is expressly incorporated herein by reference, including specifically paragraphs [00217]-[00225].

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

### Second Peptide Linker

The second peptide linker present in a pMHC polypeptide can have a length of from about 5 amino acids to about 50 amino acids, e.g., from about 5 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids.

The second peptide linker can comprise a glycine polymer (G)ₙ, a glycine-serine polymer (including, for example, (GS)ₙ, (GSGGS)ₙ (SEQ ID NO:325), (GGGGS)n (SEQ ID NO:326), and (GGGS)ₙ (SEQ ID NO:327), where n is an integer of at least one and can be an integer from 1 to 10.

Exemplary flexible peptide linkers include, e.g., (GGGGS)n (SEQ ID NO:328); also referred to as a “G4S” linker), where n is 1, 2, 3, 4, 5, 6, 7, 8, 9, or 10. In some cases, a linker comprises the amino acid sequence (GGGGS)n (SEQ ID NO:328), where n is 1, 2, 3, 4, 5, 6, 7, 8, 9 or 10. In some cases, a linker comprises the amino acid sequence (GGGGS)n (SEQ ID NO:329), where n is 2. In some cases, a linker comprises the amino acid sequence (GGGGS)n (SEQ ID NO:330), where n is 3. In some cases, a linker comprises the amino acid sequence (GGGGS)n (SEQ ID NO:331), where n is 4. In some cases, a linker comprises the amino acid sequence (GGGGS)n (SEQ ID NO:332), where n is 7. In some cases, a linker comprises the amino acid sequence AAAGG (SEQ ID NO:333). Also suitable is a linker having the amino acid sequence AAAGG (SEQ ID NO:333).

### Multimers

A pMHC polypeptide of the present disclosure can be part of a multimer comprising two or more of the pMHC polypeptides (i.e., the pMHC polypeptide is a dimeric polypeptide). A pMHC polypeptide can be a multimeric polypeptide comprising three of the pMHC polypeptides (i.e., the pMHC polypeptide is a trimeric polypeptide). A pMHC polypeptide can be a multimeric polypeptide comprising four of the pMHC polypeptides (i.e., the pMHC polypeptide is atetrameric polypeptide). Each of the pMHC polypeptides in the multimer can comprise the same amino acid sequence, such that the multimer is a homodimer, a homotrimer, or a homotetramer.

As one non-limiting example, a pMHC polypeptide can be biotinylated; a biotinylated pMHC polypeptide can be contacted with streptavidin, to form a pMHC-biotin/streptavidin complex comprising 4 pMHC-biotin polypeptides per streptavidin.

### Non-Polypeptide Moieties

A pMHC polypeptide can comprise one or more covalently or non-covalently linked moieties, where the one or more moieties are other than a polypeptide. Suitable non-polypeptide moieties include, but are not limited to, detectable labels, insoluble supports, metals (e.g., nickel), members of specific binding pairs, lipids, nanoparticles, and polymers (other than polypeptides). Other suitable non-polypeptide moieties include, e.g., moieties for use in click chemistry (e.g., an azido moiety; an alkynyl moiety (e.g., a cyclooctynyl moiety), and a phosphino moiety). Other suitable non-polypeptide moieties include non-peptide linker moieties.

In some cases, a moiety is linked to a pMHC polypeptide directly. In some cases, a moiety is linked to a pMHC polypeptide via a linker. In some cases, the linker is a cleavable linker. Suitable linkers include, e.g., a thioether linker, a maleimidocaproyl linker, an acid-sensitive linker, and a disulfide linker.

In some cases, the moiety linked to a pMHC polypeptide is a detectable label to permit detection of the pMHC polypeptide. Suitable detectable labels include, e.g., a radioisotope, a fluorophore (a fluorescent label), and a chromophore.

In some cases, the detectable label is suitable for use in in vivo imaging, e.g., suitable for use in positron emission tomography (PET), single photon emission tomography (SPECT), near infrared (NIR) optical imaging, x-ray imaging, computer-assisted tomography (CAT), or magnetic resonance imaging (MRI), or other in vivo imaging method. Examples of suitable labels for in vivo imaging include gadolinium chelates (e.g., gadolinium chelates with DTPA (diethylenetriamine penta-acetic acid), DTPA-bismethylamide (BMA), DOTA (dodecane tetraacetic acid), or HP-DO3A (1,4,7-tris(carboxymethyl)-10-(2′-hydroxypropyl)-1,4,7,10-tetraazacyclododecane)), iron chelates, magnesium chelates, manganese chelates, copper chelates, chromium chelates, iodine-based materials, and radionuclides. Suitable radionuclides include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga ⁶⁸Ga, ⁶⁴Cu, ⁶⁷Cu, ⁷⁵Br, ⁷⁷Br, ⁹⁹mTc, ¹⁴C, ¹³N, ¹⁵O, ³²P, ³³P, and ¹⁸F. In some cases, the detectable label is a positron-emitting isotope such as ¹¹C, ¹³N, ¹⁵O, ¹⁸F, ⁶⁴Cu, ⁶⁸Ga, ⁷⁸Br, ⁸²Rb, ⁸⁶Y, ⁹⁰Y, ²²Na, ²⁶Al, ⁴⁰K, ⁸³Sr, ⁸⁹Zr, or ¹²⁴I.

Suitable fluorescent labels include, e.g., an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), tetramethylrhodamine (TRITC), Texas Red, Oregon Green, Pacific Blue, Pacific Green, Pacific Orange, and a quantum dots.

In some cases, the detectable label is a member of a fluorescence-emitting dye pair, such as a fluorescence resonance energy transfer (FRET) pair or a quencher/fluor pair.

Suitable members of a specific binding pair include, e.g., biotin (which can be bound by avidin, streptavidin, or neutravidin), fluorescein (which can be bound by an anti-fluorescein antibody), digoxigenin (which can be bound by an anti-digoxigenin antibody), dinitrophenyl (DNP; which can be bound by an anti-DNP antibody), and the like.

In some cases, the moiety is a biotin moiety. As used herein, the term “biotin moiety” refers to an affinity tag that includes biotin or a biotin analog such as desthiobiotin, oxybiotin, 2′-iminobiotin, diaminobiotin, biotin sulfoxide, biocytin, etc. A biotin moiety may also include a linker, e.g., N-hydroxysulfosuccinimide (NHS)-biotin, —NHS-LC-biotin, (“succinimidyl-6-(biotinamindo)hexanoate”) -LC-biotin, -LC-LC-Biotin, -SLC-biotin, or -PEGₙ¹-biotin where n¹ is 3-12.

Suitable lipids include, e.g., 1,2-dioleoyl-3-trimethylammonium-propane (DOTAP) or 1,2-ditetradecanoyl-sn-glycero-3-phosphocholine (DMPC), and the like.

Suitable non-polypeptide polymers include poly(ethylene glycol) (PEG); dextran; polyimines; and the like. The term “PEG” as used herein means any polyethylene glycol or other polyalkylene ether polymer. In some cases, PEG is an optionally substituted linear or branched polymer of ethylene glycol or ethylene oxide. In some cases, PEG is unsubstituted. In some cases, the PEG is substituted, e.g., by one or more alkyl, alkoxy, acyl, hydroxy, or aryl groups. In some cases, “PEG” includes PEG copolymers such as PEG-polyurethane or PEG-polypropylene. In some cases, the PEG has a molecular weight of from about 130 to about 50,000, from about 150 to about 30,000, from about 150 to about 20,000, from about 150 to about 15,000, from about 150 to about 10,000, from about 150 to about 6,000, from about 150 to about 5,000, from about 150 to about 4,000, from about 150 to about 3,000, from about 300 to about 3,000, from about 1,000 to about 3,000, or from about 1,500 to about 2,500.

In some cases, the moiety is a lipid nanoparticle. For example a lipid nanoparticle can comprise one or more lipids such as 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA).

Suitable non-peptide linker moieties include, alkyl, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), epidithio (—S—S—), carbonyldioxy (—O—CO—O—), alkyldioxy (—O—(CH₂)n-O—), epoxyimino (—O—NH—), epimino (—NH—), carbonyl (—CO—), etc. Suitable non-peptide linker moieties include, e.g., poly(ethylene glycol) unit(s) (e.g., —(CH₂—CH₂—O)—); ethers, thioethers, amines, alkyls (e.g., (C₁-C₁₂)alkyl), which may be straight or branched, e.g., methyl, ethyl, n-propyl, 1-methylethyl (iso-propyl), n-butyl, n-pentyl, 1,1-dimethylethyl (t-butyl), and the like. A suitable linker moiety is dibenzocyclooctyne (DBCO).

In some cases, a pMHC polypeptide is covalently or non-covalently linked to an insoluble support. An insoluble support (also referred to as a “solid support”), used in its conventional sense, refers to a surface upon which another element, such as functional groups or molecules, may be adhered. A solid support may be configured as a substrate. Suitable solid supports can have a variety of shapes, sizes, forms, and compositions and can be derived from naturally occurring materials, naturally occurring materials that have been synthetically modified, or synthetic materials. Non-limiting examples of suitable support materials include, but are not limited to, nitrocellulose, glasses, silicas, teflons, metals (for example, gold, platinum, and the like), and other materials. Non-limiting examples of solid support substrates include, but are not limited to polymeric materials, including plastics (for example, polytetrafluoroethylene, polypropylene, polystyrene, polycarbonate, and blends thereof, and the like); polysaccharides such as agarose and dextran; polyacrylamides; polystyrenes; polyvinyl alcohols; copolymers of hydroxyethyl methacrylate and methyl methacrylate; and the like. A solid support may be homogenous or a composite structure of two or more different materials, e.g., where the solid support includes a first base material that is coated on a surface with one or more additional different coating materials. Suitable insoluble supports include, e.g., a bead, such as a magnetic bead.

Libraries of pMHC Polypeptides

The present disclosure provides a library of pMHC polypeptides of this disclosure, where at least two of the pMHC polypeptides in the library comprise different peptide epitopes. A library can comprise at least 2, at least 5, at least 10, at least 100, at least 1,000, at least 10,000, at least 100,000, at least 10⁶, at least 10⁷, at least 10⁸, or at least 10⁹, or more than 10⁹, members. A library of pMHC polypeptides can have a complexity of greater than 2. The term “complexity” refers the total number of different sequences in a library. Thus, e.g., if a library has two pMHC polypeptides that differ from one another in amino acid sequence (e.g., the two pMHC polypeptides have different peptide epitopes), then library has a complexity of 2. In some cases, a library has a complexity of at least 4, at least 8, at least 16, at least 100, at least 1,000, at least 10,000, at least 100,000, at least 10⁶ (1M (1 million)), at least 10⁷ (10M), at least 10⁸ (100M) or at least 109 (1B (1 billion)) or more. For example, in some cases, the pMHC polypeptides in a library have at least 4, at least 8, at least 16, at least 100, at least 1,000, at least 10,000, at least 100,000, at least 1M, at least 10M, at least 100M, or at least 1B, different peptide epitopes.

In some cases, a pMHC polypeptide in a library can comprise a covalently linked nucleic acid that comprises a barcode sequence; such a library is referred to as a “barcoded pMHC library.” In some cases, a pMHC polypeptide in a library is associated with a polypeptide that comprises a covalently linked nucleic acid that comprises a barcode sequence; such a library is also referred to as a “barcoded pMHC library.” For example, a streptavidin polypeptide can be conjugated to a nucleic acid comprising a barcode sequence, forming a barcoded streptavidin polypeptide; and a pMHC polypeptide that is biotinylated can be complexed with the barcoded streptavidin polypeptide.

Conjugation of a nucleic acid to a polypeptide can be achieved using any known method. For example, a nucleic acid can be modified to include a 5′-NH₂ group; and a target polypeptide can be modified to include a C-terminal Cys residue. The 5′-NH₂ group of the modified nucleic acid can be reacted with the C-terminal Cys residue on the modified target polypeptide, to form a polypeptide-nucleic acid conjugate. See, e.g., Dahotre et al. (2019) Anal. Chem. 91:2695. As another example, a protein-nucleic acid conjugate can be generated using click chemistry. For example, a target protein can be modified to contain a dibenzocyclooctyne (DBCO) moiety, and linked to an azide-modified nucleic acid, via a strain-promoted azidealkyne cycloaddition (SPAAC) reaction. See, e.g., Gong et al. (2016) Bioconjugate Chem. 27:217. A DBCO moiety can be cross-linked to an amine side chain in the target polypeptide via N-hydroxysuccinimide (NHS) functionalization. A nucleic acid can be modified to include an azide group by functionalizing the nucleic acid with 3-azidopropionic acid sulfo NHS ester. See, e.g., Wiener et al. (2020) Scientific Reports 10:1457.

The term “barcode sequence”, as used herein, refers to a unique sequence of nucleotides that can be used to identify and/or track the source of a polynucleotide. Nucleic acids comprising barcode sequences may vary widely in length and composition. A nucleic acid comprising a barcode sequence can have a length in a range of from 4 to 60 nucleotides, or from 6 to 50 nucleotides, or from 8 to 40 nucleotides. The barcode sequence itself can have a length of from 4 to 60 nucleotides, or from 6 to 50 nucleotides, or from 8 to 40 nucleotides.

In some cases, a pMHC polypeptide in a library is biotinylated. In some cases, a pMHC polypeptide in a barcoded pMHC library is biotinylated. In some cases, a pMHC library comprises tetrameric pMHC complexes comprising: i) a biotinylated pMHC polypeptide; and ii) streptavidin. In some cases, a barcoded pMHC library is a tetrameric barcoded pMHC library that comprises tetrameric barcoded pMHC complexes comprising: i) a biotinylated and barcoded pMHC polypeptide; and ii) streptavidin. In some cases, the streptavidin comprises a detectable label such as a fluorescent label. In some cases, a pMHC polypeptide in a library is modified to include a DBCO moiety, for reaction with a nucleic acid barcode that is modified to include an azide moiety.

A pMHC library is useful for detecting antigen-specific T cells. For example, a pMHC library can be used to probe the TCR repertoire in diverse, polyclonal population of T cells. The polyclonal population of T cells can be human T cells. The polyclonal population of T cells can be obtained from, e.g., patient peripheral blood mononuclear cells (PBMCs). In some cases, a detection method comprises: a) contacting a polyclonal population of T cells (e.g., contacting patient PBMCs) with a tetrameric barcoded pMHC library comprising: i) a biotinylated and barcoded pMHC polypeptide; and ii) streptavidin, where the streptavidin comprises a fluorescent label, forming a mixed population comprising T cell-pMHC complexes; b) separating unbound cells (cells not complexed with pMHC polypeptides) from cell-pMHC complexes, where such separation can be carried out using cell sorting (e.g., fluorescence activated cell sorting (FACS)); c) determining the TCR sequence of an individual T cell in the mixed population of T-cells; and d) determining the nucleotide sequence of the barcode of a barcoded pMHC polypeptide bound to the individual T cell, thereby determining the peptide specificity of the TCR in the individual T cell.

### Compositions

The present disclosure provides compositions, including pharmaceutical compositions, comprising a pMHC polypeptide, or a nucleic acid or recombinant expression vector comprising a nucleotide sequence encoding the pMHC polypeptide, or a cell (e.g., a B cell or other blood cell) comprising such nucleic acids or recombinant expression vector, together with one or more pharmaceutically acceptable additives, a variety of which are known in the art and need not be discussed in detail herein. See, for example, the ninth (or latest) edition of Sheskey et al., “Handbook of Pharmaceutical Excipients” (2020), and/or the 23^{rd} (or latest) edition of “Remington: The Science and Practice of Pharmacy”, 23rd Ed. (2020).

In some cases, a treatment method comprises administering to an individual in need thereof one or more nucleic acids or recombinant expression vectors comprising nucleotide sequences encoding a pMHC polypeptide. In some cases, the one or more nucleic acids or recombinant expression vectors are present in cells (e.g., B cells or other blood cells) that are administered to the individual, which cells are then capable of producing the pMHC polypeptide in vivo.

In some cases, a subject pharmaceutical composition will be suitable for administration to a subject, e.g., will be sterile. For example, in some cases, a subject pharmaceutical composition will be suitable for administration to a human subject, e.g., where the composition is sterile and is substantially free of detectable pyrogens and/or other toxins, or where such detectable pyrogens and/or other toxins are present at a level within acceptable limits set by an applicable regulatory agency, e.g., the USF&DA. For example, compositions may include aqueous solution, powder form, granules, tablets, pills, suppositories, capsules, suspensions, sprays, and the like. The composition may be formulated according to the various routes of administration described below.

Where a pMHC polypeptide is administered as an injectable (e.g. subcutaneously, intraperitoneally, intramuscularly, and/or intravenously) directly into a tissue, a formulation can be provided as a ready-to-use dosage form, or as non-aqueous form (e.g., a reconstitutable storage-stable powder) or aqueous form, such as liquid composed of pharmaceutically acceptable carriers and excipients. The protein-containing formulations may also be provided so as to enhance serum half-life of the pMHC polypeptide following administration. For example, the pMHC polypeptide may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

The concentration of a pMHC polypeptide in a formulation can vary widely (e.g., from less than about 0.10%, usually at or at least about 2% to as much as 20% to 50% or more by weight) and will usually be selected primarily based on fluid volumes, viscosities, and patient-based factors in accordance with the particular mode of administration selected and the patient's needs.

In some cases, a pMHC polypeptide is present in a liquid composition. Thus, this disclosure provides compositions (e.g., liquid compositions, including pharmaceutical compositions) comprising a pMHC polypeptide. In some cases, a composition comprises: a) a pMHC polypeptide; and b) saline (e.g., 0.9% NaCl). In some cases, the composition is sterile. In some cases, the composition is suitable for administration to a human subject, e.g., where the composition is sterile and is free of detectable pyrogens and/or other toxins, or where such detectable pyrogens and/or other toxins are present at a level within acceptable limits set by an applicable regulatory agency, e.g., the U.S. Food and Drug Administration (USF&DA). Thus, this disclosure provides a composition comprising: a) a pMHC polypeptide; and b) saline (e.g., 0.9% NaCl), where the composition is sterile and is free of detectable pyrogens and/or other toxins, or where such detectable pyrogens and/or other toxins are present at a level within acceptable limits set by an applicable regulatory agency, e.g., the USF&DA.

### Nucleic Acids, Expression Vectors, and Host Cells

The present disclosure provides a nucleic acid comprising a nucleotide sequence encoding a pMHC polypeptide of the present disclosure. In some cases, the nucleotide sequence encoding the pMHC polypeptide is operably linked to one or more transcriptional control elements (e.g., a promoter). In some cases, the transcriptional control element is a promoter that is functional in a eukaryotic cell. In some cases, the nucleic acid is present in a recombinant expression vector.

The present disclosure thus provides recombinant expression vectors comprising nucleic acids encoding a pMHC polypeptide. In some cases, the recombinant expression vector is a non-viral vector. In some cases, the recombinant expression vector is a viral construct, e.g., a recombinant adeno-associated virus construct, a recombinant adenoviral construct, a recombinant lentiviral construct, a recombinant retroviral construct, a non-integrating viral vector, etc.

The present disclosure further provides a genetically modified host cell, where the host cell is genetically modified with a nucleic acid or expression vector as described above.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

A pMHC polypeptide of the present disclosure can be generated by culturing a genetically modified host cell of the present disclosure in a suitable culture medium in vitro, where such culturing results in production of the pMHC polypeptide. For example, a mammalian host cell (e.g., a CHO cell) can be genetically modified with a recombinant expression vector comprising a nucleotide sequence encoding a pMHC polypeptide; and the genetically modified mammalian host cell can be cultured in vitro in a suitable culture medium, such that the genetically modified mammalian host cell produces the pMHC polypeptide. The pMHC polypeptide can be isolated, e.g., from the culture medium in which the genetically modified mammalian host cell is cultured and/or from a cell lysate of the genetically modified mammalian host cell. The pMHC polypeptide can be isolated using any of a variety of well-established methods including, e.g., affinity chromatography, and the like.

In some cases, the host cell is a B cell or other blood cell.

### Detection Methods

A pMHC polypeptide is useful for detecting the presence of a T cell that comprises a TCR that specifically binds the pMHC. Such a method is useful in, e.g., a diagnostic setting. In some cases, the pMHC polypeptide comprises a detectable label, e.g., a radiolabel, a fluorescent label, or the like.

In some cases, a detection method of the present disclosure comprises: a) contacting a T cell with a pMHC polypeptide, or a library of pMHC polypeptides (as discussed above), forming a pMHC polypeptide-T cell complex; and b) detecting the pMHC polypeptide-T cell complex. In some cases, the pMHC polypeptide is a tetrameric pMHC polypeptide comprising: i) a biotinylated pMHC polypeptide; and ii) avidin or streptavidin, where the avidin or streptavidin comprises a detectable label (e.g., a fluorescent label).

In some cases, the T cell being detected is present in a sample comprising a plurality of T cells. For example, a T cell being detected can be present in a sample comprising from 10 to 10⁹ T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹, T cells.

The T cell being detected can be present in a mixed cell population, e.g., PBMCs. In some cases, the mixed cell population is obtained from an individual before the individual is treated for a cancer. In some cases, the mixed cell population is obtained from an individual after the individual has been treated for the cancer. In some cases, the detection method provides for quantitation of the number of T cells specific for a given peptide epitope. In some cases, the number of T cells specific for a given peptide epitope is determined in an individual before and after treatment for a cancer.

### Methods of Inducing an Immune Response

The present disclosure provides a method of inducing an immune response in an individual to a pMHC polypeptide, the method comprising administering to the individual an effective amount of a pMHC polypeptide. In some cases, the method comprises administering to the individual a composition comprising: a) an effective amount of a pMHC polypeptide; and b) an adjuvant.

In some cases, the immune response comprises production of antibodies that bind to the epitope presented by the pMHC polypeptide. In some cases, the immune response comprises production of CD4⁺ T cells that comprise a TCR that binds to the pMHC polypeptide. In some cases, the immune response comprises production of CD8⁺ T cells that comprise a TCR that binds to the pMHC polypeptide. In some cases, the immune response comprises an antibody response and a T cell response.

A pMHC polypeptide, or a composition comprising a pMHC polypeptide and an adjuvant, can be administered to an individual in need thereof via any route of administration, including, e.g., intramuscular, mucosal, intravenous, subcutaneous, intranasal, and other routes of administration.

Molecules Comprising a pMHC Polypeptide

The present disclosure provides molecules (referred to herein as “pMHC complexes”) comprising: a) a pMHC polypeptide; and b) one or more components, where the one or more components comprises one or more than one of: i) a polypeptide component comprising one or more heterologous polypeptides, i.e., polypeptides that are not naturally found joined to an MHC class I heavy chain polypeptide; ii) a nucleic acid component comprising one or more nucleic acids, and (iii) a component other than a polypeptide or nucleic acid component, e.g., a drug or other active agent (other than a polypeptide or a component comprising one or more nucleic acids) that is conjugated to the pMHC polypeptide or pMHC complex. In some cases, the one or more heterologous polypeptides is not an immunomodulatory polypeptide.

In some cases, the heterologous polypeptide component is a heterologous fusion partner, i.e., a heterologous polypeptide that is produced recombinantly as part of the same polypeptide chain with the pMHC polypeptide. The heterologous fusion partner is fused to the C-terminus of the MHC class I heavy chain polypeptide of the pMHC polypeptide, either directly or through a peptide linker. A pMHC complex that comprises a heterologous fusion partner is referred to herein as a “pMHC fusion molecule”. Such heterologous fusion partners may provide one or more functions. For example, the heterologous fusion partner can comprise a targeting polypeptide that binds a target antigen and/or an Ig Fc polypeptide that provides the pMHC complex with greater half-life, manufacturability and/or stability. Where the polypeptide comprises multiple heterologous polypeptides, peptide linkers may be interposed between the heterologous polypeptides.

In some cases, the pMHC complex comprises a polypeptide component comprising one or more polypeptides that is/are not part of a fusion protein with the pMHC polypeptide, but rather is/are conjugated to the pMHC polypeptide by methods known in the art.

In some cases, the pMHC complex comprises: a) a pMHC polypeptide; and b) a PEG moiety, where suitable PEG moieties are as described above.

In some cases, the pMHC complex comprises: a) a pMHC polypeptide; and b) an amphiphilic moiety (e.g., an amphiphilic tail). The amphiphilic tail can allow the pMHC complex to be embedded in a lipid nanoparticle.

In some cases, the pMHC complex comprises: a) a pMHC polypeptide; and b) a moiety that increases the in vitro half-life of the pMHC complex, compared to the in vitro half-life of a pMHC polypeptide that does not comprise the moiety.

In some cases, the pMHC complex comprises: a) a pMHC polypeptide; and b) an attachment moiety that allows for attachment of a payload such as a nucleic acid.

In some cases, the pMHC complex comprises: a) a pMHC polypeptide; and b) a cytotoxic molecule, where suitable cytotoxic molecules include, e.g., cancer chemotherapeutic agents.

In some cases, the pMHC complex comprises: a) a pMHC polypeptide; and b) a moiety that provides for detection. Suitable detectable moieties include, but are not limited to, fluorescent proteins, epitope tags, fluorescent dyes, radiolabels, and the like.

In some cases, the pMHC complex comprises a) a pMHC polypeptide, and b) a nucleic acid component comprising one or more nucleic acids.

In some cases, the pMHC complex comprises both (i) a polypeptide component comprising one or more heterologous polypeptides (e.g., a heterologous fusion partner), and (ii) a nucleic acid component comprising one or more nucleic acids.

In some cases, the pMHC complex comprises both (i) a polypeptide component comprising one or more heterologous polypeptides, and (ii) an additional component (e.g., a drug or a label) that is neither a polypeptide nor a nucleic acid component.

In some cases, the pMHC complex comprises (i) a polypeptide component comprising one or more heterologous polypeptides, (ii) a nucleic acid component, and (iii) an additional component that is neither a polypeptide nor a nucleic acid component.

### Heterologous Polypeptides

Suitable heterologous polypeptides include, but are not limited to: a polypeptide that can enhance stability, manufacturability and/or in vivo half-life of the pMHC complex such as an immunoglobulin (Ig) Fc polypeptide, an albumin polypeptide, or the like; a targeting polypeptide such as an antibody or binding portion thereof that can cause the pMHC complex to bind to one or more target polypeptides such as a cancer-associated antigen, an antigen on a cell (e.g., CD19), or an antigen on a target tissue; an intracellular signaling domain; a Clustered Regularly Interspaced Short Palindromic Repeat (CRISPR)-associated (CRISPR-Cas) effector polypeptide; an XTEN (extended recombinant) polypeptide; and the like. In some cases, the heterologous polypeptide is not an immunomodulatory polypeptide.

In some cases, a pMHC complex (e.g., a pMHC fusion molecule) comprises: a) a pMHC polypeptide; and b) a targeting polypeptide such as an antibody or binding portion thereof that can cause the pMHC complex to bind to one or more target polypeptides such as a cancer-associated antigen, an antigen on a cell (e.g., CD19), or an antigen on a target tissue. In some cases, the antibody is a single-chain Fv polypeptide. In some cases, the antibody is a nanobody. In some cases, the antibody specifically binds a cancer-associated antigen. Cancer-associated antigens are known in the art and are discussed elsewhere herein.

As discussed above, in some cases the pMHC complex comprises a covalently linked moiety that is other than a polypeptide or nucleic acid component, e.g., a drug, prodrug, or other bioactive substance, or a detectible label such as those discussed above. Suitable drugs include, e.g., cancer chemotherapeutic agents such as: i) small molecule chemotherapeutics and microtubule-disrupting agents such as auristatin, a maytansinoid, and the like; ii) DNA-damaging agents such as a calicheamicin, a duocarmycin, doxorubicin, and the like. In some cases, the pMHC complex may comprise a targeting component such as an antibody polypeptide or binding portion thereof (e.g., that binds to an antigen selected from ERBB2, CD19, CD33, CD22, BCMA, or mesothelin), and a covalently linked drug.

In some cases, a pMHC complex (e.g., a pMHC fusion molecule) comprises: a) a pMHC polypeptide; and b) a polypeptide that enhances stability, manufacturability and/or in vivo half-life of the pMHC polypeptide or pMHC complex. Polypeptides that can increase the in vivo half-life of a pMHC polypeptide or pMHC complex include, e.g., an Ig Fc polypeptide, an albumin polypeptide, and the like.

In some cases, a pMHC complex (e.g., a pMHC fusion molecule) comprises: a) a pMHC polypeptide; and b) an Ig Fc polypeptide. Suitable Ig Fc polypeptides include a human IgG1 Fc, a human IgG2 Fc, a human IgG3 Fc, a human IgG4 Fc, etc., or a variant of a wild-type Ig Fc polypeptide. Variants include naturally-occurring variants, non-naturally-occurring variants, and combinations thereof. In some cases, an Ig Fc polypeptide comprises an amino acid sequence having at least 85%, at least 90%, at least 95%, at least 97%, at least 98%, at least 99%, or 100%, amino acid sequence identity to the amino acid sequence set forth in any one of FIGS. 16A-16L. In some cases, the Ig Fe employed in a pMHC complex (e.g., a pMHC fusion molecule) will comprise one or more substitutions of amino acids in the wild-type sequence, such that that Ig Fe that substantially does not induce cell lysis, e.g., through complement-dependent cytotoxicity (CDC) or antibody-dependent cell cytotoxicity (ADCC). For example, in some cases the Fe polypeptide present in a pMHC complex (e.g., a pMHC fusion molecule) comprises the amino acid sequence depicted in FIG. 16A (human IgG1 Fe), except for a substitution of L234 (L14 of the amino acid sequence depicted in FIG. 16A) with an amino acid other than leucine, or a substitution of L235 (L15 of the amino acid sequence depicted in FIG. 216A) with an amino acid other than leucine. Examples include an L234A (L14A) substitution; and an L235A (L15A) substitution.

In some cases, a pMHC complex (e.g., a pMHC fusion molecule) comprises: a) a pMHC polypeptide; and b) a CRISPR-Cas effector polypeptide. Suitable CRISPR-Cas effector polypeptides include Type II CRISPR-Cas effector polypeptides, Type III CRISPR Cas effector polypeptides, and Type VI CRISPR-Cas effector polypeptides. Examples of Type II CRISPR-Cas polypeptides include Cas9 polypeptides, e.g., Staphylococcus aureus Cas9, Streptococcus pyogenes Cas9, etc. Examples of Type VI CRISPR-Cas effector polypeptides include a Cas13a polypeptide, a Cas13b polypeptide, a Cas13c polypeptide, a Cas13d polypeptide, a Cas13e polypeptide, a Cas13f polypeptide, a Cas13X polypeptide, and a Cas13Y polypeptide. A suitable Type III CRISPR-Cas effector polypeptide is a Cas7-11 polypeptide. Suitable CRISPR-Cas effector polypeptides include fusion CRISPR-Cas effector polypeptides, e.g., comprising: i) a CRISPR-Cas effector polypeptide; and ii) a heterologous polypeptide, including, e.g., a reverse transcriptase, a cytidine deaminase, an adenosine deaminase, an adenosine deaminase acting on RNA (ADAR) family protein, and the like.

In some cases, a pMHC complex (e.g., a pMHC fusion molecule) comprises: a) a pMHC polypeptide; and b) an albumin polypeptide.

### Nucleic Acid Component

In some cases, a pMHC complex (e.g., a pMHC fusion molecule) comprises: a) a pMHC polypeptide; and b) a nucleic acid component comprising one or more nucleic acids. In some cases, the nucleic acid component comprises a nucleotide sequence that encodes a polypeptide of interest.

In some cases, the nucleic acid is DNA. In some cases, the nucleic acid is an expression vector (e.g., a recombinant expression vector). In some cases, the recombinant expression vector is a viral construct, e.g., a recombinant adeno-associated virus (AAV) construct, a recombinant adenoviral construct, and the like. Where the nucleic acid is DNA, the nucleotide sequences encoding the polypeptide of interest can be operably linked to a transcriptional control element such as a promoter. The transcriptional control element (e.g., a promoter) is one that is functional in a mammalian cell (e.g., a T cell). Suitable promoters include constitutive promoters and regulatable (e.g., inducible) promoters. Suitable promoters include cell type-specific promoters.

In some cases, the nucleic acid is an RNA. In some cases, the nucleic acid is a viral RNA construct. For example, in some cases, the RNA is a retroviral construct comprising a nucleotide sequence encoding a polypeptide of interest. As an example, in some cases, the RNA is a lentiviral construct comprising a nucleotide sequence encoding a polypeptide of interest.

In some cases, the nucleic acid component comprises an mRNA. In some cases, the mRNA covalently linked to the C-terminus of the pMHC polypeptide. The mRNA can include one or more of the following features: i) a 5′cap structure; ii) a poly(adenosine) (polyA) tail (i.e., a polyA tract at the 3′ end of the mRNA; iii) a 5′ untranslated region (5′ UTR); and iv) a 3′ untranslated region (3′ UTR). An mRNA can be produced using any known method, including, e.g., in vitro transcription. See, e.g., Van Hoecke and Roose (2019) J. Transl. Med. 17:54.

In some cases, the mRNA comprises one or more modifications. For example, the mRNA component of a chimeric molecule of the present disclosure can comprise one or more of: i) a modified base; ii) a modified sugar; and iii) a modified backbone. An mRNA comprises nucleosides. In some cases, the base of one or more nucleosides of the mRNA is modified. In some cases, the sugar of one or more nucleosides of the mRNA is modified. In some cases, both the base and the sugar of one or more nucleosides of the mRNA is modified. In some cases, the mRNA comprises a modified backbone.

Examples of suitable mRNA modifications include modified nucleic acid backbones and non-natural internucleoside linkages. Nucleic acids having modified backbones include those that retain a phosphorus atom in the backbone and those that do not have a phosphorus atom in the backbone.

Suitable modified backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable mRNAs having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium salts), mixed salts, and free acid forms can also be included.

In some cases, an mRNA comprises one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

Also suitable are mRNAs having morpholino backbone structures as described in, e.g., U.S. Pat. No. 5,034,506. For example, in some cases, an mRNA comprises a 6-membered morpholino ring in place of a ribose ring. In some cases, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

The mRNA component of a chimeric molecule of the present disclosure can include one or more substituted sugar moieties. Suitable polynucleotides comprise a sugar substituent group selected from: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides (e.g., mRNA) comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an mRNA, or a group for improving the pharmacodynamic properties of an mRNA, and other substituents having similar properties. A suitable modification includes 2′-methoxyethoxy (2′-O—CH₂ CH₂OCH₃, also known as 2′-O-(2-methoxyethyl) or 2′-MOE) i.e., an alkoxyalkoxy group. A further suitable modification includes 2′-dimethylaminooxyethoxy, i.e., a O(CH₂)2ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the mRNA, particularly the 3′ position of the sugar on the 3′ terminal nucleoside or in 2′-5′ linked mRNA and the 5′ position of 5′ terminal nucleotide. An mRNA may also have sugar mimetics such as cyclobutyl moieties in place of the pentofuranosyl sugar.

An RNA may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Additional suitable modified bases include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions are suitable base substitutions, e.g., when combined with 2′-O-methoxyethyl sugar modifications. Suitable modified nucleosides that can be incorporated in an mRNA include pseudouridine, 2-thiouridine, 5-methylpyridine, N¹-methylpseudouridine, and 5-methylcytidine.

As noted above, conjugation of a nucleic acid component to a pMHC polypeptide can be achieved using any known method. For example, a nucleic acid can be modified to include a 5′-NH₂ group; and a pMHC polypeptide can be modified to include a C-terminal Cys residue. The 5′-NH₂ group of the modified nucleic acid can be reacted with the C-terminal Cys residue on the modified pMHC polypeptide, to form a pMHC polypeptide-nucleic acid conjugate. See, e.g., Dahotre et al. (2019) Anal. Chem. 91:2695. As another example, a protein-nucleic acid conjugate can be generated using click chemistry. For example, a pMHC polypeptide can be modified to contain a dibenzocyclooctyne (DBCO) moiety, and linked to an azide-modified nucleic acid, via a strain-promoted azidealkyne cycloaddition (SPAAC) reaction. See, e.g., Gong et al. (2016) Bioconjugate Chem. 27:217. A DBCO moiety can be cross-linked to an amine side chain in the pMHC polypeptide via N-hydroxysuccinimide (NHS) functionalization. A nucleic acid can be modified to include an azide group by functionalizing the nucleic acid with-azidopropionic acid sulfo NHS ester. See, e.g., Wiener et al. (2020) Scientific Reports 10:1457.

In some cases, a fusion molecule comprises: a pMHC polypeptide; and b) a nucleic acid comprising a nucleotide sequence encoding a therapeutic polypeptide. Non-limiting examples of therapeutic polypeptides include, e.g., a chimeric antigen receptor (CAR), a cytokine, and the like.

In some cases, a pMHC complex (e.g., a pMHC fusion molecule) comprises: a) a pMHC polypeptide; and b) a CRISPR-Cas guide nucleic acid, or a nucleic acid encoding the CRISPR-Cas guide nucleic acid. A CRISPR-Cas guide nucleic acid comprises: i) a targeting segment comprising a nucleotide sequence that is complementary to a target nucleotide sequence in a target nucleic acid; and ii) a protein-binding segment that binds to a CRISPR-Cas effector polypeptide. In some cases, a pMHC complex (e.g., a pMHC fusion molecule) comprises: a) a pMHC polypeptide; b) a CRISPR-Cas effector polypeptide; and c) a CRISPR-Cas guide nucleic acid, or a nucleic acid encoding the CRISPR-Cas guide nucleic acid.

In some cases, a nucleic acid is linked to the pMHC polypeptide via a non-cleavable linker. In some cases, a nucleic acid is linked to the pMHC polypeptide via a cleavable linker. Suitable cleavable linkers include acid-labile linkers, peptidase-sensitive linkers (proteolytically cleavable linkers), photolabile linkers, dimethyl linkers, and disulfide-containing linkers. In some cases, the cleavable linker is cleaved by intracellular conditions. In some cases, the cleavable linker is cleaved by lysosomal conditions. In some cases, the cleavable linker is acid labile, e.g., is cleaved by low pH conditions (e.g., in low pH environment of the lysosome or endosome). In some cases, the linker is a proteolytically cleavable linker.

Nucleic acid components may include one or more constituents that protect the nucleic acid from being degraded in an individual (e.g., in the blood or other bodily fluid in the individual). Such constituents are well known to those skilled in the art. For example, in some cases, the component comprises one or more lipids, lipoplexes, or lipid nanoparticles.

### Nucleic Acids, Expression Vectors, Host Cells

The present disclosure provides a nucleic acid comprising a nucleotide sequence encoding a pMHC fusion molecule. In some cases, the nucleotide sequence encoding the pMHC fusion molecule is operably linked to one or more transcriptional control elements (e.g., a promoter). In some cases, the transcriptional control element is a promoter that is functional in a eukaryotic cell. In some cases, the nucleic acid is present in a recombinant expression vector.

The present disclosure thus provides recombinant expression vectors comprising nucleic acids encoding a pMHC fusion molecule. In some cases, the recombinant expression vector is a non-viral vector. In some cases, the recombinant expression vector is a viral construct, e.g., a recombinant adeno-associated virus construct, a recombinant adenoviral construct, a recombinant lentiviral construct, a recombinant retroviral construct, a non-integrating viral vector, etc.

The present disclosure further provides a genetically modified host cell, where the host cell is genetically modified with a nucleic acid or expression vector as described above.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

A pMHC fusion molecule can be generated by culturing a genetically modified host cell of this disclosure in a suitable culture medium in vitro, where such culturing results in production of the pMHC fusion molecule. For example, a mammalian host cell (e.g., a CHO cell) can be genetically modified with a recombinant expression vector comprising a nucleotide sequence encoding a pMHC fusion molecule; and the genetically modified mammalian host cell can be cultured in vitro in a suitable culture medium, such that the genetically modified mammalian host cell produces the pMHC fusion molecule. The pMHC fusion molecule can be isolated, e.g., from the culture medium in which the genetically modified mammalian host cell is cultured and/or from a cell lysate of the genetically modified mammalian host cell. The pMHC fusion molecule can be isolated using any of a variety of well-established methods including, e.g., affinity chromatography, and the like.

### Compositions

The present disclosure provides compositions, including pharmaceutical compositions, comprising a pMHC complex (e.g., a pMHC fusion molecule), or a nucleic acid or recombinant expression vector comprising a nucleotide sequence encoding the pMHC fusion molecule, or a cell (e.g., a B cell or other blood cell) comprising such nucleic acids or recombinant expression vector, together with one or more pharmaceutically acceptable additives, a variety of which are known in the art and need not be discussed in detail herein. See, for example, the ninth (or latest) edition of Sheskey et al., “Handbook of Pharmaceutical Excipients” (2020), and/or the 23^{rd} (or latest) edition of “Remington: The Science and Practice of Pharmacy”, 23rd Ed. (2020).

In some cases, a treatment method comprises administering to an individual in need thereof one or more nucleic acids or recombinant expression vectors comprising nucleotide sequences encoding a pMHC fusion molecule. In some cases, the one or more nucleic acids or recombinant expression vectors are present in cells (e.g., B cells or other blood cells) that are administered to the individual, which cells are then capable of producing the pMHC fusion molecule in vivo.

In some cases, a subject pharmaceutical composition will be suitable for administration to a subject, e.g., will be sterile. For example, in some cases, a subject pharmaceutical composition will be suitable for administration to a human subject, e.g., where the composition is sterile and is substantially free of detectable pyrogens and/or other toxins, or where such detectable pyrogens and/or other toxins are present at a level within acceptable limits set by an applicable regulatory agency, e.g., the USF&DA.

For example, compositions may include aqueous solution, powder form, granules, tablets, pills, suppositories, capsules, suspensions, sprays, and the like. The composition may be formulated according to the various routes of administration described below.

Where a pMHC complex (e.g., a pMHC fusion molecule) is administered as an injectable (e.g. subcutaneously, intraperitoneally, intramuscularly, and/or intravenously) directly into a tissue, a formulation can be provided as a ready-to-use dosage form, or as non-aqueous form (e.g. a reconstitutable storage-stable powder) or aqueous form, such as liquid composed of pharmaceutically acceptable carriers and excipients. The protein-containing formulations may also be provided so as to enhance serum half-life of the pMHC complex following administration. For example, the pMHC complex (e.g., pMHC fusion molecule) may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

The concentration of a pMHC complex such as a pMHC fusion molecule in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

In some cases, a pMHC complex is present in a liquid composition. Thus, this disclosure provides compositions (e.g., liquid compositions, including pharmaceutical compositions) comprising a pMHC complex. In some cases, a composition comprises: a) a pMHC complex such as a pMHC fusion molecule; and b) saline (e.g., 0.9% NaCl). In some cases, the composition is sterile. In some cases, the composition is suitable for administration to a human subject, e.g., where the composition is sterile and is free of detectable pyrogens and/or other toxins, or where such detectable pyrogens and/or other toxins are present at a level within acceptable limits set by an applicable regulatory agency, e.g., the USF&DA. Thus, this disclosure provides a composition comprising: a) a pMHC complex such as a pMHC fusion molecule; and b) saline (e.g., 0.9% NaCl), where the composition is sterile and is free of detectable pyrogens and/or other toxins, or where such detectable pyrogens and/or other toxins are present at a level within acceptable limits set by an applicable regulatory agency, e.g., the USF&DA.

### Delivery Methods

A pMHC complex such as a pMHC fusion molecule is useful for delivering one or more components present in the molecule to a T cell that comprises a TCR that binds the pMHC polypeptide present in the molecule. The T cell can be a CD4⁺ T cell. The T cell can be a CD8⁺ T cell. In some cases, the T cell is in vivo, and the method comprises administering the molecule to an individual.

### T-Cell Modulatory Polypeptides

The present disclosure provides a single-chain T-cell modulatory polypeptide (TMP) comprising: a) a pMHC polypeptide; b) one or more immunomodulatory polypeptides (MODs). This disclosure provides a single-chain TMP comprising: a) a pMHC polypeptide; b) one or more MODs); and c) an Ig Fc polypeptide or a non-immunoglobulin scaffold component.

Generally speaking, a TMP binds to a T cell having a co-MOD and a TCR that binds the peptide/MHC complex of the TMP with an affinity that is greater (e.g., 25% greater) than the affinity with which the same TMP binds a second T cell that has the same co-MOD but has a TCR that substantially does not bind the pMHC polypeptide.

In some cases, a TMP comprises, in order from N-terminus to C-terminus, the following components: a) pMHC polypeptide, where the pMHC polypeptide comprises, in order from N-terminus to C-terminus: i) a peptide epitope; ii) a first peptide linker comprising a Cys; iii) a β2M polypeptide; iv) a second peptide linker; and v) an MHC class I heavy chain polypeptide, where the MHC class I heavy chain polypeptide comprises a Cys at any one of amino acids 135-143, based on the numbering of the MHC class I heavy chain polypeptide depicted in FIG. 3A, and where amino acid 84, based on the numbering of the MHC class I heavy chain polypeptide depicted in FIG. 3A, is other than Cys; and b) one or more MODs.

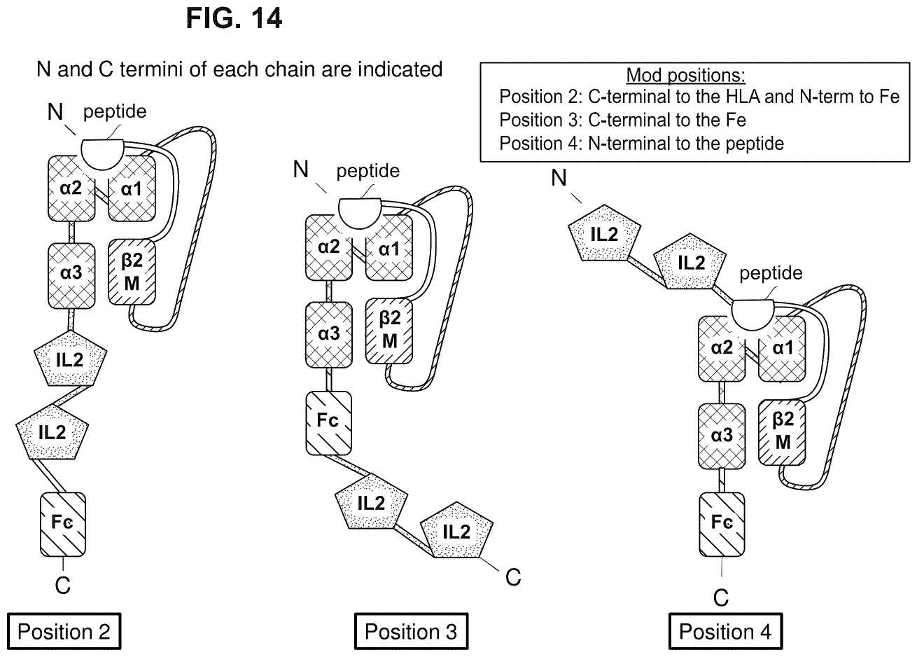

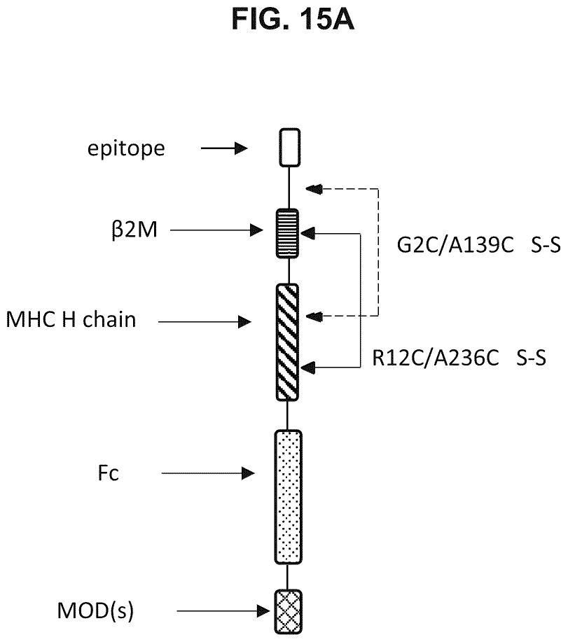

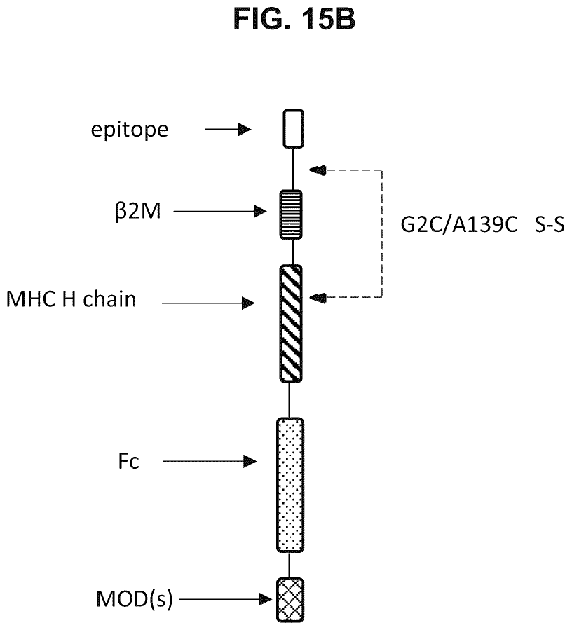

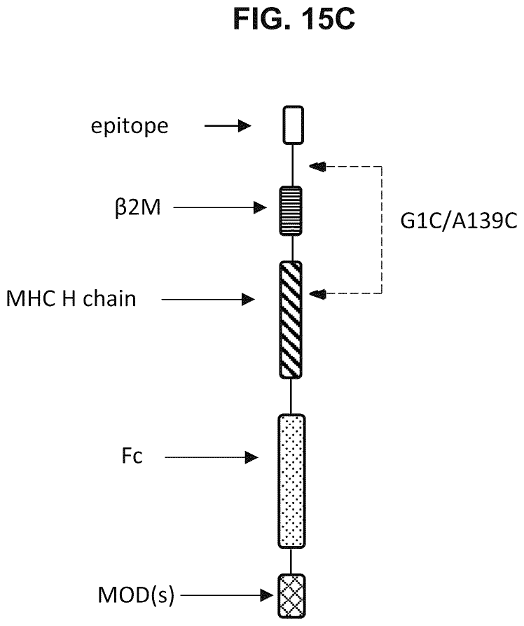

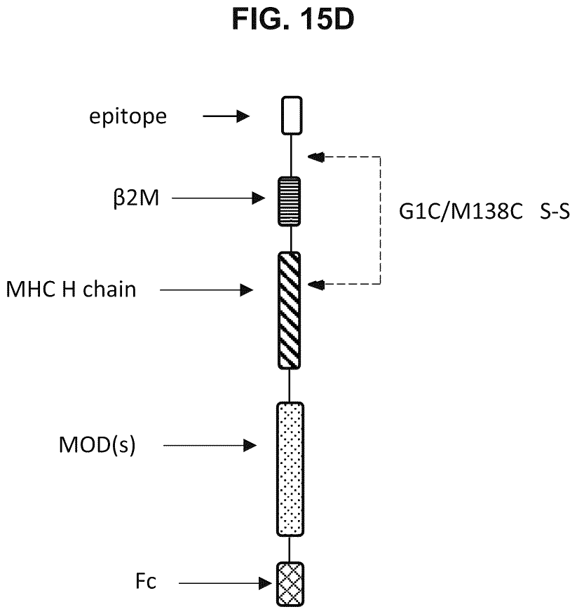

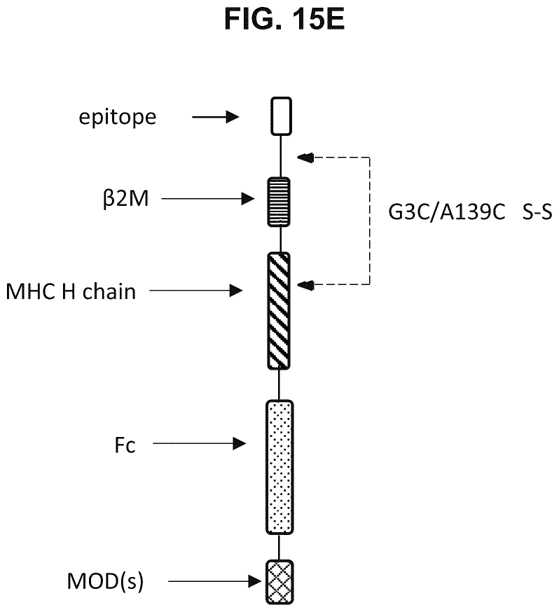

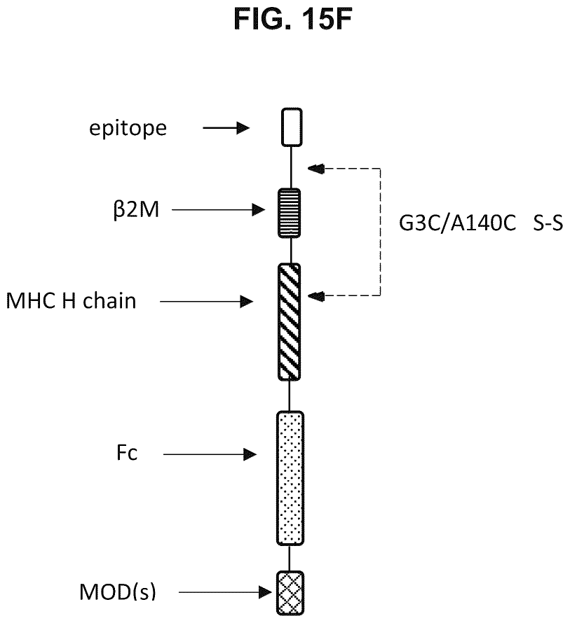

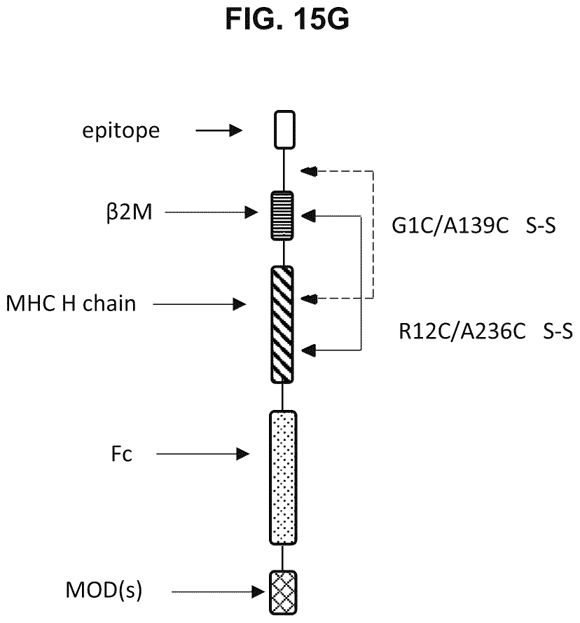

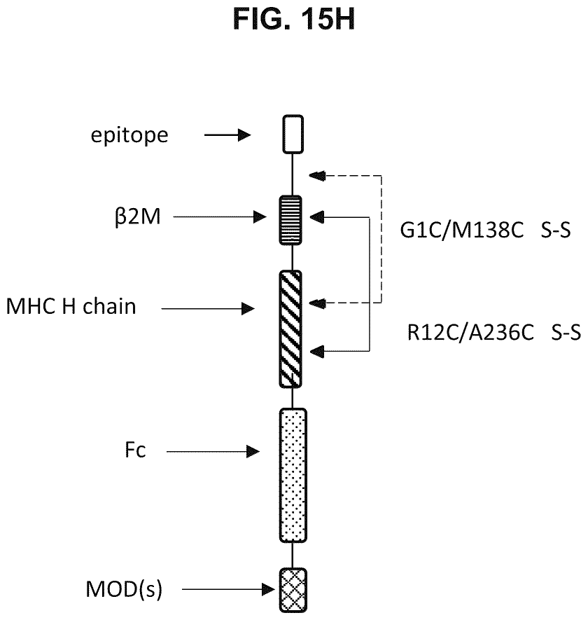

In some cases, a TMP comprises, in order from N-terminus to C-terminus, the following components: a) pMHC polypeptide, where the pMHC polypeptide comprises, in order from N-terminus to C-terminus: i) a peptide epitope; ii) a first peptide linker comprising a Cys; iii) a β2M polypeptide; iv) a second peptide linker; and v) an MHC class I heavy chain polypeptide, where the MHC class I heavy chain polypeptide comprises a Cys at any one of amino acids 135-143, based on the numbering of the MHC class I heavy chain polypeptide depicted in FIG. 3A, and where amino acid 84, based on the numbering of the MHC class I heavy chain polypeptide depicted in FIG. 3A, is other than Cys; b) one or more MODs; and c) an Ig Fc polypeptide. This arrangement of components is referred to as MOD Position 2 in FIG. 14. The TMP can include one or more additional peptide linkers. For example, the TMP can include: i) a peptide linker between the MHC class I heavy chain polypeptide and the MOD; and/or ii) a peptide linker between the MOD and the Ig Fc polypeptide. Where the TMP comprises two or more MODs in tandem, the TMP can include a peptide linker between the MODs.

In some cases, a TMP comprises, in order from N-terminus to C-terminus, the following components: a) pMHC polypeptide, where the pMHC polypeptide comprises, in order from N-terminus to C-terminus: i) a peptide epitope; ii) a first peptide linker comprising a Cys; iii) a β2M polypeptide; iv) a second peptide linker; and v) an MHC class I heavy chain polypeptide, where the MHC class I heavy chain polypeptide comprises a Cys at any one of amino acids 135-143, based on the numbering of the MHC class I heavy chain polypeptide depicted in FIG. 3A, and where amino acid 84, based on the numbering of the MHC class I heavy chain polypeptide depicted in FIG. 3A, is other than Cys; b) an Ig Fc polypeptide; and c) one or more MODs. This arrangement of components is referred to as MOD Position 3 in FIG. 14. The TMP can include one or more additional peptide linkers. For example, the TMP can include: i) a peptide linker between the MHC class I heavy chain polypeptide and the Ig Fc polypeptide; and/or ii) a peptide linker between the Ig Fc polypeptide and the MOD. Where the TMP comprises two or more MODs in tandem, the TMP can include a peptide linker between the MODs.

A MOD may comprise either a wild type (“wt”) MOD or a variant of a wt MOD. Where a MOD comprises a variant, it may exhibit reduced binding to its co-MOD, including e.g., reduced binding to one or more chains or domains of the co-MOD. In such cases, combination of the reduced affinity of the MOD for its co-MOD, and the affinity of the peptide for a TCR, may provide for enhanced selectivity of a TMP because the binding of a TMP to a T cell may be driven more by the specific binding of the pMHC of the TMP to the TCR of a target T cell than by the indiscriminate binding of the MOD to its co-MOD on a non-target T cell. Binding affinity between a MOD and its co-MOD can be determined by bio-layer interferometry (BLI) using purified MOD and purified co-MOD. Binding affinity between a MOD present in a TMP and its co-MOD can be determined by BLI using purified TMP and the co-MOD. BLI methods are well known to those skilled in the art. See, e.g., Lad et al. (2015) J. Biomol. Screen. 20(4):498-507; and Shah and Duncan (2014) J. Vis. Exp. 18:e51383. Unless otherwise stated herein, the affinity of a MOD for a co-MOD, or the affinity of a MOD on a TMP for a co-MOD, is determined using BLI as described in in published PCT application WO 2020/132138, published Jun. 25, 2020. See, e.g., paragraphs [0056]-[0057].

### Immunomodulatory Polypeptides (MODs)

In some cases, a MOD present in a TMP is a wild-type (“wt”) MOD. As discussed above, in other cases, a MOD present in a TMP is a variant of a wt. MOD that has reduced affinity for a co-MOD compared to the affinity of a corresponding wild-type MOD for the co-MOD. Suitable MODs that exhibit reduced affinity for a co-MOD can have from 1 amino acid (aa) to 20 aa differences from a wild-type MOD. For example, in some cases, a variant MOD present in a TMP differs in amino acid sequence by 1 aa, 2 aa, 3 aa, 4 aa, 5 aa, 6 aa, 7 aa, 8 aa, 9 aa, or 10 aa, from a corresponding wild-type MOD. As another example, in some cases, a variant MOD present in a TMP differs in amino acid sequence by 11 aa, 12 aa, 13 aa, 14 aa, 15 aa, 16 aa, 17 aa, 18 aa, 19 aa, or 20 aa, from a corresponding wild-type MOD.

As discussed above, a MOD may comprise a variant of a wt MOD that may exhibit reduced binding to its co-MOD, including e.g., reduced binding to one or more chains or domains of the co-MOD. For example, a variant MOD present in a TMP may bind its co-MOD with an affinity that it at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wild-type MOD for the co-MOD.

Exemplary pairs of MODs and their co-MODs include, but are not limited to those set out in Table 1, below:

TABLE 1

| Immunomodulatory | |
|---|---|
| Polypeptide (MOD) | Co-MOD |
| 4-1BBL | 4-1BB |
| PD-L1 | PD-1 |
| IL-2 | IL-2 receptor |
| CD80 | CD28 |
| CD86 | CD28 |
| OX40L (CD252) | OX40 (CD134) |
| Fas ligand | Fas |
| ICOS-L | ICOS |
| ICAM | LFA-1 |
| CD30L | CD30 |
| CD40 | CD40L |
| CD83 | CD83L |
| HVEM (CD270) | CD160 |
| JAG1 (CD339) | Notch |
| JAG1 | CD46 |
| CD80 | CTLA4 |
| CD86 | CTLA4 |
| CD70 | CD27 |
| TGFβ | TGFβ receptor |

As depicted schematically in FIG. 14, one or more MODs can be present in a TMP at any of a variety of positions. FIG. 14 depicts the position of two copies of a variant IL-2 polypeptide; however, the MOD can be any number of and any of a variety of MODs, as described herein. As depicted in FIG. 14, a MOD can be: 1) C-terminal to the MHC class I heavy chain and N-terminal to the Ig Fc polypeptide; in other words, between the MHC class I heavy chain polypeptide and the Ig Fc polypeptide, which is referred to as “Position 2” in FIG. 14; 2) C-terminal to the Ig Fc polypeptide, which is referred to as “Position 3” in FIG. 14; or 3) N-terminal to the peptide epitope, which is referred to as “Position 4” in FIG. 14.

Wild-type immunomodulatory polypeptides and variants, including reduced affinity variants, such as PD-L1, CD80, CD86, 4-1BBL and IL-2 are described in the published literature, e.g., published PCT application WO2020132138A1 and WO2019/051091, the disclosures of which as they pertain to MODs and specific variant MODs of PD-L1, CD80, CD86, 4-1BBL, IL-2 are expressly incorporated herein by reference, including specifically paragraphs [00260]-[00455] of WO2020132138A1 and paragraphs [00157]-[00352] of WO2019/051091.

Of specific interest are MODs that are variants of the cytokine IL-2. Wild-type IL-2 binds to IL-2 receptor (IL-2R) on the surface of a T cell. Wild-type IL-2 has a strong affinity for IL-2R and will bind to activate most or substantially all CD8+ T cells. For this reason, synthetic forms of wild type IL-2 such as the drug Aldesleukin (trade name Proleukin®) are known to have severe side-effects when administered to humans for the treatment of cancer because the IL-2 indiscriminately activates both target and non-target T cells.

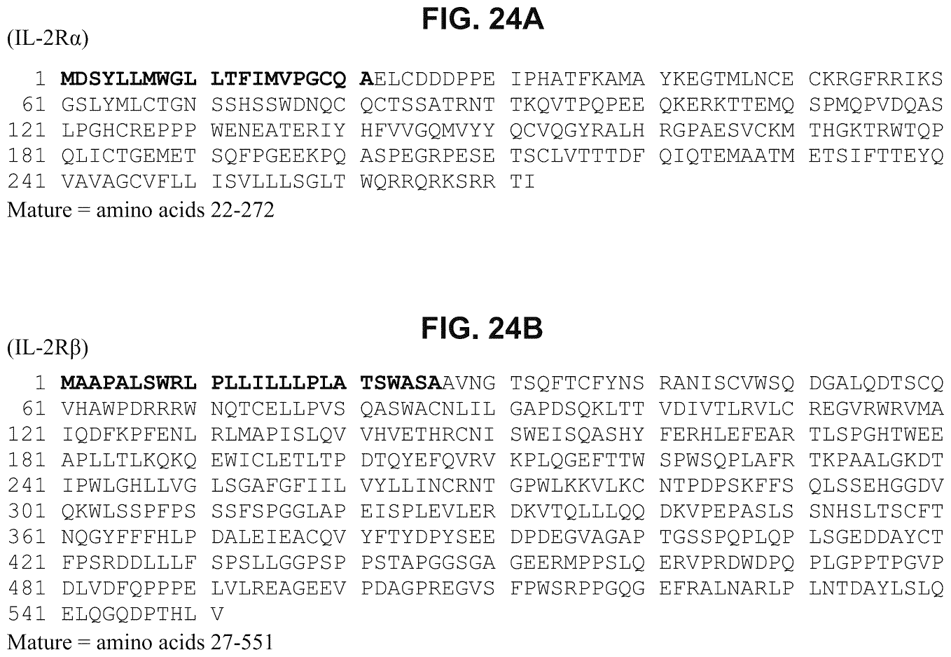

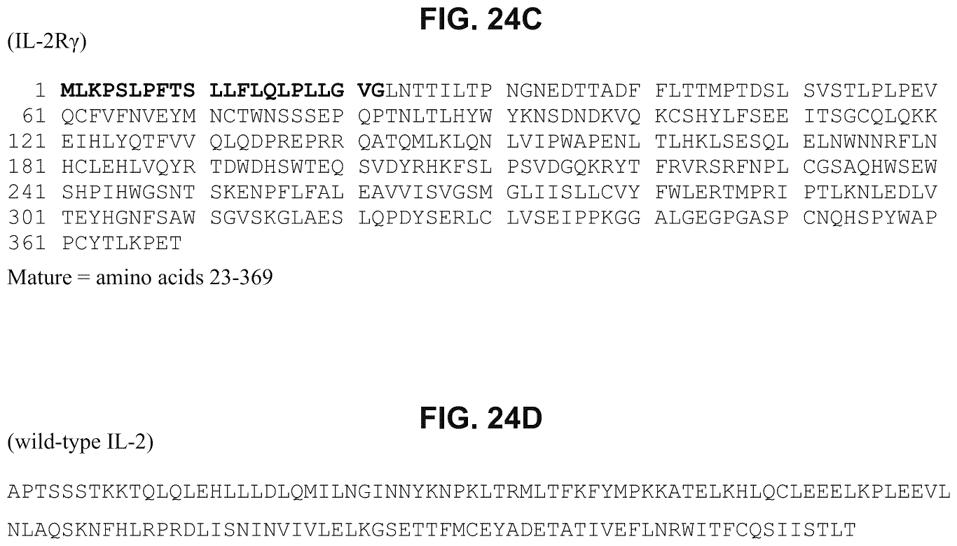

An IL-2 receptor is in some cases a heterotrimeric polypeptide comprising an alpha chain (IL-2Rα; also referred to as CD25), a beta chain (IL-2RD; also referred to as CD122; and a gamma chain (IL-2Rγ; also referred to as CD132). Amino acid sequences of human IL-2, human IL-2Rα, IL2Rβ, and IL-2Rγ are known. See, e.g., published PCT applications WO2020132138A1 and WO2019/051091, discussed above. For example, a wild-type IL-2 polypeptide can have the amino acid sequence depicted in FIG. 24D. Amino acid sequences of human IL-2Rα, human IL-2RP, and human IL-2Rγ are depicted in FIGS. 24A, 24B, and 24C, respectively, where the mature form of IL-2Rα is amino acids 22-272 of the amino acid sequence depicted in FIG. 24A, the mature form of IL-2Rβ is amino acids 27-551 of the amino acid sequence depicted in FIG. 24B, and the mature form of IL-2Rγ is amino acids 23-369 of the amino acid sequence depicted in FIG. 24C.

In some cases, an IL-2 variant MOD of this disclosure exhibits decreased binding to IL-2Rα, thereby minimizing or substantially reducing the activation of Tregs by the IL-2 variant. Alternatively, or additionally, in some cases, an IL-2 variant MOD of this disclosure exhibits decreased binding to IL-2Rβ and/or IL-2Rγ such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2Rβ and/or IL-2Rγ such that the IL-2 variant polypeptide exhibits an overall reduced affinity for IL-2R. For example, IL-2 variants having substitutions at H16 and F42 have shown decreased binding to IL-2Rα and IL-2Rβ. See, Quayle et al., Clin Cancer Res; 26(8) Apr. 15, 2020, which discloses that the binding affinity of an IL-2 polypeptide with H16A and F42A substitutions for human IL-2Rα and IL-2Rβ was decreased 110- and 3-fold, respectively, compared with wild-type IL2 binding, predominantly due to a faster off-rate for each of these interactions. TMPs comprising such variants, including variants that exhibit decreased binding to IL-2Rα and IL-2Rß, have shown the ability to preferentially bind to and activate IL-2 receptors on T cells that contain the target TCR that is specific for the peptide epitope on the TMP, and are thus less likely to deliver IL-2 to non-target T cells, i.e., T cells that do not contain a TCR that specifically binds the peptide epitope on the TMP. That is, the binding of the IL-2 variant MOD to its costimulatory polypeptide on the T cell is substantially driven by the binding of the MHC-epitope moiety rather than by the binding of the IL-2.

Suitable IL-2 variant MODs thus include a polypeptide that comprises an amino acid sequence having at least 90%, at least 95%, at least 97%, at least 98%, or at least 99% amino acid sequence identity to the wild-type IL-2 amino acid sequence depicted in FIG. 24D; and that have one or more amino acid differences from the wild-type IL-2 amino acid sequence depicted in FIG. 24D. In some cases, such a variant IL-2 polypeptide of this disclosure exhibits reduced binding affinity to IL-2R, compared to the binding affinity of an IL-2 polypeptide comprising the wild-type IL-2 amino acid sequence depicted in FIG. 24D. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90 less, at least 95 less, or more than 95 less, than the binding affinity of an IL-2 polypeptide comprising the wild-type IL-2 amino acid sequence depicted in FIG. 24D for an IL-2R (e.g., an IL-2R comprising polypeptides comprising the amino acid sequences depicted in FIGS. 24A-24C, e.g., the mature forms of the amino acid sequences depicted in FIGS. 24A-24C), when assayed under the same conditions.

Some exemplary combinations of mutations that reduce binding of an IL-2 variant polypeptide to IL-2Rα and IL-2R include the following from Table 2:

TABLE 2

| Mutation(s) to | Mutation(s) to | |
|---|---|---|
| decrease binding | decrease binding | |
| to IL-2Rα | to IL-2Rβ | Exemplary combinations |
| R38 with any | E15 with any | E15A with R38A, R38D or R38E |
| amino acid other | amino acid other | |
| than Arg, e.g., Ala, | than Glu | |
| Asp, Glu | | |
| R38 with any | H16, with any | H16A with R38A, R38D or R38E |
| amino acid other | amino acid other | H16T with R38A, R38D or R38E |
| than Arg, e.g., Ala, | than His, e.g., Ala, | H16E with R38A, R38D or R38E |
| Asp, Glu | Glu, Thr, or Asp, | H16D with R38A, R38D or R38E |
| R38 with any | D84, with any | D84H with R38A, R38D or R38E |
| amino acid other | amino acid other | D84K with R38A, R38D or R38E |
| than Arg, e.g., Ala, | than Asp, e.g., His, | D84R with R38A, R38D or R38E |
| Asp, Glu | Lys or Arg | |
| R38 with any | N88, with any | R38A with N88S, N88A, N88G, N88R, N88T, or N88D |
| amino acid other | amino acid other | R38D with N88S, N88A, N88G, N88R, N88T, or N88D |
| than Arg, e.g., Ala, | than Asn, e.g., Ser, | R38E with N88S, N88A, N88G, N88R, N88T, or N88D |
| Asp, Glu | Ala, Gly, Arg, Thr | |
| | or Asp | |
| R38 with any | V91 with any | R38A with V91E, V91A or V91T |
| amino acid other | amino acid other | R38D with V91E, V91A or V91T |
| than Arg, e.g., Ala, | than Val, e.g., Glu, | R38E with V91E, V91A or V91T |
| Asp, Glu | Ala or Thr | |
| R38 with any | I92 with any amino | R38A, I92A |
| amino acid other | acid other than Ile, | R38D, I92A |
| than Arg, e.g., Ala, | e.g., Ala | R38E, I92A |
| Asp, Glu | | |
| F42, with any | E15 with any | E15A, F42A |
| amino acid other | amino acid other | E15A, F42K |
| than Phe, e.g., Ala | than Glu | |
| or Lys, , as well as | | |
| Met, Pro, Ser, Thr, | | |
| Trp, Tyr, and Val | | |
| F42, with any | H16, with any | H16A, F42A; H16T, F42A; |
| amino acid other | amino acid other | H16E, F42A; H16D, F42A |
| than Phe, e.g., Ala | than His, e.g., Ala, | H16A, F42K; H16T, F42K; |
| or Lys, as well as | Glu, Thr, or Asp, | H16E, F42K; H16D, F42K |
| Met, Pro, Ser, Thr, | | |
| Trp, Tyr, and Val | | |
| F42, with any | D84, with any | F42A with D84H, D84K or D84R |
| amino acid other | amino acid other | F42K with D84H, D84K or D84R |
| than Phe, e.g., Ala | than Asp, e.g., His, | |
| or Lys, as well as | Lys or Arg | |
| Met, Pro, Ser, Thr, | | |
| Trp, Tyr, and Val | | |
| F42, with any | N88, with any | F42A with N88S, N88A, N88G, N88R, N88T, or N88D |
| amino acid other | amino acid other | F42K with N88S, N88A, N88G, N88R, N88T, or N88D |
| than Phe, e.g., Ala | than Asn, e.g., Ser, | |
| or Lys, as well as | Ala, Gly, Arg, Thr | |
| Met, Pro, Ser, Thr, | or Asp | |
| Trp, Tyr, and Val | | |
| F42, with any | V91 with any | F42A with V91E, V91A, or V91T |
| amino acid other | amino acid other | F42K with V91E, V91A, or V91T |
| than Phe, e.g., Ala | than Val, e.g., Glu, | |
| or Lys, as well as | Ala or Thr | |
| Met, Pro, Ser, Thr, | | |
| Trp, Tyr, and Val | | |
| F42, with any | I92 with any amino | F42A with I92A |
| amino acid other | acid other than Ile, | F42K with I92A |
| than Phe, e.g., Ala | e.g., Ala | |
| or Lys, as well as | | |
| Met, Pro, Ser, Thr, | | |
| Trp, Tyr, and Val | | |
| K43, with any | E15 with any | E15A, K43E |
| amino acid other | amino acid other | |
| than Lys, e.g., Glu | than Glu | |
| K43, with any | H16, with any | H16A, K43E; H16T, K43E; |
| amino acid other | amino acid other | H16E, K43E; H16D, K43E |
| than Lys, e.g., Glu | than His, e.g., Ala, | |
| | Glu, Thr, or Asp, | |
| K43, with any | D84, with any | K43E with D84H, D84K or D84R |
| amino acid other | amino acid other | |
| than Lys, e.g., Glu | than Asp, e.g., His, | |
| | Lys or Arg | |
| K43, with any | N88, with any | K43E with N88S, N88A, N88G, N88R, N88T, |
| amino acid other | amino acid other | or N88D |
| than Lys, e.g., Glu | than Asn, e.g., Ser, | |
| | Ala, Gly, Arg, Thr | |
| | or Asp | |
| K43, with any | V91 with any | K43E with V91E, V91A, or V91T |
| amino acid other | amino acid other | |
| than Lys, e.g., Glu | than Val, e.g., Glu, | |
| | Ala or Thr | |
| K43, with any | I92 with any amino | K43E, I92A |
| amino acid other | acid other than Ile, | |
| than Lys, e.g., Glu | e.g., Ala | |
| E62, with any | E15 with any | E15A, E62Q |
| amino acid other | amino acid other | |
| than Glu, e.g., Gln | than Glu | |
| E62, with any | H16, with any | H16A, E62Q; H16T, E62Q; |
| amino acid other | amino acid other | H16E, E62Q; H16D, E62Q |
| than Glu, e.g., Gln | than His, e.g., Ala, | |
| | Glu, Thr, or Asp, | |
| E62, with any | D84, with any | E62Q with D84H, D84K or D84R |
| amino acid other | amino acid other | |
| than Glu, e.g., Gln | than Asp, e.g., His, | |
| | Lys or Arg | |
| E62, with any | N88, with any | E62Q with N88S, N88A, N88G, N88R, N88T, |
| amino acid other | amino acid other | or N88D |
| than Glu, e.g., Gln | than Asn, e.g., Ser, | |
| | Ala, Gly, Arg, Thr | |
| | or Asp | |
| E62, with any | V91 with any | E62Q with V91E, V91A, or V91T |
| amino acid other | amino acid other | |
| than Glu, e.g., Gln | than Val, e.g., Glu, | |
| | Ala or Thr | |
| E62, with any | I92 with any amino | E62Q, I92A |
| amino acid other | acid other than Ile, | |
| than Glu, e.g., Gln | e.g., Ala | |
| F42, with any | E15 with any | E15A, F42A with D84H, D84K or D84R |
| amino acid other | amino acid other | E15A, F42K with D84H, D84K or D84R |
| than Phe, e.g., Ala | than Glu | |
| or Lys, as well as | D84, with any | |
| Met, Pro, Ser, Thr, | amino acid other | |
| Trp, Tyr, and Val | than Asp, e.g., His, | |
| | Lys and Arg | |
| F42, with any | E15 with any | E15A, F42A with N88S, N88A, N88G, N88R, |
| amino acid other | amino acid other | N88T, or N88D |
| than Phe, e.g., Ala | than Glu | E15A, F42K with N88S, N88A, N88G, N88R, |
| or Lys, as well as | N88, with any | N88T, or N88D |
| Met, Pro, Ser, Thr, | amino acid other | |
| Trp, Tyr, and Val | than Asn, e.g., Ser, | |
| | Ala, Gly, Arg, Thr, | |
| | and Asp | |
| F42, with any | E15 with any | E15A, F42A with V91E, V91A, or V91T |
| amino acid other | amino acid other | E15A, F42K with V91E, V91A, or V91T |
| than Phe, e.g., Ala | than Glu | |
| or Lys, as well as | V91 with any | |
| Met, Pro, Ser, Thr, | amino acid other | |
| Trp, Tyr, and Val | than Val, e.g., Glu, | |
| | Ala or Thr | |
| F42, with any | E15 with any | E15A, F42A with I92A |
| amino acid other | amino acid other | E15A, F42K with I92A |
| than Phe, e.g., Ala | than Glu | |
| or Lys, as well as | I92 with any amino | |
| Met, Pro, Ser, Thr, | acid other than Ile, | |
| Trp, Tyr, and Val | e.g., Ala | |
| F42, with any | H16, with any | H16A, F42A with D84H, D84K or D84R |
| amino acid other | amino acid other | H16A, F42K with D84H, D84K or D84R |
| than Phe, e.g., Ala | than His, e.g., Ala, | H16T, F42A with D84H, D84K or D84R |
| or Lys, as well as | Glu, Thr, or Asp | H16T, F42K with D84H, D84K or D84R |
| Met, Pro, Ser, Thr, | D84, with any | H16E, F42A with D84H, D84K or D84R |
| Trp, Tyr, and Val | amino acid other | H16E, F42K with D84H, D84K or D84R |
| | than Asp, e.g., His, | H16D, F42A with D84H, D84K or D84R |
| | Lys and Arg | H16D, F42K with D84H, D84K or D84R |
| F42, with any | H16, with any | H16A, F42A with N88S, N88A, N88G, N88R, |
| amino acid other | amino acid other | N88T, or N88D |
| than Phe, e.g., Ala | than His, e.g., Ala, | H16A, F42K with N88S, N88A, N88G, N88R, |
| or Lys, as well as | Glu, Thr, or Asp | N88T, or N88D |
| Met, Pro, Ser, Thr, | N88, with any | H16T, F42A with N88S, N88A, N88G, N88R, |
| Trp, Tyr, and Val | amino acid other | N88T, or N88D |
| | than Asn, e.g., Ser, | H16T, F42K with N88S, N88A, N88G, N88R, |
| | Ala, Gly, Arg, Thr, | N88T, or N88D |
| | and Asp | H16E, F42A with N88S, N88A, N88G, N88R, |
| | | N88T, or N88D |
| | | H16E, F42K with N88S, N88A, N88G, N88R, |
| | | N88T, or N88D |
| | | H16D, F42A with N88S, N88A, N88G, N88R, |
| | | N88T, or N88D |
| | | H16D, F42K with N88S, N88A, N88G, N88R, |
| | | N88T, or N88D |
| F42, with any | H16, with any | H16A, F42A with V91E, V91A, or V91T |
| amino acid other | amino acid other | H16A, F42K with V91E, V91A, or V91T |
| than Phe, e.g., Ala | than His, e.g., Ala, | H16T, F42A with V91E, V91A, or V91T |
| or Lys, as well as | Glu, Thr, or Asp | H16T, F42K with V91E, V91A, or V91T |
| Met, Pro, Ser, Thr, | V91 with any | H16E, F42A with V91E, V91A, or V91T |
| Trp, Tyr, and Val | amino acid other | H16E, F42K with V91E, V91A, or V91T |
| | than Val, e.g., Glu, | H16D, F42A with V91E, V91A, or V91T |
| | Ala or Thr | H16D, F42K with V91E, V91A, or V91T |
| F42, with any | H16, with any | H16A, F42A with I92A |
| amino acid other | amino acid other | H16A, F42K with I92A |
| than Phe, e.g., Ala | than His, e.g., Ala, | H16T, F42A with I92A |
| or Lys, as well as | Glu, Thr, or Asp | H16T, F42K with I92A |
| Met, Pro, Ser, Thr, | I92 with any amino | H16E, F42A with I92A |
| Trp, Tyr, and Val | acid other than Ile, | H16E, F42K with I92A |
| | e.g., Ala | H16D, F42A with I92A |
| | | H16D, F42K with I92A |

In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEX,LLLD LQMILNGINN YKNPKLTRML TX₂KFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:334), where X, is an amino acid other than His, and where X₂ is an amino acid other than Phe. In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEX₁LLLD LQMILNGINN YKNPKLTRML TX₂KFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:335), where: i) X₁ is Ala, Arg, Asn, Asp, Cys, Gln, Glu, Gly, Ile, Leu, Lys, Met, Phe, Pro, Ser, Thr, Trp, Tyr, or Val, e.g. Ala, Thr, Asp or Glu; and ii) X₂ is Ala, Arg, Asn, Asp, Cys, Gln, Glu, Gly, His, Ile, Leu, Lys, Met, Pro, Ser, Thr, Trp, Tyr, or Val. In some cases, X₁ is Ala and X₂ is Ala. In some cases, X₁ is Thr and X₂ is Ala. In some cases, X₁ is Asp and X₂ is Ala. In some cases, X₁ is Glu and X₂ is Ala. In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:336), i.e., the variant IL-2 polypeptide has the amino acid sequence of wild-type IL-2 but with H16A and F42A substitutions (shown in bold). Alternatively, the foregoing sequence, but with substitutions other than Ala at H16 and/or F42 may be employed, e.g., H16T, H16E or H16D may be employed instead of H16A. In some cases, a variant IL-2 polypeptide present in a TMP comprises the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:337). In some cases, a variant IL-2 polypeptide present in a TMP comprises the amino acid sequence: APTSSSTKKT QLQLETLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:338). In some cases, a variant IL-2 polypeptide present in a TMP comprises the amino acid sequence: APTSSSTKKT QLQLEDLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:339). In some cases, a variant IL-2 polypeptide present in a TMP comprises the amino acid sequence: APTSSSTKKT QLQLEELLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:340).In some cases, a TMP comprises two or more (e.g., two) copies of such a variant IL-2 polypeptide. Where a TMP comprises two copies of a variant IL-2 polypeptide, in some cases, the two copies are in tandem. Where a TMP comprises two copies of a variant IL-2 polypeptide, and where the two copies are in tandem, in some cases, the TMP comprises a peptide linker between the two copies.

In some cases, a MOD present in a TMP is a PD-L1 polypeptide. In some cases, a PD-L1 polypeptide of a TMP comprises an amino acid sequence having at least 85%, at least 90%, at least 95%, at least 97%, at least 98%, at least 99%, or 100%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:341).

In some cases, a MOD present in a TMP is a 4-1BBL polypeptide. In some cases, a 4-1BBL polypeptide of a TMP comprises an amino acid sequence having at least 85%, at least 90%, at least 95%, at least 97%, at least 98%, at least 99%, or 100%, amino acid sequence identity to the following 4-1BBL amino acid sequence: DPAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPA (SEQ ID NO:342).

In some cases, a MOD present in a TMP is an ICOS-L polypeptide. In some cases, an ICOS-L polypeptide of a TMP comprises an amino acid sequence having at least 85%, at least 90%, at least 95%, at least 97%, at least 98%, at least 99%, or 100%, amino acid sequence identity to the following ICOS-L amino acid sequence: QEKEVRAMVG SDVELSCACP EGSRFDLNDV YVYWQTSESK TVVTYHIPQN SSLENVDSRY RNRALMSPAG MLRGDFSLRL FNVTPQDEQK FHCLVLSQSL GFQEVLSVEV TLHVAANFSV PVVSAPHSPS QDELTFTCTS INGYPRPNVY WINKTDNSLL DQALQNDTVF LNMRGLYDVV SVLRIARTPS VNIGCCIENV LLQQNLTVGS QTGNDIGERD KITENPVSTG EKNAATWSIL (SEQ ID NO:343).

In some cases, a MOD present in a TMP is an OX40L polypeptide. In some cases, an OX40L polypeptide of a TMP comprises an amino acid sequence having at least 85%, at least 90%, at least 95%, at least 97%, at least 98%, at least 99%, or 100%, amino acid sequence identity to the following OX40L amino acid sequence: L QVSHRYPRIQ SIKVQFTEYK KEKGFILTSQ KEDEIMKVQN NSVIINCDGF YLISLKGYFS QEVNISLHYQ KDEEPLFQLK KVRSVNSLMV ASLTYKDKVY LNVTTDNTSL DDFHVNGGEL ILIHQNPGEF CVL (SEQ ID NO:344).

In some cases, a MOD present in a TMP is a PD-L2 polypeptide. In some cases, a PD-L2 polypeptide of a multimeric polypeptide comprises an amino acid sequence having at least 85%, at least 90%, at least 95%, at least 97%, at least 98%, at least 99%, or 100%, amino acid sequence identity to amino acids 20-273 of the PD-L2 amino acid sequence: L FTVTVPKELY IIEHGSNVTL ECNFDTGSHV NLGAITASLQ KVENDTSPHR ERATLLEEQL PLGKASFHIP QVQVRDEGQY QCIIIYGVAW DYKYLTLKVK ASYRKINTHI LKVPETDEVE LTCQATGYPL AEVSWPNVSV PANTSHSRTP EGLYQVTSVL RLKPPPGRNF SCVFWNTHVR ELTLASIDLQ SQMEPRTHPT (SEQ ID NO:345).

In some cases, a MOD present in a TMP is a CD80 polypeptide. In some cases, a CD80 polypeptide of a TMP comprises an amino acid sequence having at least 85%, at least 90%, at least 95%, at least 97%, at least 98%, at least 99%, or 100%, amino acid sequence identity to following CD80 amino acid sequence: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:346).

In some cases, a MOD present in a TMP is a CD86 polypeptide. In some cases, a CD86 polypeptide of a TMP comprises an amino acid sequence having at least 85%, at least 90%, at least 95%, at least 97%, at least 98%, at least 99%, or 100%, amino acid sequence identity to the following CD86 amino acid sequence:

(SEQ ID NO: 347)
APLKIQAYFNETADLPCQFANSQNQSLSELVVFWQDQENLVLNEVYLGKE
KFDSVHSKYMNRTSFDSDSWTLRLHNLQIKDKGLYQCIIHHKKPTGMIRI
HQMNSELSVLANFSQPEIVPISNITENVYINLTCSSIHGYPEPKKMSVLL
RTKNSTIEYDGIMQKSQDNVTELYDVSISLSVSFPDVTSNMTIFCILETD
KTRLLSSPFSIELEDPQPPPDHIP.

In some cases, a MOD present in a TMP is a FasL polypeptide, e.g., the extracellular domain of a FasL polypeptide. In some cases, a FasL polypeptide of a TMP comprises an amino acid sequence having at least 85%, at least 90%, at least 95%, at least 97%, at least 98%, at least 99%, or 100%, amino acid sequence identity to the following FasL extracellular domain amino acid sequence:

(SEQ ID NO: 348)
QLFHLQKE LAELRESTSQ MHTASSLEKQ IGHPSPPPEK
KELRKVAHLT GKSNSRSMPL EWEDTYGIVL LSGVKYKKGG
LVINETGLYF VYSKVYFRGQ SCNNLPLSHK VYMRNSKYPQ
DLVMMEGKMM SYCTTGQMWA RSSYLGAVFN LTSADHLYVN
VSELSLVNFE ESQTFFGLYK L.

### Scaffold Polypeptides

A TMP can comprise an Fc polypeptide or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:384), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the TMP, compared to a control TMP lacking the scaffold polypeptide.

### Ig Fc Polypeptides

In some cases, a TMP comprises an Ig Fc polypeptide. An Ig Fc polypeptide is also referred to herein as an “Fc polypeptide.” The Ig Fc polypeptide of a TMP can be a human IgG1 Fc, a human IgG2 Fc, a human IgG3 Fc, a human IgG4 Fc, etc., or a variant of a wild-type Ig Fc polypeptide. Variants include naturally-occurring variants, non-naturally-occurring variants, and combinations thereof.

In some cases, the Fe polypeptide present in a TMP comprises an amino acid sequence having at least about 85%, at least 90%, at least 95%, at least 97%, at least 98%, at least 99%, or 100%, amino acid sequence identity to the Fc amino acid sequence depicted in any one of FIGS. 16A-16L.

In some cases, the Fc polypeptide present in a TMP is an IgG1 Fc polypeptide, or a variant of an IgG1 Fc polypeptide. For example, in some cases, the Fc polypeptide present in a TMP comprises an amino acid sequence having at least about 85%, at least 90%, at least 95%, at least 97%, at least 98%, at least 99%, or 100%, amino acid sequence identity to the human IgG1 Fc polypeptide depicted in FIG. 16A. As another example, in some cases, the Fc polypeptide present in a TMP comprises an amino acid sequence having at least 85%, at least 90%, at least 95%, at least 97%, at least 98%, at least 99%, or 100%, amino acid sequence identity to the Fc polypeptide depicted in FIG. 16B; where the Ig Fc polypeptide comprises an Ala at position 14 and an Ala at position 15. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Fc polypeptide present in a TMP comprises the amino acid sequence depicted in FIG. 16A. In some cases, an Fc polypeptide present in a TMP comprises the amino acid sequence depicted in FIG. 16B.

In some cases, the Fc polypeptide present in a TMP is an IgG1 Fc polypeptide, or a variant of an IgG1 Fc polypeptide, where variants include naturally-occurring variants, non-naturally-occurring variants, and combinations thereof. For example, in some cases, the Fc polypeptide present in a TMP comprises an amino acid sequence having at least 85%, at least 90%, at least 95%, at least 97%, at least 98%, at least 99%, or 100%, amino acid sequence identity to the human IgG1 Fc polypeptide depicted in FIG. 16C; where the Ig Fc polypeptide comprises a Glu at position 136 and a Met at position 138. As another example, in some cases, the Fc polypeptide present in a TMP comprises an amino acid sequence having at least 85%, at least 90%, at least 95%, at least 97%, at least 98%, at least 99%, or 100%, amino acid sequence identity to the human IgG1 Fc polypeptide depicted in FIG. 16D; where the Ig Fc polypeptide has Ala at positions 14 and 15; and where the Fc polypeptide comprises a Glu at position 136 and a Met at position 138. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Fc polypeptide present in a TMP comprises the amino acid sequence depicted in FIG. 16C. In some cases, an Fc polypeptide present in a TMP comprises the amino acid sequence depicted in FIG. 16D.

In some cases, the Fc polypeptide present in a TMP comprises the amino acid sequence depicted in FIG. 16E (human IgG1 Fc comprising an L234F substitution, an L235E substitution, and a P331S substitution; where L234 corresponds to amino acid 14 of the amino acid sequence depicted in FIG. 16E; L235 corresponds to amino acid 15 of the amino acid sequence depicted in FIG. 16E; and P331 corresponds to amino acid 111 of the amino acid sequence depicted in FIG. 16E). In some cases, the Fc polypeptide present in a TMP comprises the amino acid sequence depicted in FIG. 16F, comprising an N279A substitution (N77A of the amino acid sequence depicted in FIG. 16F).

In some cases, the Ig Fe polypeptide present in a TMP does not include a C-terminal Lys present in a wild-type Ig Fc polypeptide. Thus, for example, in some cases, the Ig Fc polypeptide present in a TMP comprises an amino acid sequence having at least 85%, at least 90%, at least 95%, at least 97%, at least 98%, at least 99%, or 100%, amino acid sequence identity to the human IgG1 Fc polypeptide depicted in any one of FIG. 16H-16L, where the Ig Fc polypeptide does not include a C-terminal Lys.

### Linkers

As described above, a pMHC polypeptide present in a TMP comprises a first peptide linker and a second peptide linker. As noted above, a TMP can include one or more additional peptide linkers. For example, the TMP can include independently selected peptide linkers between one or more of: i) the MHC class I heavy chain polypeptide and the MOD; ii) the MHC class I heavy chain polypeptide and the Ig Fc polypeptide; iii) between the MOD and the Ig Fc polypeptide; and iv) where the TMP comprises two or more MODs in tandem, between the MODs. In some cases, the one or more additional linkers do not include a Cys.

As used herein, the phrase “an optional peptide linker between any two of the components of a TMP” refers to a peptide linker (other than the first peptide linker described above) between any two adjacent polypeptides within the TMP. For example, as used herein, the phrase “an optional peptide linker between any two of the components of a TMP” refers to a peptide linker between one or more of: i) the MHC class I heavy chain polypeptide and the MOD; ii) the MHC class I heavy chain polypeptide and the Ig Fc polypeptide; iii) between the MOD and the Ig Fc polypeptide; and vi) a first MOD and a second MOD. As discussed below, a peptide linker may be a flexible peptide linker, including a short flexible peptide linker, or a rigid peptide linker. As discussed below, a peptide linker may be a rigid peptide linker.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

### Flexible Peptide Linkers

Exemplary flexible peptide linkers include glycine polymers (G)ₙ, glycine-serine polymers (including, for example, (GS)ₙ, (GSGGS)ₙ (SEQ ID NO:325), (GGGGS)n (SEQ ID NO:326), and (GGGS)ₙ (SEQ ID NO:327), where n is an integer of at least one and can be an integer from 1 to 10), glycine-alanine polymers, alanine-serine polymers, and other flexible peptide linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary flexible peptide linkers can comprise amino acid sequences including, but not limited to, GGSG (SEQ ID NO:349), GGSGG (SEQ ID NO:350), GSGSG (SEQ ID NO:351), GSGGG (SEQ ID NO:352), GGGSG (SEQ ID NO:353), GSSSG (SEQ ID NO:354), GGGGS (SEQ ID NO:355), and the like.

Exemplary flexible peptide linkers include, e.g., (GGGGS)n (SEQ ID NO:328); also referred to as a “G4S” linker), where n is 1, 2, 3, 4, 5, 6, 7, 8, 9, or 10. In some cases, a linker comprises the amino acid sequence (GGGGS)n (SEQ ID NO:328), where n is 1, 2, 3, 4, 5, 6, 7, 8, 9 or 10. In some cases, a linker comprises the amino acid sequence (GGGGS)n (SEQ ID NO:329), where n is 2. In some cases, a linker comprises the amino acid sequence (GGGGS)n (SEQ ID NO:330), where n is 3. In some cases, a linker comprises the amino acid sequence (GGGGS)n (SEQ ID NO:331), where n is 4. In some cases, a linker comprises the amino acid sequence (GGGGS)n (SEQ ID NO:332), where n is 7. In some cases, a linker comprises the amino acid sequence AAAGG (SEQ ID NO://). Also suitable is a linker having the amino acid sequence AAAGG (SEQ ID NO:333). In some embodiments of a TMP of this disclosure, the β2M polypeptide can be connected to the MHC heavy chain polypeptide by a (GGGGS)n (SEQ ID NO:328) linker, where n is 1, 2, 3, 4, 5, 6, 7, 8, 9, or 10, e.g., where n=2, n=3, n=4, or n=7.

As used in this disclosure, a “short flexible peptide linker” means a flexible peptide linker that comprises fewer than 15 amino acids, i.e., from 2-14 amino acids. For example, a short flexible peptide linker can comprise from 2-4, 2-5, or 3-6 amino acids (e.g., a GGS linker), or from 4-8, 5-10 or from 10-14 amino acids. Within this range includes flexible peptide linkers comprising 2, 3, 4, 5, 6, 7, 8, 9, 10, 11, 12, 13 or 14 amino acids.

### Rigid Peptide Linkers

In some cases, a peptide linker is a rigid peptide linker. As used herein, the term “rigid peptide linker” refers to a linker comprising a contiguous stretch of two or more amino acids that effectively separates protein domains by maintaining a substantially fixed distance/spatial separation (or a minimum fixed distance/spatial separation) between the domains, thereby reducing or substantially eliminating unfavorable interactions between such domains. Rigid peptide linkers are known in the art and generally adopt a relatively well-defined conformation when in solution. Rigid peptide linkers include those which have a particular secondary and/or tertiary structure in solution; and are typically of a length sufficient to confer secondary or tertiary structure to the linker. Rigid peptide linkers include peptide linkers rich in proline, and peptide linkers having an inflexible helical structure, such as an α-helical structure. Rigid peptide linkers are described in, for example, Chen et al. (2013) Adv. Drug Deliv. Rev. 65:1357; and Klein et al. (2014) Protein Engineering, Design & Selection 27:325.

Examples of rigid peptide linkers include, e.g., (EAAAK)n (SEQ ID NO:356), A(EAAAK)n (SEQ ID NO:357), A(EAAAK)nA (SEQ ID NO:358), A(EAAAK)nALEA(EAAAK)nA (SEQ ID NO:359), (Lys-Pro)n (SEQ ID NO:360), (Glu-Pro)n (SEQ ID NO:361), (Thr-Pro-Arg)n (SEQ ID NO:362), and (Ala-Pro)n (SEQ ID NO:363) where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid peptide linkers comprising EAAAK (SEQ ID NO:364) include EAAAK (SEQ ID NO:364), (EAAAK)₂ (SEQ ID NO:365), (EAAAK)₃ (SEQ ID NO:366), A(EAAAK)₄ALEA(EAAAK)₄A (SEQ ID NO:367), and AEAAAKEAAAKA (SEQ ID NO:368). Non-limiting examples of suitable rigid peptide linkers comprising (AP)n include PAPAP (SEQ ID NO:369); also referred to herein as “(AP)2”); APAPAPAP (SEQ ID NO:370); also referred to herein as “(AP)4”); APAPAPAPAPAP (SEQ ID NO:371); also referred to herein as “(AP)6”); APAPAPAPAPAPAPAP (SEQ ID NO:372); also referred to herein as “(AP)8”); and APAPAPAPAPAPAPAPAPAP (SEQ ID NO:373); also referred to herein as “(AP)10”). Non-limiting examples of suitable rigid peptide linkers comprising (KP)n include KPKP (SEQ ID NO:374); also referred to herein as “(KP)2”); KPKPKPKP (SEQ ID NO:375); also referred to herein as “(KP)4”); KPKPKPKPKPKP (SEQ ID NO:376); also referred to herein as “(KP)6”); KPKPKPKPKPKPKPKP (SEQ ID NO:377); also referred to herein as “(KP)8”); and KPKPKPKPKPKPKPKPKPKP (SEQ ID NO:378); also referred to herein as “(KP)10”). Non-limiting examples of suitable rigid peptide linkers comprising (EP)n include EPEP (SEQ ID NO:379); also referred to herein as “(EP)2”); EPEPEPEP (SEQ ID NO:380); also referred to herein as “(EP)4”); EPEPEPEPEPEP (SEQ ID NO:381); also referred to herein as “(EP)6”); EPEPEPEPEPEPEPEP (SEQ ID NO:382); also referred to herein as “(EP)8”); and EPEPEPEPEPEPEPEPEPEP (SEQ ID NO:383); also referred to herein as “(EP)10”).

Generally speaking, where a TMP comprises a rigid peptide linker and/or a short flexible peptide linker, the TMP can include a rigid peptide linker and/or a short flexible peptide linker between any two of the components of the TMP. One or more rigid peptide linkers and/or short flexible peptide linkers may be used as follows.
(i) In a TMP comprising one or more Position 2 MODs—between one or more of: i) an MHC class I heavy chain polypeptide and a MOD; ii) a MOD and an Ig Fc polypeptide; and iii) where there are multiple MODs in tandem, between a first MOD and a second MOD.
(ii) In a TMP comprising one or more Position 3 MODs—between one or more of: i) an Ig Fc polypeptide and a MOD; and ii) where there are multiple MODs in tandem, between a first MOD and a second MOD.
(iii) In a TMP comprising one or more Position 4 MODs—between one or more of: i) a epitope and a MOD; ii) an MHC class I heavy chain polypeptide and an Ig Fc polypeptide; and iii) where there are multiple MODs in tandem, between a first MOD and a second MOD.

In a TMP having one or more Position 3 MODs, the use of a rigid peptide linker or short flexible peptide linker between the Ig Fc polypeptide and a MOD instead of a flexible peptide linker may enhance the thermal stability of the resulting TMP as compared to a TMP that is identical but for a longer, flexible peptide linker such as a (G4S)3 (i.e., (GGGGS)3) linker (SEQ ID NO:330). While not wishing to be bound by a particular theory, it is believed that the rigid peptide linker or short flexible peptide linker may reduce or prevent the interactions of a MOD with other polypeptides within the TMP that can occur with a flexible peptide linker that comprises 15 or more amino acids, resulting in enhanced thermal stability as measured using an accelerated stability assay.

Accordingly, this disclosure thus provides methods of increasing the thermal stability of a TMP comprising one or more MODS in Position 2, Position 3, or Position 4.

### DIMERIZED TMPs

In some cases, a TMP can form a dimer. That is, the present disclosure provides a polypeptide comprising a dimer of two TMPs. The present disclosure thus provides a protein that is a dimerized TMP comprising two TMPs that are covalently linked to each other. The covalent linkage of the dimer can be one or more disulfide bonds, e.g., two disulfide bonds, that form between an Ig Fc polypeptide in the first TMP and an Ig Fc polypeptide in the second TMP. As but one example, the Ig Fc can be a variant of a human IgG1 Fc polypeptide, which variant has a substantially reduced ability to effect complement-dependent cytotoxicity (CDC) or antibody-dependent cell cytotoxicity (ADCC) (e.g., the Ig Fc polypeptide of FIG. 16B, FIG. 16D, FIG. 16H, or FIG. 16J. When the TMP comprises an Ig Fc polypeptide, the TMP typically will self-assemble into a dimer by spontaneously forming one or more disulfide bonds (typically two disulfide bonds) with the Ig Fc polypeptide of another TMP. Thus, e.g., the Ig Fc polypeptides in the first TMP and the second TMP can be linked to one another by one or more, e.g., two, disulfide bonds, e.g., by two disulfide bonds. In many cases, the two TMPs will be identical to one another in amino acid sequence and comprise Ig Fc polypeptides that spontaneously form one or more disulfide bonds, e.g., two disulfide bonds, thereby forming a dimerized TMP that is a homodimer.

Accordingly, the present disclosure provides a protein comprising: a) a first TMP; and b) a second TMP, which optionally may be identical to the first TMP, where the first and second TMPs are covalently linked to one another. The covalent linkage can be one or more, e.g., two, disulfide bonds between an Ig Fc polypeptide in the first TMP and an Ig Fc polypeptide in the second TMP.

If desired, the Ig Fc polypeptides of each TMP can comprise interspecific dimerization sequences, e.g., “Knob-in-Hole” sequences that permit two different TMPs to selectively dimerize. Interspecific binding sequences favor formation of heterodimers with their cognate polypeptide sequence (i.e., the interspecific sequence and its counterpart interspecific sequence), particularly those based on Ig Fc sequence variants. Such interspecific polypeptide sequences include Knob-in-Hole, and Knob-in-Hole sequences that facilitate the formation of one or more disulfide bonds. For example, one interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Fc polypeptide with Y349C, T366S, L368A, and Y407V substitutions and a second Ig Fc polypeptide with S354C, and T366W substitutions (disulfide bonds can form between the Y349C and the S354C). See, e.g., Brinkmann and Konthermann, mAbs 9:2, 182-212 (2015). Ig Fc polypeptide sequences, either with or without knob-in-hole modifications, can be stabilized by the formation of one or more, e.g., two, disulfide bonds between the Ig Fc polypeptides (e.g., the hinge region disulfide bonds). Thus, in some cases, a dimerized TMP can be a heterodimer, comprising two TMP chains that are not identical in amino acid sequence.

Interspecific dimerization sequences also may be employed to enable TMPs to be linked to non-TMP molecules that can provide additional functionality to the TMP. For example, a TMP could be linked to a molecule that comprise polypeptides (e.g., antibodies or binding fragments thereof such as scFvs) that bind to cancer-associated antigens, thereby enabling the TMP to localize to tissues comprising the cancer-associated antigen.

### Additional Polypeptides

A polypeptide chain of a TMP can include one or more polypeptides and conjugate drugs in addition to those described above. Suitable additional polypeptides, including epitope tags and affinity domains, and drug conjugates are described in in published PCT applications WO2020132138A1 and WO2019/051091, discussed above, the disclosures of which as they pertain to epitope tags, affinity domains and drug conjugates are expressly incorporated herein by reference, including specifically paragraphs [00498]-[00508] of WO2020132138A1 and paragraphs [00353]-[00363] of WO2019/051091. The one or more additional polypeptides can be included at the N-terminus of the TMP polypeptide chain, at the C-terminus of the TMP polypeptide chain, or internally within the polypeptide chain of a TMP. As discussed above, additional polypeptides also could be conjugated to TMPs through the use of interspecific sequences.

### Exemplary TMPs and Dimerized TMPs

In the discussion below, the discussion of exemplary TMPs is intended to encompass both TMPs and dimerized TMPs comprising two TMPs where the TMPs are joined by one or more covalent bonds that join the two TMPs, e.g., one, two or more disulfide bonds that spontaneously form between the Ig Fc polypeptides in the two TMPs. Such dimerized TMPs can be either i) homodimers comprising two TMPs, where both of the TMPs have the same amino acid sequence, or ii) heterodimers comprising two TMPs, where the two TMPs differ from one another in amino acid sequence.

In some cases, a TMP comprises the following components: a) pMHC polypeptide, where the pMHC polypeptide comprises, in order from N-terminus to C-terminus: i) a peptide epitope; ii) a first peptide linker comprising a Cys; iii) a β2M polypeptide; iv) a second peptide linker; and v) an MHC class I heavy chain polypeptide, where the MHC class I heavy chain polypeptide comprises a Cys at any one of amino acids 135-143, based on the numbering of the MHC class I heavy chain polypeptide depicted in FIG. 3A, and where amino acid 84, based on the numbering of the MHC class I heavy chain polypeptide depicted in FIG. 3A, is other than Cys; b) one or more MODs; and, optionally c) an Ig Fc polypeptide or a non-Ig scaffold. The TMP may comprise one or more additional independently selected peptide linkers (other than the linkers present in the pMHC), e.g., an independently selected peptide between one or more of: i) the MHC class I heavy chain polypeptide and an Ig Fc polypeptide; ii) the MHC class I heavy chain polypeptide and a MOD; iii) an Ig Fc polypeptide and a MOD; and iv) where the TMP comprises two or more MODs in tandem, between the MODs.

In the case of a TMP comprising one or more Position 2 MODs, one or more peptide linkers may be interposed between: i) the MHC class I heavy chain polypeptide and a MOD; ii) a MOD and the Ig Fc polypeptide and the MOD; and iii) where the TMP comprises two or more MODs in tandem, between the MODs. As discussed above, in such TMPs, a flexible peptide linker, a rigid peptide linker or a short flexible peptide linker may be interposed between one or more of: i) an MHC class I heavy chain polypeptide and a MOD; ii) a MOD and an Ig Fc polypeptide; and iii) where there are multiple MODs in tandem, between a first MOD and a second MOD, and so on for additional MODs in tandem. In some cases, the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the short flexible peptide linkers will comprise from 2-4, 2-5, 3-6, 4-8, 5-10 or 10-14 amino acids. In some cases, the short flexible peptide linker is GGS. Generally speaking, flexible peptide linkers (as discussed above) will be interposed between the components that are not connected by a rigid peptide linker or short flexible peptide linker.

In the case of a TMP comprising one or more Position 3 MODs, one or more peptide linkers may be interposed between: i) the MHC class I heavy chain polypeptide and an Ig Fc polypeptide; ii) the Ig Fc polypeptide and the MOD; and iii) where the TMP comprises two or more MODs in tandem, between the MODs. As discussed above, in such TMPs, a flexible peptide linker, a rigid peptide linker or a short flexible peptide linker may be interposed between one or more of: i) an Ig Fc polypeptide and a MOD; and ii) where there are multiple MODs in tandem, between one or more of the MODs, e.g., between a first MOD and a second MOD when there are two MODs in tandem. In some cases, the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the short flexible peptide linkers will comprise from 2-4, 2-5, 3-6, 4-8, 5-10 or 10-14 amino acids. In some cases, the short flexible peptide linker is GGS. Generally speaking, flexible peptide linkers will be interposed between the components that are not connected by a rigid peptide linker or short flexible peptide linker.

As noted above, in some cases, the at least one MOD present in the TMP is a wild-type MOD. In other cases, the at least one MOD present in the TMP is a variant MOD that exhibits reduced affinity for a co-MOD, compared to the affinity of a corresponding wild-type MOD for the co-MOD.

In some cases, the peptide epitope is, e.g., an AFP peptide, a WT1 peptide, an HPV peptide, a MUC1 peptide, a MAGE A4 peptide, an NY-ESO-1 peptide, a survivin peptide, a mesothelin peptide, a PRAME polypeptide, or a viral peptide (e.g., a CMV peptide). Exemplary peptides such as these are described below.

In the above TMPs, in some cases, the HLA heavy chain that comprises an amino acid sequence having at least 85%, at least 90%, at least 95%, at least 97%, at least 98%, at least 99%, or 100% amino acid sequence identity to an HLA-A*0201 polypeptide, an HLA-A*1101 polypeptide, or HLA-A24 polypeptide, HLA-E polypeptide such as HLA-E*0101 or HLA-E*01.03, or an HLA-G polypeptide such as HLA-G*0101 or and HLA-G*01:04. In some cases, the HLA heavy chain polypeptide is an HLA-A*0301 polypeptide comprising a Cys at position 139. In some cases, the HLA heavy chain polypeptide is an HLA-A*0301 polypeptide comprising an Ala at position 84, a Cys at position 139, and an Ala at position 236. In some cases, the HLA heavy chain polypeptide is an HLA-A*0301 polypeptide comprising an Ala at position 84, a Cys at position 139, and a Cys at position 236. In some cases, for example, the HLA heavy chain polypeptide is an HLA-A*1101 polypeptide comprising a Cys at position 139. In some cases, for example, the HLA heavy chain polypeptide is an HLA-A*1101 polypeptide comprising an Ala at position 84, a Cys at position 139, and an Ala at position 236. In some cases, for example, the HLA heavy chain polypeptide is an HLA-A*1101 polypeptide comprising an Ala at position 84, a Cys at position 139, and a Cys at position 236. In some cases, the HLA heavy chain polypeptide is an HLA-E*0101 polypeptide comprising a Cys at position 139. In some cases, the HLA heavy chain polypeptide is an HLA-E*0101 polypeptide comprising an Ala at position 84, a Cys at position 139, and an Ala at position 236. In some cases, the HLA heavy chain polypeptide is an HLA-E*0101 polypeptide comprising an Ala at position 84, a Cys at position 139, and a Cys at position 236. In some cases, the HLA heavy chain polypeptide is an HLA-E*0103 polypeptide comprising a Cys at position 139. In some cases, the HLA heavy chain polypeptide is an HLA-E*0103 polypeptide comprising an Ala at position 84, a Cys at position 139, and an Ala at position 236. In some cases, the HLA heavy chain polypeptide is an HLA-E*0103 polypeptide comprising an an Ala at position 84, a Cys at position 139, and a Cys at position 236.

In some cases, a TMP comprises two MODs, where the two MODs have the same amino acid sequence, e.g., the MOD is a variant IL-2 polypeptide that exhibits reduced binding affinity for both the a and p chains of IL2R as compared to wt. IL-2 having a sequence of FIG. 24D, e.g., a variant IL-2 polypeptide comprising H16A and F42A substitutions, or a variant IL-2 polypeptide comprising H16T, D or E and F42A substitutions.

In some cases, the Ig Fc polypeptide is a variant of a human IgG1 Fc polypeptide that substantially does not induce cell lysis, e.g., an IgG1 Fc polypeptide comprising L234A and L235A (L14A and L15A) substitutions such as is shown in FIG. 16B, FIG. 16D, FIG. 16H, or FIG. 16J. Where the Ig Fc polypeptide is at the C-terminus of the TMP, i.e., MOD Position 2 described above, the Ig Fc may include a Lys at the C-terminus or the C-terminus of the Ig Fc may exclude a Lys.

In some cases, a TMP comprises a MOD at Position 3, wherein the HLA heavy chain polypeptide is a variant HLA-A*0301 polypeptide, e.g., an HLA-A*0301 polypeptide comprising an amino acid sequence having at least 85%, at least 90%, at least 95%, at least 97%, at least 98%, at least 99%, or 100% amino acid sequence identity to an HLA-A*0301 polypeptide of one of FIGS. 7B-7E, where amino acid 139 is a Cys and where amino acid 84 is other than a Cys. In some cases, the variant HLA-A*0301 polypeptide comprises an Ala at position 236. In some cases, the variant HLA-A*0301 polypeptide comprises an Ala at position 84. In some cases, the variant HLA-A*0301 polypeptide comprises a Cys at amino acid 236; and the β2M polypeptide comprises a Cys at amino acid 12. In some cases, the Ig Fc polypeptide is a variant of human IgG1 Fc polypeptide that substantially does not cause cell lysis, e.g., a human IgG1 Fc polypeptide comprising L234A and L235A substitutions as shown in FIG. 16B, FIG. 16D, FIG. 16H, or FIG. 16J, and comprising an amino acid sequence having at least 85%, at least 90%, at least 95%, at least 97%, at least 98%, at least 99%, or 100% amino acid sequence identity to the amino acid sequence depicted in any one of FIG. 16B, FIG. 16D, FIG. 16H, and FIG. 16J. In some cases, the MOD is a variant IL-2 polypeptide that exhibits reduced binding affinity for both the α and β chains of IL2R as compared to wild-type IL-2 having the amino acid sequence depicted in FIG. 2A, e.g., a variant IL-2 polypeptide comprising H16A and F42A substitutions, or a variant IL-2 polypeptide comprising H16T, D or E and F42A substitutions. In some cases, the β2M polypeptide present in a TMP comprises an amino acid sequence having at least 85%, at least 90%, at least 95%, at least 97%, at least 98%, at least 99%, or 100% amino acid sequence identity to the β2M amino acid sequence depicted in FIG. 2A. In some cases, the β2M polypeptide present in a TMP comprises an amino acid sequence having at least 85%, at least 90%, at least 95%, at least 97%, at least 98%, at least 99%, or 100% amino acid sequence identity to the β2M amino acid sequence depicted in FIG. 2B and comprises a Cys at amino acid 12. For example, in some cases, the peptide is an AFP peptide, a WT1 peptide, an HPV peptide, a MUC1 peptide, a MAGE A4 peptide, a PRAME peptide, an NY-ESO-1 peptide, a survivin peptide, a mesothelin peptide, or a viral peptide (e.g., a CMV peptide); where such peptides are described below. In some cases, the TMP comprises a rigid peptide linker between a variant IL-2 MOD and an Ig Fc polypeptide present in the TMP, where the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the TMP comprises two copies of the variant IL-2 MOD. In some cases, the TMP comprises a rigid peptide linker between: a) an Ig Fc polypeptide present in the TMP; and b) the first variant IL-2 MOD, where the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the TMP comprises a rigid peptide linker between: a) an Ig Fc polypeptide present in the TMP; and b) the first variant IL-2 MOD, where the rigid peptide linker comprises the amino acid sequence (AP)n, where n=1, 2, 3, or 4.

In some cases, a TMP comprises a MOD at Position 3, wherein the HLA heavy chain polypeptide is a variant HLA-A*1101 polypeptide, e.g., an HLA-A*0301 polypeptide comprising an amino acid sequence having at least 85%, at least 90%, at least 95%, at least 97%, at least 98%, at least 99%, or 100% amino acid sequence identity to an HLA-A*1101 polypeptide of one of FIGS. 4B-4E, where amino acid 139 is a Cys and where amino acid 84 is other than a Cys. In some cases, the variant HLA-A*1101 polypeptide comprises an Ala at position 236. In some cases, the variant HLA-A*0301 polypeptide comprises an Ala at position 84. In some cases, the variant HLA-A*1101 polypeptide comprises a Cys at amino acid 236; and the β2M polypeptide comprises a Cys at amino acid 12. In some cases, the Ig Fc polypeptide is a variant of human IgG1 Fc polypeptide that substantially does not cause cell lysis, e.g., a human IgG1 Fc polypeptide comprising L234A and L235A substitutions as shown in FIG. 16B, FIG. 16D, FIG. 16H, or FIG. 16J, and comprising an amino acid sequence having at least 85%, at least 90%, at least 95%, at least 97%, at least 98%, at least 99%, or 100% amino acid sequence identity to the amino acid sequence depicted in any one of FIG. 16B, FIG. 16D, FIG. 16H, and FIG. 16J. In some cases, the MOD is a variant IL-2 polypeptide that exhibits reduced binding affinity for both the a and p chains of IL2R as compared to wild-type IL-2 having the amino acid sequence depicted in FIG. 24D, e.g., a variant IL-2 polypeptide comprising H16A and F42A substitutions, or a variant IL-2 polypeptide comprising H16T, D or E and F42A substitutions. In some cases, the β2M polypeptide present in a TMP comprises an amino acid sequence having at least 85%, at least 90%, at least 95%, at least 97%, at least 98%, at least 99%, or 100% amino acid sequence identity to the β2M amino acid sequence depicted in FIG. 2A. In some cases, the β2M polypeptide present in a TMP comprises an amino acid sequence having at least 85%, at least 90%, at least 95%, at least 97%, at least 98%, at least 99%, or 100% amino acid sequence identity to the β2M amino acid sequence depicted in FIG. 2B and comprises a Cys at amino acid 12. In some cases, the peptide is an AFP peptide, a WT1 peptide, an HPV peptide, a MUC1 peptide, a MAGE A4 peptide, a PRAME peptide, an NY-ESO-1 peptide, a survivin peptide, a mesothelin peptide, or a viral peptide (e.g., a CMV peptide); where such peptides are described below. In some cases, the TMP comprises a rigid peptide linker between a variant IL-2 MOD and an Ig Fc polypeptide present in the TMP, where the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the TMP comprises two copies of the variant IL-2 MOD. In some cases, the TMP comprises a rigid peptide linker between: a) an Ig Fc polypeptide present in the TMP; and b) the first variant IL-2 MOD, where the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the TMP comprises a rigid peptide linker between: a) an Ig Fc polypeptide present in the TMP; and b) the first variant IL-2 MOD, where the rigid peptide linker comprises the amino acid sequence (AP)n, where n=1, 2, 3, or 4.

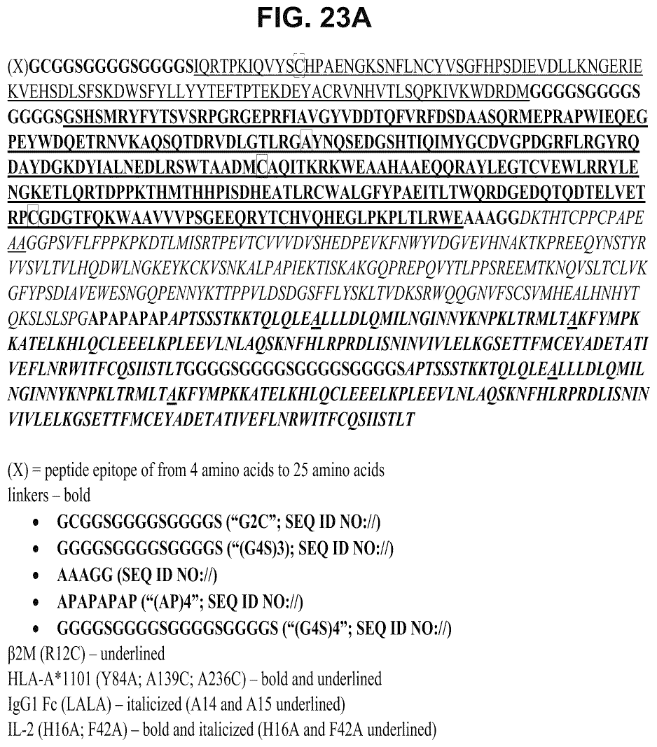

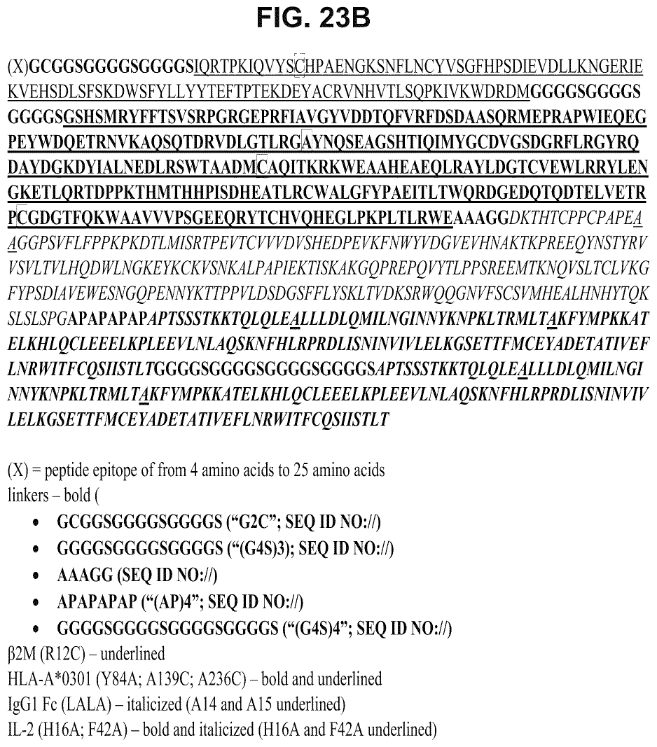

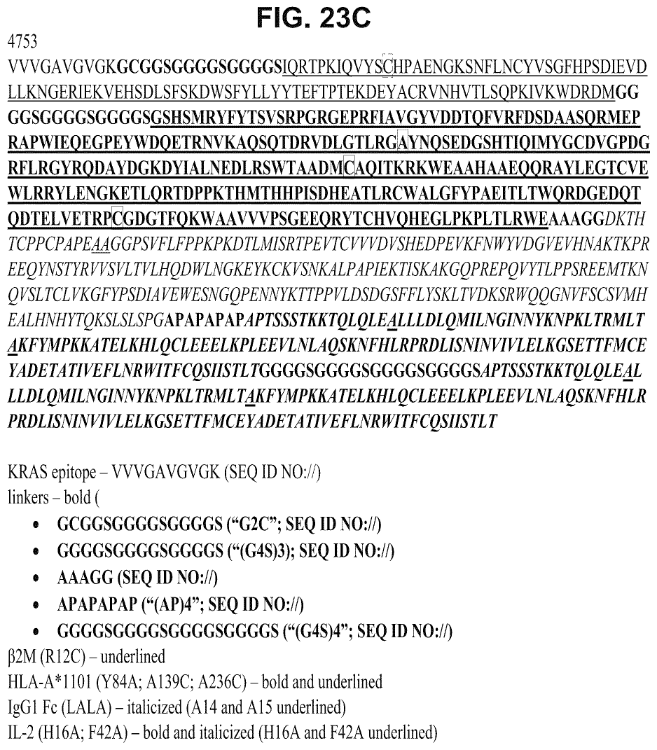

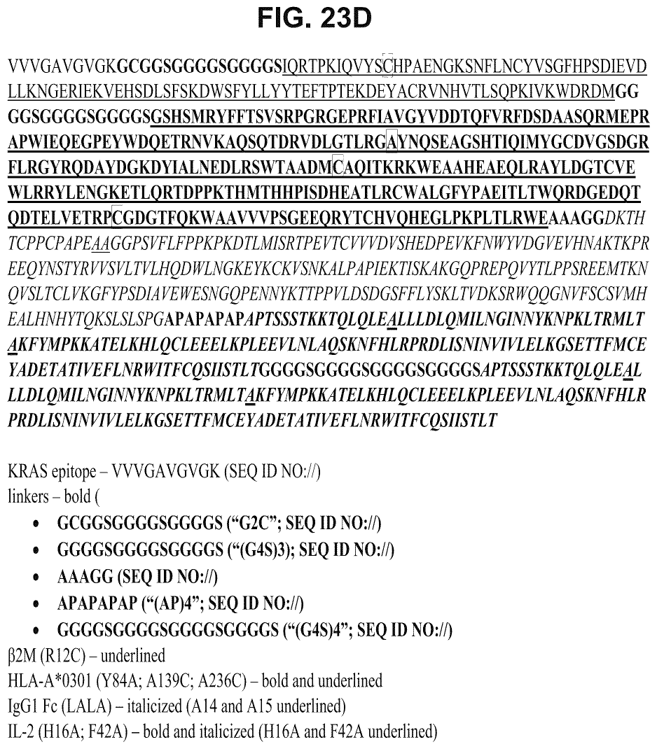

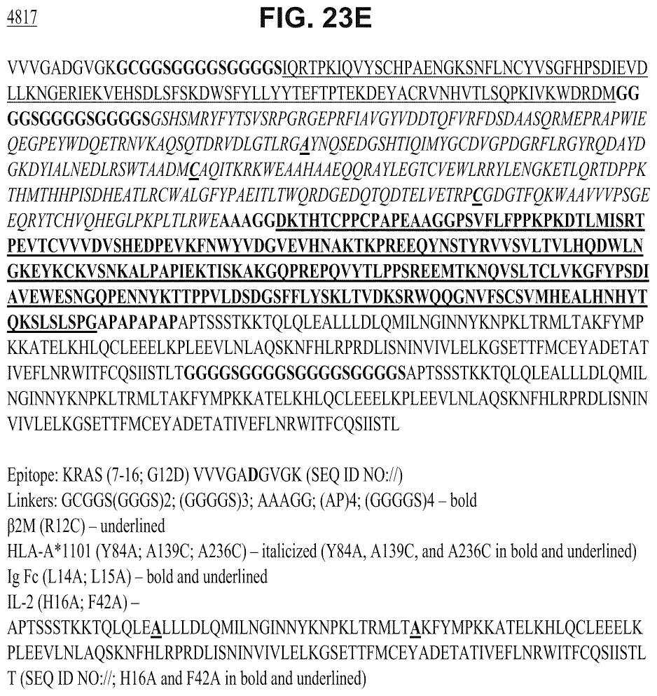

In some cases, a TMP comprises an amino acid sequence having at least 85%, at least 90%, at least 95%, at least 97%, at least 98%, or at least 99%, amino acid sequence identity to the amino acid sequence depicted in FIG. 23A, where X is a peptide epitope having a length of from 4 amino acids to 25 amino acids. In some cases, X has a length of from 4-20 aa (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa or 20 aa), including a range of from 6-15 aa, 8-12 aa, 8-16 aa, 8-10 aa, 9-11 aa, 5-10 aa, 10-15 aa, and 15-20 aa in length. In some cases, X is a peptide of from 8 to 12 amino acids in length. In some cases, X is a peptide of 8, 9, 10, or 11 amino acids in length. In some cases, the peptide epitope is an AFP peptide, a WT1 peptide, an HPV peptide, a MUC1 peptide, a MAGE A4 peptide, an NY-ESO-1 peptide, a survivin peptide, a mesothelin peptide, a PRAME peptide, or a viral peptide (e.g., a CMV peptide); where such peptides are as described below.

In some cases, a TMP comprises an amino acid sequence having at least 85%, at least 90%, at least 95%, at least 97%, at least 98%, or at least 99%, amino acid sequence identity to the amino acid sequence depicted in FIG. 23B, where X is a peptide epitope having a length of from 4 amino acids to 25 amino acids. In some cases, X has a length of from 4-20 aa (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa or 20 aa), including a range of from 6-15 aa, 8-12 aa, 8-16 aa, 8-10 aa, 9-11 aa, 5-10 aa, 10-15 aa, and 15-20 aa in length. In some cases, X is a peptide of from 8 to 12 amino acids in length. In some cases, X is a peptide of 8, 9, 10, or 11 amino acids in length. In some cases, the peptide epitope is an AFP peptide, a WT1 peptide, an HPV peptide, a MUC1 peptide, a MAGE A4 peptide, an NY-ESO-1 peptide, a survivin peptide, a mesothelin peptide, a PRAME peptide, or a viral peptide (e.g., a CMV peptide); where such peptides are as described below.

In some cases, a TMP comprises an amino acid sequence having at least 85%, at least 90%, at least 95%, at least 97%, at least 98%, at least 99%, or 100%, amino acid sequence identity to the amino acid sequence depicted in FIG. 23C, where the peptide epitope is not included when determining percent sequence identity.

In some cases, a TMP comprises an amino acid sequence having at least 85%, at least 90%, at least 95%, at least 97%, at least 98%, at least 99%, or 100%, amino acid sequence identity to the amino acid sequence depicted in FIG. 23D, where the peptide epitope is not included when determining percent sequence identity.

In some cases, a TMP comprises an amino acid sequence having at least 85%, at least 90%, at least 95%, at least 97%, at least 98%, at least 99%, or 100%, amino acid sequence identity to the amino acid sequence depicted in FIG. 23E, where the peptide epitope is not included when determining percent sequence identity.

### Nucleic Acids, Recombinant Expression Vectors, and Modified Host Cells

The present disclosure provides a nucleic acid comprising a nucleotide sequence encoding a TMP of this disclosure. In some cases, the nucleotide sequence encoding the TMP is operably linked to one or more transcriptional control elements. In some cases, the transcriptional control element is a promoter that is functional in a eukaryotic cell. In some cases, the nucleic acid is present in a recombinant expression vector.

The present disclosure thus provides recombinant expression vectors comprising nucleic acids encoding a TMP. In some cases, the recombinant expression vector is a non-viral vector. In some cases, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors are well known to persons skilled in the art. See, e.g., published PCT application WO2020132138A1 and WO2019/051091, the disclosures of which as they pertain to such expression vectors are expressly incorporated herein by reference, including specifically paragraphs [00515]-[00520] of WO2020132138A1 and paragraphs [00401]-[00406] of WO2019/051091.

The present disclosure further provides a genetically modified host cell, where the host cell is genetically modified with a nucleic acid or expression vector as described above.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC β2M.

In some cases, the host cell is a mammalian cell that has been genetically modified such that it does not synthesize endogenous MHC class I heavy chain. In some cases, the host cell is a mammalian cell that has been genetically modified such that it does not synthesize endogenous MHC β2M and such that it does not synthesize endogenous MHC class I heavy chain.

### Methods of Generating a T-Cell Modulatory Polypeptide

A TMP of this disclosure can be generated by culturing a genetically modified host cell as described above in a suitable culture medium in vitro, where such culturing results in production of the TMP. For example, a mammalian host cell (e.g., a CHO cell) can be genetically modified with a recombinant expression vector comprising a nucleotide sequence encoding a TMP; and the genetically modified mammalian host cell can be cultured in vitro in a suitable culture medium, such that the genetically modified mammalian host cell produces the TMP. The TMP can be isolated, e.g., from the culture medium in which the genetically modified mammalian host cell is cultured and/or from a cell lysate of the genetically modified mammalian host cell. The TMP can be isolated using any of a variety of well-established methods. Where the TMP comprises an Ig Fc polypeptide at its C terminus, intracellular processing may remove a C-terminal Lys residue from the C-terminus of the Ig Fc polypeptide. And as noted above, two TMPs that each comprise an Ig Fc polypeptide (e.g., an IgG1 Fc) may spontaneously form a homodimer of the two TMPs, wherein the individual TMPs are joined by one or more, e.g., two, disulfide bonds between their respective Ig Fc portions.

### Compositions

The present disclosure provides compositions, including pharmaceutical compositions, comprising a TMP or dimerized TMP as disclosed herein. The present disclosure provides compositions, including pharmaceutical compositions, comprising a nucleic acid or a recombinant expression vector, or cells such as B cells or blood cells that are capable of producing the TMP or dimerized TMP in vivo.

### Compositions Comprising a TMP or Dimerized TMP

A composition can comprise, in addition to a TMP or dimerized TMP, one or more pharmaceutically acceptable additives, a variety of which are known in the art and need not be discussed in detail herein. See, for example, the ninth (or latest) edition of Sheskey et al., “Handbook of Pharmaceutical Excipients” (2020), and/or the 23^{rd} (or latest) edition of “Remington: The Science and Practice of Pharmacy”, 23rd Ed. (2020).

Where a TMP or dimerized TMP is administered as an injectable (e.g. subcutaneously, intraperitoneally, intramuscularly, and/or intravenously) directly into a tissue, a formulation can be provided as a ready-to-use dosage form that may be directly injected or infused into the patient or admixed with a saline solution for infusion, or possibly as a non-aqueous form (e.g., a reconstitutable storage-stable powder) or aqueous form, such as liquid composed of pharmaceutically acceptable additives. Formulations may also be provided so as to enhance serum half-life of the TMP following administration. For example, the TMP or dimerized TMP may be provided in a liposome formulation, prepared as a colloid, or other conventional techniques for extending serum half-life. The preparations may also be provided in controlled release or slow-release forms.

The concentration of a TMP or dimerized TMP in a liquid composition formulation can vary widely (e.g., from less than about 0.1%, usually at or at least about 2% to as much as 20% to 50% or more by weight). Included within this range is a concentration of from about 5 to about 15 mg/mL, from about 8 to about 12 mg/mL, from about 9 to about 11 mg/mL, including about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL and about 15 mg/mL. The concentration may depend on numerous factors, including the stability of the TMP in the liquid composition.

In some cases, a TMP or dimerized TMP is present in a liquid composition. In some cases, a composition comprises: a) a TMP or dimerized TMP; and b) saline (e.g., 0.9% NaCl). In some cases, the composition is sterile and suitable for administration to a human subject.

### Methods of Modulating T Cell Activity

The present disclosure provides a method of selectively modulating the activity of an epitope-specific T cell (e.g., a T cell specific for a peptide epitope present in a TMP, such as a cancer-associated peptide or a viral peptide), the method comprising contacting the T cell with a TMP or dimerized TMP, where contacting the T cell with a TMP or dimerized TMP selectively modulates the activity of the epitope-specific T cell. In some cases, the contacting occurs in vitro. In some cases, the contacting occurs in vivo.

Where a TMP or dimerized TMP includes a MOD that is an activating polypeptide, and the peptide is a cancer-associated peptide, contacting the T cell with the TMP or dimerized TMP activates the epitope-specific T cell, increasing the cytotoxic activity of the T cell toward a cancer cell expressing the cancer-associated peptide epitope and/or increasing the number of the epitope-specific T cells. Where a TMP or dimerized TMP includes a MOD that is an activating polypeptide, and the peptide is a viral peptide, contacting the T cell with the TMP or dimerized TMP activates the epitope-specific T cell, increasing the cytotoxic activity of the T cell toward a virus-infected cell expressing the viral peptide and/or increasing the number of the epitope-specific T cells.

In some cases, a TMP or dimerized TMP, when administered to an individual in need thereof, induces both an epitope-specific T cell response and an epitope non-specific T cell response. In other words, in some cases, a TMP, when administered to an individual in need thereof (i) induces an epitope-specific T cell response by modulating the activity of a first T cell that displays both a TCR specific for the peptide epitope present in the TMP and a co-MOD that binds to the MOD present in the TMP; and (ii) induces an epitope non-specific T cell response by modulating the activity of a second T cell that displays a TCR specific for an epitope other than the peptide epitope present in the TMP, and a co-MOD that binds to the MOD present in the TMP. The ratio of the epitope-specific T cell response to the epitope-non-specific T cell response is at least 2:1, at least 5:1, at least 10:1, at least 15:1, at least 20:1, at least 25:1, at least 50:1, or at least 100:1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100:1, or more than 100:1. This ratio is determined by measuring the increase in the number of T cells specific for the target peptide epitope (e.g., cancer-associated peptide; viral peptide) versus the increase in the number of T cells that are not specific for that target epitope. For example, conventional flow cytometry methods may be employed. “Modulating the activity” of a T cell can include one or more of the following when an activating MOD such as a variant IL-2 is present: i) activating a cytotoxic (e.g., CD8+) T cell; ii) inducing cytotoxic activity of a cytotoxic (e.g., CD8+) T cell; iii) inducing production and release of a cytotoxin (e.g., a perforin; a granzyme; a granulysin) by a cytotoxic (e.g., CD8+) T cell; iv) inducing proliferation of a cytotoxic (e.g., CD8+) T cell.

As discussed above, in some cases, a TMP or dimerized TMP, when administered to an individual in need thereof, induces a proliferation of epitope-specific T cells. The increase in the percentage of epitope-specific T cells can be measured by conventional flow cytometry methods. Thus, e.g., the percent of total CD8+ T cells that are specific for the peptide epitope may be increased following contact with the TMP by at least about 10%, at least about 15%, at least about 20%, at least about 25%, at least about 50%, at least about 75%, at least about 2-fold, at least about 2.5-fold, at least about 3-fold, at least about 4-fold, or higher than 4-fold.

This disclosure provides a method of delivering a MOD selectively to target T cell, the method comprising contacting a mixed population of T cells with a TMP or dimerized TMP, where the mixed population of T cells comprises the target T cell and non-target T cells, where the target T cell is specific for the peptide epitope present within the TMP or dimerized TMP, and where the contacting step delivers the one or more MODs present within the TMP or dimerized TMP to the target T cell. In some cases, the population of T cells is in vitro. In some cases, the population of T cells is in vivo in an individual. In some cases, the method comprises administering the TMP or dimerized TMP to the individual. In some case, the target T cell is a cytotoxic T cell. In some cases, the mixed population of T cells is an in vitro population of mixed T cells obtained from an individual, and the contacting step results in activation and/or proliferation of the target T cell, generating a population of activated and/or proliferated target T cells; in some of these instances, the method further comprises administering the population of activated and/or proliferated target T cells to the individual.

The present disclosure provides a method of detecting, in a mixed population of T cells obtained from an individual, the presence of a target T cell that binds an epitope of interest (e.g., a cancer epitope; a viral epitope), the method comprising: a) contacting in vitro the mixed population of T cells with a TMP or dimerized TMP, wherein the TMP or dimerized TMP comprises the peptide epitope of interest; and b) detecting activation and/or proliferation of T cells in response to said contacting, wherein activated and/or proliferated T cells indicates the presence of the target T cell.

### Treatment Methods

This disclosure provides a method of treatment of an individual, the method comprising administering to the individual an amount of: i) a TMP or dimerized TMP, ii) one or more nucleic acids encoding the TMP, or iii) a B cell or other blood cell comprising the TMP, effective to treat the individual. Also provided is a TMP or dimerized TMP for use in a method of treatment of the human or non-human animal body. In some cases, a treatment method of this disclosure comprises administering to an individual in need thereof one or more nucleic acids or recombinant expression vectors comprising nucleotide sequences encoding a TMP or dimerized TMP. In some cases, the one or more nucleic acids or recombinant expression vectors are present in cells (e.g., B cells or other blood cells) that are administered to the individual, which cells are then capable of producing the TMP or dimerized TMP in vivo. In some cases, a treatment method of this disclosure comprises administering to an individual in need thereof one or more nucleic acids (e.g., mRNA molecules) comprising nucleotide sequences encoding a TMP or dimerized TMP, or a cell, e.g., a B cell or other blood cell such as a red blood cell comprising one or more of such nucleic acids. In some cases, a treatment method comprises administering to an individual in need thereof a TMP or dimerized TMP. Conditions that can be treated include, e.g., cancer, such as a cancer that expresses an AFP peptide, a WT1 peptide, an HPV peptide, a MUC1 peptide, a MAGE A4 peptide, an NY-ESO-1 peptide, a survivin peptide, a mesothelin peptide, a PRAME peptide, or a viral peptide (e.g., a CMV peptide). Conditions that can be treated include infectious diseases, e.g., diseases caused by a viral infection.

### Treating Cancer

A TMP can be administered to an individual in need thereof to treat a cancer in the individual, for example, where the cancer expresses, or overexpresses, the peptide present in the TMP (e.g., an AFP peptide, a WT1 peptide, an HPV peptide, a MUC1 peptide, a MAGE A4 peptide, an NY-ESO-1 peptide, a survivin peptide, a mesothelin peptide, a PRAME peptide, or a viral peptide (e.g., a CMV peptide)). For example, the cancer can be one in which the cancer cells express or over-express an AFP protein, a WT1 protein, an HPV protein, a MUC1 protein, a MAGE A4 protein, an NY-ESO-1 protein, a survivin protein, a PRAME protein, or a mesothelin protein. The present disclosure provides a method of treating cancer in an individual, the method comprising administering to the individual an effective amount of a TMP or dimerized TMP, or one or more nucleic acids (e.g., expression vectors; mRNA; etc.) comprising nucleotide sequences encoding the TMP (or a cell, e.g., a B cell or other blood cell, such as a red blood cell, comprising a nucleic acid or recombinant expression vector), where the TMP or dimerized TMP comprises a peptide that displays a T-cell epitope, and where the TMP or dimerized TMP comprises an activating MOD. In some cases, an effective amount of a TMP or dimerized TMP is an amount that, when administered in one or more doses to an individual in need thereof, either as a monotherapy or as part of a combination therapy (e.g., as part of a combination therapy with an immune checkpoint inhibitor, as discussed below), prevents a substantial increase in the overall tumor burden in the individual or reduces the overall tumor burden in the individual, i.e., the amount of cancer in the body, or alternatively, causes the total tumor burden in the patient to remain relatively stable for a sufficient period of time for the patient to have a confirmed “stable disease” as determined by standard RECIST criteria.

In some cases, an effective amount of a TMP or dimerized TMP is an amount that, when administered in one or more doses to an individual in need thereof, either as a monotherapy or as part of a combination therapy (e.g., as part of a combination therapy with an immune checkpoint inhibitor), as discussed below, causes the tumor size to be reduced by a sufficient amount, and for a sufficient period of time, for the patient to have a confirmed “partial response” as determined by standard RECIST criteria.

In some cases, an effective amount of a TMP or dimerized TMP is an amount that, when administered in one or more doses to an individual in need thereof, either as a monotherapy or as part of a combination therapy (e.g., as part of a combination therapy with an immune checkpoint inhibitor), causes the tumor size to be reduced by a sufficient amount, and for a sufficient period of time, for the patient to have a confirmed “complete response” as determined by standard RECIST criteria.

In some cases, an effective amount of a TMP or dimerized TMP is an amount that, when administered in one or more doses to an individual in need thereof, either as a monotherapy or as part of a combination therapy (e.g., as part of a combination therapy with an immune checkpoint inhibitor), reduces the number of cancer cells in the individual, including to substantially undetectable levels.

In some cases, an effective amount of a TMP or dimerized TMP is an amount that, when administered in one or more doses to an individual in need thereof (an individual having a tumor), either as a monotherapy or as part of a combination therapy (e.g., as part of a combination therapy with an immune checkpoint inhibitor), reduces the tumor volume in the individual. For example, in some cases, an effective amount of a TMP or dimerized TMP is an amount that, when administered in one or more doses to an individual in need thereof (an individual having a tumor), either as a monotherapy or as part of a combination therapy (e.g., as part of a combination therapy with an immune checkpoint inhibitor), reduces the tumor volume in the individual by at least 10%, at least 15%, at least 20%, at least 25%, at least 30%, at least 40%, at least 50%, at least 60%, at least 70%, at least 80%, at least 90%, or at least 95%, compared to the tumor volume in the individual before administration of the TMP or dimerized TMP, or in the absence of administration with the TMP or dimerized TMP. Tumor volume is determined using the formula (length×width×width)/2, where length represents the largest tumor diameter and width represents the perpendicular tumor diameter.

In some cases, an effective amount of a TMP or dimerized TMP is an amount that, when administered in one or more doses to an individual in need thereof, increases survival time of the individual, or the median overall survival of a group of individuals. For example, in some cases, an effective amount of a TMP or dimerized TMP is an amount that, when administered in one or more doses to an individual or a group of individuals in need thereof, either as a monotherapy or as part of a combination therapy (e.g., as part of a combination therapy with an immune checkpoint inhibitor), increases survival time of the individual or the median overall survival of the group of individuals by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual or median overall survival time of the group of individuals in the absence of administration with the TMP or dimerized TMP.

In some cases, an effective amount of a TMP or dimerized TMP is an amount that, when administered in one or more doses to an individual in need thereof, either as a monotherapy or as part of a combination therapy (e.g., as part of a combination therapy with an immune checkpoint inhibitor), reduces the level of circulating tumor DNA (“ctDNA”) in the patient by at least 10%, at least 15%, at least 20%, at least 25%, at least 30%, at least 40%, at least 50%, at least 60%, at least 70%, at least 80%, at least 90%, or at least 95%, compared to the ctDNA levels in the individual before administration of the TMP or dimerized TMP, or in the absence of administration with the TMP or dimerized TMP. The level of ctDNA can be determined using any known method; see, e.g., Cescon et al. (2020) Nature Cancer 1:276.

Cancers that can be treated with a method of this disclosure include cancers in which the cancer cells express, e.g., an AFP protein, a WT1 protein, an HPV protein, a MUC1 protein, a MAGE A4 protein, an NY-ESO-1 protein, a survivin protein, a mesothelin protein, a MART1 protein, a PRAME protein, or a viral protein (e.g., a CMV peptide, an influenza virus peptide, an EBV peptide, etc.).

For example, a TMP or dimerized TMP that includes an AFP peptide can be used to treat cancers such as hepatocellular carcinoma, pancreatic cancer, stomach cancer, colorectal cancer, hepatoblastoma, or an ovarian yolk sac tumor.

As another example, a TMP or dimerized TMP that includes a WT-1 peptide can be used to treat cancers such as WT-1-expressing cancers. WT1-expressing cancers include a leukemia, a desmoplastic small round cell tumor, a gastric cancer, a colon cancer, a lung cancer, a breast cancer, a germ cell tumor, an ovarian cancer, a uterine cancer, a thyroid cancer, a liver cancer, a renal cancer, a Kaposi's sarcoma, a sarcoma, a hepatocellular carcinoma, a Wilms' tumor, an acute myelogenous leukemia (AML), a myelodysplastic syndrome (MDS), an a non-small cell lung cancer (NSCLC), a myeloma, pancreatic cancer, colorectal cancer, a mesothelioma, a soft tissue sarcoma, a neuroblastoma, and a nephroblastoma.

As another example, a TMP or dimerized TMP that includes an HPV peptide can be used to treat cancers such as HPV-expressing (HPV+) cancers. HPV⁺ cancers include head and neck cancers, cervical cancer, prostate cancer, ovarian cancer, genitoanal cancers, and the like.

As another example, a TMP or dimerized TMP that includes a MUC-1 peptide can be used to treat cancers that express, or over-express, MUC-1. Examples include adenocarcinomas and hematological malignancies. Examples include, e.g., multiple myeloma; B-cell lymphoma; breast cancer; lung cancer; ovarian carcinoma; pancreatic cancer; colorectal cancer; prostate cancer; renal cancer; acute myelogenous leukemia; mesothelioma; thyroid cancer; head and neck cancer; stomach cancer; urothelial cancer; cervical cancer; and ovarian endometrial cancer.

As another example, a TMP or dimerized TMP that includes a MAGE A4 peptide can be used to treat a MAGE-A4-positive cancer. MAGE-A4-positive cancers include, e.g., melanoma, bladder cancer, head and neck cancer, lung cancer, esophageal cancer, breast cancer, colon cancer, and ovarian cancer.

As another example, a TMP or dimerized TMP that includes an NY-ESO-1 peptide can be used to treat lung cancer, esophageal cancer, breast cancer, pancreatic cancer, colorectal cancer, and ovarian cancer.

As another example, a TMP or dimerized TMP that includes a survivin peptide can be used to treat esophageal cancer (e.g., esophageal squamous cell carcinoma), melanoma, breast cancer, and leukemia.

As another example, a TMP or dimerized TMP that includes a mesothelin peptide can be used to treat mesothelin-expressing cancers such as mesothelioma, pancreatic cancer, ovarian cancer, and lung adenocarcinoma.

As another example, a TMP or dimerized TMP that includes a PRAME peptide can be used to treat PRAME-expressing cancers such as a melanoma, acute myeloid leukemia, chronic myelogenous leukemia, and acute lymphoblastic leukemia.

### Treating Infectious Disease

The present disclosure provides a method of treating an infection in an individual, e.g., an infection by a pathogenic virus. A method of this disclosure can increase the number and/or activity of epitope-specific T cells in an individual. In some instances, the epitope-specific T cell is a T cell that is specific for an epitope present on a virus-infected cell, and contacting the epitope-specific T cell with the TMP increases cytotoxic activity of the T cell toward the virus-infected cell. In some instances, the epitope-specific T cell is a T cell that is specific for an epitope present on a virus-infected cell, and contacting the epitope-specific T cell with the TMP increases the number of the epitope-specific T cells.

Thus, the present disclosure provides a method of treating a virus infection in an individual, the method comprising administering to the individual an effective amount of a TMP or dimerized TMP, or one or more nucleic acids comprising nucleotide sequences encoding the TMP (or a cell, e.g., a B cell or other blood cell such as a red blood cell comprising a nucleic acid or recombinant expression vector), where the TMP comprises a T-cell epitope that is a viral epitope, and where the TMP comprises a stimulatory (“activating”) MOD. In some cases, an effective amount of a TMP or dimerized TMP is an amount that, when administered in one or more doses to an individual in need thereof, reduces the number of virus-infected cells in the individual. For example, in some cases, an effective amount of a TMP or dimerized TMP is an amount that, when administered in one or more doses to an individual in need thereof, reduces the number of virus-infected cells in the individual by at least 10%, at least 15%, at least 20%, at least 25%, at least 30%, at least 40%, at least 50%, at least 60%, at least 70%, at least 80%, at least 90%, or at least 95%, compared to the number of virus-infected cells in the individual before administration of the TMP or dimerized TMP, or in the absence of administration with the TMP or dimerized TMP. In some cases, an effective amount of a TMP or dimerized TMP is an amount that, when administered in one or more doses to an individual in need thereof, reduces the number of virus-infected cells in the individual to undetectable levels.

### Dosages

A suitable dosage of a TMP or dimerized TMP can be determined by an attending physician or other qualified medical personnel, based on various clinical factors. As is well known in the medical arts, dosages for any one patient depend upon many factors, including the patient's size, body surface area, age, the particular polypeptide or nucleic acid (or a cell, e.g., a B cell or a blood cell such as a red blood cell comprising a nucleic acid or recombinant expression vector) to be administered, sex of the patient, time (including the type and number of MODs in the TMP), and route of administration, general health, and other drugs being administered concurrently. Depending on these various factors, a TMP or dimerized TMP may be administered in amounts between 0.1 mg/kg body weight and 20 mg/kg body weight per dose, e.g. between 0.1 mg/kg body weight to 10 mg/kg body weight, e.g. between 0.5 mg/kg body weight to 5 mg/kg body weight, between 1 mg/kg body weight to 5 mg/kg body weight; between 5 mg/kg body weight to 10 mg/kg body weight; between 10 mg/kg body weight to 15 mg/kg body weight; between 15 mg/kg body weight to 20 mg/kg body weight, however, doses above this exemplary range are envisioned, especially considering the aforementioned factors. If the regimen is a continuous infusion, it can also be in the range of 1 g to 10 mg per kilogram of body weight per minute. Where the TMP includes one or more variant IL-2 MODs discussed above such as the variants that comprise H16 and F42 substitutions, exemplary amounts of TMP or dimerized TMP include from 1 mg/kg body weight to 5 mg/kg body weight, from 5 mg/kg body weight to 10 mg/kg body weight, from about 1 mg/kg body weight to about 5 mg/kg body weight, and from about 5 mg/kg body weight to about 10 mg/kg body weight. When the TMP comprises four of such variant IL-2 MODs, exemplary amounts of TMP or dimerized TMP include 1 mg/kg, 2 mg/kg, 3 mg/kg, 4 mg/kg or 5 mg/kg, e.g., 2 mg/kg, 3 mg/kg or 4 mg/kg.

Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a TMP or dimerized TMP is administered in maintenance doses, ranging from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, or amounts exceeding 20 mg/kg of body weight.

Those of skill will readily appreciate that dose levels can vary as a function of the specific TMP or dimerized TMP, the severity of the symptoms and the susceptibility of the subject to side effects. Preferred dosages for a given compound are readily determinable by those of skill in the art by a variety of means.

In some cases, multiple doses of a TMP or dimerized TMP, a nucleic acid, or a recombinant expression vector (or a cell, e.g., a B cell or other blood cell such as a red blood cell comprising a nucleic acid or recombinant expression vector) are administered. The frequency of administration of a TMP or dimerized TMP, a nucleic acid, or a recombinant expression vector can vary depending on any of a variety of factors, e.g., severity of the symptoms, etc. For example, in some cases, a TMP or dimerized TMP, a nucleic acid, or a recombinant expression vector (or a cell, e.g., a B cell or a blood cell such as a red blood cell comprising a nucleic acid or recombinant expression vector) is administered once per month, twice per month, three times per month, every other week (qow), once per week (qw), once every two weeks, once every three weeks, or once every four weeks. It also is possible to administer a TMP more often than once per week, e.g., twice per week (biw), three times per week (tiw), four times per week, five times per week, six times per week, every other day (qod), or daily (qd), including more often than once per day, e.g., twice a day (qid), or three times a day (tid). Where the TMP or dimerized TMP is administer intravenously, administration once every week, once every two weeks, once every three weeks or once every four weeks or once every month may be commonly employed at the beginning of treatment. When a TMP is administered with an immune checkpoint inhibitor (CPI), e.g., an anti-PD1 CPI such pembrolizumab, nivolumab, cemiplimab or durvalumab, then it may be desirable to administer the TMP on the same schedule as the CPI, e.g., once every three weeks.

The duration of administration of a TMP or dimerized TMP, a nucleic acid, or a recombinant expression vector (or a cell, e.g., a B cell or a blood cell such as a red blood cell comprising a nucleic acid or recombinant expression vector), e.g., the period of time over which a TMP or dimerized TMP, a nucleic acid, or a recombinant expression vector is administered, can vary, depending on any of a variety of factors, e.g., patient response, etc. For example, a TMP or dimerized TMP, a nucleic acid, or a recombinant expression vector (or a cell, e.g., a B cell or a blood cell such as a red blood cell comprising a nucleic acid or recombinant expression vector) can be administered over a period of time ranging from about one day to about one week, two weeks, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. The length of time may depend on whether the TMP is administered in a neoadjuvant setting, in which case it may be administered only once or a few times, e.g., two, three or four times, for only a week or a few weeks before a main treatment such as surgery. In an adjuvant setting or a setting in which the patient is being treated for a cancer that has newly appeared or is recurrent or metastatic, the treatment duration may continue indefinitely or until the patient exhibits progressive disease as determined by standard RECIST criteria.

### Routes of Administration

An active agent (a TMP or dimerized TMP, a nucleic acid, or a recombinant expression vector, or a cell, e.g., a B cell or other blood cell such as a red blood cell comprising a nucleic acid or recombinant expression vector) is administered to an individual using any available method and route suitable for systemic and localized routes of administration.

A TMP or dimerized TMP of this disclosure typically will be delivered via intravenous administration, but other conventional and pharmaceutically acceptable routes of administration may be used, including intratumoral, peritumoral, intramuscular, or subcutaneous. Other routes of administration also are possible, including intralymphatic, intratracheal, intracranial, intradermal, topical application, intraarterial, rectal, nasal, oral, and other enteral and parenteral routes of administration. Routes of administration may be combined, if desired, or adjusted depending upon the TMP or dimerized TMP and/or the desired effect. A TMP or dimerized TMP, or a nucleic acid or recombinant expression vector, or a cell, e.g., a B cell or a blood cell such as a red blood cell comprising a nucleic acid or recombinant expression vector, can be administered in a single dose or in multiple doses.

### Combination Therapy

A TMP or dimerized TMP can be administered to an individual in need thereof in combination with one or more additional therapeutic agents or therapeutic treatment. A suitable dosage amount of the TMP or dimerized TMP will be the same as the dosage amount for monotherapy with the TMP or dimerized TMP (described above) or may be less or more than the monotherapy dose.

A TMP or dimerized TMP can be administered to an individual in need thereof at the same time, or at different times, as the one or more additional therapeutic agent is administered.

Thus, for example, a treatment method can comprise co-administration of a TMP or dimerized TMP and at least one additional therapeutic agent. By “co-administration” is meant that both a TMP or dimerized TMP and at least one additional therapeutic agent are administered to an individual, although not necessarily at the same time, in order to achieve a therapeutic effect that is the result of having administered both the TMP or dimerized TMP and the at least one additional therapeutic agent. The administration of the TMP or dimerized TMP and the at least one additional therapeutic agent can be substantially simultaneous, e.g., the TMP or dimerized TMP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the at least one additional therapeutic agent. In some cases, a TMP or dimerized TMP is administered to an individual who is undergoing treatment with, or who has undergone treatment with, the at least one additional therapeutic agent. The administration of the TMP or dimerized TMP and the at least one additional therapeutic agent can occur at different times and/or at different frequencies.

### Combination Therapies in Cancer Treatment

A TMP or dimerized TMP can be administered to an individual in need thereof in combination with one or more additional therapeutic agents or therapeutic treatment. A suitable dosage of the TMP or dimerized TMP typically will be the same as the dosage amount for monotherapy with the TMP (described above) or may be less or more than the monotherapy dose. Suitable additional therapeutic agents include, e.g.: i) an immune checkpoint inhibitor; ii) a cancer chemotherapeutic agent; and iii) one or more additional TMPs or dimerized TMPs. Suitable additional therapeutic treatments include, e.g., radiation, surgery (e.g., surgical resection of a tumor), and the like.

A TMP or dimerized TMP can be administered to an individual in need thereof at the same time, or at different times, as the one or more additional therapeutic agent is administered.

As noted above, a treatment method can comprise co-administration of a TMP or dimerized TMP and an immune checkpoint inhibitor such as an antibody specific for an immune checkpoint. By “co-administration” is meant that both a TMP or dimerized TMP and an antibody specific for an immune checkpoint are administered to an individual, although not necessarily at the same time, in order to achieve a therapeutic effect that is the result of having administered both the TMP or dimerized TMP and the immune checkpoint inhibitor. The administration of the TMP or dimerized TMP and the antibody specific for an immune checkpoint can be substantially simultaneous, e.g., the TMP or dimerized TMP can be administered to an individual within about 1 minute to about 24 hours (e.g., within about 1 minute, within about 5 minutes, within about 15 minutes, within about 30 minutes, within about 1 hour, within about 2 hours, within about 4 hours, within about 8 hours, within about 12 hours, or within about 24 hours) of administration of the antibody specific for an immune checkpoint. Alternatively, the TMP or dimerized TMP and immune checkpoint inhibitor can be administered on different schedules, including different days and different weeks, and different frequencies. In some cases, a TMP or dimerized TMP is administered to an individual who is undergoing treatment with, or who has undergone treatment with, an antibody specific for an immune checkpoint.

Exemplary immune checkpoint inhibitors include inhibitors that target immune checkpoint polypeptide such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122, and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT, and VISTA.

In some cases, the immune checkpoint inhibitor is an antibody specific for an immune checkpoint. Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (Medlmmune/AZ), CP-870,893 (Genentech), Mogamulizumab (Kyowa Hakko Kirin), Varlilumab (CelIDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, AMP-224, cemiplimab and durvalumab. In some cases, the anti-PD-1 monoclonal antibody is nivolumab, pembrolizumab, cemiplimab, durvalumab or PDR001. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, see, e.g., Ribas et al. (2013) J. Clin. Oncol. 31:616-22. In some cases, the immune checkpoint inhibitor is an anti-PD-L1 antibody. In some cases, the anti-PD-L1 monoclonal antibody is BMS-935559, MEDI4736, MPDL3280A (also known as RG7446), KN035, or MSB0010718C. In some embodiments, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149.

Among such checkpoint inhibitors, antibodies to PD-1, PD-L1 and CTLA-4 are the most common, with at least nivolumab, tremelimumab, pembrolizumab, ipilimumab, cemiplimab, atezolizumab, avelumab, tisleizumab and durvalumab having been approved by the FDA and/or regulatory agencies outside of the U.S. The TMPs and dimerized TMPs of this disclosure also may be co-administered with combinations of checkpoint inhibitors, e.g., a combination of (i) an antibody to PD-1 or PD-L1, and (ii) an antibody to CTLA-4.

In some cases, the at least one additional therapeutic agent comprises one or more additional TMPs or dimerized TMPs. In some cases, the method comprises administering to an individual in need thereof: a) a first composition comprising a first TMP; and b) a second composition comprising a second TMP, where the second TMP is a TMP that is different from the first TMP, e.g., comprising a different peptide epitope and/or one or more different MODs.

### Combination Therapies for Treating Viral Infections

In some cases, the method comprises administering to an individual in need thereof: a) a first composition comprising a TMP or dimerized TMP; and b) a second composition comprising a second anti-viral therapeutic agent. Anti-viral agents are known in the art, and any known anti-viral agent can be used as the second therapeutic agent.

### Subjects Suitable for Treatment

Subjects suitable for treatment with a method of this disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment. Subjects suitable for treatment include individuals having a cancer in which the cancer cells express, or overexpress, a cancer-associated antigen such as an AFP antigen, a WTi antigen, an HPV antigen, a MUC1 antigen, a MAGE A4 antigen, an NY-ESO-1 antigen, a survivin antigen, a mesothelin antigen, or a viral antigen (e.g., a CMV antigen).

For example, where a TMP or dimerized TMP includes an AFP peptide, subjects suitable for treatment with such a TMP or dimerized TMP include individuals having hepatocellular carcinoma, pancreatic cancer, stomach cancer, colorectal cancer, hepatoblastoma, or an ovarian yolk sac tumor.

As another example, where a TMP or dimerized TMP includes a WT-1 peptide, subjects suitable for treatment with such a TMP or dimerized TMP include individuals having leukemia, a desmoplastic small round cell tumor, a gastric cancer, a colon cancer, a lung cancer, a breast cancer, a germ cell tumor, an ovarian cancer, a uterine cancer, a thyroid cancer, a liver cancer, a renal cancer, a Kaposi's sarcoma, a sarcoma, a hepatocellular carcinoma, a Wilms' tumor, an acute myelogenous leukemia (AML), a myelodysplastic syndrome (MDS), an a non-small cell lung cancer (NSCLC), a myeloma, pancreatic cancer, colorectal cancer, a mesothelioma, a soft tissue sarcoma, GBM, a neuroblastoma, or a nephroblastoma.

As another example, where a TMP or dimerized TMP includes an HPV peptide, subjects suitable for treatment with such a TMP or dimerized TMP include individuals having head and neck cancers, cervical cancer, prostate cancer, ovarian cancer, or a genitoanal cancers. Where a TMP or dimerized TMP comprises an HPV peptide epitope, the TMP or dimerized TMP can be administered to an individual in need thereof to treat cervical cancer in the individual. Where a TMP or dimerized TMP comprises an HPV peptide epitope, the TMP or dimerized TMP can be administered to an individual in need thereof to treat prostate cancer in the individual. Where a TMP or dimerized TMP comprises an HPV peptide epitope, the TMP or dimerized TMP can be administered to an individual in need thereof to treat ovarian cancer in the individual. In some cases, a TMP or dimerized TMP is administered to an individual who has been infected with HPV and who has atypical cells of undetermined significance (ACUS). In some cases, a TMP or dimerized TMP is administered to an individual who has been infected with HPV and who has had an abnormal pap smear result. In some cases, a TMP or dimerized TMP is administered to an individual who has been infected with HPV and who has been diagnosed with a precursor of cervical cancer, e.g., squamous intraepithelial lesion.

As another example, where a TMP or dimerized TMP that includes a MUC-1 peptide, subjects suitable for treatment with such a TMP or dimerized TMP include individuals having multiple myeloma, B-cell lymphoma, breast cancer, lung cancer, ovarian carcinoma, pancreatic cancer, colorectal cancer, prostate cancer, renal cancer, acute myelogenous leukemia, mesothelioma, thyroid cancer, head and neck cancer, stomach cancer, urothelial cancer, cervical cancer, or ovarian endometrial cancer.

As another example, where a TMP or dimerized TMP includes a MAGE A4 peptide, subjects suitable for treatment with such a TMP or dimerized TMP include individuals having melanoma, bladder cancer, head and neck cancer, lung cancer, esophageal cancer, breast cancer, colon cancer, or ovarian cancer. As another example, where a TMP or dimerized TMP includes an NY-ESO-1 peptide, subjects suitable for treatment with such a TMP or dimerized TMP include individuals having lung cancer, esophageal cancer, breast cancer, pancreatic cancer, colorectal cancer, or ovarian cancer.

As another example, where a TMP or dimerized TMP includes a survivin peptide, subjects suitable for treatment with such a TMP or dimerized TMP include individuals having esophageal cancer (e.g., esophageal squamous cell carcinoma), melanoma, breast cancer, or leukemia.

As another example, where a TMP or dimerized TMP includes a mesothelin peptide, subjects suitable for treatment with such a TMP or dimerized TMP include individuals having mesothelioma, pancreatic cancer, ovarian cancer, or lung adenocarcinoma.

In some cases, the subject is an individual who is undergoing treatment with an immune checkpoint inhibitor. In some cases, the subject is an individual who has undergone treatment with an immune checkpoint inhibitor, but whose disease has progressed despite having received such treatment. In some cases, the subject is an individual who is undergoing treatment with, or who has undergone treatment with, a cancer chemotherapeutic agent. In some cases, the subject is an individual who is preparing to undergo treatment with, is undergoing treatment with, or who has undergone treatment with, an immune checkpoint inhibitor. In some cases, the subject is an individual who is preparing to undergo treatment with, is undergoing treatment with, or who has undergone treatment with, a cancer chemotherapeutic agent, radiation treatment, surgery, and/or treatment with another therapeutic agent.

### Detection Methods

A TMP or dimerized TMP is useful for diagnostic applications and therapeutic applications. As discussed below, when used for diagnostic applications, a TMP or dimerized TMP also can comprise a detectable label so that binding of the TMP or dimerized TMP to a target T cell is detected by detecting the detectable label.

The present disclosure thus provides a method of detecting the presence and/or activation of an antigen-specific T-cell. The methods comprise contacting a T cell with a TMP or dimerized TMP; and detecting binding of the TMP or dimerized TMP to the T cell, and/or activation of the T cell. The present disclosure provides a method of detecting an antigen-specific T cell, the method comprising contacting a T cell with a TMP or dimerized TMP, wherein binding of the TMP or dimerized TMP to the T cell indicates that the T cell is specific for the peptide epitope present in the TMP or dimerized TMP, that is, the T cell comprises a T cell receptor that is specific for the peptide epitope present in the TMP or dimerized TMP.

In some cases, the TMP or dimerized TMP comprises a detectable label. Suitable detectable labels include, but are not limited to, a radioisotope, a fluorescent polypeptide, or an enzyme that generates a fluorescent product, and an enzyme that generates a colored product. Where the TMP or dimerized TMP comprises a detectable label, binding of the TMP or dimerized TMP to the T cell is detected by detecting the detectable label.

In some cases, a TMP or dimerized TMP comprises a detectable label suitable for use in in vivo imaging, e.g., suitable for use in positron emission tomography (PET), single photon emission tomography (SPECT), near infrared (NIR) optical imaging, x-ray imaging, computer-assisted tomography (CAT), or magnetic resonance imaging (MRI), or other in vivo imaging method. Examples of suitable labels for in vivo imaging include gadolinium chelates (e.g., gadolinium chelates with DTPA (diethylenetriamine penta-acetic acid), DTPA-bismethylamide (BMA), DOTA (dodecane tetraacetic acid), or HP-DO3A (1,4,7-tris(carboxymethyl)-10-(2′-hydroxypropyl)-1,4,7,10-tetraazacyclododecane)), iron chelates, magnesium chelates, manganese chelates, copper chelates, chromium chelates, iodine-based materials, and radionuclides. Suitable radionuclides include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga ⁶⁸Ga, ⁶⁴Cu, ⁶⁷Cu, ⁷⁵Br, ⁷⁷Br, ⁹⁹mTc, ¹⁴C, ¹³N, ¹⁵O, ³²P, ³³P, and ¹⁸F. In some cases, the detectable label is a positron-emitting isotope such as ¹¹C, ¹³N, ¹⁵O, ¹⁸F, ⁶⁴Cu, ⁶⁸Ga, ⁷⁸Br, ⁸²Rb, ⁸⁶Y, ⁹⁰Y, ²²Na, ²⁶Al, ⁴⁰K, ⁸³Sr, ⁸⁹Zr, or ¹²⁴I. In some cases, the detectable label is ⁶⁴Cu. See, e.g., Woodham, Andrew et al., In vivo detection of antigen-specific CD8+ T cells by immuno-positron emission tomography, Nature Methods Articles (2020) https://doi.org/10.1038/s41592-020-0934-5.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some cases, binding of a TMP or dimerized TMP to a T cell is detected using a detectably labeled antibody specific for the TMP or dimerized TMP. An antibody specific for the TMP or dimerized TMP can comprise a detectable label such as a radioisotope, a fluorescent polypeptide, or an enzyme that generates a fluorescent product, or an enzyme that generates a colored product.

In some cases, the T cell being detected is present in a sample comprising a plurality of T cells. For example, a T cell being detected can be present in a sample comprising from 10 to 10⁹ T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹, T cells.

### HLA/Peptide Binding Assays

Whether a given peptide (e.g., a peptide that comprises a cancer-associated epitope) binds a class I HLA (comprising an HLA heavy chain and a β2M polypeptide), and, when bound to the HLA complex, can effectively present an epitope to a TCR, can be determined using any of a number of well-known methods. Assays include binding assays and T-cell activation assays, including cell-based binding assays, biochemical binding assays, T-cell activation assays, ELISPOT assays, cytotoxicity assays and Detection of Antigen-specific T cells with peptide-HLA tetramers. Such assays are described in the published scientific literature as well as in published PCT application WO2020132138A1, the disclosure of which as it pertains to specific binding assays is expressly incorporated herein by reference, including specifically paragraphs [00217]-[00225].

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002)Nature Reviews Immunol. 2:263.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A peptide-major histocompatibility complex (pMHC) polypeptide comprising, in order from N-terminus to C-terminus:
a) a peptide epitope having a length of from 4 amino acids to 25 amino acids;
b) a first peptide linker, wherein the first peptide linker comprises a cysteine (Cys);
c) a beta-2 microglobulin (β2M) polypeptide;
d) a second peptide linker; and
e) a major histocompatibility complex (MHC) class I heavy chain polypeptide, wherein the MHC class I heavy chain polypeptide comprises a Cys at any one of amino acids 135-143, based on the numbering of the MHC class I heavy chain polypeptide depicted in FIG. 3A, and wherein amino acid 84, based on the numbering of the MHC class I heavy chain polypeptide depicted in FIG. 3A, is other than Cys,
wherein the pMHC polypeptide comprises a disulfide bond formed between the Cys present in the first peptide linker and the Cys at any one of amino acids 135-143 of the MHC class I heavy chain polypeptide, and
wherein the pMHC polypeptide presents the epitope for binding to a T cell receptor.

Aspect 2. The pMHC polypeptide of aspect 1, wherein the MHC class I heavy chain polypeptide comprises a Cys at amino acid 138, 139, or 140 based on the numbering of the MHC class I heavy chain polypeptide depicted in FIG. 3A.

Aspect 3. The pMHC polypeptide of aspect 1, wherein the MHC class I heavy chain polypeptide comprises a Cys at amino acid 139 based on the numbering of the MHC class I heavy chain polypeptide depicted in FIG. 3A.

Aspect 4. The pMHC polypeptide of any one of aspects 1-3, wherein the peptide has a length of from 8 amino acids to 12 amino acids.

Aspect 5. The pMHC polypeptide of any one of aspects 1-4, wherein the first linker comprises the sequence CGGGS(GGGGS)n (SEQ ID NO:232), GCGGS(GGGGS)n (SEQ ID NO:233), or GGCGS(GGGGS)n (SEQ ID NO:234), wherein n is an integer from 1-10.

Aspect 6. The pMHC polypeptide of any one of aspects 1-4, wherein the first linker comprises the sequence GCGGS(GGGGS)n (SEQ ID NO:233), wherein n is an integer from 1-4.

Aspect 7. The pMHC polypeptide of any one of aspects 1-4, wherein the first linker comprises the sequence GCGGS(GGGGS)n (SEQ ID NO:239), wherein n is 2.

Aspect 8. The pMHC polypeptide of any one of aspects 1-7, wherein the β2M polypeptide comprises an amino acid sequence having at least 95% amino acid sequence identity to the β2M amino acid sequence depicted in FIG. 2A.

Aspect 9. The pMHC polypeptide of any one of aspects 1-8, wherein the β2M polypeptide comprises a Cys at amino acid 12, wherein the MHC class I heavy chain polypeptide comprises a Cys at amino acid 236, and wherein the pMHC polypeptide further comprises a disulfide bond formed between the Cys at amino acid 12 of the β2M polypeptide and the Cys at amino acid 236 of the MHC class I heavy chain polypeptide.

Aspect 10. The pMHC polypeptide of any one of aspects 1-9, wherein the MHC class I heavy chain polypeptide comprises an amino acid sequence having at least 95% amino acid sequence identity to an HLA-A polypeptide, an HLA-B polypeptide, an HLA-C polypeptide, or an HLA-E polypeptide.

Aspect 11. The pMHC polypeptide of aspect 10, wherein the HLA-A polypeptide is an HLA-A*0301, an HLA-A*1101 polypeptide, an HLA-A*3303 polypeptide, an HLA-A*0201 polypeptide, or an HLA-A*2401 polypeptide.

Aspect 12. The pMHC polypeptide of any one of aspects 1-11, wherein the MHC class I heavy chain polypeptide comprises an amino acid sequence having at least 95% amino acid sequence identity to the amino acid sequence depicted in any one of FIG. 3B-3E, FIG. 4B-4E, FIG. 5B-5E, FIG. 6B-6E, FIG. 7B-7E, FIG. 11B-11E, or FIG. 12B-12E.

Aspect 13. The pMHC polypeptide of any one of aspects 1-12, wherein the second peptide linker has a length of from 4 amino acids to 25 amino acids.

Aspect 14. The pMHC polypeptide of any one of aspects 1-13, wherein the peptide epitope is a cancer-associated peptide or a peptide associated with a viral infection.

Aspect 15. The pMHC polypeptide of aspect 14, wherein the cancer-associated peptide is selected from a peptide of an alpha-feto protein (AFP) polypeptide, a Wilms tumor-1 (WT-1) polypeptide, a human papilloma virus (HPV) polypeptide, a MUC-1 polypeptide, a melanoma-associated antigen-4 (MAGE-A4) polypeptide, an NY-ESO-1 polypeptide, a survivin polypeptide, a mesothelin polypeptide, and a Preferentially Expressed Antigen of Melanoma (PRAME) polypeptide, and wherein the peptide associated with a viral infection is selected from cytomegalovirus (CMV), Epstein-Barr virus (EBV), human papilloma virus, adenovirus, and coronaviruses such as a SARS-CoV-2 virus.

Aspect 16. The pMHC polypeptide of any one of aspects 1-15, wherein the polypeptide does not comprise a polypeptide other than the peptide epitope, the peptide linkers, the β2M polypeptide, and the MHC class I heavy chain polypeptide.

Aspect 17. The pMHC polypeptide of any one of aspects 1-16, wherein the pMHC has a length of from about 400 amino acids to about 450 amino acids.

Aspect 18. The pMHC polypeptide of aspect 17, wherein the pMHC has a length of from 410 amino acids to 420 amino acids.

Aspect 19. A pMHC polypeptide, wherein the pMHC polypeptide is a multimer comprising two or more of the pMHC polypeptides of any one of aspects 1-18.

Aspect 20. A pMHC polypeptide of aspect 19, wherein the multimeric polypeptide is a dimer comprising two of the pMHC polypeptides.

Aspect 21. A pMHC polypeptide of aspect 19, wherein the multimeric polypeptide is a trimer comprising three of the pMHC polypeptides.

Aspect 22. A pMHC polypeptide of aspect 19, wherein the multimeric polypeptide is a tetramer comprising four of the pMHC polypeptides.

Aspect 23. The pMHC polypeptide of any one of aspects 1-22, comprising one or more covalently or non-covalently linked moieties, wherein the one or more moieties is other than a polypeptide.

Aspect 24. The pMHC polypeptide of aspect 23, wherein the moiety is a detectable label.

Aspect 25. The pMHC polypeptide of aspect 24, wherein the detectable label is a fluorophore, a radioactive label, or a chromophore.

Aspect 26. The pMHC polypeptide of aspect 23, wherein the moiety is an insoluble support.

Aspect 27. The pMHC polypeptide of aspect 26, wherein the insoluble support is a bead.

Aspect 28. The pMHC polypeptide of aspect 27, wherein the bead is a magnetic bead.

Aspect 29. The pMHC polypeptide of aspect 23, wherein the moiety is a member of a specific binding pair.

Aspect 30. The pMHC polypeptide of aspect 29, wherein the member of the specific binding pair is biotin.

Aspect 31. The pMHC polypeptide of aspect 23, wherein the moiety comprises a lipid, a nanoparticle, or a polymer.

Aspect 32. A library of the pMHC polypeptides of any one of aspects 1-22, wherein at least two of the pMHC polypeptides in the library comprise different peptide epitopes.

Aspect 33. The library of aspect 32, wherein the pMHC polypeptides include a covalently linked nucleic acid barcode.

Aspect 34. A pharmaceutical composition comprising the pMHC polypeptide of any one of aspects 1-31.

Aspect 35. The pharmaceutical composition of aspect 34, wherein the pharmaceutical composition comprises an adjuvant.

Aspect 36. A nucleic acid comprising a nucleotide sequence encoding the pMHC polypeptide of any one of aspects 1-18.

Aspect 37. The nucleic acid of aspect 36, wherein the nucleotide sequence is operably linked to one or more transcriptional control elements.

Aspect 38. A recombinant expression vector comprising a nucleic acid of aspect 36 or aspect 37.

Aspect 39. A host cell genetically modified with the nucleic acid of aspect 36 or aspect 37 or the recombinant expression vector of aspect 38.

Aspect 40. A method of producing the pMHC polypeptide of any one of aspects 1-18, comprising culturing in vitro the host cell of aspect 39 under conditions in which the host cell produces the pMHC polypeptide.

Aspect 41. A method of detecting a T cell comprising a T cell receptor (TCR) that binds a peptide epitope presented by the pMHC polypeptide of any one of aspects 1-31, or the library of aspect 32 or 33, the method comprising: a) contacting the T cell with the pMHC polypeptide of any one of aspects 1-31, or the library of aspect 32 or 33, thereby forming a pMHC polypeptide-T cell complex; and b) detecting the pMHC polypeptide-T cell complex.

Aspect 42. The method of aspect 41, wherein the pMHC polypeptide comprises a detectable label.

Aspect 43. A method of inducing an immune response to a peptide epitope in an individual, the method comprising administering to the individual a composition of aspect 34 or aspect 35.

Aspect 44. A pMHC complex comprising:
a) a pMHC polypeptide of any one of aspects 1-31; and
b) one or more components, wherein the one or more components comprises one or more of: i) one or more heterologous polypeptides; ii) a nucleic acid component comprising one or more nucleic acids; and iii) a component other than a polypeptide or nucleic acid component.

Aspect 45. The molecule of aspect 44, wherein the pMHC complex is a pMHC fusion molecule comprising one or more heterologous polypeptides.

Aspect 46. The pMHC complex of aspect 44 or 45, wherein the one or more heterologous polypeptides comprises an immunoglobulin (Ig) Fc polypeptide, optionally wherein the Ig Fc polypeptide substantially does not induce cell lysis.

Aspect 47. The pMHC complex of aspect 46, wherein the Ig Fc polypeptide comprises an Ala at amino acid 14 and an Ala at amino acid 15, based on the amino acid number of the Ig Fc amino acid sequence depicted in FIG. 16A.

Aspect 48. The pMHC complex of aspect 46 or 47, wherein the Ig Fc polypeptide comprises an amino acid sequence having at least 95% amino acid sequence identity to the amino acid sequence depicted in FIG. 16H.

Aspect 49. The pMHC complex of any one of aspects 44-48, wherein the one or more heterologous polypeptides comprises an antigen-binding region of an antibody.

Aspect 50. The pMHC complex of aspect 49, wherein the antigen-binding region is a single-chain Fc polypeptide or a nanobody.

Aspect 51. The pMHC complex of aspect 49 or aspect 50, wherein the antigen-binding region specifically binds a cancer-associated antigen present on the surface of a cancerous cell.

Aspect 52. The pMHC complex of aspect 44, wherein the component comprising a nucleic acid comprises a nucleotide sequence that encodes a polypeptide.

Aspect 53. The pMHC complex of aspect 52, wherein the nucleic acid is a DNA molecule.

Aspect 54. The pMHC complex of aspect 52, wherein the nucleic acid is an RNA molecule.

Aspect 55. The pMHC complex of aspect 52, wherein the nucleic acid encodes a chimeric antigen receptor (CAR).

Aspect 56. The pMHC complex of aspect 44, wherein the one or more heterologous polypeptides comprises a CRISPR-Cas effector polypeptide.

Aspect 57. The pMHC complex of aspect 44 or aspect 56, wherein the component comprising a nucleic acid comprises a CRISPR-Cas guide nucleic acid.

Aspect 58. A nucleic acid comprising a nucleotide sequence encoding a molecule of any one of aspects 44-51 and aspect 56.

Aspect 59. A nucleic acid of aspect 58, wherein the nucleotide sequence is operably linked to one or more transcriptional control elements.

Aspect 60. A recombinant expression vector comprising a nucleic acid of aspect 58 or aspect 59.

Aspect 61. A host cell genetically modified with the nucleic acid of aspect 58 or aspect 59 or the recombinant expression vector of aspect 60.

Aspect 62. A method of producing the pMHC complex of any one of aspects 44-51, comprising culturing in vitro the host cell of aspect 61 under conditions in which the host cell produces the pMHC complex.

Aspect 63. A composition comprising the pMHC complex of any one of aspects 44-57.

Aspect 64. A method of delivering a polypeptide and/or a nucleic acid to a T cell, the method comprising contacting the T cell with the pMHC complex of any one of aspects 44-57, wherein the T cell comprises a T cell receptor that binds the pMHC of the pMHC complex, and wherein said contacting provides for delivery of the one or more components to the T cell.

Aspect 65. A single-chain T-cell modulatory polypeptide (TMP) comprising:
a) a peptide-major histocompatibility complex (pMHC) polypeptide according to any one of aspects 1-18;
b) one or more immunomodulatory polypeptides; and
c) an immunoglobulin (Ig) Fc polypeptide or a non-immunoglobulin scaffold component.

Aspect 66. The TMP of aspect 65, wherein the TMP comprises from N-terminus to C-terminus:
a1) the pMHC,
b1) the one or more immunomodulatory polypeptides, and
c1) an Ig Fc polypeptide; or
a2) the pMHC,
b2) an Ig Fc polypeptide, and
c2) the one or more immunomodulatory polypeptides, and
wherein the TMP may further comprise an independently selected linker interposed between any two of the components of the TMP.

Aspect 67. The TMP of aspect 65 or aspect 66, wherein the TMP comprises from N-terminus to C-terminus:
a1) the pMHC,
b1) an optional linker,
c1) an Ig Fc polypeptide,
d1) an optional linker, and
e1) an immunomodulatory polypeptide; or
a2) the pMHC,
b2) an optional linker,
c2) the Ig Fc polypeptide,
d2) an optional linker,
e2) a first immunomodulatory polypeptide,
f2) an optional linker, and
g2) a second immunomodulatory polypeptide.

Aspect 68. The TMP of aspect 67, wherein the TMP comprises at least one short flexible peptide linker and/or at least one rigid peptide linker, and wherein each short flexible peptide linker comprises from 2-4, 2-5, 3-6, 4-8, 5-10 or 10-14 amino acids, and wherein each rigid peptide linker is independently selected from the group consisting of:
i) (AP)n (SEQ ID NO:363), where n is an integer from 1-10;
ii) (EP)n (SEQ ID NO:361), where n is an integer from 1-10;
iii) (KP)n (SEQ ID NO:360), where n is an integer from 1-10; and
iv) a peptide comprising EAAAK (SEQ ID NO:364).

Aspect 69. The TMP of any one of aspects 65-68, wherein the TMP comprises an Ig Fc polypeptide, optionally wherein the Ig Fc polypeptide substantially does not induce cell lysis.

Aspect 70. The TMP of aspect 69, wherein the Ig Fc polypeptide comprises an Ala at amino acid 14 and an Ala at amino acid 15, based on the amino acid number of the Ig Fc amino acid sequence depicted in FIG. 16A.

Aspect 71. The TMP of aspect 69 or aspect 70, wherein the Ig Fc polypeptide comprises an amino acid sequence having at least 95% amino acid sequence identity to the amino acid sequence depicted in FIG. 16H.

Aspect 72. The TMP of any one of aspects 65-71, wherein at least one of the one or more immunomodulatory polypeptides is a wild-type or variant of an activating immunomodulatory polypeptide, optionally selected from IL-2, a 4-1BBL, CD80, CD86, and combinations thereof, and optionally wherein at least one of the at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide.

Aspect 73. The TMP of aspect 72, wherein the at least one immunomodulatory polypeptide is a variant of IL-2 that exhibits decreased binding affinity for IL-2Rα and IL-2Rβ, optionally wherein the variant IL-2 polypeptide comprises an amino acid other than histidine at position 16 and an amino acid other than phenylalanine at position 42.

Aspect 74. The TMP of aspect 73, wherein the at least one immunomodulatory polypeptide comprises i) an H16A substitution and an F42A substitution; ii) an H16T substitution and an F42A substitution, iii) an H16D and F42A substitution, or iv) an H16E substitution and an F42A substitution.

Aspect 75. A protein comprising two TMPs according to any one of aspects 65-74, wherein the two TMPs each comprise and Ig Fc polypeptide,
wherein the two TMPs have the same amino acid sequence, and
wherein the two TMPs are joined by one or more disulfide bonds that join the Ig Fc component of one TMP to the Ig Fc component of the other TMP, optionally wherein the two TMPs are joined by two disulfide bonds that join the Ig Fc component of one TMP to the Ig Fc component of the other TMP.

Aspect 76. A pharmaceutical composition comprising the TMP of any one of aspects 65-74, or the protein of aspect 75.

Aspect 77. A nucleic acid comprising a nucleotide sequence encoding the TMP according to any one of aspects 65-74.

Aspect 78. The nucleic acid of aspect 77, wherein the nucleotide sequence is operably linked to one or more transcriptional control elements.

Aspect 79. A recombinant expression vector comprising the nucleic acid of aspect 77 or aspect 78.

Aspect 80. A host cell genetically modified with the nucleic acid of aspect 77 or aspect 78, or the recombinant expression vector of aspect 79.

Aspect 81. A method of producing a TMP according to any one of aspects 65-74, or the protein of aspect 75, the method comprising culturing in vitro the host cell of aspect 80 under conditions in which the host cell produces the TMP.

Aspect 82. A method of modulating the activity of a T cell, the method comprising contacting the T cell with a TMP according to any one of aspects 65-74 or the protein of aspect 75.

Aspect 83. A method of aspect 82 wherein said contacting is in vitro.

Aspect 84. A method of aspect 82, wherein said contacting is in vivo.

Aspect 85. A method of treating cancer in an individual, the method comprising administering to the individual an effective amount of the TMP of any one of aspects 65-74 or the protein of aspect 75, wherein the peptide epitope is a cancer-associated peptide epitope.

Aspect 86. The method of aspect 85, further comprising administering one or more additional therapeutic agents.

Aspect 87. The method of aspect 86, wherein the one or more additional therapeutic agents comprises an immune checkpoint inhibitor.

Aspect 88. The method of aspect 87, wherein the immune checkpoint inhibitor is an antibody specific for PD-1, PD-L1, or CTLA4.

Aspect 89. The method of aspect 88, wherein the antibody is a single-chain Fv or a nanobody.

Aspect 90. A method of treating a viral infection in an individual, the method comprising administering to the individual a TMP of any one of aspects 65-74 or the protein of aspect 75, wherein the peptide epitope is a peptide associated with a viral infection.

### EXAMPLES

The following examples are put forth so as to provide those of ordinary skill in the art with a complete disclosure and description of how to make and use the present disclosure, and are not intended to limit the scope of the disclosure, nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Production and Temperature Stability of TMPs with Intrachain Disulfide Bond

Single-chain TMPs, and a split-chain TMP, were constructed and their stability tested in an accelerated stability study. Constructs included either the “KRAS G12V” peptide epitope (VVVGAVGVGK; SEQ ID NO: 195) or the “KRAS G12D” peptide epitope (VVVGADGVGK; SEQ ID NO: 184); and either an HLA-A*0301 or an HLA-A*1101 allele MHC class I heavy chain polypeptide.

As shown in Table 3, below, single-chain constructs 4804, 4744, 4708, and 4742 included a single intrachain disulfide bond, between a Cys at amino acid 12 in the β2M polypeptide and a Cys at amino acid 236 in the HLA class I heavy chain polypeptide. The single-chain construct 4753 includes 2 intrachain disulfide bonds: a) a first intrachain disulfide bond between: i) a Cys in the linker between the KRAS G12V peptide epitope and the β2M polypeptide, where the linker comprises the sequence GCGGS(GGGGS)2 (SEQ ID NO:239); and ii) a Cys at amino acid 139 in the HLA class I heavy chain polypeptide; and b) a second intrachain disulfide bond between a Cys at amino acid 12 in the β2M polypeptide and a Cys at amino acid 236 in the HLA class I heavy chain polypeptide.

TABLE 3

| | | | |
|---|---|---|---|
| Construct | Epitope | HLA allele | S—S bond(s) |

Single chain constructs

| | | | |
|---|---|---|---|
| 4808 | KRAS G12V | A*0301 | R12C:A236C |
| 4744 | KRAS G12D | A*0301 | R12C:A236C |
| 4708 | KRAS G12V | A*1101 | R12C:A236C |
| 4742 | KRAS G12D | A*1101 | R12C:A236C |
| 4753 | KRAS G12V | A*1101 | G2C:A139C |
| | | | R12C:A236C |

Split chain construct

| | | | |
|---|---|---|---|
| 4718-4029 | KRAS G12D | A*1101 | G2C:Y84C |
| | | | R12C:A236C |

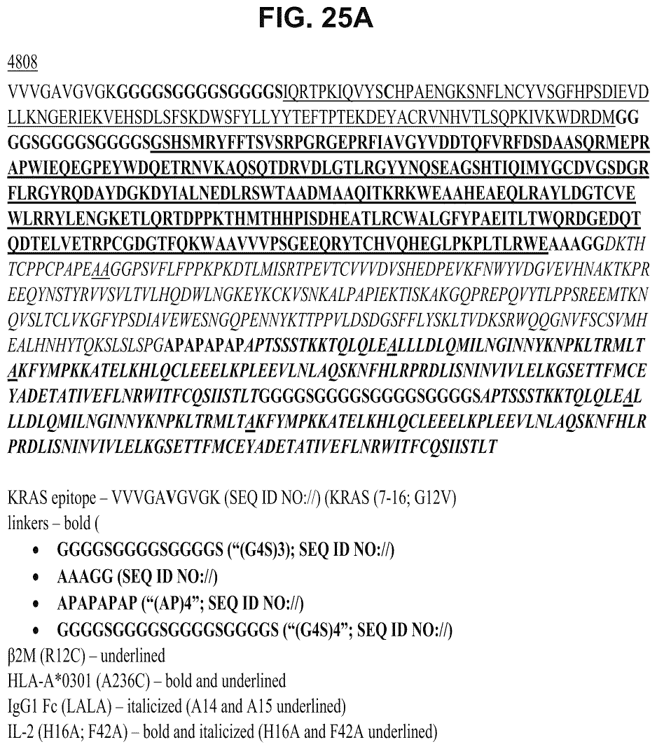

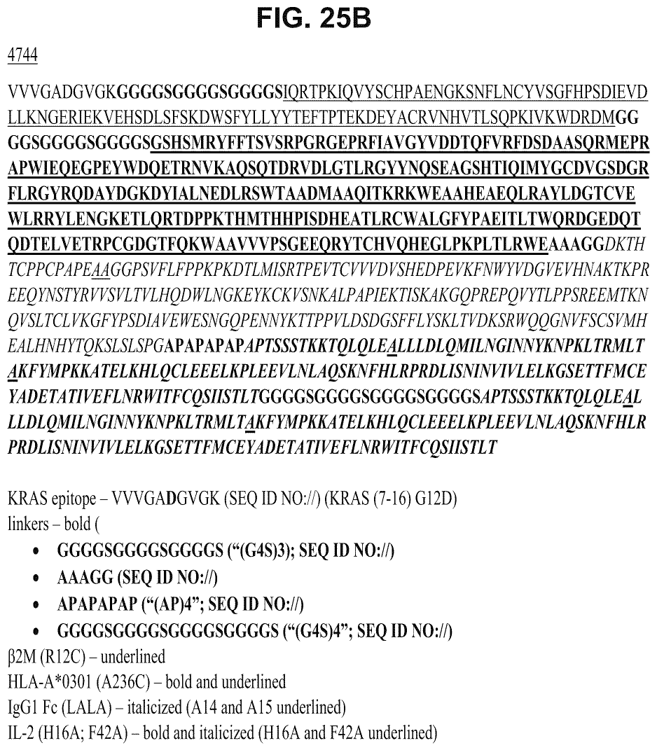

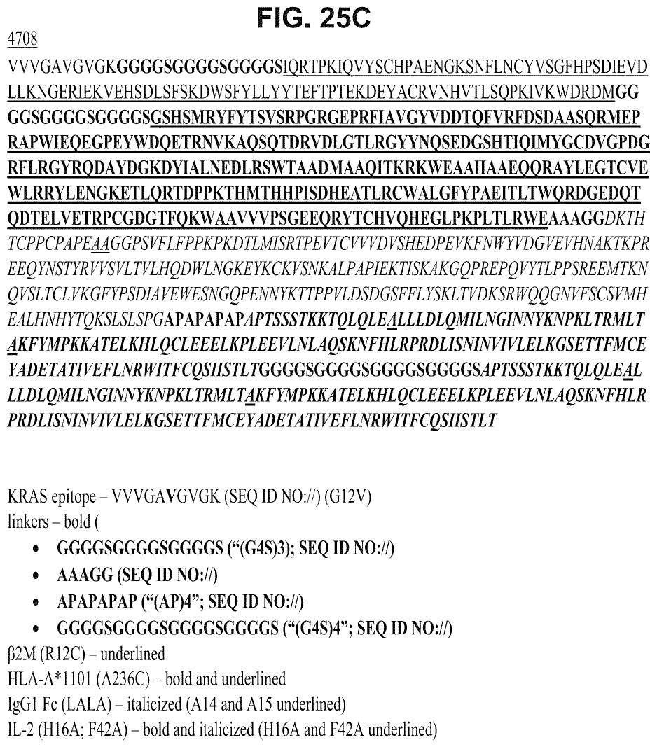

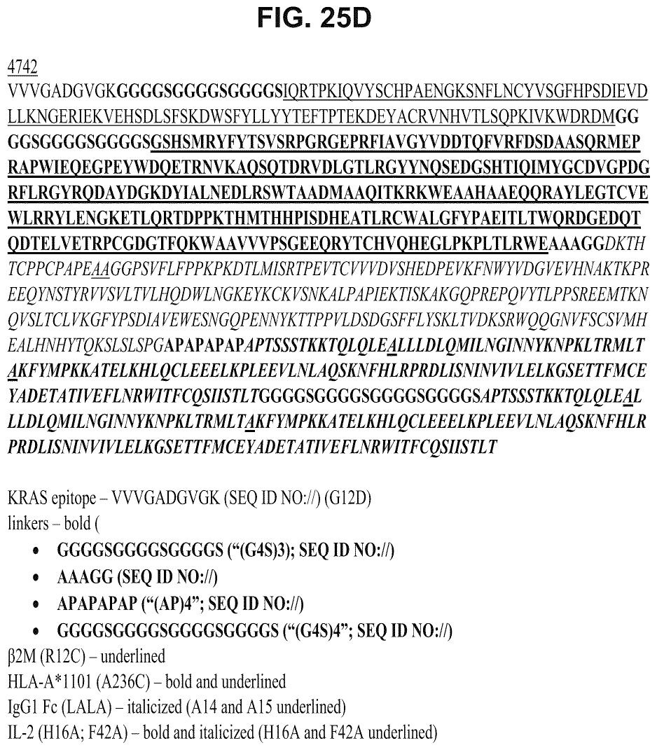

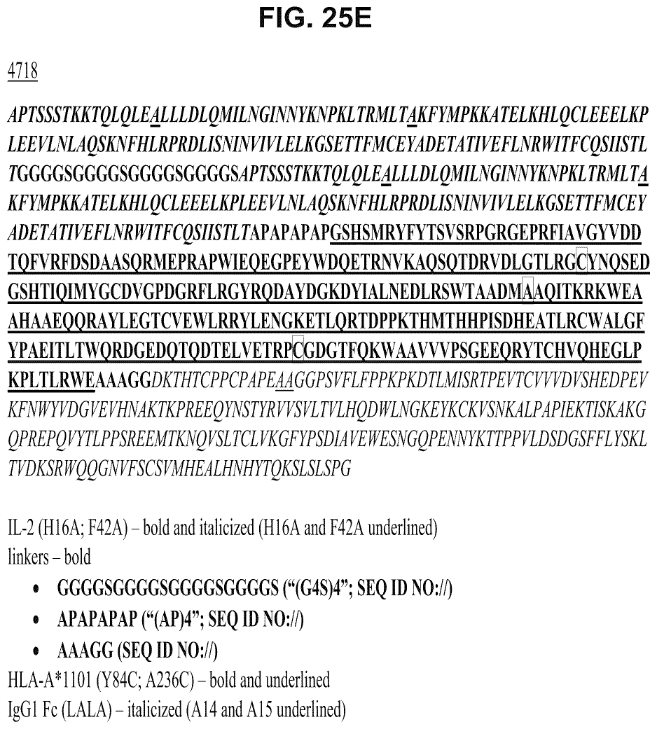

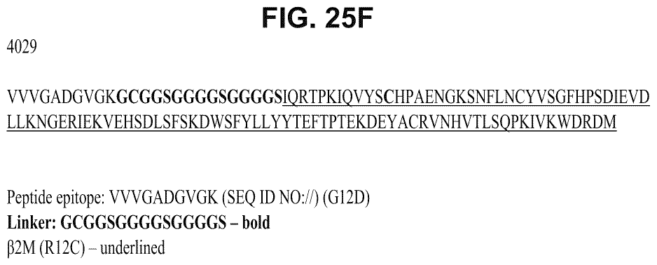

The amino acid sequences of constructs 4808, 4744, 4708, 4742, 4753, 4718, and 4029 are provided in FIGS. 25A-25F. The amino acid sequence of construct 4753 is provided in FIG. 23C.

Thermal stability of the TMPs was assessed using an accelerated stability assay conducted at 4° C., 37° C., and at 42° C. Compositions of dimerized TMPs were kept at the indicated temperatures in a solution (phosphate-buffered saline (PBS) containing 500 mM NaCl, pH 7.4), at a concentration of 10 mg of dimerized TMP/mL solution, for a period of time of 14 days. A 1 day, 7 days, and 14 days, the percent monomer remaining in the solution was determined using size exclusion chromatography. The PBS solution was as follows: 10.14 mM sodium phosphate dibasic, 1.76 mM potassium phosphate monobasic, 2.7 mM KCl, and 0.5 M NaCl; pH 7.4.

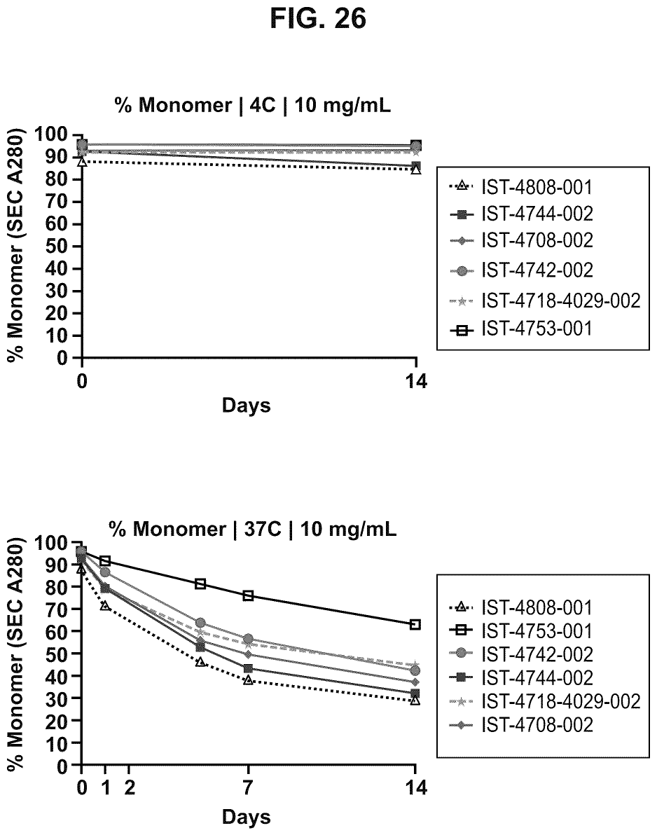

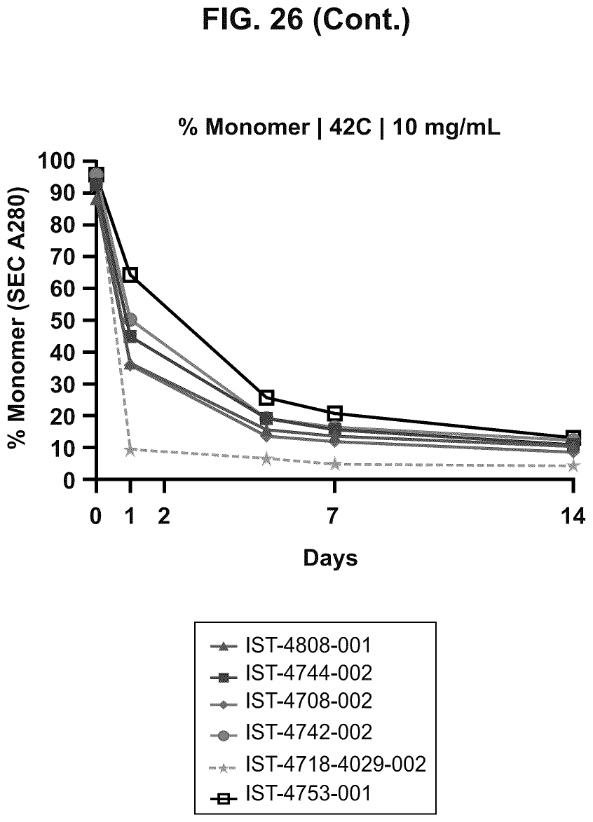

The data are shown in FIG. 26.

The percent monomer recovery at 14 days is summarized in Table 4, below:

TABLE 4

| Construct | 4 C. | 37 C. | 42 C. |
|---|---|---|---|
| 4808 | 96.0 | 33.0 | 11.7 |
| 4744 | 93.0 | 34.5 | 11.8 |
| 4708 | 100.2 | 39.8 | 9.1 |
| 4742 | 99.2 | 44.1 | 12.8 |
| 4718-4029 | 99.9 | 48.5 | 4.7 |
| 4753 | 99.6 | 65.7 | 13.6 |

The data indicate that 4753 has acceptable stability at 4C and 37C for 14 days.

As shown in Table 5, below, the 4753 construct exhibits good production and stability to freeze-thaw.

TABLE 5

| | 4753 |
|---|---|
| Titer (mg/L) | 162 |
| Post ProA SEC (% monomer) | 71 |
| Concentration (mg/mL) | 17.6 |
| Final SEC (% monomer) | 97.5 |
| Production yield (mg/L) | 50 |
| Endotoxin (EU/mg) | <0.1 |
| 3x Freeze-thaw | No change |
| Intact mass | Confirmed |
| Epitope clipping (%) | 0 |

### Example 2: In Vitro Activity of TMPs with Intrachain Disulfide Bond

The in vitro biological activity of the 4753 construct was analyzed, and compared with that of the 4708, and the 4742 constructs.

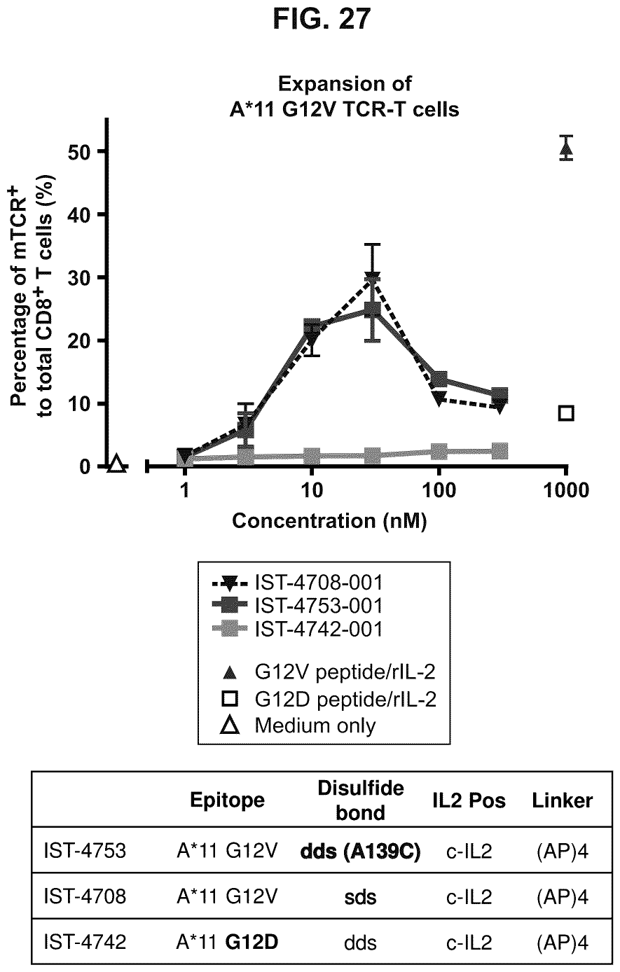

T cells with TCRs specific for KRAS G12V complexed with HLA-A*1101 and β2M were spiked into autologous peripheral blood mononuclear cells (PBMCs), then treated with increasing concentrations of the TMP and cultured for 10 days. After the 10-day culture period, the cells were collected and stained with surface markers and anti-mTCRβ antibodies, to detect T cells with TCRs specific for KRAS G12V complexed with HLA-A*1101 and β2M. T cells with TCRs specific for KRAS G12V complexed with HLA-A*1101 and β2M are referred to as “A*11 G12V TCR-T cells” in FIG. 27.

As shown in FIG. 27, all of the constructs that include the G12D peptide epitope induced expansion of G12V-specific CD8⁺ T cells (“A*11 G12V TCR-T cells”) in an antigen-specific and dose-dependent manner. Controls (medium only; G12D/recombinant IL-2) did not induce expansion of A*11 G12V TCR-T cells. The construct 4742, which includes the G12D peptide, also did not induce expansion of A*11 G12V TCR-T cells. G12V peptide/recombinant IL-2 (rIL-2) was included as a positive control and induced expansion of A*11 G12V TCR-T cells.

### Example 3: In Vitro Activity of TMPs with Intrachain Disulfide Bond

The in vitro biological activity of the 4817 construct and the 4753 construct was analyzed. The amino acid sequence of the 4753 construct is provided in FIG. 23C; the amino acid sequence of the 4817 construct is provided in FIG. 23E. Constructs 4753 and 4817 differ in amino acid sequence only in the KRAS peptide epitope: the KRAS epitope in construct 4817 is KRAS (7-16; G12D) VVVGADGVGK (SEQ ID NO: 184), while the KRAS epitope in construct 4753 is KRAS (7-16; G12V) VVVGAVGVGK (SEQ ID NO: 195). The features of constructs 4753 and 4817 are summarized in Table 6, below.

TABLE 6

| Construct | Epitope | HLA allele | S—S bond(s) |
|---|---|---|---|
| 4817 | KRAS G12D | A*1101 | G2C:A139C |
| | | | R12C:A236C |
| 4753 | KRAS G12V | A*1101 | G2C:A139C |
| | | | R12C:A236C |

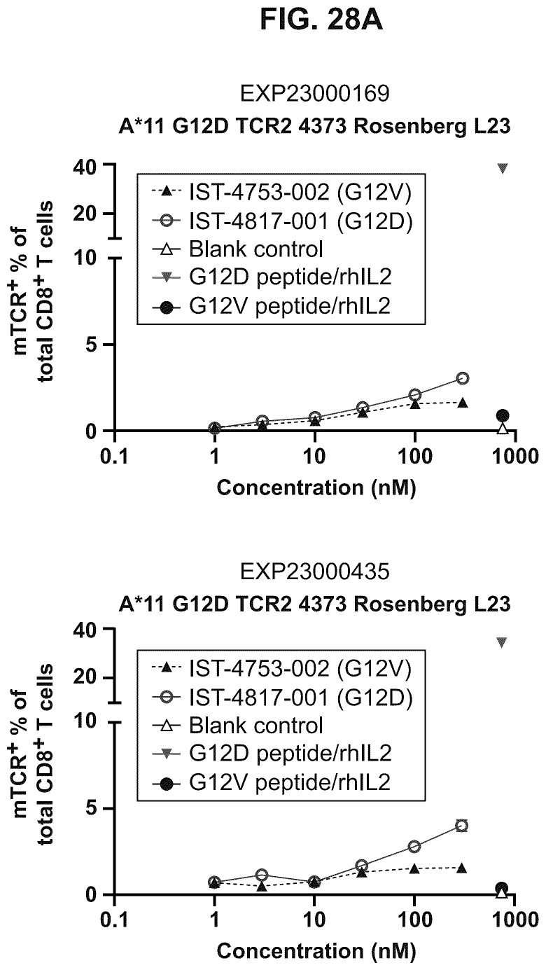

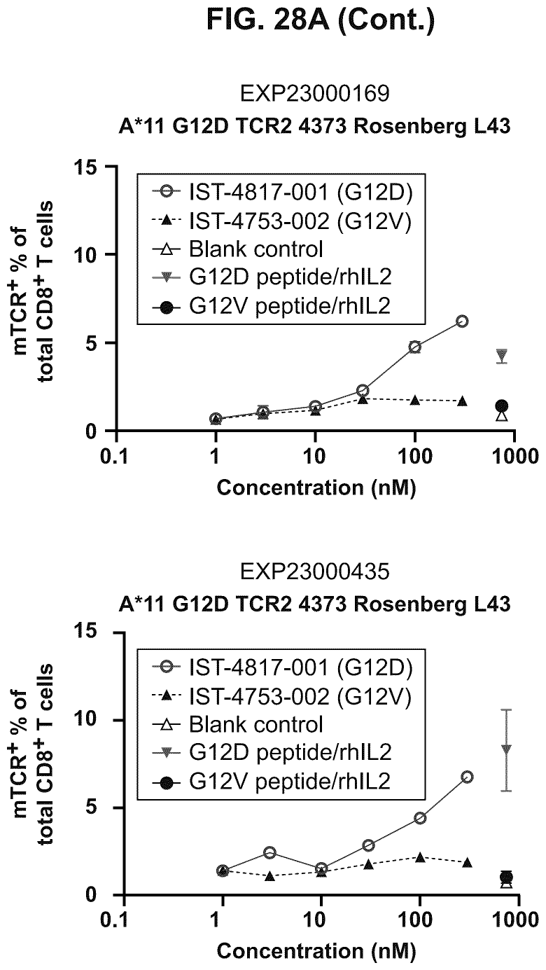

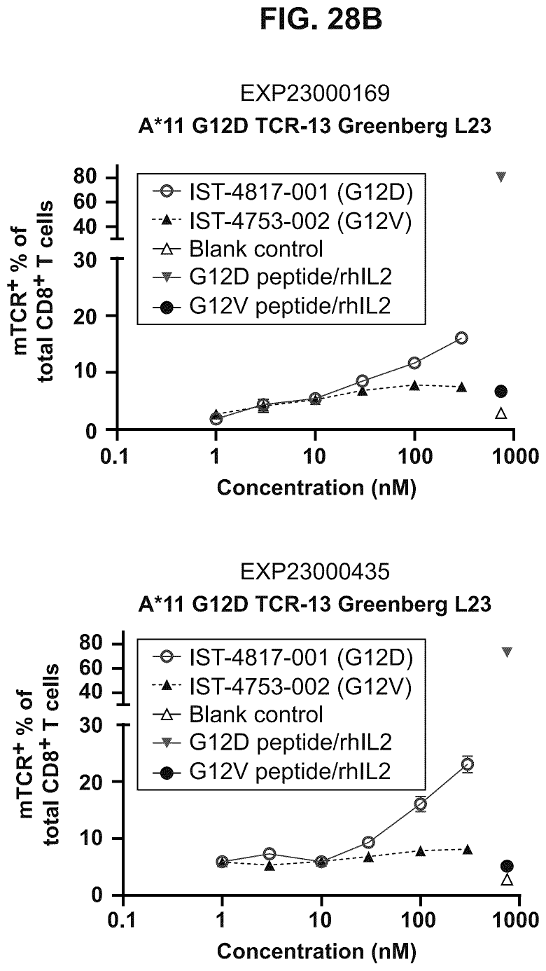

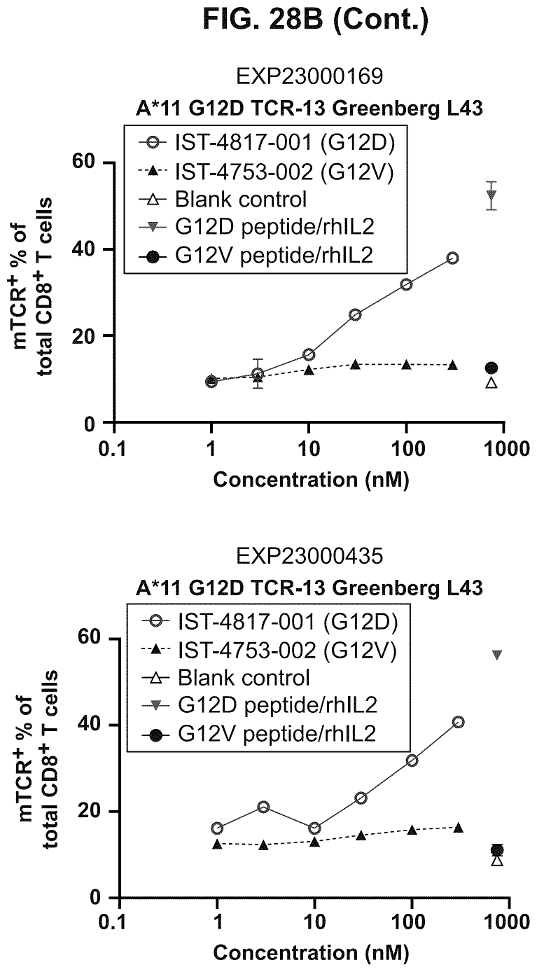

T cells with TCRs specific for KRAS G12D complexed with HLA-A*1101 and β2M were spiked into autologous PBMCs, then treated with increasing concentrations of the TMP and cultured for 10 days. After the 10-day culture period, the cells were collected and stained with surface markers and anti-mTCRβ antibodies, to detect T cells with TCRs specific for KRAS G12D complexed with HLA-A*1101 and β2M. Two different TCRs were used: “TCR2 4373 Rosenberg” (FIG. 28A); and “TCR-13 Greenberg” (FIG. 28B).

As shown in FIG. 28A, the construct 4817 expands T-cells with a TCR specific for KRAS G12D (complexed with HLA-A*1101 and β2M) in an antigen-specific manner. Similarly, as shown in FIG. 28B, the construct 4817 expands T-cells with a TCR specific for KRAS G12D (complexed with HLA-A*1101 and β2M polypeptides to form a pMHC) in an antigen-specific manner.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope. All such modifications are intended to be within the scope of the claims appended hereto.

What is claimed is:

1. A peptide-major histocompatibility complex (pMHC) polypeptide comprising, in order from N-terminus to C-terminus:
a) a peptide epitope having a length of from 4 amino acids to 25 amino acids;
b) a first peptide linker, wherein the first peptide linker comprises a cysteine (Cys);
c) a beta-2 microglobulin (β2M) polypeptide;
d) a second peptide linker; and
e) a major histocompatibility complex (MHC) class I heavy chain polypeptide, wherein the MHC class I heavy chain polypeptide comprises a Cys at any one of amino acids 135-143, based on the numbering of the MHC class I heavy chain polypeptide depicted in FIG. 3A, and wherein amino acid 84, based on the numbering of the MHC class I heavy chain polypeptide depicted in FIG. 3A, is other than Cys,
wherein the pMHC polypeptide comprises a disulfide bond formed between the Cys present in the first peptide linker and the Cys at any one of amino acids 135-143 of the MHC class I heavy chain polypeptide, and
wherein the pMHC polypeptide presents the epitope for binding to a T cell receptor.

2. The pMHC polypeptide of claim 1, wherein the MHC class I heavy chain polypeptide comprises a Cys at amino acid 138, 139, or 140 based on the numbering of the MHC class I heavy chain polypeptide depicted in FIG. 3A.

3. The pMHC polypeptide of claim 1, wherein the MHC class I heavy chain polypeptide comprises a Cys at amino acid 139 based on the numbering of the MHC class I heavy chain polypeptide depicted in FIG. 3A.

4. The pMHC polypeptide of any one of claims 1-3, wherein the peptide has a length of from 8 amino acids to 12 amino acids.

5. The pMHC polypeptide of any one of claims 1-4, wherein the first linker comprises the sequence CGGGS(GGGGS)n, GCGGS(GGGGS)n, or GGCGS(GGGGS)n, wherein n is an integer from 1-10.

6. The pMHC polypeptide of any one of claims 1-4, wherein the first linker comprises the sequence GCGGS(GGGGS)n, wherein n is an integer from 1-4.

7. The pMHC polypeptide of any one of claims 1-4, wherein the first linker comprises the sequence GCGGS(GGGGS)n, wherein n is 2.

8. The pMHC polypeptide of any one of claims 1-7, wherein the β2M polypeptide comprises an amino acid sequence having at least 95% amino acid sequence identity to the β2M amino acid sequence depicted in FIG. 2A.

9. The pMHC polypeptide of any one of claims 1-8, wherein the β2M polypeptide comprises a Cys at amino acid 12,
wherein the MHC class I heavy chain polypeptide comprises a Cys at amino acid 236, and
wherein the pMHC polypeptide further comprises a disulfide bond formed between the Cys at amino acid 12 of the β2M polypeptide and the Cys at amino acid 236 of the MHC class I heavy chain polypeptide.

10. The pMHC polypeptide of any one of claims 1-9, wherein the MHC class I heavy chain polypeptide comprises an amino acid sequence having at least 95% amino acid sequence identity to an HLA-A polypeptide, an HLA-B polypeptide, an HLA-C polypeptide, or an HLA-E polypeptide.

11. The pMHC polypeptide of claim 10, wherein the HLA-A polypeptide is an HLA-A*0301, an HLA-A*1101 polypeptide, an HLA-A*3303 polypeptide, an HLA-A*0201 polypeptide, or an HLA-A*2401 polypeptide.

12. The pMHC polypeptide of any one of claims 1-11, wherein the MHC class I heavy chain polypeptide comprises an amino acid sequence having at least 95% amino acid sequence identity to the amino acid sequence depicted in any one of FIG. 3B-3E, FIG. 4B-4E, FIG. 5B-5E, FIG. 6B-6E, FIG. 7B-7E, FIG. 11B-11E, or FIG. 12B-12E.

13. The pMHC polypeptide of any one of claims 1-12, wherein the second peptide linker has a length of from 4 amino acids to 25 amino acids.

14. The pMHC polypeptide of any one of claims 1-13, wherein the peptide epitope is a cancer-associated peptide or a peptide associated with a viral infection.

15. The pMHC polypeptide of claim 14, wherein the cancer-associated peptide is selected from a peptide of an alpha-feto protein (AFP) polypeptide, a Wilms tumor-1 (WT-1) polypeptide, a human papilloma virus (HPV) polypeptide, a MUC-1 polypeptide, a melanoma-associated antigen-4 (MAGE-A4) polypeptide, an NY-ESO-1 polypeptide, a survivin polypeptide, a mesothelin polypeptide, and a Preferentially Expressed Antigen of Melanoma (PRAME) polypeptide, and
wherein the peptide associated with a viral infection is selected from cytomegalovirus (CMV), Epstein-Barr virus (EBV), human papilloma virus, adenovirus, and coronaviruses such as a SARS-CoV-2 virus.

16. The pMHC polypeptide of any one of claims 1-15, wherein the polypeptide does not comprise a polypeptide other than the peptide epitope, the peptide linkers, the β2M polypeptide, and the MHC class I heavy chain polypeptide.

17. The pMHC polypeptide of any one of claims 1-16, wherein the pMHC has a length of from about 400 amino acids to about 450 amino acids.

18. The pMHC polypeptide of claim 17, wherein the pMHC has a length of from 410 amino acids to 420 amino acids.

19. A pMHC polypeptide, wherein the pMHC polypeptide is a multimer comprising two or more of the pMHC polypeptides of any one of claims 1-18.

20. A pMHC polypeptide of claim 19, wherein the multimeric polypeptide is a dimer comprising two of the pMHC polypeptides.

21. A pMHC polypeptide of claim 19, wherein the multimeric polypeptide is a trimer comprising three of the pMHC polypeptides.

22. A pMHC polypeptide of claim 19, wherein the multimeric polypeptide is a tetramer comprising four of the pMHC polypeptides.

23. The pMHC polypeptide of any one of claims 1-22, comprising one or more covalently or non-covalently linked moieties, wherein the one or more moieties is other than a polypeptide.

24. The pMHC polypeptide of claim 23, wherein the moiety is a detectable label.

25. The pMHC polypeptide of claim 24, wherein the detectable label is a fluorophore, a radioactive label, or a chromophore.

26. The pMHC polypeptide of claim 23, wherein the moiety is an insoluble support.

27. The pMHC polypeptide of claim 26, wherein the insoluble support is a bead.

28. The pMHC polypeptide of claim 27, wherein the bead is a magnetic bead.

29. The pMHC polypeptide of claim 23, wherein the moiety is a member of a specific binding pair.

30. The pMHC polypeptide of claim 29, wherein the member of the specific binding pair is biotin.

31. The pMHC polypeptide of claim 23, wherein the moiety comprises a lipid, a nanoparticle, or a polymer.

32. A library of the pMHC polypeptides of any one of claims 1-22, wherein at least two of the pMHC polypeptides in the library comprise different peptide epitopes.

33. The library of claim 32, wherein the pMHC polypeptides include a covalently linked nucleic acid barcode.

34. A pharmaceutical composition comprising the pMHC polypeptide of any one of claims 1-31.

35. The pharmaceutical composition of claim 34, wherein the pharmaceutical composition comprises an adjuvant.

36. A nucleic acid comprising a nucleotide sequence encoding the pMHC polypeptide of any one of claims 1-18.

37. The nucleic acid of claim 36, wherein the nucleotide sequence is operably linked to one or more transcriptional control elements.

38. A recombinant expression vector comprising a nucleic acid of claim 36 or claim 37.

39. A host cell genetically modified with the nucleic acid of claim 36 or claim 37 or the recombinant expression vector of claim 38.

40. A method of producing the pMHC polypeptide of any one of claims 1-18, comprising culturing in vitro the host cell of claim 39 under conditions in which the host cell produces the pMHC polypeptide.

41. A method of detecting a T cell comprising a T cell receptor (TCR) that binds a peptide epitope presented by the pMHC polypeptide of any one of claims 1-31, or the library of claim 32 or 33, the method comprising: a) contacting the T cell with the pMHC polypeptide of any one of claims 1-31, or the library of claim 32 or 33, thereby forming a pMHC polypeptide-T cell complex; and b) detecting the pMHC polypeptide-T cell complex.

42. The method of claim 41, wherein the pMHC polypeptide comprises a detectable label.

43. A method of inducing an immune response to a peptide epitope in an individual, the method comprising administering to the individual a composition of claim 34 or claim 35.

44. A pMHC complex comprising:
a) a pMHC polypeptide of any one of claims 1-31; and
b) one or more components, wherein the one or more components comprises one or more of:
i) one or more heterologous polypeptides;
ii) a nucleic acid component comprising one or more nucleic acids; and
iii) a component other than a polypeptide or nucleic acid component.

45. The molecule of claim 44, wherein the pMHC complex is a pMHC fusion molecule comprising one or more heterologous polypeptides.

46. The pMHC complex of claim 44 or 45, wherein the one or more heterologous polypeptides comprises an immunoglobulin (Ig) Fc polypeptide, optionally wherein the Ig Fc polypeptide substantially does not induce cell lysis.

47. The pMHC complex of claim 46, wherein the Ig Fc polypeptide comprises an Ala at amino acid 14 and an Ala at amino acid 15, based on the amino acid number of the Ig Fc amino acid sequence depicted in FIG. 16A.

48. The pMHC complex of claim 46 or 47, wherein the Ig Fc polypeptide comprises an amino acid sequence having at least 95% amino acid sequence identity to the amino acid sequence depicted in FIG. 16H.

49. The pMHC complex of any one of claims 44-48, wherein the one or more heterologous polypeptides comprises an antigen-binding region of an antibody.

50. The pMHC complex of claim 49, wherein the antigen-binding region is a single-chain Fc polypeptide or a nanobody.

51. The pMHC complex of claim 49 or claim 50, wherein the antigen-binding region specifically binds a cancer-associated antigen present on the surface of a cancerous cell.

52. The pMHC complex of claim 44, wherein the component comprising a nucleic acid comprises a nucleotide sequence that encodes a polypeptide.

53. The pMHC complex of claim 52, wherein the nucleic acid is a DNA molecule.

54. The pMHC complex of claim 52, wherein the nucleic acid is an RNA molecule.

55. The pMHC complex of claim 52, wherein the nucleic acid encodes a chimeric antigen receptor (CAR).

56. The pMHC complex of claim 44, wherein the one or more heterologous polypeptides comprises a CRISPR-Cas effector polypeptide.

57. The pMHC complex of claim 44 or claim 56, wherein the component comprising a nucleic acid comprises a CRISPR-Cas guide nucleic acid.

58. A nucleic acid comprising a nucleotide sequence encoding a molecule of any one of claims 44-51 and claim 56.

59. A nucleic acid of claim 58, wherein the nucleotide sequence is operably linked to one or more transcriptional control elements.

60. A recombinant expression vector comprising a nucleic acid of claim 58 or claim 59.

61. A host cell genetically modified with the nucleic acid of claim 58 or claim 59 or the recombinant expression vector of claim 60.

62. A method of producing the pMHC complex of any one of claims 44-51, comprising culturing in vitro the host cell of claim 61 under conditions in which the host cell produces the pMHC complex.

63. A composition comprising the pMHC complex of any one of claims 44-57.

64. A method of delivering a polypeptide and/or a nucleic acid to a T cell, the method comprising contacting the T cell with the pMHC complex of any one of claims 44-57, wherein the T cell comprises a T cell receptor that binds the pMHC of the pMHC complex, and wherein said contacting provides for delivery of the one or more components to the T cell.

65. A single-chain T-cell modulatory polypeptide (TMP) comprising:
a) a peptide-major histocompatibility complex (pMHC) polypeptide according to any one of claims 1-18;
b) one or more immunomodulatory polypeptides; and
c) an immunoglobulin (Ig) Fc polypeptide or a non-immunoglobulin scaffold component.

66. The TMP of claim 65, wherein the TMP comprises from N-terminus to C-terminus:
a1) the pMHC,
b1) the one or more immunomodulatory polypeptides, and
c1) an Ig Fc polypeptide; or
a2) the pMHC,
b2) an Ig Fc polypeptide, and
c2) the one or more immunomodulatory polypeptides, and
wherein the TMP may further comprise an independently selected linker interposed between any two of the components of the TMP.

67. The TMP of claim 65 or claim 66, wherein the TMP comprises from N-terminus to C-terminus:
a1) the pMHC,
b1) an optional linker,
c1) an Ig Fc polypeptide,
d1) an optional linker, and
e1) an immunomodulatory polypeptide; or
a2) the pMHC,
b2) an optional linker,
c2) the Ig Fc polypeptide,
d2) an optional linker,
e2) a first immunomodulatory polypeptide,
f2) an optional linker, and
g2) a second immunomodulatory polypeptide.

68. The TMP of claim 67,
wherein the TMP comprises at least one short flexible peptide linker and/or at least one rigid peptide linker, and
wherein each short flexible peptide linker comprises from 2-4, 2-5, 3-6, 4-8, 5-10 or 10-14 amino acids, and
wherein each rigid peptide linker is independently selected from the group consisting of:
i) (AP)n, where n is an integer from 1-10;
ii) (EP)n, where n is an integer from 1-10;
iii) (KP)n, where n is an integer from 1-10; and
iv) a peptide comprising EAAAK.

69. The TMP of any one of claims 65-68, wherein the TMP comprises an Ig Fc polypeptide, optionally wherein the Ig Fc polypeptide substantially does not induce cell lysis.

70. The TMP of claim 69, wherein the Ig Fc polypeptide comprises an Ala at amino acid 14 and an Ala at amino acid 15, based on the amino acid number of the Ig Fc amino acid sequence depicted in FIG. 16A.

71. The TMP of claim 69 or claim 70, wherein the Ig Fc polypeptide comprises an amino acid sequence having at least 95% amino acid sequence identity to the amino acid sequence depicted in FIG. 16H.

72. The TMP of any one of claims 65-71, wherein at least one of the one or more immunomodulatory polypeptides is a wild-type or variant of an activating immunomodulatory polypeptide, optionally selected from IL-2, a 4-1BBL, CD80, CD86, and combinations thereof, and optionally wherein at least one of the at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide.

73. The TMP of claim 72, wherein the at least one immunomodulatory polypeptide is a variant of IL-2 that exhibits decreased binding affinity for IL-2Rα and IL-2Rβ, optionally wherein the variant IL-2 polypeptide comprises an amino acid other than histidine at position 16 and an amino acid other than phenylalanine at position 42.

74. The TMP of claim 73, wherein the at least one immunomodulatory polypeptide comprises i) an H16A substitution and an F42A substitution; ii) an H16T substitution and an F42A substitution, iii) an H16D and F42A substitution, or iv) an H16E substitution and an F42A substitution.

75. A protein comprising two TMPs according to any one of claims 65-74, wherein the two TMPs each comprise and Ig Fc polypeptide,
wherein the two TMPs have the same amino acid sequence, and
wherein the two TMPs are joined by one or more disulfide bonds that join the Ig Fc component of one TMP to the Ig Fc component of the other TMP, optionally wherein the two TMPs are joined by two disulfide bonds that join the Ig Fc component of one TMP to the Ig Fc component of the other TMP.

76. A pharmaceutical composition comprising the TMP of any one of claims 65-74, or the protein of claim 75.

77. A nucleic acid comprising a nucleotide sequence encoding the TMP according to any one of claims 65-74.

78. The nucleic acid of claim 77, wherein the nucleotide sequence is operably linked to one or more transcriptional control elements.

79. A recombinant expression vector comprising the nucleic acid of claim 77 or claim 78.

80. A host cell genetically modified with the nucleic acid of claim 77 or claim 78, or the recombinant expression vector of claim 79.

81. A method of producing a TMP according to any one of claims 65-74, or the protein of claim 75, the method comprising culturing in vitro the host cell of claim 80 under conditions in which the host cell produces the TMP.

82. A method of modulating the activity of a T cell, the method comprising contacting the T cell with a TMP according to any one of claims 65-74 or the protein of claim 75.

83. A method of claim 82 wherein said contacting is in vitro.

84. A method of claim 82, wherein said contacting is in vivo.

85. A method of treating cancer in an individual, the method comprising administering to the individual an effective amount of the TMP of any one of claims 65-74 or the protein of claim 75, wherein the peptide epitope is a cancer-associated peptide epitope.

86. The method of claim 85, further comprising administering one or more additional therapeutic agents.

87. The method of claim 86, wherein the one or more additional therapeutic agents comprises an immune checkpoint inhibitor.

88. The method of claim 87, wherein the immune checkpoint inhibitor is an antibody specific for PD-1, PD-L1, or CTLA4.

89. The method of claim 88, wherein the antibody is a single-chain Fv or a nanobody.

90. A method of treating a viral infection in an individual, the method comprising administering to the individual a TMP of any one of claims 65-74 or the protein of claim 75, wherein the peptide epitope is a peptide associated with a viral infection.


SEQUENCE LISTING (384 sequences)

SEQ ID NO: 1 - protein, 188 aa, synthetic construct [sequence omitted]

SEQ ID NO: 2 - protein, 10 aa, synthetic construct
VVVGAGDVGK

SEQ ID NO: 3 - protein, 9 aa, synthetic construct
VVGAGDVGK

SEQ ID NO: 4 - protein, 10 aa, synthetic construct
VVVGARGVGK

SEQ ID NO: 5 - protein, 9 aa, synthetic construct
VVGARGVGK

SEQ ID NO: 6 - protein, 9 aa, synthetic construct
LVVVGADGV

SEQ ID NO: 7 - protein, 9 aa, synthetic construct
LVVVGAVGV

SEQ ID NO: 8 - protein, 9 aa, synthetic construct
LVVVGACGV

SEQ ID NO: 9 - protein, 10 aa, synthetic construct
KLVVVGADGV

SEQ ID NO: 10 - protein, 10 aa, synthetic construct
KLVVVGAVGV

SEQ ID NO: 11 - protein, 10 aa, synthetic construct
KLVVVGACGV

SEQ ID NO: 12 - protein, 9 aa, synthetic construct
LLVVGADGV

SEQ ID NO: 13 - protein, 9 aa, synthetic construct
LLVVGAVGV

SEQ ID NO: 14 - protein, 9 aa, synthetic construct
LLVVGACGV

SEQ ID NO: 15 - protein, 10 aa, synthetic construct
FLVVVGADGV

SEQ ID NO: 16 - protein, 10 aa, synthetic construct
FLVVVGAVGV

SEQ ID NO: 17 - protein, 10 aa, synthetic construct
FLVVVGACGV

SEQ ID NO: 18 - protein, 10 aa, synthetic construct
KLVVVGAGDV

SEQ ID NO: 19 - protein, 10 aa, synthetic construct
KLVVVGARGV

SEQ ID NO: 20 - protein, 10 aa, synthetic construct
GAGDVGKSAL

SEQ ID NO: 21 - protein, 9 aa, synthetic construct
AGDVGKSAL

SEQ ID NO: 22 - protein, 9 aa, synthetic construct
DVGKSALTI

SEQ ID NO: 23 - protein, 10 aa, synthetic construct
GAVGVGKSAL

SEQ ID NO: 24 - protein, 9 aa, synthetic construct
AVGVGKSAL

SEQ ID NO: 25 - protein, 9 aa, synthetic construct
YKLVVVGAV

SEQ ID NO: 26 - protein, 9 aa, synthetic construct
ARGVGKSAL

SEQ ID NO: 27 - protein, 10 aa, synthetic construct
GARGVGKSAL

SEQ ID NO: 28 - protein, 10 aa, synthetic construct
EYKLVVVGAR

SEQ ID NO: 29 - protein, 10 aa, synthetic construct
RGVGKSALTI

SEQ ID NO: 30 - protein, 9 aa, synthetic construct
LVVVGARGV

SEQ ID NO: 31 - protein, 10 aa, synthetic construct
GADGVGKSAL

SEQ ID NO: 32 - protein, 9 aa, synthetic construct
ACGVGKSAL

SEQ ID NO: 33 - protein, 10 aa, synthetic construct
GACGVGKSAL

SEQ ID NO: 34 - protein, 9 aa, synthetic construct
VVGAVGVGK

SEQ ID NO: 35 - protein, 10 aa, synthetic construct
VVVGAVGVGK

SEQ ID NO: 36 - protein, 9 aa, synthetic construct
VGAVGVGKS

SEQ ID NO: 37 - protein, 10 aa, synthetic construct
VGAVGVGKSA

SEQ ID NO: 38 - protein, 10 aa, synthetic construct
AVGVGKSALT

SEQ ID NO: 39 - protein, 9 aa, synthetic construct
GAVGVGKSA

SEQ ID NO: 40 - protein, 10 aa, synthetic construct
LVVVGAVGVG

SEQ ID NO: 41 - protein, 9 aa, synthetic construct
KLVVVGAVG

SEQ ID NO: 42 - protein, 99 aa, Homo sapiens [sequence omitted]

SEQ ID NO: 43 - protein, 99 aa, synthetic construct
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 44 - protein, 275 aa, Homo sapiens [sequence omitted]

SEQ ID NO: 45 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 46 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 47 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 48 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 49 - protein, 275 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 50 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 51 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 52 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 53 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 54 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 55 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 56 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 57 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 58 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 59 - protein, 275 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 60 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 61 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGAYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 62 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 63 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGAYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 64 - protein, 275 aa, Homo sapiens
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 65 - protein, 275 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 66 - protein, 275 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGAYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 67 - protein, 275 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 68 - protein, 275 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGAYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 69 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 70 - protein, 275 aa, Homo sapiens; identical to SEQ ID NO: 54

SEQ ID NO: 71 - protein, 275 aa, Homo sapiens
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 72 - protein, 275 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 73 - protein, 275 aa, Homo sapiens; identical to SEQ ID NO: 64

SEQ ID NO: 74 - protein, 275 aa, Homo sapiens; identical to SEQ ID NO: 49

SEQ ID NO: 75 - protein, 275 aa, Homo sapiens [sequence omitted]

SEQ ID NO: 76 - protein, 275 aa, Homo sapiens; identical to SEQ ID NO: 59

SEQ ID NO: 77 - protein, 275 aa, Homo sapiens
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 78 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRXYNQSEAGSHTLQMMFGCDVGSDGRFLRGYHQYAYDGKDYIALKEDLRSWTAADMCAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 79 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRXYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 80 - protein, 275 aa, synthetic construct
GSHSMRYFSTSVSRPGRGEPRFIAVGYVDDTQFVRFDSDAASQRMEPRAPWIEQEGPEYWDEETGKVKAQSQTDRENLRIALRXYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 81 - protein, 275 aa, synthetic construct
GSHSMRYFFTSVSRPGRGEPRFIAVGYVDDTQFVRFDSDAASQKMEPRAPWIEQEGPEYWDQETRNMKAHSQTDRANLGTLRGXYNQSEDGSHTIQIMYGCDVGPDGRFLRGYRQDAYDGKDYIALNEDLRSWTAADMCAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 82 - protein, 275 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGXYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 83 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGXYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 84 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGXYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 85 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGXYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 86 - protein, 275 aa, synthetic construct
GSHSMRYFYTSVSRPGRGEPRFIAVGYVDDTQFVRFDSDAASQRMEPRAPWIEQEGPEYWDRNTRKVKAQSQTDRVDLGTLRGXYNQSEDGSHTIQRMYGCDVGPDGRFLRGYQQDAYDGKDYIALNEDLRSWTAADMCAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 87 - protein, 276 aa, Homo sapiens
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 88 - protein, 276 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 89 - protein, 275 aa, Homo sapiens
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVWPSGEEQRYTCHVQHEGLPKPLTLRWEP

SEQ ID NO: 90 - protein, 276 aa, Homo sapiens
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 91 - protein, 276 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 92 - protein, 276 aa, Homo sapiens [sequence omitted]

SEQ ID NO: 93 - protein, 276 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 94 - protein, 276 aa, synthetic construct
GSHSMRYFYTSVSRPGRGEPRFISVGYVDDTQFVRFDSDAASPREEPRAPWIEQEGPEYWDRNTQICKTNTQTYRENLRTALRXYNQSEAGSHTLQRMYGCDVGPDGRLLRGHNQFAYDGKDYIALNEDLSSWTAADTCAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 95 - protein, 276 aa, synthetic construct
GSHSMRYFYTAMSRPGRGEPRFIAVGYVDDTQFVRFDSDAASPRTEPRAPWIEQEGPEYWDRNTQIFKTNTQTYRENLRIALRXYNQSEAGSHIIQRMYGCDLGPDGRLLRGHDQSAYDGKDYIALNEDLSSWTAADTCAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 96 - protein, 275 aa, synthetic construct
GSHSMRYFYTAMSRPGRGEPRFIAVGYVDDTQFVRFDSDAASPRMAPRAPWIEQEGPEYWDRNTQISKTNTQTYRESLRNLRGXYNQSEAGSHIIQRMYGCDVGPDGRLLRGYDQSAYDGKDYIALNEDLSSWTAADTCAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVWPSGEEQRYTCHVQHEGLPKPLTLRWEP

SEQ ID NO: 97 - protein, 276 aa, synthetic construct
GSHSMRYFYTAMSRPGRGEPRFIAVGYVDDTQFVRFDSDAASPRMAPRAPWIEQEGPEYWDRETQKYKRQAQTDRVSLRNLRGXYNQSEAGSHTLQRMYGCDVGPDGRLLRGHDQSAYDGKDYIALNEDLSSWTAADTCAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 98 - protein, 276 aa, synthetic construct
GSHSMRYFHTAMSRPGRGEPRFITVGYVDDTLFVRFDSDATSPRKEPRAPWIEQEGPEYWDRETQISKTNTQTYRESLRNLRGXYNQSEAGSHTLQRMYGCDVGPDGRLLRGHNQYAYDGKDYIALNEDLRSWTAADTCAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 99 - protein, 276 aa, synthetic construct
GSHSMRYFYTSVSRPGRGEPRFISVGYVDDTQFVRFDSDAASPREEPRAPWIEQEGPEYWDRNTQIYKAQAQTDRESLRNLRGXYNQSEAGSHTLQSMYGCDVGPDGRLLRGHDQYAYDGKDYIALNEDLRSWTAADTCAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 100 - protein, 276 aa, synthetic construct
GSHSMRYFDTAMSRPGRGEPRFISVGYVDDTQFVRFDSDAASPREEPRAPWIEQEGPEYWDRNTQIFKTNTQTDRESLRNLRGXYNQSEAGSHTLQSMYGCDVGPDGRLLRGHNQYAYDGKDYIALNEDLRSWTAADTCAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 101 - protein, 275 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 102 - protein, 275 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 103 - protein, 275 aa, Homo sapiens
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWK

SEQ ID NO: 104 - protein, 275 aa, Homo sapiens
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWE

SEQ ID NO: 105 - protein, 275 aa, Homo sapiens
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWE

SEQ ID NO: 106 - protein, 275 aa, Homo sapiens
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWG

SEQ ID NO: 107 - protein, 275 aa, Homo sapiens
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWE

SEQ ID NO: 108 - protein, 275 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWE

SEQ ID NO: 109 - protein, 275 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWE

SEQ ID NO: 110 - protein, 275 aa, synthetic construct
CSHSMRYFDTAVSRPGRGEPRFISVGYVDDTQFVRFDSDAASPRGEPRAPWVEQEGPEYWDRETQNYKRQAQADRVSLRNLRGXYNQSEDGSHTLQRMYGCDLGPDGRLLRGYDQSAYDGKDYIALNEDLRSWTAADTCAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 111 - protein, 275 aa, synthetic construct
CSHSMRYFDTAVSRPGRGEPRFISVGYVDDTQFVRFDSDAASPRGEPRAPWVEQEGPEYWDRETQKYKRQAQADRVSLRNLRGXYNQSEDGSHTLQRMSGCDLGPDGRLLRGYDQSAYDGKDYIALNEDLRSWTAADTCAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 112 - protein, 275 aa, synthetic construct
GSHSMRYFSTSVSWPGRGEPRFIAVGYVDDTQFVRFDSDAASPRGEPREPWVEQEGPEYWDRETQKYKRQAQADRVNLRKLRGXYNQSEDGSHTLQRMFGCDLGPDGRLLRGYNQFAYDGKDYIALNEDLRSWTAADTCAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWK

SEQ ID NO: 113 - protein, 275 aa, synthetic construct
CSHSMKYFFTSVSRPGRGEPRFISVGYVDDTQFVRFDSDAASPRGEPRAPWVEQEGPEYWDRETQKYKRQAQTDRVSLRNLRGXYNQSEAGSHTLQWMCGCDLGPDGRLLRGYDQYAYDGKDYIALNEDLRSWTAADTCAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWE

SEQ ID NO: 114 - protein, 275 aa, synthetic construct
CSHSMRYFDTAVSRPGRGEPRFISVGYVDDTQFVRFDSDAASPRGEPRAPWVEQEGPEYWDRETQKYKRQAQADRVNLRKLRGXYNQSEDGSHTLQWMYGCDLGPDGRLLRGYDQSAYDGKDYIALNEDLRSWTAADTCAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWE

SEQ ID NO: 115 - protein, 275 aa, synthetic construct
CSHSMRYFYTAVSRPGRGEPRFIAVGYVDDTQFVQFDSDAASPRGEPRAPWVEQEGPEYWDRETQKYKRQAQTDRVSLRNLRGXYNQSEAGSHTLQRMYGCDLGPDGRLLRGYNQFAYDGKDYIALNEDLRSWTAADTCAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWG

SEQ ID NO: 116 - protein, 275 aa, synthetic construct
CSHSMRYFYTAVSRPGRGEPHFIAVGYVDDTQFVRFDSDAASPRGEPRAPWVEQEGPEYWDRETQNYKRQAQTDRVNLRKLRGXYNQSEAGSHIIQRMYGCDLGPDGRLLRGHDQLAYDGKDYIALNEDLRSWTAADTCAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWE

SEQ ID NO: 117 - protein, 275 aa, synthetic construct
GSHSMRYFYTAVSRPGRGEPHFIAVGYVDDTQFVRFDSDAASPRGEPRAPWVEQEGPEYWDRETQKYKRQAQTDRVSLRNLRGXYNQSEARSHIIQRMYGCDVGPDGRLLRGYDQYAYDGKDYIALNEDLRSWTAADTCAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWE

SEQ ID NO: 118 - protein, 275 aa, synthetic construct
GSHSMRYFYTAVSRPGRGEPHFIAVGYVDDTQFVRFDSDAASPRGEPRAPWVEQEGPEYWDRETQKYKRQAQTDRVSLRNLRGXYNQSEAGSHIIQRMYGCDVGPDGRLLRGYDQYAYDGKDYIALNEDLRSWTAADTCAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWE

SEQ ID NO: 119 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 120 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 121 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 122 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 123 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 124 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 125 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 126 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 127 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 128 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 129 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 130 - protein, 275 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGAYNQSEASSHTLQWMIGCDLGSDGRLLRGYEQYAYDGKDYLALNEDLRSWTAADTCAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 131 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 132 - protein, 275 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGAYNQSEASSHTLQWMIGCDLGSDGRLIRGYEQYAYDGKDYLALNEDLRSWTAADTCAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 133 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 227 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 142 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 226 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 146 - protein, 502 aa, Homo sapiens
MDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 147 - protein, 519 aa, Homo sapiens
MDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 148 - protein, 522 aa, Homo sapiens
MDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 149 - protein, 302 aa, Homo sapiens
MEKGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 150 - protein, 288 aa, Homo sapiens
MEKGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 151 - protein, 158 aa, Human papillomavirus
MHQKRTAMFQDPQERPRKLPQLCTELQTTIHDIILECVYCKQQLLRREVYDFAFRDLCIVYRDGNPYAVCDKCLKFYSKISEYRHYCYSVYGTTLEQQYNKPLCDLLIRCINCQKPLCPEEKQRHLDKKQRFHNIRGRWTGRCMSCCRSSRTRRETQL

SEQ ID NO: 152 - protein, 141 aa, Human papillomavirus
MHGDTPTLHEYMLDLQPETTDLYCYEQLNYSSEEEDEIDGPAGQAEPDRAHYNIVTFCCKCDSTLRLCVQSTHVDIRTLEDLLMGTLGIVCPICSQKPYSTMADPAGTNGEEGTGCNGWFYVEAVVEKKTGDAISDDENEN

SEQ ID NO: 153 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 154 - protein, 264 aa, Homo sapiens
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 155 - protein, 255 aa, Homo sapiens
MTPGTQSPFFLLLLLTVLTVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 156 - protein, 203 aa, Homo sapiens
MTPGTQSPFFLLLLLTVLTVVTGSGHASSTPGGEKETSATQRSSVPSSTEKNAIPAPTTTKSCRETFLKCFCRFINKGVFWASPILSSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 157 - protein, 150 aa, Homo sapiens
MTPGTQSPFFLLLLLTVLTVVTGSGHASSTPGGEKETSATQRSSVPSSTEKNAFNSSLEDPSTDYYQELQRDISEMAVCQCRRKNYGQLDIFPARDTYHPMSEYPTYHTHGRYVPPSSTDRSPYEKVSAGNGGSSLSYTNPAVAATSANL

SEQ ID NO: 158 - protein, 159 aa, Homo sapiens
MTPGTQSPFFLLLLLTVLTATTAPKPATVVTGSGHASSTPGGEKETSATQRSSVPSSTEKNAFNSSLEDPSTDYYQELQRDISEMAVCQCRRKNYGQLDIFPARDTYHPMSEYPTYHTHGRYVPPSSTDRSPYEKVSAGNGGSSLSYTNPAVAATSANL

SEQ ID NO: 159 - protein, 158 aa, Homo sapiens
MTPGTQSPFFLLLLLTVLTVVTGSGHASSTPGGEKETSATQRSSVPSSTEKNAIPAPTTTKSCRETFLKCFCRFINKGVFWASPILSSVWGWGARLGHRAAGAGLCSGCAGHCLSHCLGCLSVPPKELRAAGHLSSPGYLPSYERVPHLPHPWALCAP

SEQ ID NO: 160 - protein, 475 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 161 - protein, 282 aa, Homo sapiens
MTPGTQSPFFLLLLLTVLTATTAPKPATVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 162 - protein, 219 aa, Homo sapiens
MTPGTQSPFFLLLLLTVLTATTAPKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVWGWGARLGHRAAGAGLCSGCAGHCLSHCLGCLSVPPKELRAAGHLSSPGYLPSYERVPHLPHPWALCAP

SEQ ID NO: 163 - protein, 238 aa, Homo sapiens
MTPGTQSPFFLLLLLTVLTATTAPKPATVVTGSGHASSTPGGEKETSATQRSSVPSSTEKNAIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 164 - protein, 217 aa, Homo sapiens
MTPGTQSPFFLLLLLTVLTGGEKETSATQRSSVPSSTEKNAIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 165 - protein, 241 aa, Homo sapiens
MTPGTQSPFFLLLLLTVLTATTAPKPATVVTGSGHASSTPGGEKETSATQRSSVPSSTEKNAFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 166 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAEKVRRAIEQLAAMD

SEQ ID NO: 167 - protein, 137 aa, Homo sapiens
MGAPTLPPAWQPFLKDHRISTFKNWPFLEGCACTPERMAEAGFIHCPTENEPDLAQCFFCFKELEGWEPDDDPMQRKPTIRRKNLRKLRRKCAVPSSSWLPWIEASGRSCLVPEWLHHFQGLFPGATSLPVGPLAMS

SEQ ID NO: 168 - protein, 165 aa, Homo sapiens
MGAPTLPPAWQPFLKDHRISTFKNWPFLEGCACTPERMAEAGFIHCPTENEPDLAQCFFCFKELEGWEPDDDPIGPGTVAYACNTSTLGGRGGRITREEHKKHSSGCAFLSVKKQFEELTLGEFLKLDRERAKNKIAKETNNKKKEFEETAEKVRRAIEQLAAMD

SEQ ID NO: 169 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 170 - protein, 493 aa, Homo sapiens
MSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 171 - protein, 930 aa, synthetic construct
XGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 172 - protein, 930 aa, synthetic construct
XGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGAYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 173 - protein, 939 aa, synthetic construct
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 174 - protein, 939 aa, synthetic construct
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGAYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 175 - protein, 938 aa, synthetic construct
VVVGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTL

SEQ ID NO: 176 - protein, 15 aa, synthetic construct
GCGGSGGGGSGGGGS

SEQ ID NO: 177 - protein, 15 aa, synthetic construct
GGGGSGGGGSGGGGS

SEQ ID NO: 178 - protein, 5 aa, synthetic construct
AAAGG

SEQ ID NO: 179 - protein, 8 aa, synthetic construct
APAPAPAP

SEQ ID NO: 180 - protein, 20 aa, synthetic construct
GGGGSGGGGSGGGGSGGGGS

SEQ ID NO: 181 - protein, 13 aa, synthetic construct
GCGGSGGGSGGGS

SEQ ID NO: 182 - protein, 15 aa, synthetic construct
GGGGSGGGGSGGGGS

SEQ ID NO: 183 - protein, 20 aa, synthetic construct
GGGGSGGGGSGGGGSGGGGS

SEQ ID NO: 184 - protein, 10 aa, synthetic construct
VVVGADGVGK

SEQ ID NO: 185 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 186 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 187 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 188 - protein, 133 aa, Homo sapiens [sequence omitted]

SEQ ID NO: 189 - protein, 939 aa, synthetic construct
VVVGAVGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 190 - protein, 939 aa, synthetic construct
VVVGADGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 191 - protein, 939 aa, synthetic construct
VVVGAVGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 192 - protein, 939 aa, synthetic construct
VVVGADGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 193 - protein, 800 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTAPAPAPAPGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 194 - protein, 124 aa, synthetic construct
VVVGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 195 - protein, 10 aa, synthetic construct
VVVGAVGVGK

SEQ ID NO: 196 - protein, 9 aa, synthetic construct
CADMAAQTT

SEQ ID NO: 197 - protein, 9 aa, synthetic construct
ACDMAAQTT

SEQ ID NO: 198 - protein, 9 aa, synthetic construct
AACMAAQTT

SEQ ID NO: 199 - protein, 9 aa, synthetic construct
AADCAAQTT

SEQ ID NO: 200 - protein, 9 aa, synthetic construct
AADMCAQTT

SEQ ID NO: 201 - protein, 9 aa, synthetic construct
AADMACQTT

SEQ ID NO: 202 - protein, 9 aa, synthetic construct
AADMAACTT

SEQ ID NO: 203 - protein, 9 aa, synthetic construct
AADMAAQCT

SEQ ID NO: 204 - protein, 9 aa, synthetic construct
AADMAAQTC

SEQ ID NO: 205 - protein, 9 aa, synthetic construct
CADMAAQIT

SEQ ID NO: 206 - protein, 9 aa, synthetic construct
ACDMAAQIT

SEQ ID NO: 207 - protein, 9 aa, synthetic construct
AACMAAQIT

SEQ ID NO: 208 - protein, 9 aa, synthetic construct
AADCAAQIT

SEQ ID NO: 209 - protein, 9 aa, synthetic construct
AADMCAQIT

SEQ ID NO: 210 - protein, 9 aa, synthetic construct
AADMACQIT

SEQ ID NO: 211 - protein, 9 aa, synthetic construct
AADMAACIT

SEQ ID NO: 212 - protein, 9 aa, synthetic construct
AADMAAQCT

SEQ ID NO: 213 - protein, 9 aa, synthetic construct
AADMAAQIC

SEQ ID NO: 214 - protein, 9 aa, synthetic construct
CADTAAQIT

SEQ ID NO: 215 - protein, 9 aa, synthetic construct
ACDTAAQIT

SEQ ID NO: 216 - protein, 9 aa, synthetic construct
AACTAAQIT

SEQ ID NO: 217 - protein, 9 aa, synthetic construct
AADCAAQIT

SEQ ID NO: 218 - protein, 9 aa, synthetic construct
AADTCAQIT

SEQ ID NO: 219 - protein, 9 aa, synthetic construct
AADTACQIT

SEQ ID NO: 220 - protein, 9 aa, synthetic construct
AADTAACIT

SEQ ID NO: 221 - protein, 9 aa, synthetic construct
AADTAAQCT

SEQ ID NO: 222 - protein, 9 aa, synthetic construct
AADTAAQIC

SEQ ID NO: 223 - protein, 9 aa, synthetic construct
CVDTAAQIS

SEQ ID NO: 224 - protein, 9 aa, synthetic construct
ACDTAAQIS

SEQ ID NO: 225 - protein, 9 aa, synthetic construct
AVCTAAQIS

SEQ ID NO: 226 - protein, 9 aa, synthetic construct
AVDCAAQIS

SEQ ID NO: 227 - protein, 9 aa, synthetic construct
AVDTCAQIS

SEQ ID NO: 228 - protein, 9 aa, synthetic construct
AVDTACQIS

SEQ ID NO: 229 - protein, 9 aa, synthetic construct
AVDTAACIS

SEQ ID NO: 230 - protein, 9 aa, synthetic construct
AVDTAAQCS

SEQ ID NO: 231 - protein, 9 aa, synthetic construct
AVDTAAQIC

SEQ ID NO: 232 - protein, 55 aa, synthetic construct
  VARIANT 6..55: note = Wherein up to 9 copies of GGGGS may be deleted
CGGGSGGGGSGGGGSGGGGSGGGGSGGGGSGGGGSGGGGSGGGGSGGGGSGGGGS

SEQ ID NO: 233 - protein, 55 aa, synthetic construct
  VARIANT 6..55: note = Wherein up to 9 copies of GGGGS may be deleted
GCGGSGGGGSGGGGSGGGGSGGGGSGGGGSGGGGSGGGGSGGGGSGGGGSGGGGS

SEQ ID NO: 234 - protein, 55 aa, synthetic construct
  VARIANT 6..55: note = Wherein up to 9 copies of GGGGS may be deleted
GGCGSGGGGSGGGGSGGGGSGGGGSGGGGSGGGGSGGGGSGGGGSGGGGSGGGGS

SEQ ID NO: 235 - protein, 10 aa, synthetic construct
CGGGSGGGGS

SEQ ID NO: 236 - protein, 15 aa, synthetic construct
CGGGSGGGGSGGGGS

SEQ ID NO: 237 - protein, 20 aa, synthetic construct
CGGGSGGGGSGGGGSGGGGS

SEQ ID NO: 238 - protein, 10 aa, synthetic construct
GCGGSGGGGS

SEQ ID NO: 239 - protein, 15 aa, synthetic construct
GCGGSGGGGSGGGGS

SEQ ID NO: 240 - protein, 20 aa, synthetic construct
GCGGSGGGGSGGGGSGGGGS

SEQ ID NO: 241 - protein, 10 aa, synthetic construct
GGCGSGGGGS

SEQ ID NO: 242 - protein, 15 aa, synthetic construct
GGCGSGGGGSGGGGS

SEQ ID NO: 243 - protein, 20 aa, synthetic construct
GGCGSGGGGSGGGGSGGGGS

SEQ ID NO: 244 - protein, 9 aa, synthetic construct
KYIQESQAL

SEQ ID NO: 245 - protein, 9 aa, synthetic construct
EYYLQNAFL

SEQ ID NO: 246 - protein, 9 aa, synthetic construct
AYTKKAPQL

SEQ ID NO: 247 - protein, 9 aa, synthetic construct
EYSRRHPQL

SEQ ID NO: 248 - protein, 9 aa, synthetic construct
RSCGLFQKL

SEQ ID NO: 249 - protein, 9 aa, synthetic construct
AYEEDRETF

SEQ ID NO: 250 - protein, 9 aa, synthetic construct
FMNKFIYEI

SEQ ID NO: 251 - protein, 10 aa, synthetic construct
GLSPNLNRFL

SEQ ID NO: 252 - protein, 8 aa, synthetic construct
CMTWNQMN

SEQ ID NO: 253 - protein, 9 aa, synthetic construct
NYMNLGATL

SEQ ID NO: 254 - protein, 9 aa, synthetic construct
CYTWNQMNL

SEQ ID NO: 255 - protein, 9 aa, synthetic construct
CMTWNQMNL

SEQ ID NO: 256 - protein, 9 aa, synthetic construct
NQMNLGATL

SEQ ID NO: 257 - protein, 9 aa, synthetic construct
NLMNLGATL

SEQ ID NO: 258 - protein, 9 aa, synthetic construct
RVPGVAPTL

SEQ ID NO: 259 - protein, 9 aa, synthetic construct
RYPGVAPTL

SEQ ID NO: 260 - protein, 9 aa, synthetic construct
RYFPNAPYL

SEQ ID NO: 261 - protein, 9 aa, synthetic construct
RYPSCQKKF

SEQ ID NO: 262 - protein, 9 aa, synthetic construct
VYDFAFRDL

SEQ ID NO: 263 - protein, 9 aa, synthetic construct
RAHYNIVTF

SEQ ID NO: 264 - protein, 9 aa, synthetic construct
CDSTLRLCV

SEQ ID NO: 265 - protein, 10 aa, synthetic construct
LCVQSTHVDI

SEQ ID NO: 266 - protein, 9 aa, synthetic construct
STAPPAHGV

SEQ ID NO: 267 - protein, 9 aa, synthetic construct
STAPPVHNV

SEQ ID NO: 268 - protein, 9 aa, synthetic construct
SLAPPVHNV

SEQ ID NO: 269 - protein, 9 aa, synthetic construct
SLAPPAHGV

SEQ ID NO: 270 - protein, 9 aa, synthetic construct
SAPDTRPAP

SEQ ID NO: 271 - protein, 20 aa, synthetic construct
VTSAPDTRPAPGSTAPPAHG

SEQ ID NO: 272 - protein, 20 aa, synthetic construct
PDTRPAPGSTAPPAHGVTSA

SEQ ID NO: 273 - protein, 9 aa, synthetic construct
LLLLTVLTV

SEQ ID NO: 274 - protein, 9 aa, synthetic construct
STAPPAHGV

SEQ ID NO: 275 - protein, 9 aa, synthetic construct
STAPPAHGV

SEQ ID NO: 276 - protein, 9 aa, synthetic construct
STAPPVHNV

SEQ ID NO: 277 - protein, 9 aa, synthetic construct
SLAPPVHNV

SEQ ID NO: 278 - protein, 9 aa, synthetic construct
SLAPPAHGV

SEQ ID NO: 279 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 280 - protein, 10 aa, synthetic construct
GVYDGREHTV

SEQ ID NO: 281 - protein, 9 aa, synthetic construct
NYKRCFPVI

SEQ ID NO: 282 - protein, 9 aa, synthetic construct
EVDPASNTY

SEQ ID NO: 283 - protein, 8 aa, synthetic construct
SESLKMIF

SEQ ID NO: 284 - protein, 9 aa, synthetic construct
SESLICMIF

SEQ ID NO: 285 - protein, 10 aa, synthetic construct
GVYDGREHTV

SEQ ID NO: 286 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 287 - protein, 11 aa, synthetic construct
SLLMWITQCFL

SEQ ID NO: 288 - protein, 9 aa, synthetic construct
SLLMWITQC

SEQ ID NO: 289 - protein, 9 aa, synthetic construct
QLSLLMWIT

SEQ ID NO: 290 - protein, 14 aa, synthetic construct
SLLMWITQCFLPVF

SEQ ID NO: 291 - protein, 11 aa, synthetic construct
MLMAQEALAFL

SEQ ID NO: 292 - protein, 11 aa, synthetic construct
YLAMPFATPME

SEQ ID NO: 293 - protein, 10 aa, synthetic construct
ASGPGGGAPR

SEQ ID NO: 294 - protein, 10 aa, synthetic construct
LAAQERRVPR

SEQ ID NO: 295 - protein, 10 aa, synthetic construct
TVSGNILTIR

SEQ ID NO: 296 - protein, 13 aa, synthetic construct
APRGPHGGAASGL

SEQ ID NO: 297 - protein, 11 aa, synthetic construct
MPFATPMEAEL

SEQ ID NO: 298 - protein, 12 aa, synthetic construct
KEFTVSGNLLTI

SEQ ID NO: 299 - protein, 9 aa, synthetic construct
MPFATPMEA

SEQ ID NO: 300 - protein, 11 aa, synthetic construct
FATPMEAELAR

SEQ ID NO: 301 - protein, 9 aa, synthetic construct
LAMPFATPM

SEQ ID NO: 302 - protein, 9 aa, synthetic construct
ARGPESRLL

SEQ ID NO: 303 - protein, 10 aa, synthetic construct
ELTLGEFLKL

SEQ ID NO: 304 - protein, 15 aa, synthetic construct
TLGEFLKLDRERAKN

SEQ ID NO: 305 - protein, 6 aa, synthetic construct
QMFFCF

SEQ ID NO: 306 - protein, 15 aa, synthetic construct
DLAQMFFCFKELEGW

SEQ ID NO: 307 - protein, 10 aa, synthetic construct
AQMFFCFKEL

SEQ ID NO: 308 - protein, 9 aa, synthetic construct
QMFFCFKEL

SEQ ID NO: 309 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 310 - protein, 9 aa, synthetic construct
SLLFLLFSL

SEQ ID NO: 311 - protein, 9 aa, synthetic construct
VLPLTVAEV

SEQ ID NO: 312 - protein, 9 aa, synthetic construct
ELAVALAQK

SEQ ID NO: 313 - protein, 9 aa, synthetic construct
ALQGGGPPY

SEQ ID NO: 314 - protein, 9 aa, synthetic construct
FYPGYLCSL

SEQ ID NO: 315 - protein, 9 aa, synthetic construct
LYPKARLAF

SEQ ID NO: 316 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 317 - protein, 10 aa, synthetic construct
ELAGIGILTV

SEQ ID NO: 318 - protein, 9 aa, synthetic construct
VLDGLDVLL

SEQ ID NO: 319 - protein, 10 aa, synthetic construct
SLYSFPEPEA

SEQ ID NO: 320 - protein, 10 aa, synthetic construct
ALYVDSLFFL

SEQ ID NO: 321 - protein, 9 aa, synthetic construct
SLLQHLIGL

SEQ ID NO: 322 - protein, 9 aa, synthetic construct
GILGFVFTL

SEQ ID NO: 323 - protein, 9 aa, synthetic construct
IVTDFSVIK

SEQ ID NO: 324 - protein, 10 aa, synthetic construct
AVFDRKSDAK

SEQ ID NO: 325 - protein, 5 aa, synthetic construct
  VARIANT >5: note = Wherein the entire sequence of amino acids 1-5 may be repeated one or more times
GSGGS

SEQ ID NO: 326 - protein, 5 aa, synthetic construct
  VARIANT >5: note = Wherein the entire sequence of amino acids 1-5 may be repeated one or more times
GGGGS

SEQ ID NO: 327 - protein, 4 aa, synthetic construct
  VARIANT >4: note = Wherein the entire sequence of amino acids 1-4 may be repeated one or more times
GGGS

SEQ ID NO: 328 - protein, 50 aa, synthetic construct
  VARIANT 6..50: note = Wherein up to 9 copies of GGGGS may be deleted
GGGGSGGGGSGGGGSGGGGSGGGGSGGGGSGGGGSGGGGSGGGGSGGGGS

SEQ ID NO: 329 - protein, 10 aa, synthetic construct
GGGGSGGGGS

SEQ ID NO: 330 - protein, 15 aa, synthetic construct
GGGGSGGGGSGGGGS

SEQ ID NO: 331 - protein, 20 aa, synthetic construct
GGGGSGGGGSGGGGSGGGGS

SEQ ID NO: 332 - protein, 35 aa, synthetic construct
GGGGSGGGGSGGGGSGGGGSGGGGSGGGGSGGGGS

SEQ ID NO: 333 - protein, 5 aa, synthetic construct
AAAGG

SEQ ID NO: 334 - protein, 133 aa, synthetic construct
  VARIANT 16: note = Wherein X is any amino acid other than His
  VARIANT 42: note = Wherein X is any amino acid other than Phe
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 335 - protein, 133 aa, synthetic construct; identical to SEQ ID NO: 334
  VARIANT 16: note = Wherein X is is Ala, Arg, Asn, Asp, Cys, Gln, Glu, Gly, Ile, Leu, Lys, Met, Phe, Pro, Ser, Thr, Trp, Tyr, or Val
  VARIANT 42: note = Wherein X is Ala, Arg, Asn, Asp, Cys, Gln, Glu, Gly, His, Ile, Leu, Lys, Met, Pro, Ser, Thr, Trp, Tyr, or Val

SEQ ID NO: 336 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 337 - protein, 133 aa, synthetic construct; identical to SEQ ID NO: 336

SEQ ID NO: 338 - protein, 133 aa, synthetic construct
APTSSSTKKTQLQLETLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 339 - protein, 133 aa, synthetic construct
APTSSSTKKTQLQLEDLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 340 - protein, 133 aa, synthetic construct
APTSSSTKKTQLQLEELLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 341 - protein, 219 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 342 - protein, 167 aa, synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 343 - protein, 240 aa, synthetic construct
QEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSIL

SEQ ID NO: 344 - protein, 134 aa, synthetic construct
LQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 345 - protein, 201 aa, synthetic construct
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 346 - protein, 208 aa, synthetic construct [sequence omitted]

SEQ ID NO: 347 - protein, 224 aa, synthetic construct
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 348 - protein, 179 aa, synthetic construct
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 349 - protein, 4 aa, synthetic construct
GGSG

SEQ ID NO: 350 - protein, 5 aa, synthetic construct
GGSGG

SEQ ID NO: 351 - protein, 5 aa, synthetic construct
GSGSG

SEQ ID NO: 352 - protein, 5 aa, synthetic construct
GSGGG

SEQ ID NO: 353 - protein, 5 aa, synthetic construct
GGGSG

SEQ ID NO: 354 - protein, 5 aa, synthetic construct
GSSSG

SEQ ID NO: 355 - protein, 5 aa, synthetic construct
GGGGS

SEQ ID NO: 356 - protein, 100 aa, synthetic construct
  VARIANT 6..100: note = Wherein up to 19 copies of EAAAK may be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 357 - protein, 101 aa, synthetic construct
  VARIANT 7..101: note = Wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 358 - protein, 102 aa, synthetic construct
  VARIANT 7..101: note = Wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 359 - protein, 206 aa, synthetic construct
  VARIANT 7..101: note = Wherein up to 19 copies of EAAAK may be deleted
  VARIANT 106..205: note = Wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKALEAEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 360 - protein, 40 aa, synthetic construct
  VARIANT 3..40: note = Wherein up to 19 copies of KP may be deleted
KPKPKPKPKPKPKPKPKPKPKPKPKPKPKPKPKPKPKPKP

SEQ ID NO: 361 - protein, 40 aa, synthetic construct
  VARIANT 3..40: note = Wherein up to 19 copies of EP may be deleted
EPEPEPEPEPEPEPEPEPEPEPEPEPEPEPEPEPEPEPEP

SEQ ID NO: 362 - protein, 60 aa, synthetic construct
  VARIANT 4..60: note = Wherein up to 19 copies of TPR may be deleted
TPRTPRTPRTPRTPRTPRTPRTPRTPRTPRTPRTPRTPRTPRTPRTPRTPRTPRTPRTPR

SEQ ID NO: 363 - protein, 40 aa, synthetic construct
  VARIANT 3..40: note = Wherein up to 19 copies of AP may be deleted
APAPAPAPAPAPAPAPAPAPAPAPAPAPAPAPAPAPAPAP

SEQ ID NO: 364 - protein, 5 aa, synthetic construct
EAAAK

SEQ ID NO: 365 - protein, 10 aa, synthetic construct
EAAAKEAAAK

SEQ ID NO: 366 - protein, 15 aa, synthetic construct
EAAAKEAAAKEAAAK

SEQ ID NO: 367 - protein, 46 aa, synthetic construct
AEAAAKEAAAKEAAAKEAAAKALEAEAAAKEAAAKEAAAKEAAAKA

SEQ ID NO: 368 - protein, 12 aa, synthetic construct
AEAAAKEAAAKA

SEQ ID NO: 369 - protein, 5 aa, synthetic construct
PAPAP

SEQ ID NO: 370 - protein, 8 aa, synthetic construct
APAPAPAP

SEQ ID NO: 371 - protein, 12 aa, synthetic construct
APAPAPAPAPAP

SEQ ID NO: 372 - protein, 16 aa, synthetic construct
APAPAPAPAPAPAPAP

SEQ ID NO: 373 - protein, 20 aa, synthetic construct
APAPAPAPAPAPAPAPAPAP

SEQ ID NO: 374 - protein, 4 aa, synthetic construct
KPKP

SEQ ID NO: 375 - protein, 8 aa, synthetic construct
KPKPKPKP

SEQ ID NO: 376 - protein, 12 aa, synthetic construct
KPKPKPKPKPKP

SEQ ID NO: 377 - protein, 16 aa, synthetic construct
KPKPKPKPKPKPKPKP

SEQ ID NO: 378 - protein, 20 aa, synthetic construct
KPKPKPKPKPKPKPKPKPKP

SEQ ID NO: 379 - protein, 4 aa, synthetic construct
EPEP

SEQ ID NO: 380 - protein, 8 aa, synthetic construct
EPEPEPEP

SEQ ID NO: 381 - protein, 12 aa, synthetic construct
EPEPEPEPEPEP

SEQ ID NO: 382 - protein, 16 aa, synthetic construct
EPEPEPEPEPEPEPEP

SEQ ID NO: 383 - protein, 20 aa, synthetic construct
EPEPEPEPEPEPEPEPEPEP

SEQ ID NO: 384 - protein, 5 aa, synthetic construct
  VARIANT 4: note = Wherein X is any amino acid other than Pro
VPGXG